(19) United States
(12) Patent Application Publication
(10) Pub. No.: US 2023/0017045 A1
(43) Pub. Date: Jan. 19, 2023

(54) PYRIDINE AND PYRIMIDINE CARBOXYLATE HERBICIDES AND METHODS OF USE THEREOF

(71) Applicant: Dow AgroSciences LLC, Indianapolis, IN (US)

(72) Inventors: Jared Bell, Indianapolis, IN (US); Ann M. Buysse, Carmel, IN (US); John F. Daeuble, Carmel, IN (US); Joseph D. Eckelbarger, Carmel, IN (US); Jeffrey B. Epp, Noblesville, IN (US); Nicholas M. Irvine, Westfield, IN (US); Jeremy Kister, Carmel, IN (US); William C. Lo, Fishers, IN (US); Michael R. Loso, Carmel, IN (US); Christian T. Lowe, Westfield, IN (US); John C. Rohanna, Collegeville, PA (US); Norbert M. Satchivi, Carmel, IN (US); Thomas L. Siddall, Zionsville, IN (US); Kimberly M. Steward, Zionsville, IN (US); Carla N. Yerkes, Crawfordsville, IN (US)

(73) Assignee: Dow AgroSciences LLC, Indianapolis, IN (US)

(21) Appl. No.: 16/759,075

(22) PCT Filed: Oct. 26, 2018
(86) PCT No.: PCT/US18/57626

§ 371 Date: Apr. 24, 2020

Related U.S. Application Data
(60) Provisional application No. 62/577,972, filed on Oct. 27, 2017.

(51) Int. Cl. C07D 213/79; C07D 405/10; A01N 43/40; A01N 43/54; A01N 25/32; C07D 409/10; C07D 413/10; A01N 43/82; A01N 43/76; A01N 43/78; C07D 417/10; C07D 239/28; C07D 401/10; A01P 13/00

(52) U.S. Cl. CPC C07D 213/79; C07D 405/10; A01N 43/40; A01N 43/54; A01N 25/32; C07D 409/10; A01P 13/00; A01N 43/82; A01N 43/76; A01N 43/78; C07D 417/10; C07D 239/28; C07D 401/10; C07D 413/10

(57) ABSTRACT

Provided herein are pyridine and pyrimidine carboxylates and their derivatives, and compositions and methods of use thereof as herbicides.

### CROSS REFERENCE TO RELATED APPLICATIONS

This application claims the benefit and priority of U.S. Provisional Patent Application Ser. No. 62/577,972 filed Oct. 27, 2017, the entire disclosure of which is expressly incorporated by reference herein in its entirety.

### FIELD

Provided herein are herbicidal compounds, compositions containing the same, and methods of use thereof for controlling undesirable vegetation.

### BACKGROUND

The occurrence of undesirable vegetation or weeds, is a constant problem facing farmers in crops, pastures, and other settings. Weeds compete with crops and can therefore negatively impact crop yield. The use of chemical herbicides is an important tool in controlling undesirable vegetation.

There remains a need for new chemical herbicides that offer a broader spectrum of weed control, selectivity, minimal crop damage, storage stability, ease of handling, higher activity against weeds, and/or a means to address herbicide-tolerance that develops with respect to herbicides currently in use.

### SUMMARY

Provided herein are compounds of Formula (I):

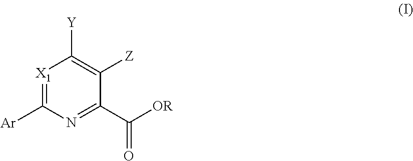

wherein
Ar is

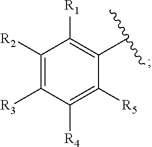

X₁ is N or CR₆;

R is hydrogen, substituted or unsubstituted alkyl, substituted or unsubstituted phenyl, substituted or unsubstituted benzyl, substituted or unsubstituted pyridinylmethyl, substituted or unsubstituted alkenyl, or substituted or unsubstituted alkynyl;

R₁, R₂, R₃, R₄, and R₅ are independently selected from the group consisting of hydrogen, halogen, hydroxy, substituted or unsubstituted alkyl, substituted or unsubstituted haloalkyl, substituted or unsubstituted alkenyl, substituted or unsubstituted haloalkenyl, substituted or unsubstituted alkynyl, substituted or unsubstituted cycloalkyl, substituted or unsubstituted halocycloalkyl, substituted or unsubstituted aryl, substituted or unsubstituted aryloxy, substituted or unsubstituted alkoxy, substituted or unsubstituted haloalkoxy, substituted or unsubstituted alkylsulfanyl, substituted or unsubstituted haloalkylsulfanyl, substituted or unsubstituted alkylsulfinyl, substituted or unsubstituted haloalkylsulfinyl, substituted or unsubstituted alkylsulfonyl, substituted or unsubstituted haloalkylsulfonyl, substituted or unsubstituted alkylsulfonyl(oxy), substituted or unsubstituted haloalkylsulfonyl(oxy), amino, alkylamino, dialkylamino, amido, formyl, 2,2-dimethylhydrazono, methoxyimino, hydroxyimino, alkylcarbonyl, alkoxycarbonyl, nitro, and cyano, or R₁ and R₂, R₂ and R₃, R₃ and R₄ or R₄ and R₅ together can form a substituted or unsubstituted 5- or 6-membered aliphatic or aromatic ring, containing 0 to 3 heteroatoms selected from the group consisting of O, N, and S;

R₆ is hydrogen, halogen, substituted or unsubstituted alkyl, substituted or unsubstituted haloalkyl, substituted or unsubstituted alkynyl, substituted or unsubstituted alkoxy, substituted or unsubstituted alkylsulfanyl, hydroxy, amino, cyano, or acylamino;

Y is selected from the group consisting of hydrogen, halogen, substituted or unsubstituted alkyl, substituted or unsubstituted haloalkyl, substituted or unsubstituted alkenyl, substituted or unsubstituted haloalkenyl, substituted or unsubstituted alkynyl, substituted or unsubstituted alkoxy, substituted or unsubstituted haloalkoxy, substituted or unsubstituted alkylsulfanyl, substituted or unsubstituted alkylsulfinyl, substituted or unsubstituted alkylsulfonyl, nitro, and cyano;

Z is selected from the group consisting of hydrogen, halogen, alkyl, haloalkyl, alkynyl, alkoxy, haloalkoxy, alkylsulfanyl, amino, nitro, and cyano;

with the proviso that when X₁=N, Y is not alkoxy;

with the proviso that the compound of Formula (I) is not

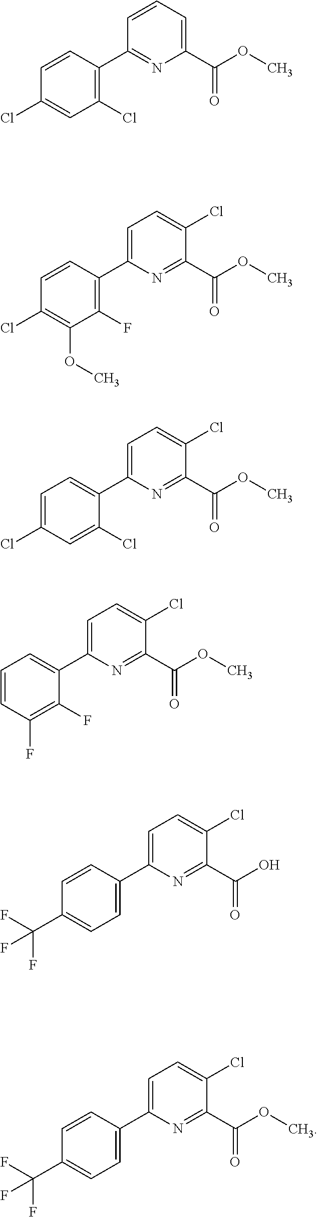 [SMILES: COC(=O)c1cccc(-c2ccc(Cl)cc2Cl)n1 ; COC(=O)c1nc(-c2ccc(Cl)c(OC)c2F)ccc1Cl ; COC(=O)c1nc(-c2ccc(Cl)cc2Cl)ccc1Cl ; COC(=O)c1nc(-c2cccc(F)c2F)ccc1Cl ; O=C(O)c1nc(-c2ccc(C(F)(F)F)cc2)ccc1Cl ; COC(=O)c1nc(-c2ccc(C(F)(F)F)cc2)ccc1Cl]

Also provided herein are compounds of Formula (I):

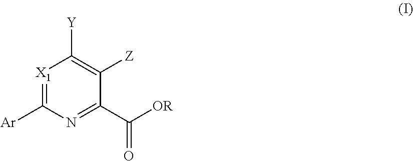

wherein
Ar is

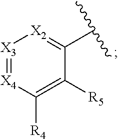

X₁ is N or CR₆; X₂ is N or CR₁; X₃ is N or CR₂; X₄ is N or CR₃;

R is hydrogen, substituted or unsubstituted alkyl, phenyl, substituted or unsubstituted benzyl, substituted or unsubstituted pyridinylmethyl, substituted or unsubstituted alkenyl, or substituted or unsubstituted alkynyl;

R₁, R₂, R₃, R₄, and R₅ are independently selected from the group consisting of hydrogen, halogen, hydroxy, substituted or unsubstituted alkyl, substituted or unsubstituted haloalkyl, substituted or unsubstituted alkenyl, substituted or unsubstituted haloalkenyl, substituted or unsubstituted alkynyl, substituted or unsubstituted cycloalkyl, substituted or unsubstituted halocycloalkyl, substituted or unsubstituted aryl, substituted or unsubstituted aryloxy, substituted or unsubstituted alkoxy, substituted or unsubstituted haloalkoxy, substituted or unsubstituted alkylsulfanyl, substituted or unsubstituted haloalkylsulfanyl, substituted or unsubstituted alkylsulfinyl, substituted or unsubstituted haloalkylsulfinyl, substituted or unsubstituted alkylsulfonyl, substituted or unsubstituted haloalkylsulfonyl, substituted or unsubstituted alkylsulfonyl(oxy), substituted or unsubstituted haloalkylsulfonyl(oxy), amino, alkylamino, dialkylamino, amido, formyl, 2,2-dimethylhydrazono, methoxyimino, hydroxyimino, alkylcarbonyl, alkoxycarbonyl, nitro, and cyano, or R₁ and R₂, R₂ and R₃, R₃ and R₄ or R₄ and R₅ together can form a substituted or unsubstituted 5- or 6-membered aliphatic or aromatic ring, containing 0 to 3 heteroatoms selected from the group consisting of O, N, and S;

R₆ is hydrogen, halogen, substituted or unsubstituted alkyl, substituted or unsubstituted haloalkyl, substituted or unsubstituted alkynyl, substituted or unsubstituted alkoxy, substituted or unsubstituted alkylsulfanyl, hydroxy, amino, cyano, or acylamino;

Y is selected from the group consisting of hydrogen, halogen, substituted or unsubstituted alkyl, substituted or unsubstituted haloalkyl, substituted or unsubstituted alkenyl, substituted or unsubstituted haloalkenyl, substituted or unsubstituted alkynyl, substituted or unsubstituted alkoxy, substituted or unsubstituted haloalkoxy, substituted or unsubstituted alkylsulfanyl, substituted or unsubstituted alkylsulfinyl, substituted or unsubstituted alkylsulfonyl, nitro, and cyano; and

Z is selected from the group consisting of hydrogen, halogen, alkyl, haloalkyl, alkynyl, alkoxy, haloalkoxy, alkylsulfanyl, amino, nitro, and cyano;

with the proviso that when X₁ is N, then Y is not alkoxy.

In various aspects, compounds may include compounds wherein R is selected from the group consisting of hydrogen, C₁-C₈ alkyl, substituted C₁-C₈ alkyl, C₂-C₈ alkenyl, C₂-C₈ alkynyl, benzyl, substituted benzyl, and pyridinylmethyl.

Some compounds according to various aspects may include compounds where R1 or R5 is selected from the group consisting of hydrogen, halogen, cyano, amino, amido, nitro, C1-C8 alkyl, C1-C8 haloalkyl, C2-C8 alkenyl, C2-C8 alkynyl, C1-C8 alkoxy, C1-C8 alkylsulfanyl, C1-C8 alkylsulfinyl, C1-C8 alkylsulfonyl, formyl,

Also, some aspects may include compounds where R2 or R4 is selected from the group consisting of hydrogen, halogen, nitro C1-C8 alkyl, C1-C8 haloalkyl, C1-C8 alkoxy, C1-C8 alkylsulfanyl, and C1-C8 alkylsulfinyl.

In various aspects, R3 may selected from the group consisting of halogen, substituted or unsubstituted haloalkyl, substituted or unsubstituted cycloalkyl, substituted or unsubstitutedhalocycloalkyl, substituted or unsubstitutedhaloalkysulfanyl, substituted or unsubstitutedalkylsulfonyl(oxy), and cyano.

Some compounds according to various aspects include compounds wherein R1 and R2, R2 and R3, R3 and R4 or R4 and R5 together can form a substituted or unsubstituted 5- or 6-membered aliphatic or aromatic ring, containing 0 to 3 heteroatoms. Such as those selected from the group consisting of O, N, or S. For example, such as those selected from the group consisting of

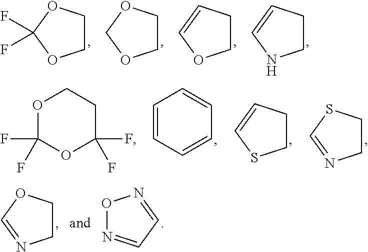

In some aspects, R may be selected from the group consisting of hydrogen, C1-C8 alkyl, substituted C1-C8 alkyl, C2-C8 alkenyl, C2-C8 alkynyl, benzyl, substituted benzyl, and pyridinylmethyl.

In various aspects, wherein R1, R2, R3, R4, and R5 may be independently selected from the group consisting of hydrogen, halogen, cyano, amino, amido, nitro, C1-C8 alkyl, C1-C8 haloalkyl, C2-C8 alkenyl, C2-C8 alkynyl, C1-C8 alkoxy, C1-C8 alkylsulfanyl, C1-C8 alkylsulfinyl, C1-C8 alkylsulfonyl, and formyl.

In some aspects, R2 or R4 may be selected from the group consisting of hydrogen, halogen, nitro C1-C8 alkyl, C1-C8 haloalkyl, C1-C8 alkoxy, C1-C8 alkylsulfanyl, and C1-C8 alkylsulfinyl.

Also included herein are compounds, according to various aspects, where R is selected from the group consisting of hydrogen, methyl, cyanomethyl, 2-methylallyl, propargyl, benzyl, substituted benzyl, and pyridinylmethyl; R1 or R5 is selected from the group consisting of hydrogen, halogen, cyano, (C1-C4)alkyl, and (C1-C4)alkoxy; R2 or R4 is selected from the group consisting of hydrogen, halogen, (C2-C4)alkynyl, and (C1-C4)haloalkyl; R3 is selected from the group consisting of halogen, substituted (C3-C6)cycloalkyl, (C1-C4)haloalkylsulfanyl, (C1-C4)haloalkyl, and (C1-C4)haloalkoxy or R1 and R2, R2 and R3, R3 and R4 or R4 and R5 together can form a substituted or unsubstituted 5- or 6-membered aliphatic or aromatic ring, containing 0 to 3 heteroatoms selected from the group consisting of O, N, and S; R6 is selected from the group consisting of hydrogen, halogen, and hydroxy; Y is H; and Z is a halogen.

In some aspects, R may be selected from the group consisting of hydrogen, methyl, cyanomethyl, 2-methylallyl, propargyl, benzyl, substituted benzyl, and pyridinylmethyl; R₁, R₂, R₃, R₄, and R₅ are independently selected from the group consisting of hydrogen, halogen, (C₁-C₄)haloalkyl, and (C₁-C₄)alkoxy; R₆ is selected from the group consisting of hydrogen, halogen, and hydroxy; Y is H; and Z is a halogen.

In some aspects, compositions may comprise (include) any of the foregoing compounds and an agriculturally acceptable adjuvant or carrier.

In some aspects, compositions may include any of the foregoing and an additional herbicidal compound.

In various aspects, compositions may include any of the foregoing and a safener.

Also included herein, according to various aspects, are method for controlling undesirable vegetation, which includes (a) contacting the undesirable vegetation or area adjacent to the undesirable vegetation, or (b) pre-emergently contacting soil or water, with any of the foregoing compounds or compositions. Such methods and aspects may include methods where the compositions are applied pre-emergent, post-emergent, or both pre-emergent and post-emergent.

### DETAILED DESCRIPTION

### I. Definitions

As used herein, control of or controlling undesirable vegetation can be understood to include killing or preventing the vegetation, or causing some other adversely modifying effect to the vegetation e.g., necrosis, chlorosis, stunting, deviations from natural growth or development, regulation, desiccation, retardation, and the like.

As used herein, herbicide, herbicide composition, and herbicidal active ingredient can be understood to include a compound that controls undesirable vegetation when applied in an appropriate amount.

As used herein, a herbicidally effective or vegetation controlling amount can be understood to include an amount of herbicidal active ingredient the application of which controls the relevant undesirable vegetation.

As used herein, applying a herbicide, herbicidal composition, or herbicidal active ingredient can be understood to include delivering it directly to the targeted vegetation or to the locus thereof or to the area where control of undesired vegetation is desired. Methods of application include, but are not limited to, contacting the undesirable vegetation or area adjacent to the undesirable vegetation pre-emergently, post-emergently, on the foliage, on the soil, and/or in-water.

As used herein, plants and vegetation include, but are not limited to, dormant seeds, germinant seeds, emerging seedlings, plants emerging from vegetative propagules, immature vegetation, mature vegetation, reproductive vegetation, and established vegetation.

As used herein, immature vegetation may be understood to include small vegetative plants prior to reproductive stage, and mature vegetation may be understood to include vegetative plants during and after reproductive stage.

As used herein, “safener” may be understood to include molecules used in combination with herbicides to reduce the effect of the herbicide on crop plants and to improve selectivity between crop plants and weed species being targeted by the herbicide.

As used herein, “adjuvant” may be understood to include a substance in a herbicide formulation or added to the spray tank to improve herbicidal activity or application characteristics. Spray adjuvants can be grouped into two broad categories: activator adjuvants and special purpose adjuvants.

As used herein, agriculturally acceptable salts and esters may be understood to include salts and esters of compounds of Formula (I) that exhibit herbicidal activity, or that are or can be converted in plants, water, or soil, to the referenced herbicide. Exemplary agriculturally acceptable esters are those that are or can be hydrolyzed, oxidized, metabolized, or otherwise converted, e.g., in plants, water, or soil, to the corresponding pyridine carboxylic acid which, depending upon the pH, may be in the dissociated or undissociated form.

Suitable agriculturally acceptable salts include those derived from alkali or alkaline earth metals and those derived from ammonia and amines. Preferred cations include sodium, potassium, magnesium, and aminium cations of the formula:

R¹⁰R¹¹R¹²R¹³N⁺

wherein R¹⁰, R¹¹, R¹² and R¹³ each, independently represents hydrogen or C₁-C₁₂ alkyl, C₃-C₁₂ alkenyl, or C₃-C₁₂ alkynyl, each of which is optionally substituted by one or more substituents such as hydroxy, C₁-C₄ alkoxy, C₁-C₄ alkylthio, or phenyl groups, provided that R¹⁰, R¹¹, R¹² and R¹³ are sterically compatible. Additionally, any two R¹⁰, R¹¹, R¹² and R¹³ together may represent an aliphatic difunctional moiety containing one to twelve carbon atoms and up to two oxygen or sulfur atoms. Salts of the compounds of Formula (I) can be prepared by treatment of compounds of Formula (I) with a metal hydroxide, such as sodium hydroxide, with an amine, such as ammonia, trimethylamine, diethanolamine, 2-methyl-thiopropylamine, bisallylamine, 2-butoxyethylamine, morpholine, cyclododecylamine, 2-methylheptylamine, or benzylamine, or with a tetraalkylammonium hydroxide, such as tetramethylammonium hydroxide or choline hydroxide. Amine salts of compounds of Formula (I) are useful forms or derivatives of compounds of Formula (I) because they are water-soluble and lend themselves to the preparation of desirable aqueous based herbicidal compositions.

Suitable agriculturally acceptable esters include straight chain or branched chain alkyl groups. Typical C₁-C₁₂ alkyl groups include, but are not limited to, methyl, ethyl, propyl, 1-methylethyl, butyl, 1,1-dimethylethyl, 1-methylpropyl, pentyl, hexyl, heptyl, 1-methyl-hexyl, octyl, 2-ethylhexyl, 2-methylheptyl, nonyl, decyl and dodecyl. Methyl and ethyl are often preferred. Alkyl esters substituted with groups such as halogens or CN are also included. Other preferred esters include C₁-C₈ heterocyclylalkyl esters, including pyridin-2-ylmethyl, pyridin-3-ylmethyl, and pyridin-4-ylmethyl; C₇-C₁₀ arylalkyl esters, including benzyl, substituted benzyl, and phenethyl, such as 2,4-dichlorobenzyl, 3-(trifluoromethyl)benzyl, and 3-(trifluoromethyl)benzyl; alkenyl esters, such as 2-methylallyl; and alkynyl esters, such as propargyl.

As used herein, “alkyl” may be understood to include saturated, straight-chained or branched hydrocarbon moieties. Unless otherwise specified, C₁-C₁₂ alkyl groups are intended. Examples include, but are not limited to, methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, and 1-ethyl-2-methyl-propyl.

As used herein, “haloalkyl” may be understood to include straight-chained or branched alkyl groups, where in these groups the hydrogen atoms may partially or entirely be substituted with one or more halogen atom(s). Unless otherwise specified, C₁-C₈ groups are intended. Examples include, but are not limited to, chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, and 1,1,1-trifluoroprop-2-yl.

As used herein, “cycloalkyl” may be understood to include saturated, cyclic hydrocarbon moieties. Unless otherwise specified, C₃-C₈ cycloalkyl groups are intended. Examples include cyclopropyl, 2,2-dimethyl-cyclopropyl, cyclobutyl, cyclopentyl, cyclohexyl, cycloheptyl and cyclooctyl.

As used herein, “halocycloalkyl” may be understood to include a cycloalkyl group as defined above wherein the carbon atoms are partially or entirely substituted with one or more halogen atoms.

As used herein, “alkenyl” may be understood to include unsaturated, straight-chained, or branched hydrocarbon moieties containing one or more double bond(s). Unless otherwise specified, C₂-C₈ alkenyl groups are intended. Alkenyl groups may contain more than one unsaturated bond. Examples include, but are not limited to, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 2-methylallyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl.

As used herein, “haloalkenyl” may be understood to include straight-chained or branched alkenyl groups, where in these groups the hydrogen atoms may partially or entirely be substituted with one or more halogen atom(s). Unless otherwise specified, C₂-C₈ groups are intended. Examples include, but are not limited to 1-chloroethenyl, 1-chloro-1-propenyl, 2-chloro-1-propenyl, 1-chloro-2-propenyl, 2-chloro-2-propenyl, 1-chloro-1-butenyl, 2-chloro-1-butenyl, 3-chloro-1-butenyl, 1-chloro-2-butenyl, 2-chloro-2-butenyl, 3-chloro-2-butenyl, 1-chloro-3-butenyl, 2-chloro-3-butenyl, 3-chloro-3-butenyl, 1-fluoroethenyl, 1-fluoro-1-propenyl, 2-fluoro-1-propenyl, 1-fluoro-2-propenyl, 2-fluoro-2-propenyl, 1-fluoro-1-butenyl, 2-fluoro-1-butenyl, 3-fluoro-1-butenyl, 1-fluoro-2-butenyl, 2-fluoro-2-butenyl, 3-fluoro-2-butenyl, 1-fluoro-3-butenyl, 2-fluoro-3-butenyl, and 3-fluoro-3-butenyl.

As used herein, “alkynyl” represents straight-chained or branched hydrocarbon moieties containing one or more triple bond(s). Unless otherwise specified, C₂-C₈ alkynyl groups are intended. Alkynyl groups may contain more than one unsaturated bond. Examples include, but are not limited to, C₂-C₈-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl.

As used herein, “haloalkynyl” may be understood to include straight-chained or branched alkynyl groups, where in these groups the hydrogen atoms may partially or entirely be substituted with one or more halogen atom(s). Unless otherwise specified, C₂-C₅ groups are intended. Examples include, but are not limited to, 1-chloro-2-butynyl, 1-chloro-3-butynyl, 2-chloro-3-butynyl, 1,1-dichloro-2-propynyl, 1-chloro-2-propynyl, 3-chloro-1-pentynyl, 4-chloro-1-pentynyl, 1-chloro-2-pentynyl, 4-chloro-2-pentynyl, 1-chloro-3-pentynyl, 2-chloro-3-pentynyl, 1-chloro-4-pentynyl, 2-chloro-4-pentynyl, 3-chloro-4-pentynyl, 1,1-dichloro-2-butynyl, 1,1-dichloro-3-butynyl, 1,2-dichloro-3-butynyl, 2,2-dichloro-3-butynyl, 3,3-dichloro-1-butynyl, 1-fluoro-2-butynyl, 1-fluoro-3-butynyl, 2-fluoro-3-butynyl, and 1,1-difluoro-2-propynyl, 1-fluoro-2-propynyl, 3-fluoro-1-pentynyl, 4-fluoro-1-pentynyl, 1-fluoro-2-pentynyl, 4-fluoro-2-pentynyl, 1-fluoro-3-pentynyl, 2-fluoro-3-pentynyl, 1-fluoro-4-pentynyl, 2-fluoro-4-pentynyl, 3-fluoro-4-pentynyl, 1,1-difluoro-2-butynyl, 1,1-difluoro-3-butynyl, 1,2-difluoro-3-butynyl, 2,2-difluoro-3-butynyl, and 3,3-difluoro-1-butynyl.

As used herein, “alkoxy” may be understood to include a group of the formula R—O—, where R is alkyl as defined above. Unless otherwise specified, alkoxy groups wherein R is a C₁-C₈ alkyl group are intended. Examples include, but are not limited to, methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-dimethyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-pentoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

As used herein, “haloalkoxy” may be understood to include a group of the formula R—O—, where R is haloalkyl as defined above. Unless otherwise specified, haloalkoxy groups wherein R is a C₁-C₈ alkyl group are intended. Examples include, but are not limited to, chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy,

1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, and 1,1,1-trifluoroprop-2-oxy.

As used herein, “alkylthio” or “alkylsulfanyl” may be understood to include a group of the formula R—S— where R is alkyl as defined above. Unless otherwise specified, alkylthio or alkylsulfanyl groups wherein R is a C₁-C₈ alkyl group are intended. Examples include, but are not limited to, methylthio, ethylthio, propylthio, 1-methylethylthio, butylthio, 1-methyl-propylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio, and 1-ethyl-2-methylpropylthio.

As used herein, “haloalkylthio” or “haloalkylsulfanyl” may be understood to include an alkylthio group as defined above wherein the carbon atoms are partially or entirely substituted with one or more halogen atoms. Unless otherwise specified, haloalkylthio or haloalkylsulfanyl groups wherein R is a C₁-C₈ alkyl group are intended. Examples include, but are not limited to, chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2-difluoroethylthio, 2,2-dichloro-2-fluoroethylthio, 2,2,2-trichloroethylthio, pentafluoroethylthio, and 1,1,1-trifluoroprop-2-ylthio.

As used herein, “aryl,” as well as derivative terms such as “aryloxy,” may be understood to include a phenyl, indanyl, or naphthyl group. In some aspects, phenyl is preferred. Unless otherwise specified, the aryl groups may be unsubstituted or substituted with one or more substituents selected from, e.g., halogen, hydroxy, nitro, cyano, formyl, C₁-C₆ alkyl, C₂-C₆ alkenyl, C₂-C₆ alkynyl, C₁-C₆ alkoxy, C₁-C₆ haloalkyl, C₁-C₆ haloalkoxy, C₁-C₆ acyl, C₁-C₆ alkylthio or alkylsulfanyl, C₁-C₆ alkylsulfinyl, C₁-C₆ alkylsulfonyl, (C₁-C₆ alkoxy)carbonyl, C₁-C₆ carbamoyl, hydroxycarbonyl, (C₁-C₆ alkyl)carbonyl, aminocarbonyl, (C₁-C₆ alkylamino)carbonyl, (di(C₁-C₆ alkyl)amino)carbonyl, provided that the substituents are sterically compatible and the rules of chemical bonding and strain energy are satisfied. In some aspects, preferred substituents include, for example, halogen, C₁-C₂ alkyl, and C₁-C₂ haloalkyl.

As used herein, “heterocyclyl” may be understood to include a phenyl, indanyl, or naphthyl group. In some aspects, phenyl is preferred. Unless otherwise specified, the aryl groups may be unsubstituted or substituted with one or more substituents selected from, e.g., halogen, hydroxy, nitro, cyano, formyl, C₁-C₆ alkyl, C₂-C₆ alkenyl, C₂-C₆ alkynyl, C₁-C₆ alkoxy, C₁-C₆ haloalkyl, C₁-C₆ haloalkoxy, C₁-C₆ acyl, C₁-C₆ alkylthio or alkylsulfanyl, C₁-C₆ alkylsulfinyl, C₁-C₆ alkylsulfonyl, (C₁-C₆ alkoxy)carbonyl, C₁-C₆ carbamoyl, hydroxycarbonyl, (C₁-C₆ alkyl)carbonyl, aminocarbonyl, (C₁-C₆ alkylamino)carbonyl, (di(C₁-C₆ alkyl)amino)carbonyl, provided that the substituents are sterically compatible and the rules of chemical bonding and strain energy are satisfied. In some aspects, preferred substituents include, for example, halogen, C₁-C₂ alkyl, and C₁-C₂ haloalkyl.

As used herein, “arylalkyl”, “arylalkenyl”, and “arylalkynyl” may be understood to include an alkyl, alkenyl, or alkynyl group substituted with an aryl group as defined herein.

As used herein, “heterocyclylalkyl” may be understood to include an alkyl, group substituted with a heterocyclyl group as defined herein. In some aspects, a substituted or unsubstituted pyridinylmethyl group is preferred.

As used herein, “alkoxycarbonyl” may be understood to include a group of the formula

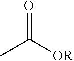

wherein R is alkyl.

As used herein, “alkylamino” or “dialkylamino” may be understood to include an amino group substituted with one or two alkyl groups, which may be the same or different.

As used herein, “alkylcarbamyl” may be understood to include a carbamyl group substituted on the nitrogen with an alkyl group.

As used herein, “alkylsulfinyl” may be understood to include —S(O)R, wherein R is alkyl (e.g., C₁-C₁₀ alkyl).

As used herein, “haloalkylsulfinyl” may be understood to include an alkylsulfinyl group as defined above wherein the carbon atoms are partially or entirely substituted with one or more halogen atoms.

As used herein, “alkylsulfonyl” may be understood to include —SO₂R, wherein R is alkyl (e.g., C₁-C₁₀ alkyl).

As used herein, “haloalkylsulfonyl” may be understood to include an alkylsulfonyl group as defined above wherein the carbon atoms are partially or entirely substituted with one or more halogen atoms.

As used herein, “carbamyl” (also referred to as carbamoyl or aminocarbonyl) may be understood to include a group of the formula

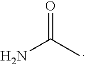

As used herein, “haloalkylamino” may be understood to include an alkylamino group wherein the alkyl carbon atoms are partially or entirely substituted with one or more halogen atoms.

As used herein, “Me” refers to a methyl group.
As used herein, the term “halogen,” including derivative terms such as “halo,” refers to fluorine, chlorine, bromine, or iodine (or fluoride, chloride, bromide, or iodide).

As used herein, plants and vegetation include, but are not limited to, germinant seeds, emerging seedlings, plants emerging from vegetative propagules, immature vegetation, and established vegetation.

As used herein, “substituted” means replacing one or more hydrogen atoms on the parent chain of hydrocarbon or heteroatoms with a different atom or group of atoms. Examples of substitutents include, but are not limited to, halogen, hydroxyl, nitro, cyano, amino, formyl, acyl, carboxyl, amide, alkyl, alkenyl, alkynyl, keto, thiol, sulfonic acid, sulfonate ester, sulfoxide, sulfone, alkoxy, phosphonic acid, and phosphate.

### II. Compounds

Compounds of Formula (I) and agriculturally acceptable derivatives thereof are described herein:

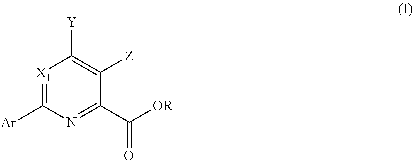

wherein
Ar is an aromatic or heteroaromatic group.

In some aspects, Ar is an aromatic group having the formula:

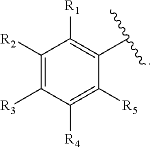

In some aspects, Ar is an heteroaromatic group having the formula:

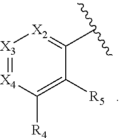

X₁ is N or CR₆; X₂ is N or CR₁; X₃ is N or CR₂; X₄ is N or CR₃;

R is hydrogen, substituted or unsubstituted alkyl, substituted or unsubstituted phenyl, substituted or unsubstituted benzyl, substituted or unsubstituted alkenyl, or substituted or unsubstituted alkynyl;

R₁, R₂, R₃, R₄, and R₅ are independently selected from hydrogen, halogen, substituted or unsubstituted alkyl, substituted or unsubstituted haloalkyl, substituted or unsubstituted alkenyl, substituted or unsubstituted haloalkenyl, substituted or unsubstituted alkynyl, substituted or unsubstituted alkoxy, substituted or unsubstituted haloalkoxy, substituted or unsubstituted thioalkyl, aminoalkyl, nitro, and cyano, or R₁ and R₂, R₂ and R₃, R₃ and R₄ or R₄ and R₅ together can form a substituted or unsubstituted 5- or 6-membered aliphatic, aromatic, or heteroaromatic ring;
R₆ is H, halogen, substituted or unsubstituted alkyl, substituted or unsubstituted haloalkyl, substituted or unsubstituted alkynyl, substituted or unsubstituted thioalkyl, hydroxy, amino, cyano, and acylamino;
Y is selected from hydrogen, halogen, substituted or unsubstituted alkyl, substituted or unsubstituted haloalkyl, substituted or unsubstituted alkenyl, substituted or unsubstituted haloalkenyl, substituted or unsubstituted alkynyl, substituted or unsubstituted alkoxy, substituted or unsubstituted haloalkoxy, substituted or unsubstituted thioalkyl, nitro, cyano, or S(O)ₙalkyl, wherein n is 0, 1, or 2;
Z is selected from halogen, alkyl, haloalkyl, alkynyl, alkoxy, haloalkoxy, thioalkyl, and cyano; with the proviso that when X₁ is N, Y is not alkoxy,

In some aspects, R is H, alkyl (e.g., methyl or ethyl), substituted alkyl, benzyl, and alkynyl (e.g., ethynyl).

In some aspects, R is as defined above as and Z is hydrogen, amino, halogen (e.g., chlorine or bromine), alkyl (e.g., methyl), alkynyl (e.g., ethynyl), cyano, haloalkyl (e.g., trifluoromethyl), and alkoxy (e.g., methoxy).

In some aspects, R and Z are as defined above and Y is hydrogen, halogen (e.g., chlorine, fluorine, or bromine), alkynyl (e.g., ethynyl), cyano, alkyl (e.g., methyl), alkoxy (e.g., methoxy or ethoxy).

In some aspects, R, Z, and Y are as defined above and X₁ is N or CR₆, wherein R₆ is hydrogen, alkynyl (e.g., ethynyl), cyano, alkyl (e.g., methyl), halogen (e.g., chlorine, fluorine or iodine), hydroxyl, haloalkyl (e.g., trifluoromethyl), thioalkyl (e.g., thiomethyl), amino, and acetamide.

In some aspects, the pyridine or pyrimidine ring has the following structure:

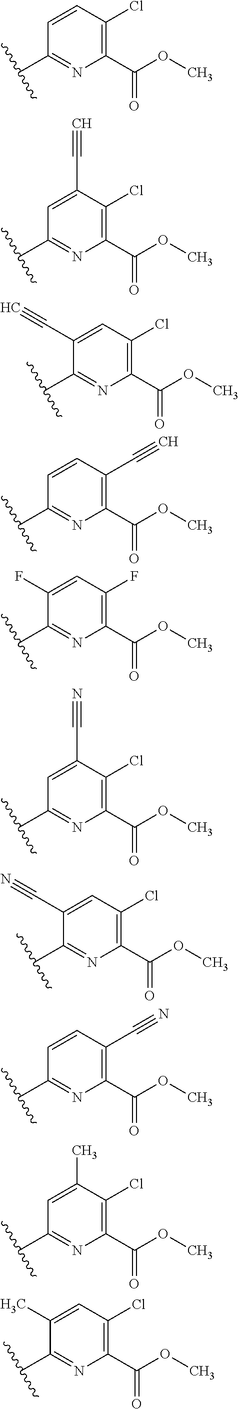

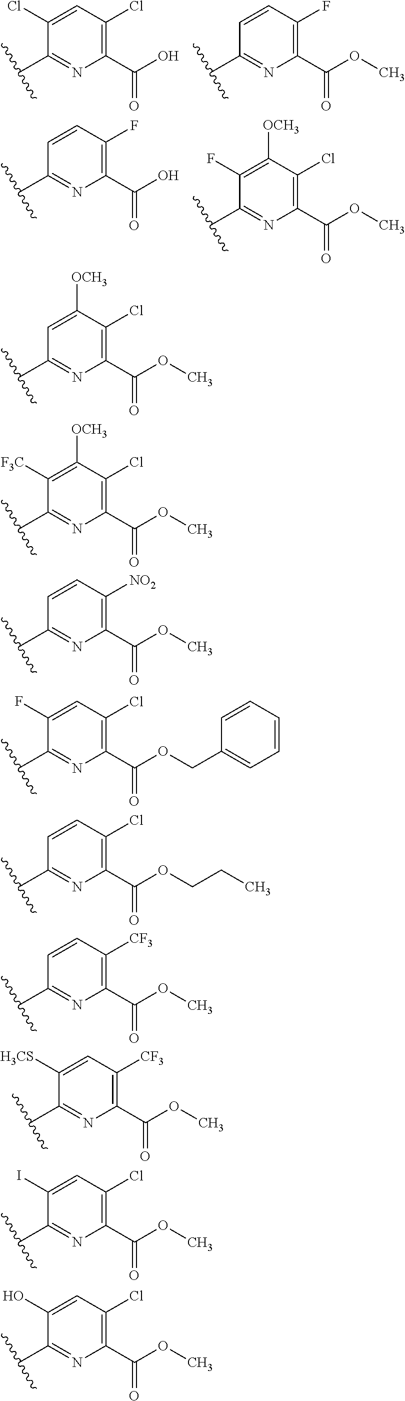

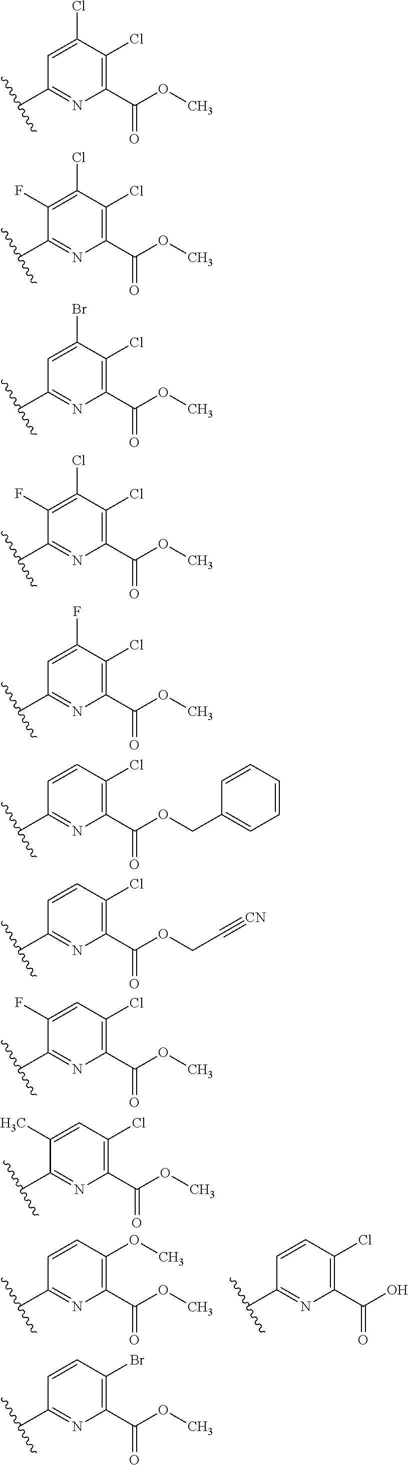

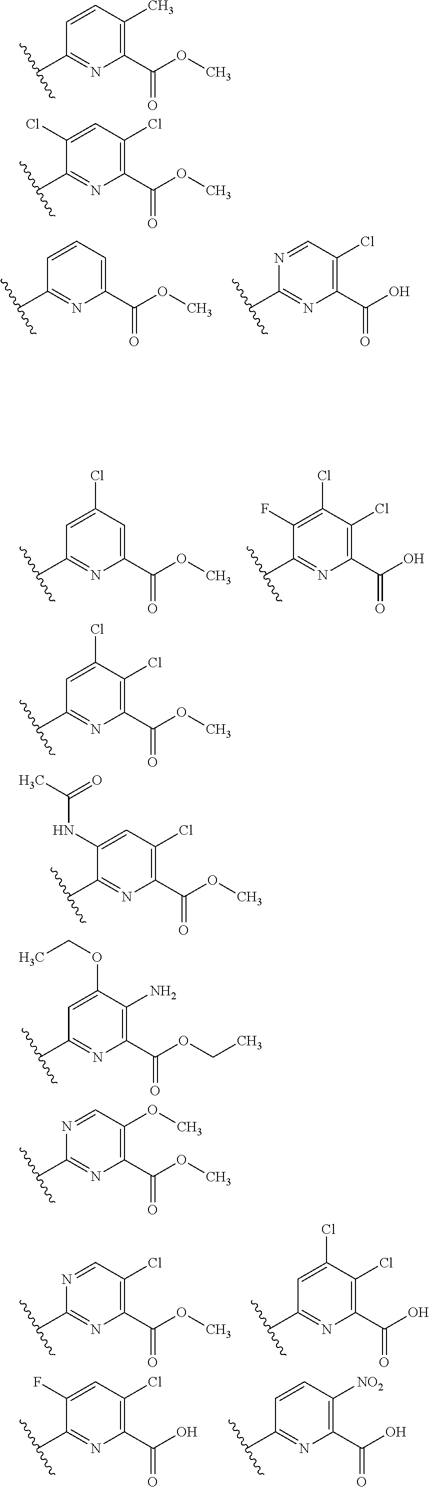

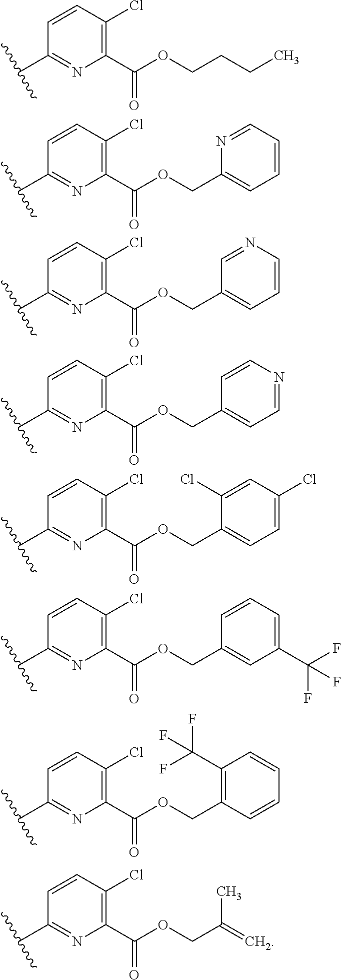

In some aspects, R, Z, Y, and X are as defined above and Ar is phenyl or biphenyl. In some aspects, Ar is substituted or unsubstituted phenyl.

In some aspects, Ar is a mono-substituted phenyl, such as a 4-substituted phenyl. In some aspects, R₃ (4-position) is halogen (e.g., bromine, chlorine, iodine, or fluorine), alkyl (e.g., methyl), alkoxy (e.g., methoxy or t-butoxy), alkylthio (e.g., methylthio), alkylsulfinyl (e.g., methylsulfinyl), alkylsulfonyl (e.g., methylsulfonyl), alkylsulfonyl(oxy) (e.g., (methylsulfonyl)(oxy)), haloalkoxy (e.g., trifluoromethoxy, difluoromethoxy, fluoromethoxy), haloalkyl (e.g., 1-fluoroethyl, trifluoromethyl, difluoromethyl), haloalkylthio (e.g., (trifluoromethyl)thio), substituted alkyl (e.g., 1-hydroxyethyl), alkylcarbonyl (e.g., acetyl), formyl, substituted or unsubstituted cycloalkyl (e.g., cyclopropyl, 2,2-difluorocyclopropyl), ethenyl (e.g., vinyl), substituted or unsubstituted alkynyl (e.g., ethynyl or trimethylsilyl ethynyl), nitro, amino, or cyano. Examples include, but are not limited to

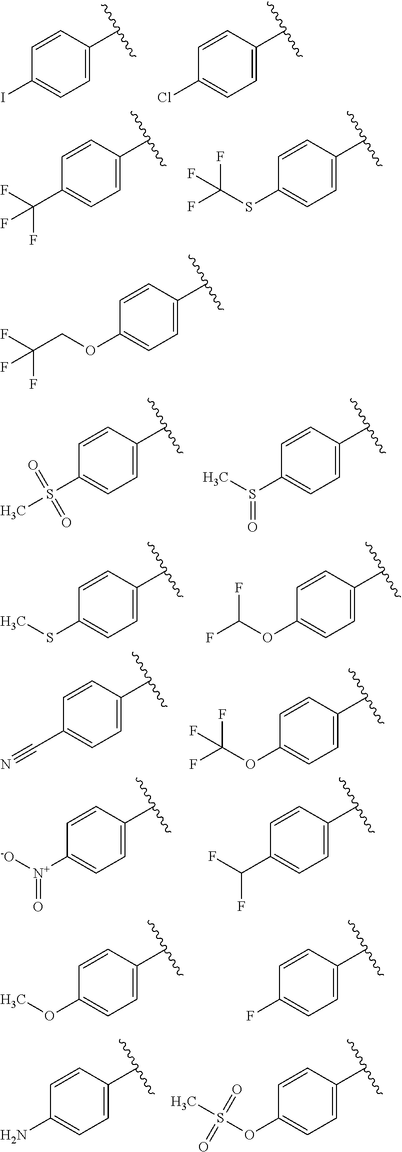

In some aspects, Ar is a di-substituted phenyl, for example, phenyl substituted at the 2 and 4 positions (relative to position 1, the point of attachment to the pyridine, pyrimidine, pyridinium, or pyrimidinium ring). In some aspects, R₅ (2-position) is halogen (e.g., bromine, chlorine or fluorine), alkynyl (e.g., ethynyl), substituted alkynyl (e.g., trimethylsilyl ethynyl), haloalkyl (e.g., trifluoromethyl), cyano, amido, alkoxy (e.g., methoxy), or alkyl (e.g., methyl). In some aspects, R₅ is as defined above and R₃ (4-position) is halogen (e.g., bromine, chlorine or fluorine), alkyl (e.g., methyl), substituted or unsubstituted alkoxy (e.g., methoxy, t-butoxy, or trifluoromethoxy), alkylsulfonyl (e.g., methylsulfonyl), alkylsulfonyl(oxy) (e.g., (methylsulfonyl)(oxy)), haloalkylsulfonyl(oxy) (e.g., (trifluoromethylsulfonyl)(oxy)), haloalkylthio (e.g., (trifluoromethyl)thio), haloalkyl (e.g., 1-fluoroethyl, trifluoromethyl, difluoromethyl), substituted alkyl (e.g., 1-hydroxyethyl, 1,1-difluoro-2-methoxyethyl, carboxydifluoromethyl, cyanodifluoromethyl, 2-amino-1,1-difluoro-2-oxoethyl), alkylcarbonyl (e.g., acetyl), formyl, 2,2-dimethylhydrazono, methoxyimino, hydroxyimino, substituted or unsubstituted cycloalkyl (e.g., cyclopropyl, 2,2-difluorocyclopropyl, 1-cyanocyclopropyl), ethenyl (e.g., vinyl), substituted or unsubstituted alkynyl (e.g., ethynyl or trimethylsilyl ethynyl), hydroxy, amino, substituted or unsubstituted phenoxy (e.g., 4-fluorophenoxy), or cyano.

In some aspects, Ar is as shown below:

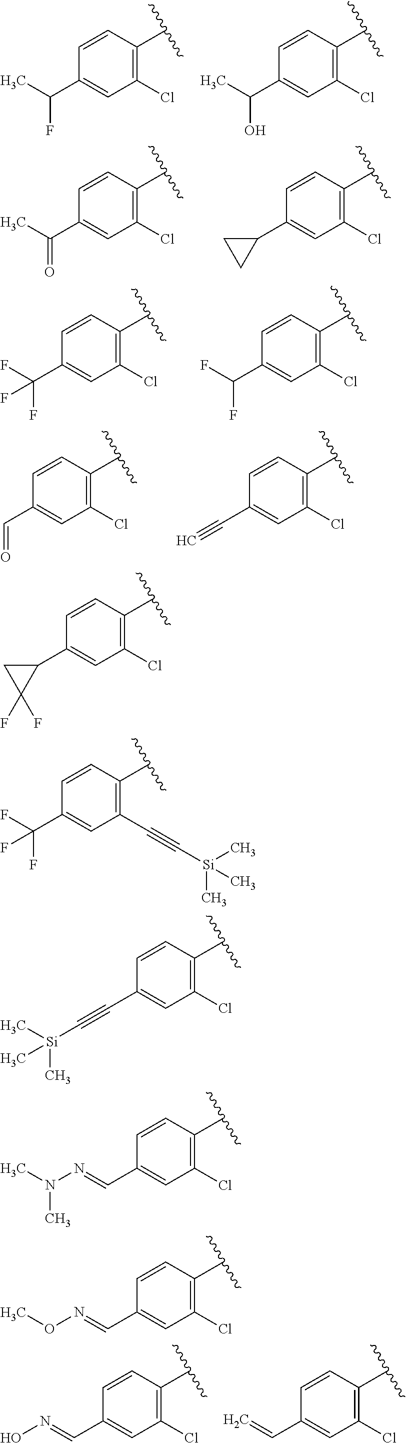

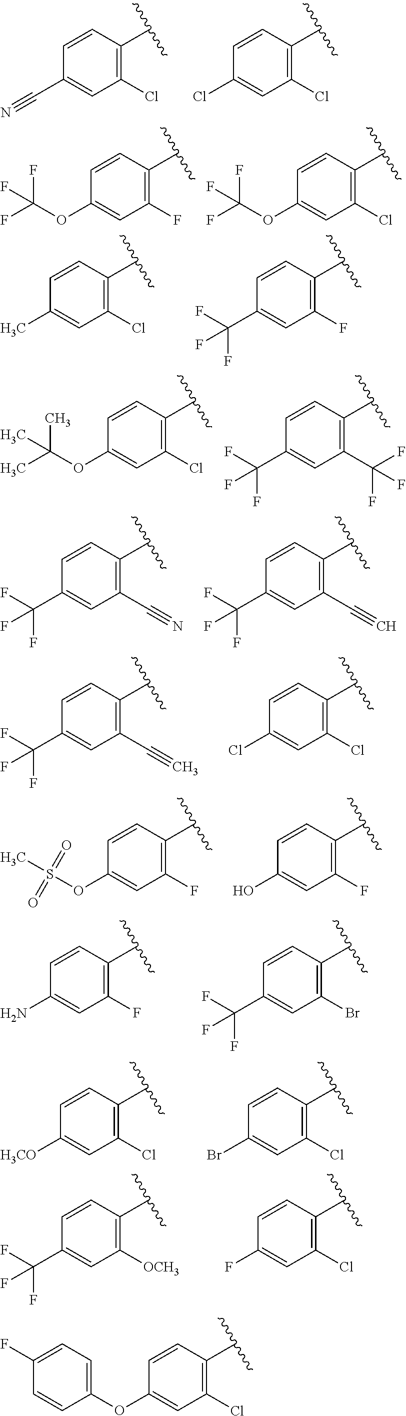

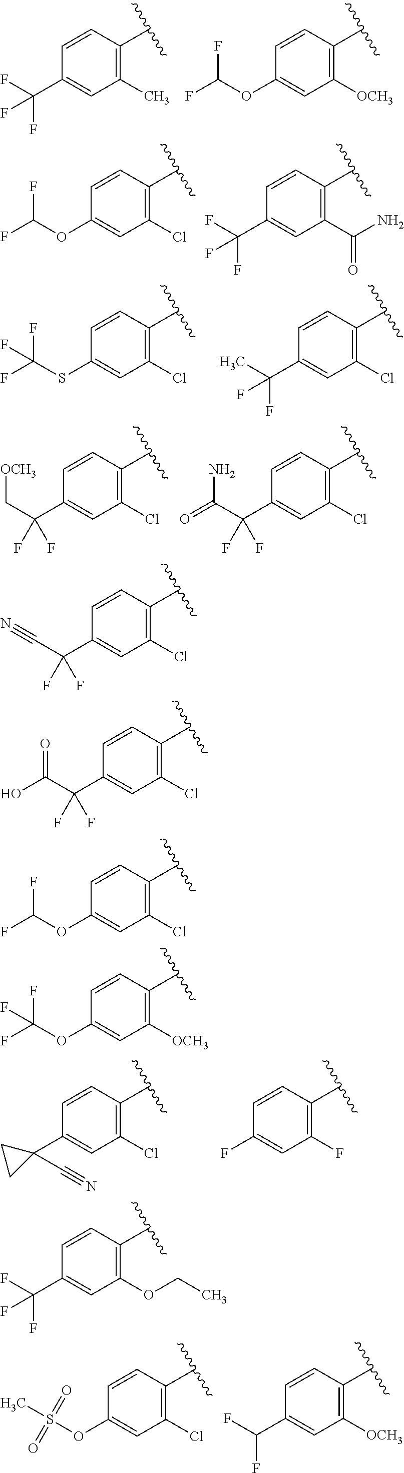

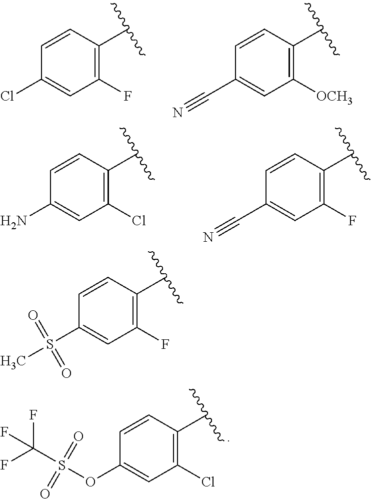 [SMILES: *c1ccc(Cl)cc1F ; *c1ccc(C#N)cc1OC ; *c1ccc(N)cc1Cl ; *c1ccc(C#N)cc1F ; *c1ccc(S(C)(=O)=O)cc1F ; *c1ccc(OS(=O)(=O)C(F)(F)F)cc1Cl]

In other aspects, Ar is a 2,3-disubstituted phenyl. Examples include, but are not limited to,

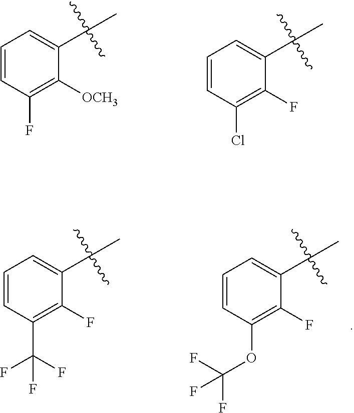 [SMILES: *c1cccc(F)c1OC ; *c1cccc(Cl)c1F ; *c1cccc(C(F)(F)F)c1F ; *c1cccc(OC(F)(F)F)c1F]

In other aspects, Ar is a 2,5-disubstituted phenyl, for example, but not limited to

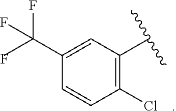

In other aspects, Ar is a 2,6-disubstituted phenyl, for example, but not limited to

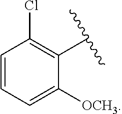 [SMILES: *c1c(Cl)cccc1OC]

In other aspects, Ar is a 3,4-disubstituted phenyl. Examples include, but are not limited to

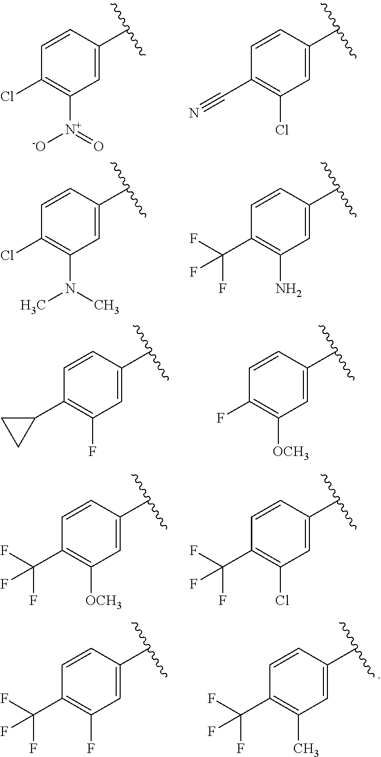

In other aspects, Ar is a biphenyl. One or both of the phenyl rings can be substituted or unsubstituted, for example, but are not limited to

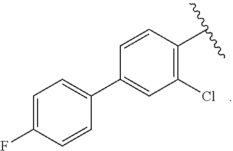 [SMILES: *c1ccc(-c2ccc(F)cc2)cc1Cl ; "."]

In some aspects, Ar is a trisubstituted phenyl, such as a 2,3,4-trisubtituted phenyl, a 2,4,5-trisubstituted phenyl, a 2,4,6-trisubstituted phenyl, or a 2,3,5-trisubstituted phenyl. In some aspects, R₁-R₅ are independently hydrogen, halogen (e.g., bromine, chlorine, iodine, or fluorine), alkyl (e.g., methyl), alkoxy (e.g., methoxy, or t-butoxy), haloalkoxy (e.g., trifluoromethoxy), haloalkyl (e.g., 1-fluoroethyl, trifluoromethyl, difluoromethyl), substituted alkyl (e.g., 1-hydroxyethyl), alkylcarbonyl (e.g., acetyl), formyl, 2,2-dimethylhydrazono, methoxyimino, hydroxyimino, alkylthio or alkylsulfanyl (e.g., methylsulfanyl), alkylsulfinyl (e.g., methylsulfinyl), alkylsulfonyl (e.g., methylsulfonyl), substituted or unsubstituted cycloalkyl (e.g., cyclopropyl, 2,2-difluorocyclopropyl), ethenyl (e.g., vinyl), substituted or unsubstituted alkynyl (e.g., ethynyl or trimethylsilyl ethynyl), hydroxy, nitro, substituted or unsubstituted amino (e.g., amino, methylamino dimethylamino, diethylamino), or cyano. Examples include, but are not limited to,

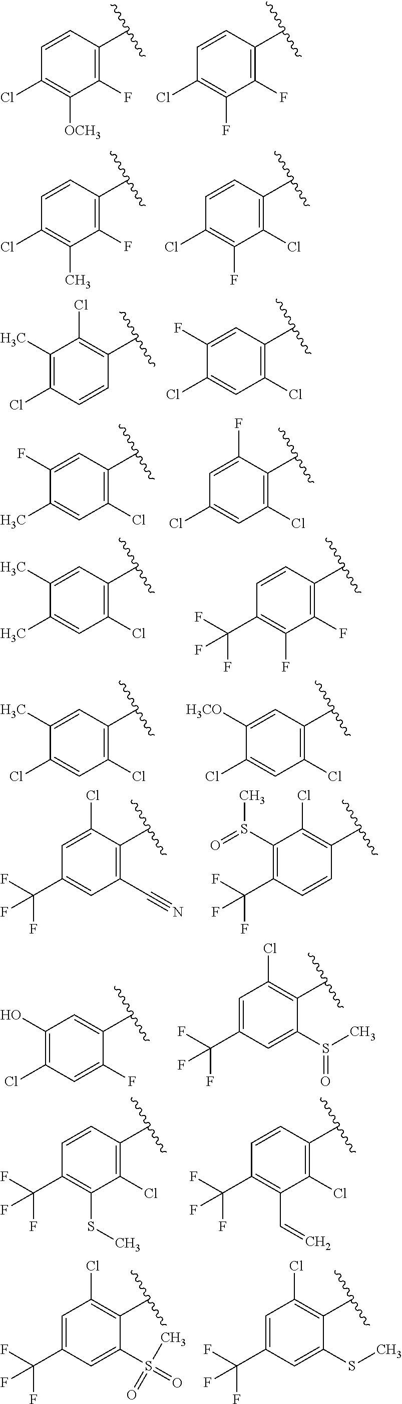

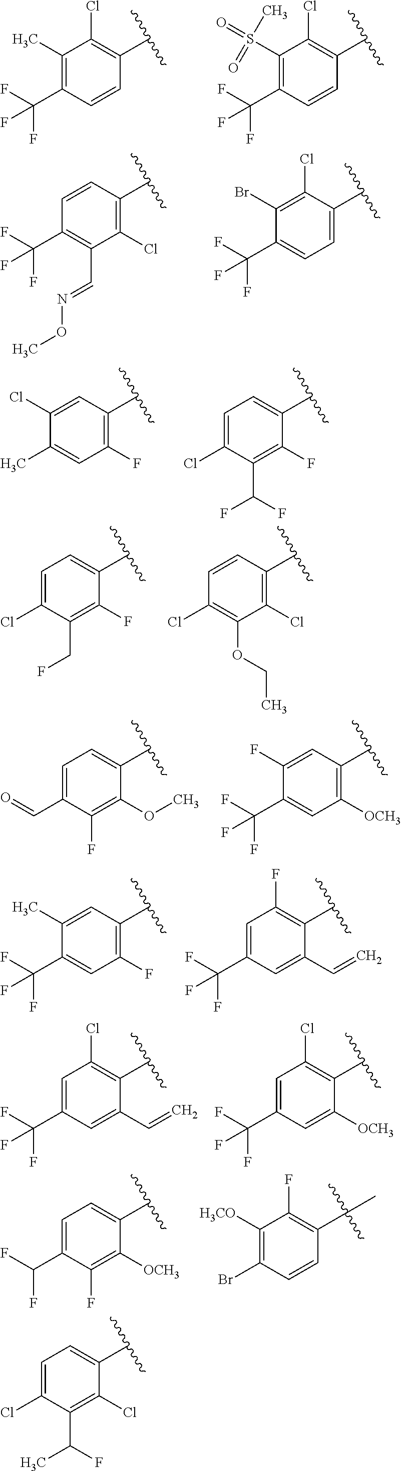

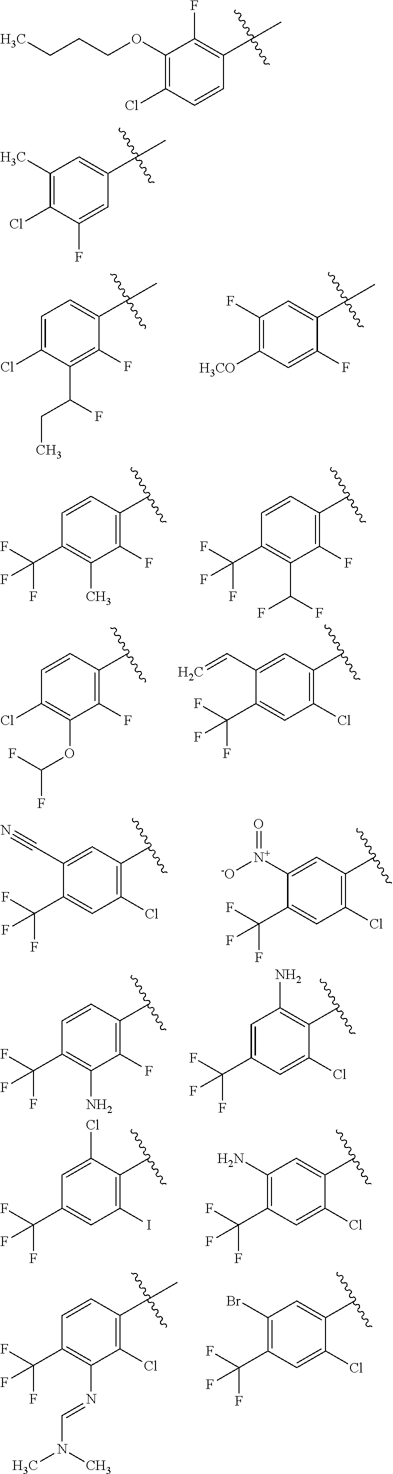

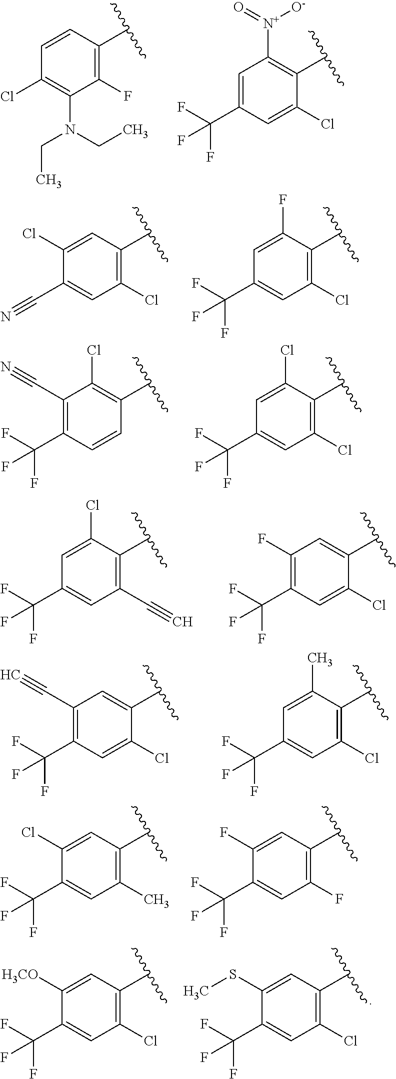

In some aspects, Ar is a tetrasubstituted phenyl. Examples include, but are not limited to,

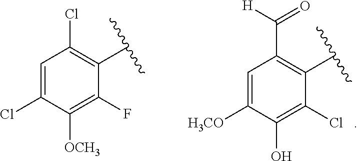 [SMILES: *c1c(Cl)cc(Cl)c(OC)c1F ; *c1c(C=O)cc(OC)c(O)c1Cl]

In some aspects, Ar is phenyl and R₃ and R₄ or R₄ and R₅ taken together form a 5- or 6-membered aliphatic or aromatic ring, containing 0 to 3 heteroatoms selected from oxygen (O), nitrogen (N), and sulfur (S). Examples include, but are not limited to,

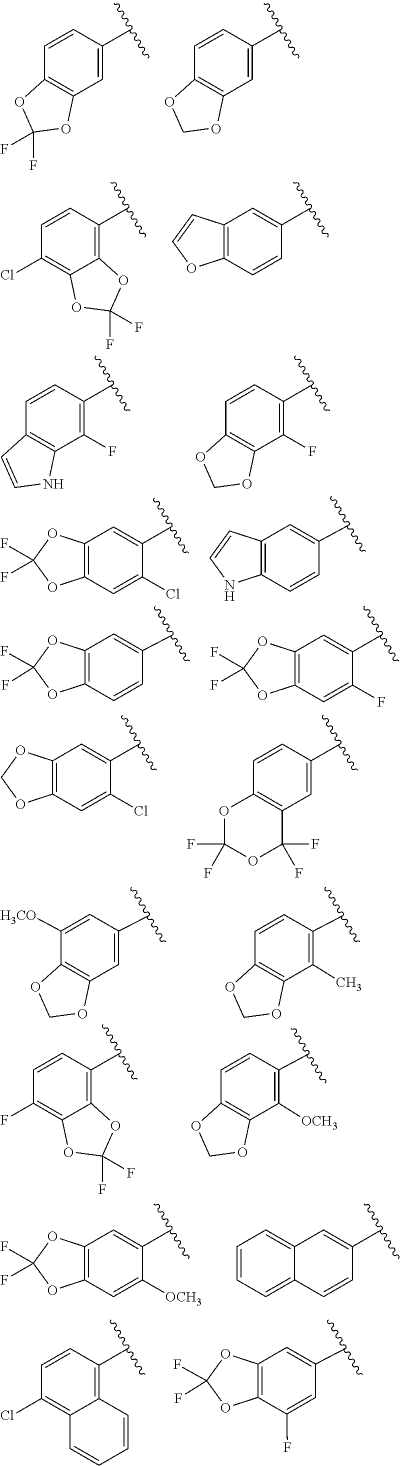

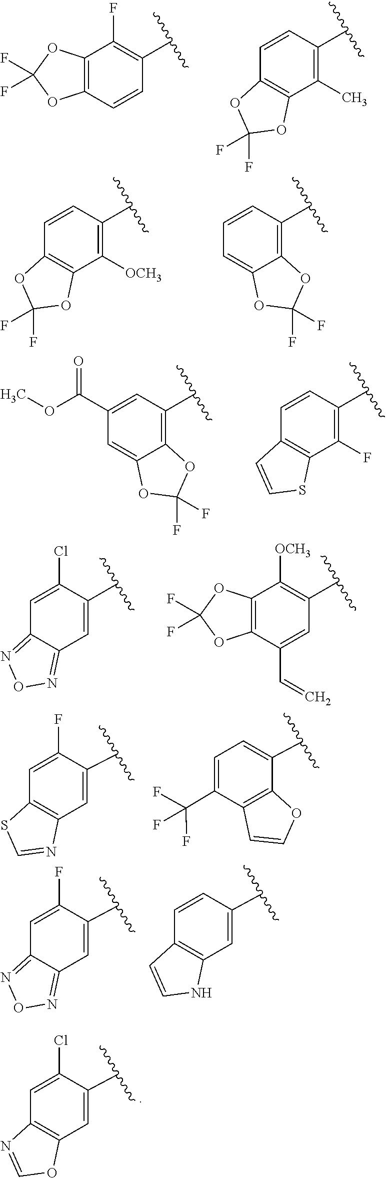

In some aspects, Ar is a heterocyclic ring. In some aspects, Ar is a substituted or unsubstituted pyridine ring. In some aspects, Ar is a 2,3,5-trisubstituted, a 3,4,5-trisubstituted, a 2,3-disubstituted, a 2,4-disubstituted, a 2,5-disubstituted, a 3,5-disubstituted, a 6-substituted, or a 2,6-disubstituted pyridyl (relative to the nitrogen on the pyridine or pyrimidine ring). In some aspects, R₁-R₅ are independently hydrogen, halogen (e.g., chlorine) and haloalkyl (e.g., trifluoromethyl). Examples include, but are not limited to,

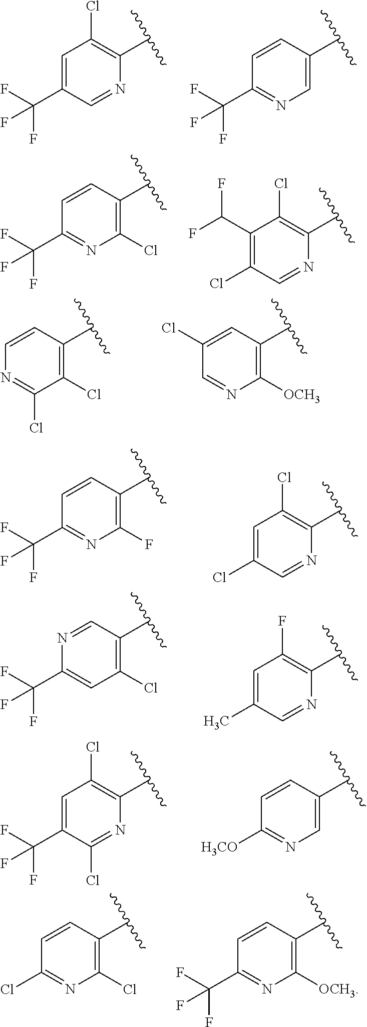

### III. Methods of Preparation

Exemplary procedures to synthesize the compounds of Formula (I) are provided below.

In step a of Scheme 1, an aryl or heteroaryl halide 1.1, wherein R₄, R₅, X₂, X₃, and X₄ are as previously defined and Yₓ is Br or I, can be converted to the corresponding boronic acid or boronate 1.2, wherein R₄, R₅, Rₓ, X₂, X₃, and X₄ are as previously defined, using methods known in the art, including but not limited to halogen-metal exchange followed by reaction with a boron source such as trimethylborate or metal-catalyzed cross-coupling with a boron source such as, but not limited to, bis(pinacolato)diboron. Metals used in the halogen-metal exchange reaction can be lithium or magnesium, and those in the cross-coupling reaction can be palladium, nickel, or copper.

The pyridine carboxylate 1.4, wherein R, R₄, R₅, X₁, X₂, X₃, X₄, Y and Z are as previously defined, can be synthesized under Suzuki cross-coupling conditions of a pyridine or pyrimidine halide 1.3, wherein R, X₁, Y, Y₂, and Z are as previously disclosed with an appropriate boronic acid or boronate 1.2, wherein R₄, R₅, Rₓ, X₂, X₃, and X₄ are as previously defined in the presence of a catalyst, with or without an added ligand, and a base in a variety of solvents at an elevated temperature as in step b of Scheme 1. In one aspect, the catalysts can be palladium catalysts, such as palladium (II) catalysts (e.g., palladium (II) acetate Pd(OAc)₂, palladium(II) chloride (PdCl₂)), and tetrakis(triphenylphosphine)palladium (0) (Pd(PPh₃)₄); nickel catalysts, such as NiCl₂(dppf) and G₃DenP-Ni; iron catalysts; copper catalysts; and ruthenium catalysts.

Suitable ligands for the catalyst system include, but are not limited to, trialkylphosphines and triarylphosphines. These include, but are not limited to, tri-tert-butylphosphine, tricyclohexylphosphine, di-tert-butylphenylphosphine, dicyclohexylphenylphosphine, triphenylphosphine, 4-diphenylphosphinomethyl polystyrene resin crosslinked, sodium diphenylphosphinobenzene-3-sulfonate with 2% DVB, tri(p-tolyl)phosphine, (±)-2,2′-bis(diphenylphosphino)-1,1′-binaphthyl.

The pH can be adjusted using one or more bases, such as potassium carbonate, potassium bicarbonate, the sodium carbonates (including bicarbonates), potassium acetate, sodium acetate, potassium phosphate bases (mono, di and tribasic), sodium tetraborate, potassium hydroxide, sodium hydroxide, cesium fluoride and potassium fluoride and organic bases such as triethylamine, triisopropylamine, diisopropylamine, diethylamine, and diisopropylethylamine. In another aspect, the reaction mixture can be pretreated with carbon dioxide (CO₂) to adjust the pH prior to the Suzuki coupling reaction.

Alternatively, the Suzuki coupling can be conducted in the presence of CO₂, e.g., bubbling CO₂ into the mixture reaction. The reaction can be performed in a mixture of organic solvents containing methyl isobutyl ketone (MIBK), dimethoxyethane (DME), acetonitrile (MeCN or CH₃CN), toluene, benzyl alcohol, and methanol (MeOH), including mixtures with and without water. Compound 1.4 can be further elaborated using methods known in the art.

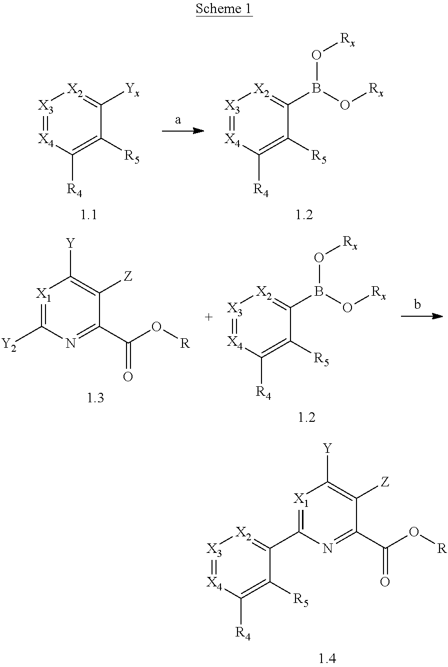

In step a of Scheme 2, the pyridine carboxylate 1.4, wherein R, R₄, X₂, and X₃ are as previously defined; each R₃, R₅, Y, and Z independently is Br; and R₆ independently is I, which can be converted to an intermediate, wherein R, R₄, X₂, and X₃ are as previously defined; each R₃, R₅, Y, and Z independently is —C≡CSi(CH₃)₃; and R₆ independently is —C≡CSi(CH₃)₃, via a Sonogashira cross-coupling with a trimethylsilylacetylene, in the presence of a base such as triethylamine and catalysts such as Pd(PPh₃)₂Cl₂ and copper(I) iodide, in a polar, aprotic solvent such as THF, at a temperature from about 50° C. to about 75° C. The silyl protecting group can be removed by methods known by those skilled in the art, including but not limited to, tetrabutylammonium fluoride in a polar, aprotic solvent such as THF, at a temperature from about −10° C. to about 10° C. to provide 2.1, wherein R, R₄, X₂, and X₃ are as previously defined; each R₃, R₅, Y, and Z independently is —C≡CH; and R₆ independently is —C≡CH. The pyridine carboxylate 1.4, wherein R, R₄, R₅, R₆, X₂, X₃, Y, and Z are as previously defined and R₃ is Br, can be transformed into 2.2, wherein R, R₄, R₅, R₆, X₂, X₃, Y, and Z are as previously defined and R₃ is a cyclopropyl or a substituted or unsubstituted phenyl, under Suzuki cross-coupling conditions, such as by treatment with an appropriate boronic acid or boronate, in the presence of a palladium catalyst such as Pd(PPh₃)₄ or Pd(PPh₃)₂Cl₂, in the presence of a base such as potassium phosphate or potassium fluoride, in a variety of solvents such as toluene or acetonitrile-water mixtures, at a temperature from about 80° C. to about 120° C., as in step c of Scheme 2. In step d of Scheme 2, the pyridine carboxylate 1.4, wherein R, R₄, R₅, X₂, and X₃ are as previously defined; each R₃, Y, and Z independently is Br; and R₆ independently is I, can be converted to 2.3, wherein R, R₄, R₅, X₂, and X₃ are as previously defined; R₃, Y, and Z independently is —C≡N; and R₆ independently is —C≡N, by treatment with zinc(II) cyanide, in the presence of a palladium catalyst such as Pd(PPh₃)₄ or Pd(PPh₃)₂Cl₂, in a polar, aprotic solvent such as N,N-dimethylformamide (DMF), at a temperature from about 140° C. to about 160° C. Compounds 2.1, 2.2, and 2.3 can be further elaborated using methods known in the art.

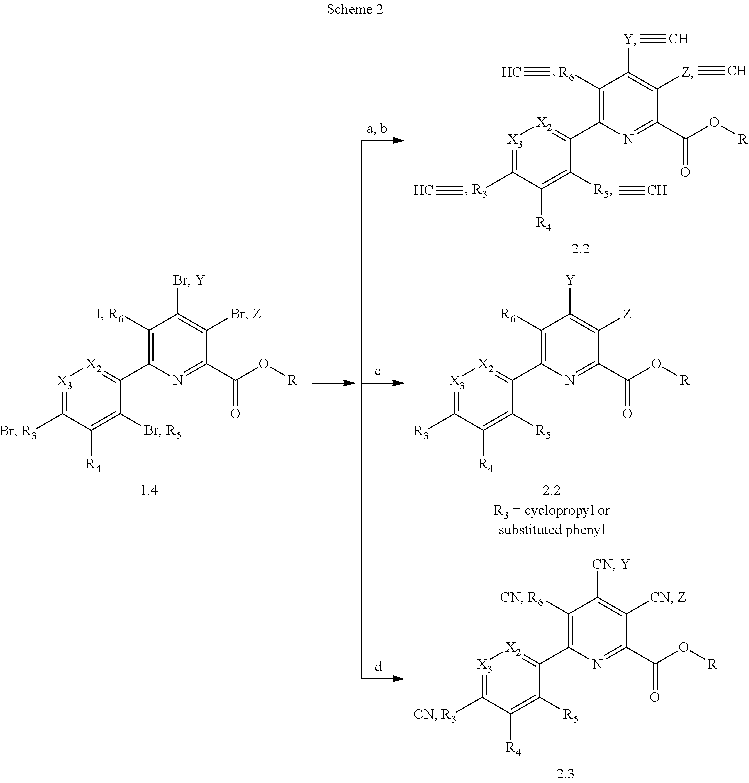

In step a of Scheme 3, the pyridine carboxylate 1.4, wherein R, R₃, R₄, R₅, X₂, and X₃ are as previously defined; each Y and Z independently is Br or I; and R₆ independently is I, can be converted to 3.1, wherein R, R₃, R₄, R₅, X₂, and X₃ are as previously defined; each Y and Z independently is Br or I; and R₆ independently is CH₃, via palladium-catalyzed cross-coupling with methylboronic acid, in the presence of a base such as potassium phosphate and a catalyst such as tetrakis(triphenylphosphine)palladium(0) (Pd(PPh₃)₄), in a non-polar, aprotic solvent such as toluene, at a temperature from about 90° C. to about 110° C. The pyridine carboxylate 1.4, wherein R, R₃, R₄, R₅, R₆, X₂, X₃, and Y are as previously defined and Z is Br or I can be transformed into 3.2, wherein R, R₃, R₄, R₅, R₆, X², X³, and Y are as previously defined and Z is CF₃, by treatment with methyl-2,2-difluoro-2-(fluorosulfonyl)acetate, in the presence of a catalytic amount of copper(I) iodide in a polar, aprotic solvent such as DMF, at a temperature from about 90° C. to about 110° C. under microwave conditions as in step b of Scheme 3. Both 3.1 and 3.2 can be further elaborated using methods known in the art.

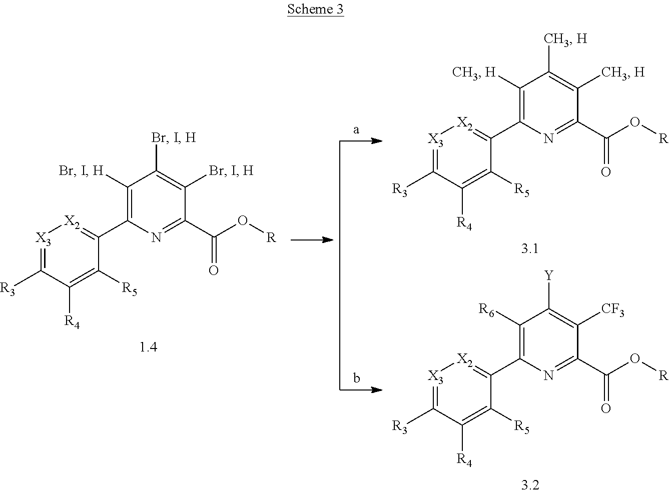

In step a of Scheme 4, the pyridine carboxylate 1.4, wherein R, R₃, R₄, R₅, R₆, X₂, X₃, and Z are as previously defined and Y is NH₂ can be converted to 4.1, wherein R, R₃, R₄, R₅, R₆, X₂, X₃, and Z are as previously defined and Y is Cl or Br, under Sandmeyer reaction conditions with a chlorine or bromine source such as copper(II) chloride or copper(II) bromide, respectively, in the presence of tert-butyl nitrite in a polar, aprotic solvent such as acetonitrile at a temperature of about 15° C. to about 40° C. The pyridine carboxylate 1.4, wherein R, R₃, R₄, R₅, R₆, X₂, X₃, and Z are as previously defined and Y is NH₂ can be transformed into 4.2, wherein R, R₃, R₄, R₅, R₆, X₂, X₃, and Z are as previously defined and Y is H, by treatment with isoamyl nitrite in a polar, aprotic solvent such as tetrahydrofuran (THF) at a temperature of about 45° C. to about 65° C. or with sodium nitrite in the presence of an acid such as sulfuric acid, in a polar, protic solvent system such as ethanol-toluene, at a temperature from about 70° C. to about 90° C. as in step b of Scheme 4. Both 4.1 and 4.2 can be further elaborated using methods known in the art.

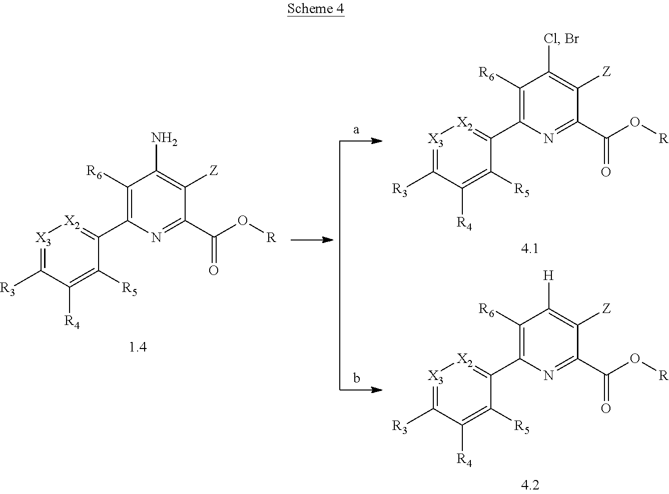

In step a of Scheme 5, the pyridine carboxylate 1.4, wherein R, R₃, R₄, R₅, X₂, X₃, Y and Z are as previously defined and R₆ is H can be transformed into 4.1, wherein R, R₃, R₄, R₅, X₂, X₃, Y, and Z are as previously defined and R₆ is I, via reaction with periodic acid and iodine in a polar, protic solvent such as methanol, at a temperature from about 50° C. to about 75° C. The pyridine carboxylate 1.4, wherein R, R₃, R₄, R₅, R₆, X₂, X₃, and Z are as previously defined and Y is Br or Cl, can be converted to 5.2, wherein R, R₃, R₄, R₅, R₆, X₂, X₃, and Z are as previously defined and Y is OCH₃, by treatment with sodium methoxide, in a polar, protic solvent such as methanol, at a temperature from about 15° C. to about 40° C. as in step b of Scheme 5. In step c of Scheme 5, the pyridine carboxylate 1.4, wherein R, R₃, R₄, R₅, X₂, X₃, Y, and Z are as previously defined and R₆ is Br or Cl, can be treated with sodium thiomethoxide, in a polar, aprotic solvent such as DMF, at a temperature from about 40° C. to about 65° C. to afford 5.3, wherein R, R₃, R₄, R₅, X₂, X₃, Y, and Z are as previously defined and R₆ is SCH₃. Compounds 5.1, 5.2, and 5.3 can be further elaborated using methods known in the art.

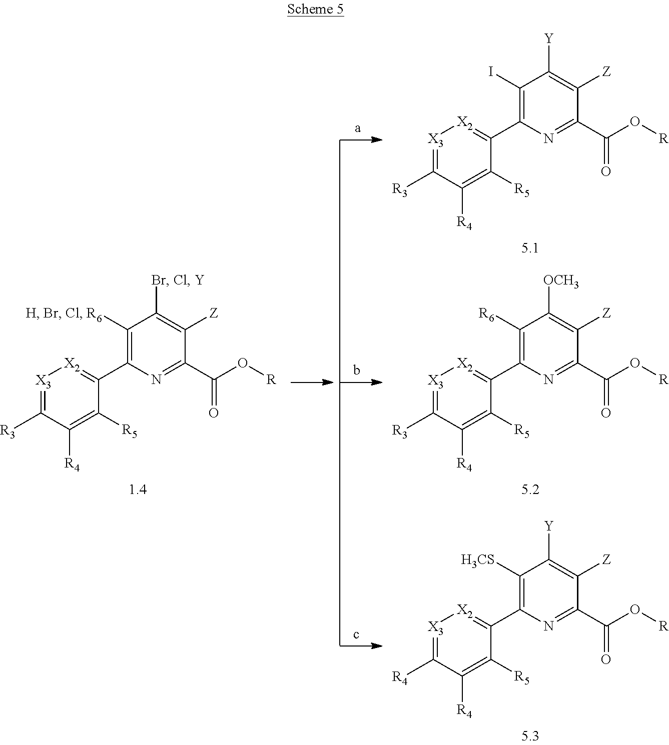

In step a of Scheme 6, the pyridine carboxylate 1.4, wherein R, R₃, R₄, R₅, R₆, X₂, X₃, Y, and Z are as previously defined and R₃ is Br can be transformed into the corresponding vinyl 6.1, wherein R, R₃, R₄, R₅, R₆, X₂, X₃, Y, and Z are as previously defined and R₃ is vinyl, via palladium-catalyzed cross-coupling with a vinyl source such as trifluorovinyl borate potassium salt, in the presence of a base such as potassium carbonate and a catalyst such as Pd(PPh₃)₂Cl₂, in a polar, aprotic solvent such as dimethyl sulfoxide, at a temperature from about 75° C. to about 100° C. The pyridine carboxylate 1.4, wherein R, R³, R⁴, R⁵, R⁶, X², X³, Y, and Z are as previously defined and R₃ is Br can be transformed into the corresponding ketone 6.2, wherein R₁, R₂, R₃, R₄, R₅, R₆, X₂, and X₃ are as previously defined and R₃ is C(O)CH₃, by treatment with tributyl (1-ethoxyvinyl)stannane, in the presence of a palladium catalyst such as Pd(PPh₃)₂Cl₂, in a polar, aprotic solvent such as dichloroethane (DCE), at a temperature from about 100° C. to about 140° C. as in step b of Scheme 6. Both 6.1 and 6.2 can be further elaborated using methods known in the art (including, but not limited to, oxidation/reduction, cyclopropanation, fluorination) to provide other groups at R₃.

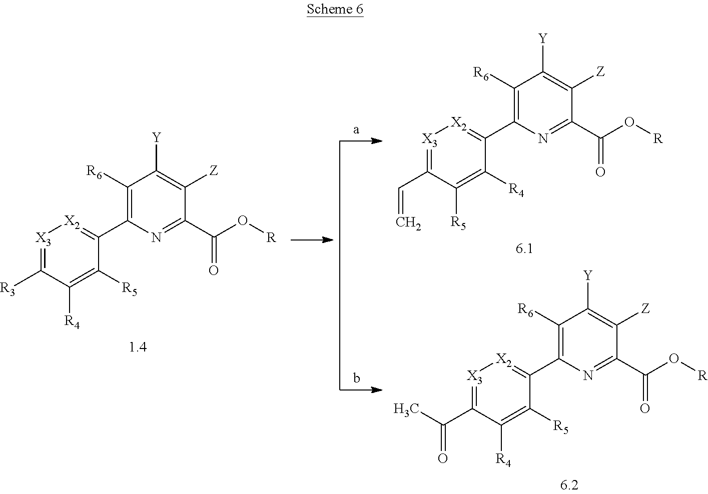

### Compositions and Methods

In some aspects, the compounds provided herein are employed in mixtures containing a herbicidally effective amount of the compound along with at least one agriculturally acceptable adjuvant or carrier. Exemplary adjuvants or carriers include those that are not phytotoxic or significantly phytotoxic to valuable crops, e.g., at the concentrations employed in applying the compositions for selective weed control in the presence of crops, and/or do not react or significantly react chemically with the compounds provided herein or other composition ingredients. Such mixtures can be designed for application directly to weeds or their locus or can be concentrates or formulations that are diluted with additional carriers and adjuvants before application. They can be solids, such as, for example, dusts, granules, water dispersible granules, or wettable powders, or liquids, such as, for example, emulsifiable concentrates, solutions, emulsions or suspensions. They can also be provided as a premix or tank mixed.

Suitable agricultural adjuvants and carriers that are useful in preparing the herbicidal mixtures of the disclosure are well known to those skilled in the art. Some of these adjuvants include, but are not limited to, crop oil concentrate (mineral oil (85%)+emulsifiers (15%)); nonylphenol ethoxylate; benzylcocoalkyldimethyl quaternary ammonium salt; blend of petroleum hydrocarbon, alkyl esters, organic acid, and anionic surfactant; C₉-C₁₁ alkylpolyglycoside; phosphated alcohol ethoxylate; natural primary alcohol (C₁₂-C₁₆) ethoxylate; di-sec-butylphenol EO-PO block copolymer; polysiloxane-methyl cap; nonylphenol ethoxylate+urea ammonium nitrate; emulsified methylated seed oil; tridecyl alcohol (synthetic) ethoxylate (8EO); tallow amine ethoxylate (15 EO); PEG(400) dioleate-99.

Liquid carriers that can be employed include water and organic solvents. The organic solvents include, but are not limited to, petroleum fractions or hydrocarbons such as mineral oil, aromatic solvents, paraffinic oils, and the like; vegetable oils such as soybean oil, rapeseed oil, olive oil, castor oil, sunflower seed oil, coconut oil, corn oil, cottonseed oil, linseed oil, palm oil, peanut oil, safflower oil, sesame oil, tung oil and the like; esters of the above vegetable oils; esters of monoalcohols or dihydric, trihydric, or other lower polyalcohols (4-6 hydroxy containing), such as 2-ethylhexyl stearate, n-butyl oleate, isopropyl myristate, propylene glycol dioleate, di-octyl succinate, di-butyl adipate, di-octyl phthalate and the like; esters of mono-, di- and poly-carboxylic acids and the like. Specific organic solvents include toluene, xylene, petroleum naphtha, crop oil, acetone, methyl ethyl ketone, cyclohexanone, trichloroethylene, perchloroethylene, ethyl acetate, amyl acetate, butyl acetate, propylene glycol monomethyl ether and diethylene glycol monomethyl ether, methyl alcohol, ethyl alcohol, isopropyl alcohol, amyl alcohol, ethylene glycol, propylene glycol, glycerine, N-methyl-2-pyrrolidinone, N,N-dimethyl alkylamides, dimethyl sulfoxide, liquid fertilizers, and the like. In some aspects, water is the carrier for the dilution of concentrates.

Suitable solid carriers include but are not limited to talc, pyrophyllite clay, silica, attapulgus clay, kaolin clay, kieselguhr, chalk, diatomaceous earth, lime, calcium carbonate, bentonite clay, Fuller's earth, cottonseed hulls, wheat flour, soybean flour, pumice, wood flour, walnut shell flour, lignin, and the like.

In some aspects, one or more surface-active agents are utilized in the compositions of the present disclosure. Such surface-active agents are, in some aspects, employed in both solid and liquid compositions, e.g., those designed to be diluted with carrier before application. The surface-active agents can be anionic, cationic or nonionic in character and can be employed as emulsifying agents, wetting agents, suspending agents, or for other purposes. Surfactants conventionally used in the art of formulation and which may also be used in the present formulations are described, inter alia, in McCutcheon's Detergents and Emulsifiers Annual, MC Publishing Corporation: Ridgewood, N J, 1998, and in Encyclopedia of Surfactants, Vol. I-III, Chemical Publishing Company: New York, 1980-81. Typical surface-active agents include but are not limited to salts of alkyl sulfates, such as diethanolammonium lauryl sulfate; alkylarylsulfonate salts, such as calcium dodecylbenzenesulfonate; alkylphenol-alkylene oxide addition products, such as nonylphenol-C₁₈ ethoxylate; alcohol-alkylene oxide addition products, such as tridecyl alcohol-C₁₆ ethoxylate; soaps, such as sodium stearate; alkylnaphthalene-sulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; salts of mono- and dialkyl phosphate esters; vegetable or seed oils such as soybean oil, rapeseed/canola oil, olive oil, castor oil, sunflower seed oil, coconut oil, corn oil, cottonseed oil, linseed oil, palm oil, peanut oil, safflower oil, sesame oil, tung oil and the like; and esters of the above vegetable oils, e.g., methyl esters.

In some aspects, these materials, such as vegetable or seed oils and their esters, can be used interchangeably as an agricultural adjuvant, as a liquid carrier or as a surface active agent.

Other exemplary additives for use in the compositions provided herein include but are not limited to compatibilizing agents, antifoam agents, sequestering agents, neutralizing agents and buffers, corrosion inhibitors, dyes, odorants, spreading agents, penetration aids, sticking agents, dispersing agents, thickening agents, freezing point depressants, antimicrobial agents, and the like. The compositions may also contain other compatible components, for example, other herbicides, plant growth regulants, fungicides, insecticides, and the like and can be formulated with liquid fertilizers or solid, particulate fertilizer carriers such as ammonium nitrate, urea and the like.

The concentration of the active ingredients in the herbicidal compositions of this disclosure is generally from about 0.001 to about 98 percent by weight. Concentrations from about 0.01 to about 90 percent by weight are often employed. In compositions designed to be employed as concentrates, the active ingredient is generally present in a concentration from about 5 to about 98 weight percent, preferably about 10 to about 90 weight percent. Such compositions are typically diluted with an inert carrier, such as water, before application. The diluted compositions usually applied to weeds or the locus of weeds generally contain about 0.0001 to about 1 weight percent active ingredient and preferably contain about 0.001 to about 0.05 weight percent.

The present compositions can be applied to weeds or their locus by the use of conventional ground or aerial dusters, sprayers, and granule applicators, by addition to irrigation or flood water, and by other conventional means known to those skilled in the art.

In some aspects, the compounds and compositions described herein are applied as a post-emergence application, pre-emergence application, in-water application to flooded paddy rice or water bodies (e.g., ponds, lakes and streams), or burn-down application.

In some aspects, the compounds and compositions provided herein are utilized to control weeds in crops, including but not limited to citrus, apple, rubber, oil, palm, forestry, direct-seeded, water-seeded and transplanted rice, wheat, barley, oats, rye, sorghum, com/maize, pastures, grasslands, rangelands, fallowland, turf, tree and vine orchards, aquatics, or row-crops, as well as non-crop settings, e.g., industrial vegetation management (IVM) or rights-of-way. In some aspects, the compounds and compositions are used to control woody plants, broadleaf and grass weeds, or sedges.

In some aspects, the compounds and compositions provided herein are utilized to control undesirable vegetation in rice. In certain aspects, the undesirable vegetation is Brachiaria platyphylla (Groseb.) Nash (broadleaf signalgrass, BRAPP), Digitaria sanguinalis (L.) Scop. (large crabgrass, DIGSA), Echinochloa crus-galli (L.) P. Beauv. (barnyardgrass, ECHCG), Echinochloa colonum (L.) LINK (junglerice, ECHCO), Echinochloa oryzoides (Ard.) Fritsch (early watergrass, ECHOR), Echinochloa oryzicola (Vasinger) Vasinger (late watergrass, ECHPH), Ischaemum rugosum Salisb. (saramollagrass, ISCRU), Leptochloa chinensis (L.) Nees (Chinese sprangletop, LEFCH), Leptochloa fascicularis (Lam.) Gray (bearded sprangletop, LEFFA), Leptochloa panicoides (Presl.) Hitchc. (Amazon sprangletop, LEFPA), Panicum dichotomiflorum (L.) Michx. (fall Panicum, PANDI), Paspalum dilatatum Poir. (dallisgrass, PASDI), Cyperus difformis L. (smallflower flatsedge, CYPDI), Cyperus esculentus L. (yellow nutsedge, CYPES), Cyperus iria L. (rice flatsedge, CYPIR), Cyperus rotundus L. (purple nutsedge, CYPRO), Eleocharis species (ELOSS), Fimbristylis miliacea (L.) Vahl (globe fringerush, FIMMI), Schoenoplectus juncoides Roxb. (Japanese bulrush, SCPJU), Schoenoplectus maritimus L. (sea clubrush, SCPMA), Schoenoplectus mucronatus L. (ricefield bulrush, SCPMU), Aeschynomene species, (jointvetch, AESSS), Alternanthera philoxeroides (Mart.) Griseb. (alligatorweed, ALRPH), Alisma plantago-aquatica L. (common waterplantain, ALSPA), Amaranthus species, (pigweeds and amaranths, AMASS), Ammannia coccinea Rottb. (redstem, AMMCO), Eclipta alba (L.) Hassk. (American false daisy, ECLAL), Heteranthera limosa (SW.) Willd./Vahl (ducksalad, HETLI), Heteranthera reniformis R. & P. (roundleaf mudplantain, HETRE), Ipomoea hederacea (L.) Jacq. (ivyleaf morningglory, IPOHE), Lindernia dubia (L.) Pennell (low false pimpernel, LIDDU), Monochoria korsakowii Regel & Maack (Monochoria, MOOKA), Monochoria vaginalis (Burm. F.) C. Presl ex Kuhth, (Monochoria, MOOVA), Murdannia nudiflora (L.) Brenan (doveweed, MUDNU), Polygonum pensylvanicum L., (Pennsylvania smartweed, POLPY), Polygonum persicaria L. (ladysthumb, POLPE), Polygonum hydropiperoides Michx. (mild smartweed, POLHP), Rotala indica (Willd.) Koehne (Indian toothcup, ROTIN), Sagittaria species, (arrowhead, SAGSS), Sesbania exaltata (Raf.) Cory/Rydb. Ex Hill (hemp Sesbania, SEBEX), or Sphenoclea zeylanica Gaertn. (gooseweed, SPDZE).

In some aspects, the compounds and compositions provided herein are utilized to control undesirable vegetation in cereals. In certain aspects, the undesirable vegetation is Alopecurus myosuroides Huds. (blackgrass, ALOMY), Apera spica-venti (L.) Beauv. (windgrass, APESV), Avena fatua L. (wild oat, AVEFA), Bromus tectorum L. (downy brome, BROTE), Lolium multiflorum Lam. (Italian ryegrass, LOLMU), Phalaris minor Retz. (littleseed canarygrass, PHAMI), Poa annua L. (annual bluegrass, POAAN), Setaria pumila (Poir.) Roemer & J. A. Schultes (yellow foxtail, SETLU), Setaria viridis (L.) Beauv. (green foxtail, SETVI), Cirsium arvense (L.) Scop. (Canada thistle, CIRAR), Galium aparine L. (catchweed bedstraw, GALAP), Kochia scoparia (L.) Schrad. (Kochia, KCHSC), Lamium purpureum L. (purple deadnettle, LAMPU), Matricaria recutita L. (wild chamomile, MATCH), Matricaria matricarioides (Less.) Porter (pineappleweed, MATMT), Papaver rhoeas L. (common poppy, PAPRH), Polygonum convolvulus L. (wild buckwheat, POLCO), Salsola tragus L. (Russian thistle, SASKR), Stellaria media (L.) Vill. (common chickweed, STEME), Veronica persica Poir. (Persian speedwell, VERPE), Viola arvensis Murr. (field violet, VIOAR), or Viola tricolor L. (wild violet, VIOTR).

In some aspects, the compounds and compositions provided herein are utilized to control undesirable vegetation in range and pasture. In certain aspects, the undesirable vegetation is Ambrosia artemisiifolia L. (common ragweed, AMBEL), Cassia obtusifolia (sickle pod, CASOB), Centaurea maculosa auct. non Lam. (spotted knapweed, CENMA), Cirsium arvense (L.) Scop. (Canada thistle, CIRAR), Convolvulus arvensis L. (field bindweed, CONAR), Euphorbia esula L. (leafy spurge, EPHES), Lactuca serriola L./Torn. (prickly lettuce, LACSE), Plantago lanceolata L. (buckhorn plantain, PLALA), Rumex obtusifolius L. (broadleaf dock, RUMOB), Sida spinosa L. (prickly Sida, SIDSP), Sinapis arvensis L. (wild mustard, SINAR), Sonchus arvensis L. (perennial sowthistle, SONAR), Solidago species (goldenrod, SOOSS), Taraxacum officinale G. H. Weber ex Wiggers (dandelion, TAROF), Trifolium repens L. (white clover, TRFRE), or Urtica dioica L. (common nettle, URTDI).

In some aspects, the compounds and compositions provided herein are utilized to control undesirable vegetation found in row crops. In certain aspects, the undesirable vegetation is Alopecurus myosuroides Huds. (blackgrass, ALOMY), Avena fatua L. (wild oat, AVEFA), Brachiaria platyphylla (Groseb.) Nash (broadleaf signalgrass, BRAPP), Digitaria sanguinalis (L.) Scop. (large crabgrass, DIGSA), Echinochloa crus-galli (L.) P. Beauv. (barnyardgrass, ECHCG), Echinochloa colonum (L.) Link (junglerice, ECHCO), Lolium multiflorum Lam. (Italian ryegrass, LOLMU), Panicum dichotomiflorum Michx. (fall Panicum, PANDI), Panicum miliaceum L. (wild-proso millet, PANMI), Setaria faberi Herrm. (giant foxtail, SETFA), Setaria viridis (L.) Beauv. (green foxtail, SETVI), Sorghum halepense (L.) Pers. (Johnsongrass, SORHA), Sorghum bicolor (L.) Moench ssp. Arundinaceum (shattercane, SORVU), Cyperus esculentus L. (yellow nutsedge, CYPES), Cyperus rotundus L. (purple nutsedge, CYPRO), Abutilon theophrasti Medik. (velvetleaf, ABUTH), Amaranthus species (pigweeds and amaranths, AMASS), Ambrosia artemisiifolia L. (common ragweed, AMBEL), Ambrosia psilostachya DC. (western ragweed, AMBPS), Ambrosia trifida L. (giant ragweed, AMBTR), Asclepias syriaca L. (common milkweed, ASCSY), Chenopodium album L. (common lambsquarters, CHEAL), Cirsium arvense (L.) Scop. (Canada thistle, CIRAR), Commelina benghalensis L. (tropical spiderwort, COMBE), Datura stramonium L. (jimsonweed, DATST), Daucus carota L. (wild carrot, DAUCA), Euphorbia heterophylla L. (wild poinsettia, EPHHL), Erigeron bonariensis L. (hairy fleabane, ERIBO), Erigeron canadensis L. (Canadian fleabane, ERICA), Helianthus annuus L. (common sunflower, HELAN), Jacquemontia tamnifolia (L.) Griseb. (smallflower morningglory, IAQTA), Ipomoea hederacea (L.) Jacq. (ivyleaf morningglory, IPOHE), Ipomoea lacunosa L. (white morningglory, IPOLA), Lactuca serriola L./Torn. (prickly lettuce, LACSE), Portulaca oleracea L. (common purslane, POROL), Sida spinosa L. (prickly Sida, SIDSP), Sinapis arvensis L. (wild mustard, SINAR), Solanum ptychanthum Dunal (eastern black nightshade, SOLPT), or Xanthium strumarium L. (common cocklebur, XANST).

In some aspects, application rates of about 1 to about 4,000 grams/hectare (g/ha) are employed in post-emergence operations. In some aspects, rates of about 1 to about 4,000 g/ha are employed in pre-emergence operations.

In some aspects, the compounds, compositions, and methods provided herein are used in conjunction with one or more other herbicides to control a wider variety of undesirable vegetation. When used in conjunction with other herbicides, the presently claimed compounds can be formulated with the other herbicide or herbicides, tank mixed with the other herbicide or herbicides or applied sequentially with the other herbicide or herbicides. Some of the herbicides that can be employed in conjunction with the compounds of the present disclosure include: 4-CPA, 4-CPB, 4-CPP, 2,4-D, 2,4-D choline salt, 2,4-D esters and amines, 2,4-DB, 3,4-DA, 3,4-DB, 2,4-DEB, 2,4-DEP, 3,4-DP, 2,3,6-TBA, 2,4,5-T, 2,4,5-TB, acetochlor, acifluorfen, aclonifen, acrolein, alachlor, allidochlor, alloxydim, allyl alcohol, alorac, ametridione, ametryn, amibuzin, amicarbazone, amidosulfuron, aminocyclopyrachlor, aminopyralid, amiprofos-methyl, amitrole, ammonium sulfamate, anilofos, anisuron, asulam, atraton, atrazine, azafenidin, azimsulfuron, aziprotryne, barban, BCPC, beflubutamid, benazolin, bencarbazone, benfluralin, benfuresate, bensulfuron-methyl, bensulide, benthiocarb, bentazon-sodium, benzadox, benzfendizone, benzipram, benzobicyclon, benzofenap, benzofluor, benzoylprop, benzthiazuron, bicyclopyrone, bifenox, bilanafos, bispyribac-sodium, bixlozone, borax, bromacil, bromobonil, bromobutide, bromofenoxim, bromoxynil, brompyrazon, butachlor, butafenacil, butamifos, butenachlor, buthidazole, buthiuron, butralin, butroxydim, buturon, butylate, cacodylic acid, cafenstrole, calcium chlorate, calcium cyanamide, cambendichlor, carbasulam, carbetamide, carboxazole, chlorprocarb, carfentrazone-ethyl, CDEA, CEPC, chlomethoxyfen, chloramben, chloranocryl, chlorazifop, chlorazine, chlorbromuron, chlorbufam, chloreturon, chlorfenac, chlorfenprop, chlorflurazole, chlorflurenol, chloridazon, chlorimuron, chlomitrofen, chloropon, chlorotoluron, chloroxuron, chloroxynil, chlorpropham, chlorsulfuron, chlorthal, chlorthiamid, cinidon-ethyl, cinmethylin, cinosulfuron, cisanilide, clethodim, cliodinate, clodinafop-propargyl, clofop, clomazone, clomeprop, cloprop, cloproxydim, clopyralid, cloransulam-methyl, CMA, copper sulfate, CPMF, CPPC, credazine, cresol, cumyluron, cyanatryn, cyanazine, cycloate, cyclopyranil, cyclosulfamuron, cycloxydim, cycluron, cyhalofop-butyl, cyperquat, cyprazine, cyprazole, cypromid, daimuron, dalapon, dazomet, delachlor, desmedipham, desmetryn, di-allate, dicamba, dichlobenil, dichloralurea, dichlormate, dichlorprop, dichlorprop-P, diclofop, diclosulam, diethamquat, diethatyl, difenopenten, difenoxuron, difenzoquat, diflufenican, diflufenzopyr, dimefuron, dimepiperate, dimethachlor, dimethametryn, dimethenamid, dimethenamid-P, dimexano, dimidazon, dinitramine, dinofenate, dinoprop, dinosam, dinoseb, dinoterb, diphenamid, dipropetryn, diquat, disul, dithiopyr, diuron, DMPA, DNOC, DSMA, EBEP, eglinazine, endothal, epronaz, EPTC, erbon, esprocarb, ethalfluralin, ethbenzamide, ethametsulfuron, ethidimuron, ethiolate, ethobenzamid, etobenzamid, ethofumesate, ethoxyfen, ethoxysulfuron, etinofen, etnipromid, etobenzanid, EXD, fenasulam, fenoprop, fenoxaprop, fenoxaprop-P-ethyl, fenoxaprop-P-ethyl+isoxadifen-ethyl, fenoxasulfone, fenteracol, fenthiaprop, fentrazamide, fenuron, ferrous sulfate, flamprop, flamprop-M, flazasulfuron, florasulam, florpyrauxifen-benzyl, fluazifop, fluazifop-P-butyl, fluazolate, flucarbazone, flucetosulfuron, fluchloralin, flufenacet, flufenican, flufenpyr-ethyl, flumetsulam, flumezin, flumiclorac-pentyl, flumioxazin, flumipropyn, fluometuron, fluorodifen, fluoroglycofen, fluoromidine, fluoronitrofen, fluothiuron, flupoxam, flupropacil, flupropanate, flupyrsulfuron, fluridone, flurochloridone, fluroxypyr, flurtamone, fluthiacet, fomesafen, foramsulfuron, fosamine, furyloxyfen, glufosinate, glufosinate-ammonium, glyphosate, halauxifen-methyl, halosafen, halosulfuron-methyl, haloxydine, haloxyfop-methyl, haloxyfop-P-methyl, hexachloroacetone, hexaflurate, hexazinone, imazamethabenz, imazamox, imazapic, imazapyr, imazaquin, imazethapyr, imazosulfuron, indanofan, indaziflam, iodobonil, iodomethane, iodosulfuron, iofensulfuron, ioxynil, ipazine, ipfencarbazone, iprymidam, isocarbamid, isocil, isomethiozin, isonoruron, isopolinate, isopropalin, isoproturon, isouron, isoxaben, isoxachlortole, isoxaflutole, isoxapyrifop, karbutilate, ketospiradox, lancotrione, lactofen, lenacil, linuron, MAA, MAMA, MCPA esters and amines, MCPA-thioethyl, MCPB, mecoprop, mecoprop-P, medinoterb, mefenacet, mefluidide, mesoprazine, mesosulfuron, mesotrione, metam, metamifop, metamitron, metazachlor, metazosulfuron, metflurazon, methabenzthiazuron, methalpropalin, methazole, methiobencarb, methiozolin, methiuron, methometon, methoprotryne, methyl bromide, methyl isothiocyanate, methyldymron, metobenzuron, metobromuron, metolachlor, metosulam, metoxuron, metribuzin, metsulfuron, molinate, monalide, monisouron, monochloroacetic acid, monolinuron, monuron, morfamquat, MSMA, naproanilide, napropamide, napropamide-M, naptalam, neburon, nicosulfuron, nipyraclofen, nitralin, nitrofen, nitrofluorfen, norflurazon, noruron, OCH, orbencarb, ortho-dichlorobenzene, orthosulfamuron, oryzalin, oxadiargyl, oxadiazon, oxapyrazon, oxasulfuron, oxaziclomefone, oxyfluorfen, paraflufen-ethyl, parafluron, paraquat, pebulate, pelargonic acid, pendimethalin, penoxsulam, pentachlorophenol, pentanochlor, pentoxazone, perfluidone, pethoxamid, phenisopham, phenmedipham, phenmedipham-ethyl, phenobenzuron, phenylmercury acetate, picloram, picolinafen, pinoxaden, piperophos, potassium arsenite, potassium azide, potassium cyanate, pretilachlor, primisulfuron-methyl, procyazine, prodiamine, profluazol, profluralin, profoxydim, proglinazine, prohexadione-calcium, prometon, prometryn, propachlor, propanil, propaquizafop, propazine, propham, propisochlor, propoxycarbazone, propyrisulfuron, propyzamide, prosulfalin, prosulfocarb, prosulfuron, proxan, prynachlor, pydanon, pyraclonil, pyraflufen, pyrasulfotole, pyrazogyl, pyrazolynate, pyrazosulfuron-ethyl, pyrazoxyfen, pyribenzoxim, pyributicarb, pyriclor, pyridafol, pyridate, pyriftalid, pyriminobac, pyrimisulfan, pyrithiobac-methyl, pyroxasulfone, pyroxsulam, quinclorac, quinmerac, quinoclamine, quinonamid, quizalofop, quizalofop-P-ethyl, rhodethanil, rimsulfuron, saflufenacil, S-metolachlor, sebuthylazine, secbumeton, sethoxydim, siduron, simazine, simeton, simetryn, SMA, sodium arsenite, sodium azide, sodium chlorate, sulcotrione, sulfallate, sulfentrazone, sulfometuron, sulfosate, sulfosulfuron, sulfuric acid, sulglycapin, swep, TCA, tebutam, tebuthiuron, tefuryltrione, tembotrione, tepraloxydim, terbacil, terbucarb, terbuchlor, terbumeton, terbuthylazine, terbutryn, tetrafluron, thenylchlor, thiazafluron, thiazopyr, thidiazimin, thidiazuron, thiencarbazone-methyl, thifensulfuron, thiobencarb, tiocarbazil, tioclorim, tolypyralate, topramezone, tralkoxydim, triafamone, tri-allate, triasulfuron, triaziflam, tribenuron, tricamba, triclopyr esters and amines, tridiphane, trietazine, trifloxysulfuron, trifludimoxazin, trifluralin, triflusulfuron, trifop, trifopsime, trihydroxytriazine, trimeturon, tripropindan, tritac, tritosulfuron, vernolate and xylachlor.

The compounds and compositions of the present disclosure can generally be employed in combination with one or more herbicide safeners, such as AD-67 (MON 4660), benoxacor, benthiocarb, brassinolide, cloquintocet (e.g., mexyl), cyometrinil, daimuron, dichlormid, dicyclonon, dimepiperate, disulfoton, fenchlorazole-ethyl, fenclorim, flurazole, fluxofenim, furilazole, harpin proteins, isoxadifen-ethyl, jiecaowan, jiecaoxi, mefenpyr-diethyl, mephenate, MG-191, naphthalic anhydride (NA), oxabetrinil, R29148 and N-phenylsulfonylbenzoic acid amides, to enhance their selectivity.

In some aspects, the compositions and methods described herein can be used in combination with one or more seed treatments known to be employed in the safening of rice and compounds of formula (I), including naphthalic anhydride and CAS registry number 129531-12-0 (N-(2-methoxybenzoyl)-4-[(methylaminocarbonyl)amino]benzenesulfonamide or metcamifen), which has the following structure,

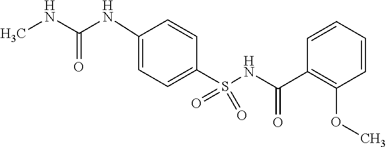

and CAS registry number 98967-94-3 (methyl 3-((5,7-dimethyl-[1,2,4]triazolo[1,5-a]pyrimidine)-2-sulfonamido)thiophene-2-carboxylate), which has the following structure,

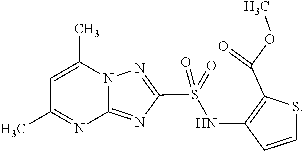 [SMILES: COC(=O)c1sccc1NS(=O)(=O)c1nc2nc(C)cc(C)n2n1]

The compounds, compositions, and methods described herein be used to control undesirable vegetation on glyphosate-tolerant-, glufosinate-tolerant-, dicamba-tolerant-phenoxy auxin-tolerant-, pyridyloxy auxin-tolerant-, aryloxyphenoxypropionate-tolerant-, acetyl CoA carboxylase (ACCase) inhibitor-tolerant-, imidazolinone-tolerant-, acetolactate synthase (ALS) inhibitor-tolerant-, 4-hydroxyphenyl-pyruvate dioxygenase (HPPD) inhibitor-tolerant-, protoporphyrinogen oxidase (PPO) inhibitor-tolerant-, triazine-tolerant-, and bromoxynil-tolerant-crops (such as, but not limited to, soybean, cotton, canola/oilseed rape, rice, cereals, corn, turf, etc), for example, in conjunction with glyphosate, glufosinate, dicamba, phenoxy auxins, pyridyloxy auxins, aryloxyphenoxypropionates, ACCase inhibitors, imidazolinones, ALS inhibitors, HPPD inhibitors, PPO inhibitors, triazines, and bromoxynil. The compositions and methods may be used in controlling undesirable vegetation in crops possessing multiple or stacked traits conferring tolerance to multiple chemistries and/or inhibitors of multiple modes-of-action.

The compounds and compositions provided herein may also be employed to control herbicide resistant or tolerant weeds. Exemplary resistant or tolerant weeds include, but are not limited to, biotypes resistant or tolerant to ALS inhibitors, photosystem II inhibitors, ACCase inhibitors, synthetic auxins, photosystem I inhibitors, 5-enolpyruvylshikimate-3-phosphate (EPSP) synthase inhibitors, microtubule assembly inhibitors, lipid synthesis inhibitors, PPO inhibitors, carotenoid biosynthesis inhibitors, very long chain fatty acid (VLCFA) inhibitors, phytoene desaturase (PDS) inhibitors, glutamine synthetase inhibitors, HPPD inhibitors, mitosis inhibitors, cellulose biosynthesis inhibitors, herbicides with multiple modes-of-action such as quinclorac, and unclassified herbicides such as arylaminopropionic acids, difenzoquat, endothall, and organoarsenicals. Exemplary resistant or tolerant weeds include, but are not limited to, biotypes with resistance or tolerance to multiple herbicides, multiple chemical classes, and multiple herbicide modes-of-action.

The described aspects and following examples are for illustrative purposes and are not intended to limit the scope of the claims. Other modifications, uses, or combinations with respect to the compositions described herein will be apparent to a person of ordinary skill in the art without departing from the spirit and scope of the claimed subject matter.

### EXAMPLES

### Example 1: Preparation of methyl 3-chloro-6-(2-chloro-4-(1-fluoroethyl)phenyl)-picolinate (F14)

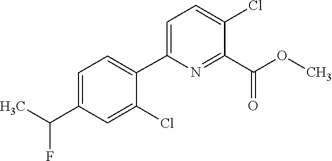 [SMILES: COC(=O)c1nc(-c2ccc(C(C)F)cc2Cl)ccc1Cl]

Methyl 3-chloro-6-(2-chloro-4-(1-hydroxyethyl)phenyl)picolinate (F1; 80 milligrams (mg), 0.246 millimoles (mmol)) was dissolved in dichloromethane (DCM; 2 mL), and the mixture was cooled to 0° C. Bis(2-methoxyethyl)aminosulfur trifluoride (Deoxo-Fluor®; 437 mg, 1.97 mmol) was added, and the reaction mixture was stirred for 3 hours (h) at room temperature. The reaction mixture was quenched with cold water and sodium bicarbonate (NaHCO₃) and extracted with ethyl acetate (EtOAc). The combined organic layers were washed with brine solution, dried over sodium sulfate (Na₂SO₄), and concentrated. Purification of the residue by medium-performance liquid chromatography (MPLC) provided the title compound as an off-white solid (55 mg, 70%).

### Example 2: Preparation of methyl 3-chloro-6-(2-chloro-4-(1-hydroxyethyl)phenyl)picolinate (F1)

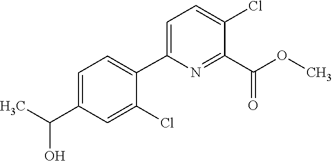 [SMILES: COC(=O)c1nc(-c2ccc(C(C)O)cc2Cl)ccc1Cl]

Methyl 6-(4-acetyl-2-chlorophenyl)-3-chloropicolinate (F26; 150 mg, 0.46 mmol) was dissolved in methanol (MeOH, 2 mL) and cooled to 0° C. Sodium borohydride (NaBH₄; 170 mg, 0.75 mmol) was added, and the mixture was stirred for 1 h at room temperature. The reaction mixture was quenched with cold water and extracted with EtOAc. The combined organic layer was washed with brine, dried over Na₂SO₄, and concentrated. Purification by MPLC afforded the title compound as an off-white solid (60 mg, 50%).

### Example 3: Preparation of methyl 6-(4-acetyl-2-chlorophenyl)-3-chloropicolinate (F26)

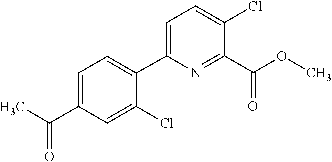 [SMILES: COC(=O)c1nc(-c2ccc(C(C)=O)cc2Cl)ccc1Cl]

Methyl 6-(4-bromo-2-chlorophenyl)-3-chloropicolinate (F27; 1 g, 2.7 mmol) was taken up in 1,2-dichloroethane (DCE; 10 mL) and degassed. Tributyl (1-ethoxyvinyl)stannane (3 g, 8.1 mmol) and dichlorobis(triphenylphosphine)palladium(II) (Pd(PPh₃)₂Cl₂; 190 mg, 0.27 mmol) were added, and the mixture was heated to 130° C. for 4 h in a sealed tube or under microwave conditions. The reaction mixture was filtered to remove the residue, and the filtrate was diluted with cold water and extracted with EtOAc. The organic layer was concentrated, the resulting crude compound was taken up in aqueous hydrochloric acid (6 normal (N) HCl), and the mixture was stirred overnight. The residue was removed by filtration, and the filtrate was diluted with cold water. The mixture was extracted with EtOAc, and the combined organic layer was washed with brine solution, dried over Na₂SO₄, and concentrated. Purification by MPLC afforded the title compound as an off-white solid (280 mg, 30%).

### Example 4: Preparation of methyl 3-chloro-6-(2-chloro-4-cyclopropylphenyl)picolinate (F15)

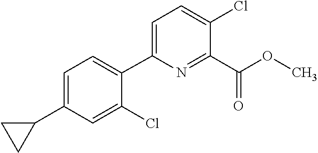 [SMILES: COC(=O)c1nc(-c2ccc(C3CC3)cc2Cl)ccc1Cl]

Methyl 6-(4-bromo-2-chlorophenyl)-3-chloropicolinate (F27; 200 mg, 0.55 mmol) was taken in toluene (4 mL) and degassed. Cyclopropyl boronic acid (96 mg, 1.1 mmol), potassium phosphate (K₃PO₄; 350 mg, 1.65 mmol) and tetrakis(triphenylphosphine)palladium(0) (Pd(PPh₃)₄; 64 mg, 0.055 mmol) were added. The reaction mixture was heated to 90° C. for about 8 h. The reaction mixture was diluted with EtOAc, and the insoluble residue was removed by filtration. The organic layer was washed with water, dried over Na₂SO₄, and concentrated. Purification by MPLC yielded the title compound as a pale yellow solid (200 mg, 40%).

### Example 5: Preparation of methyl 3-chloro-6-(3-chloro-4′-fluoro-[1, 1′-biphenyl]-4-yl) picolinate (F25)

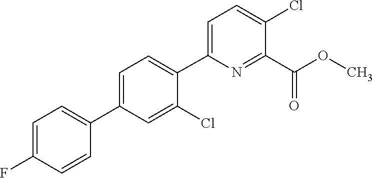 [SMILES: COC(=O)c1nc(-c2ccc(-c3ccc(F)cc3)cc2Cl)ccc1Cl]

Methyl 6-(4-bromo-2-chlorophenyl)-3-chloropicolinate (F27; 200 mg, 0.55 mmol) was dissolved in acetonitrile (CH₃CN; 6 mL) and water (2 mL). The mixture was degassed, and 4-fluorophenylboronic acid (92 mg, 0.66 mmol), potassium fluoride (KF; 96 mg, 1.65 mmol) and Pd(PPh₃)₂Cl₂ (39 mg, 0.055 mmol) were added. The reaction mixture was heated to 120° C. for ˜6 h, cooled, and filtered. The filtrate was diluted with cold water and extracted with EtOAc. The combined organic layers were washed with brine solution, dried over Na₂SO₄, and concentrated. Purification by MPLC afforded the title compound as an off-white solid (90 mg, 40%).

### Example 6: Preparation of methyl 3-chloro-6-(2-chloro-4-(trifluoromethyl)phenyl)-4-ethynylpicolinate (F40)

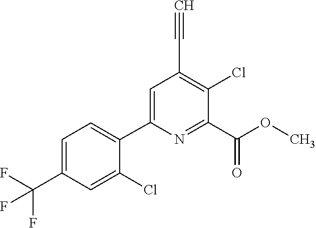 [SMILES: C#Cc1cc(-c2ccc(C(F)(F)F)cc2Cl)nc(C(=O)OC)c1Cl]

To a solution of methyl 4-bromo-3-chloro-6-(2-chloro-4-(trifluoromethyl)phenyl)picolinate (F41; 0.2 g, 0.46 mmol) in tetrahydrofuran (THF) was added triphenylphosphine (0.003 g, 0.012 mmol) and triethylamine (Et₃N; 0.069 g, 0.69 mmol). The reaction mixture was purged with argon for 15 minutes (min). Trimethylsilylacetylene (0.067 g, 0.69 mmol), copper iodide (0.002 g, 0.009 mmol) and Pd(PPh₃)₂Cl₂ (0.016 g, 0.023 mmol) were added, and the reaction mixture was stirred at room temperature for about 6 h. The reaction mixture was filtered through Celite®, and the filtrate was concentrated under reduced pressure. The resulting black residue was dissolved in dry THF (5 mL) and cooled to 0° C. Tetra-n-butylammonium fluoride (TBAF; 0.5 mL, 0.53 mmol) was added, and the mixture was stirred at 0° C. for 30 min. The reaction mixture was diluted with water and extracted with EtOAc. The organic layer was washed with brine solution, dried over Na₂SO₄, and concentrated. Purification by flash chromatography using 10% EtOAc in hexane as eluent provided the title compound as a white solid (0.04 g, 23%).

### Example 7: Preparation of methyl 3-chloro-6-(2-ethynyl-4-(trifluoromethyl) phenyl) picolinate (F16)

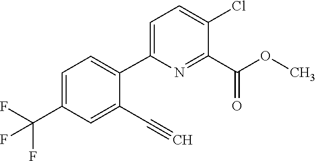 [SMILES: C#Cc1cc(C(F)(F)F)ccc1-c1ccc(Cl)c(C(=O)OC)n1]

TBAF (0.32 g, 5.4 mmol) was added to a solution of methyl 3-chloro-6-(4-(trifluoromethyl)-2-((trimethylsilyl)ethynyl)phenyl)picolinate (C1; 0.17 g, 0.41 mmol) in THF (4 mL) at 0° C. Stirring was continued at room temperature for 1 h. The reaction mixture was diluted with ice cold water and was extracted with EtOAc. The combined organic layer was dried over Na₂SO₄, and concentrated. Purification by MPLC yielded the title compound as pale brown solid (30 mg, 30%).

### Example 8 Preparation of methyl 3-chloro-6-(4-(trifluoromethyl)-2-((trimethylsilyl)ethynyl)phenyl)picolinate (C1)

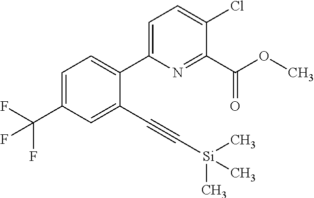 [SMILES: COC(=O)c1nc(-c2ccc(C(F)(F)F)cc2C#C[Si](C)(C)C)ccc1Cl]

Methyl 6-(2-bromo-4-trifluoromethyl)phenyl-3-chloropicolinate (F30; 0.4 g, 1.01 mmol) was taken up in THF (5 mL) and degassed. Trimethylsilylacetylene (0.2 g, 2.05 mmol), copper iodide (19 mg, 0.101 mmol), Pd(PPh₃)₂Cl₂ (70 mg, 0.1 mmol), and Et₃N (4 mL). were added, and the reaction mixture was stirred for 10 min at room temperature and heated to 70° C. for about 2 h. The reaction mixture was filtered over a bed of Celite®. Ice cold water was added to the filtrate, and the mixture was extracted with EtOAc. The combined organic layer was dried over Na₂SO₄, and concentrated. Purification by MPLC afforded the title compound as a pale brown solid (170 mg, 40%).: ¹H NMR (300 MHz, CDCl₃) δ 8.15 (d, J=8.54 Hz, 1H), 7.93 (d, J=8.16 Hz, 1H), 7.86-7.82 (m, 1H), 7.69-7.62 (m, 1H), 7.33 (d, J=2.08 Hz, 1H), 4.02 (s, 3H), 0.20 (s, 9H).

The following compound was prepared in like manner to the procedure outlined in Example 8:

### Methyl 3-chloro-6-(2-chloro-4-((trimethylsilyl)ethynyl)phenyl)picolinate (C2)

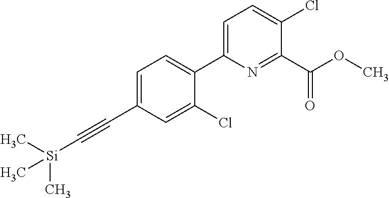

Using the appropriate starting materials, the title compound was isolated: ¹H NMR (400 MHz, CDCl₃) δ 8.15 (d, J=8.32 Hz, 1H), 7.93 (d, J=8.25 Hz, 1H), 7.86-7.82 (m, 1H), 7.66 (dd, J=1.88, 8.20 Hz, 1H), 7.36-7.28 (m, 1H), 4.03 (s, 3H), 0.20 (s, 9H).

### Example 9: Preparation of methyl 3-chloro-6-(2-chloro-4-(difluoromethyl)phenyl)picolinate (F19)

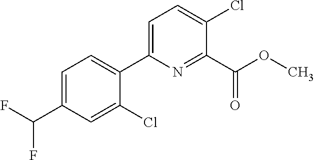 [SMILES: COC(=O)c1nc(-c2ccc(C(F)F)cc2Cl)ccc1Cl]

Methyl 3-chloro-6-(2-chloro-4-formylphenyl)picolinate (F22; 150 mg, 0.48 mmol) was taken up in DCM (2 mL) and the mixture was cooled to 0° C. Diethylaminosulfur trifluoride (DAST; 390 mg, 2.42 mmol) was added, and the mixture was stirred for 3 h at room temperature. The reaction mixture was quenched with cold water and NaHCO₃ and extracted with EtOAc. The combined organic layer was washed with brine, dried over Na₂SO₄, and concentrated to get the crude compound. Purification by MPLC afforded the title compound as an off-white solid (85 mg, 60%).

### Example 10: Preparation of methyl 3-chloro-6-(2-chloro-4-((2,2-dimethylhydrazono) methyl)phenyl) picolinate (F20)

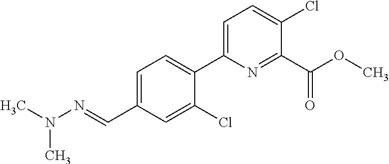

Methyl 3-chloro-6-(2-chloro-4-formylphenyl)picolinate (F22; 100 mg, 0.32 mmol) was taken up in DMF (2 mL). N,N-Dimethylhydrazine (81 mg, 0.96 mmol) was added and the reaction mixture was heated to 60° C. for about 2 h. The reaction mixture was diluted with cold water and extracted with EtOAc. The combined organic layer was washed with brine, dried over Na₂SO₄, and concentrated. Purification by MPLC provided the title compound as an off-white solid (55 mg, 50%).

### Example 11: Preparation of methyl 3-chloro-6-(2-chloro-4-((methoxyimino)methyl)phenyl)picolinate (F21)

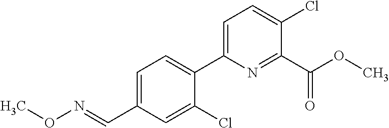

Methyl 3-chloro-6-(2-chloro-4-formylphenyl)picolinate (F22; 140 mg, 0.45 mmol) was taken up in DMF (2 mL). Methoxylamine hydrochloride (192 mg, 2.30 mmol) was added and the reaction mixture was heated to 60° C. for about 2 h. The reaction mixture was diluted with cold water and extracted with EtOAc. The combined organic layer was washed with brine, dried over Na₂SO₄, and concentrated. Purification by MPLC afforded the title compound as an off-white solid (110 mg, 70%).

### Example 12: Preparation of methyl 3-chloro-6-(2-chloro-4-formylphenyl)picolinate (F22)

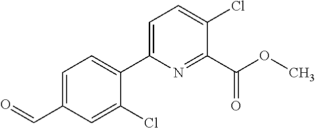 [SMILES: COC(=O)c1nc(-c2ccc(C=O)cc2Cl)ccc1Cl]

Methyl 3-chloro-6-(2-chloro-4-vinylphenyl)picolinate (F23; 800 mg, 1.61 mmol) was dissolved in 9:1 DCM-MeOH (10 mL) at −78° C. and purged with ozone gas for 30 min. The reaction mixture was allowed to warm to 0° C., and dimethyl sulfide (DMS; 4 mL) was added. The reaction mixture was stirred for 1 h, and cold water was added. The mixture was extracted with EtOAc, and the combined organic layer was washed with brine, dried over Na₂SO₄, and concentrated. Purification by MPLC provided the title compound as an off-white solid (600 mg, 65%).

### Example 13: Preparation of methyl 3-chloro-6-(2-chloro-4-(2,2 difluorocyclopropyl)phenyl) picolinate (F13)

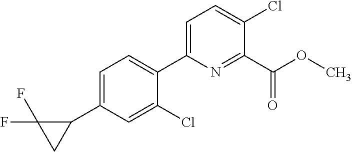 [SMILES: COC(=O)c1nc(-c2ccc(C3CC3(F)F)cc2Cl)ccc1Cl]

Methyl 3-chloro-6-(2-chloro-4-vinylphenyl)picolinate (F23; 250 mg, 0.80 mmol) was dissolved in CH₃CN, and sodium iodide (600 mg, 8.0 mmol) and trimethylsilyl trifluoromethane (560 mg, 8.0 mmol) were added. The reaction mixture was heated to 60° C. for about 6 h. The reaction mixture was diluted with cold water and extracted with EtOAc. The combined organic layer was washed with brine, dried over Na₂SO₄, and concentrated. Purification by MPLC afforded the title compound as an off-white solid (110 mg, 55%).

### Example 14: Preparation of methyl 3-chloro-6-(2-chloro-4-vinylphenyl)picolinate (F23)

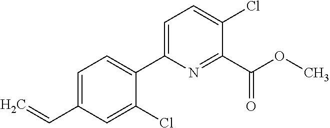 [SMILES: C=Cc1ccc(-c2ccc(Cl)c(C(=O)OC)n2)c(Cl)c1]

Methyl 6-(4-bromo-2-chlorophenyl)-3-chloropicolinate (F27; 250 mg, 0.69 mmol) was taken up in dimethyl sulfoxide (4 mL). Potassium carbonate (280 mg, 2.06 mmol) was added, and the mixture was degassed for 10 min. Trifluorovinyl borate potassium salt (285 mg, 2.86 mmol) and Pd(PPh₃)₂Cl₂ (50 mg, 0.069 mmol) were added, and the reaction mixture was heated to 90° C. for about 6 h. The mixture was filtered, and the filtrate was diluted with cold water and extracted with EtOAc. The combined organic layer was washed with brine, dried over Na₂SO₄, and concentrated. Purification by MPLC yielded the title compound as an off-white solid (160 mg, 55%).

### Example 15: Preparation of methyl 3-chloro-6-(2-chloro-4-cyanophenyl)picolinate (F59)

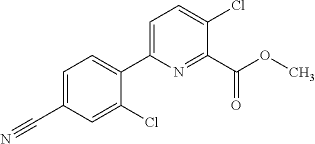 [SMILES: COC(=O)c1nc(-c2ccc(C#N)cc2Cl)ccc1Cl]

Methyl 6-(4-bromo-2-chlorophenyl)-3-chloropicolinate (F27; 200 mg, 0.55 mmol) was dissolved in DMF (4 mL). The mixture was degassed, and zinc cyanide (130 mg, 1.11 mmol) and Pd(PPh₃)₂Cl₂ (60 mg, 0.055 mmol) were added. The reaction mixture was heated to 150° C. for about 6 h, cooled, and filtered. The filtrate was diluted with cold water and extracted with EtOAc. The combined organic layer was washed with brine, dried over Na₂SO₄, and concentrated. Purification by MPLC provided the title compound as an off-white solid (70 mg, 40%).

### Example 16: Preparation of methyl 3-chloro-6-(2-chloro-4-(trifluoromethyl)phenyl)-4-methylpicolinate (F33)

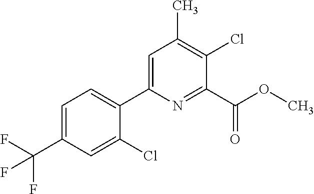 [SMILES: COC(=O)c1nc(-c2ccc(C(F)(F)F)cc2Cl)cc(C)c1Cl]

To a solution of methyl 4-bromo-3-chloro-6-(2-chloro-4-(trifluoromethyl)phenyl)picolinate (F41; 0.4 g, 0.93 mmol) in dry toluene (10 mL) were added methylboronic acid (0.08 g, 1.4 mmol) and potassium phosphate (0.63 g, 2.8 mmol) at room temperature. Argon was purged through the reaction mixture for 15 min. Pd(PPh₃)₄ (0.11 g, 0.093 mmol) was added, and the reaction mixture was heated at 100° C. for 12 h. Water was added, and the reaction mixture was extracted with EtOAc. The combined organic layer was washed with brine, dried over Na₂SO₄, and concentrated. Purification by flash chromatography with 10% EtOAc in hexane as eluent afforded the title compound as a white solid (0.16 g, 47%).

### Example 17: Preparation of methyl 3-chloro-6-(2-chloro-4-(trifluoromethyl)phenyl)-5-fluoro-4-methoxypicolinate (F42)

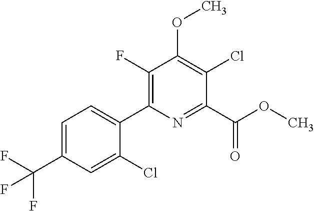 [SMILES: COC(=O)c1nc(-c2ccc(C(F)(F)F)cc2Cl)c(F)c(OC)c1Cl]

To a solution of methyl 3,4-dichloro-6-(2-chloro-4-(trifluoromethyl)phenyl)-5-fluoropicolinate (F43; 0.18 g, 0.45 mmol) in dry MeOH (2 mL) was added a freshly prepared sodium methoxide solution (0.029 g, 0.53 mmol) in MeOH at 0° C., and the mixture was allowed to stir at room temperature for 2 h. The reaction mixture was cooled to 0° C., quenched with 1 N HCl, and extracted with EtOAc. The organic layer was washed with brine, dried over Na₂SO₄, and concentrated. Purification by flash chromatography with 10% EtOAc in hexane as eluent provided the title compound as a white solid (0.06 g, 33%).

### Example 18: Preparation of methyl 4-amino-3-chloro-6-(2-chloro-4-(trifluoromethyl)phenyl)-5-(trifluoromethyl)picolinate (C3)

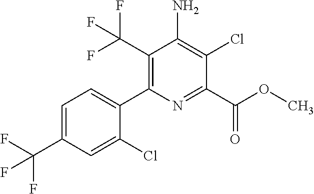 [SMILES: COC(=O)c1nc(-c2ccc(C(F)(F)F)cc2Cl)c(C(F)(F)F)c(N)c1Cl]

A solution of methyl 4-amino-3-chloro-6-(2-chloro-4-(trifluoromethyl)phenyl)-5-iodopicolinate (C5; 0.2 g, 0.41 mmol) in dry DMF was purged with argon for about 10 min. Methyl-2,2-difluoro-2-(fluorosulfonyl)acetate (0.3 g, 1.6 mmol) and copper(I) iodide (CuI; 0.015 g, 0.08 mmol) were added, and the reaction mixture was heated in a Biotage microwave reactor for 1 h at 100° C. Water was added to the reaction mixture, and the mixture was extracted with EtOAc. The organic layer was washed with brine, dried over Na₂SO₄, and concentrated. Purification by flash chromatography with 10% EtOAc in hexane as eluent yielded the title compound as a white solid (0.08 g, 46%).

### Example 19: Preparation of methyl 3-chloro-6-(2-chloro-4-(trifluoromethyl)phenyl)-5-(methylthio)picolinate (F7)

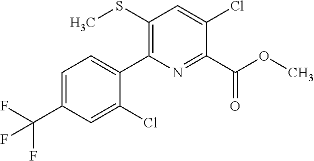 [SMILES: COC(=O)c1nc(-c2ccc(C(F)(F)F)cc2Cl)c(SC)cc1Cl]

To a solution of methyl 4-amino-3-chloro-6-(2-chloro-4-(trifluoromethyl)phenyl)-5-(methylthio)picolinate (C4; 0.12 g, 0.3 mmol) in dry THF (5 mL) was added isoamylnitrite (0.08 g, 0.7 mmol) at room temperature. The resulting mixture was heated at 60° C. for 12 h. The reaction mixture was cooled to room temperature and partitioned between water and EtOAc. The organic layer was washed with brine, dried over Na₂SO₄, and concentrated. Purification by flash chromatography with 5% EtOAc in hexane as eluent provided the title compound as a white solid (0.08 g, 67%).

### Example 20: Preparation of methyl 3-chloro-6-(2-chloro-4-(trifluoromethyl)phenyl)-5-hydroxypicolinate (F34)

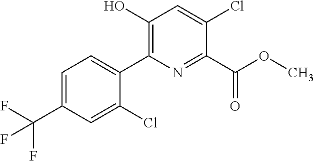 [SMILES: COC(=O)c1nc(-c2ccc(C(F)(F)F)cc2Cl)c(O)cc1Cl]

To a solution of methyl 4-amino-3-chloro-6-(2-chloro-4-(trifluoromethyl)phenyl)-5-fluoropicolinate (C6; 0.35 g, 0.91 mmol) in ethanol (EtOH; 10 mL) were added toluene (3 mL) and sulfuric acid (1.5 mL), and the resulting mixture was cooled to 0° C. Solid sodium nitrite (1.25 g, 18.1 mmol) was added, and the mixture was heated at 80° C. for 3 h. The solvent was removed from the reaction mixture, the residue was neutralized with NaHCO₃. The mixture was extracted with EtOAc. The organic layer was washed with brine, dried over Na₂SO₄, and concentrated. Purification by preparative high-performance liquid chromatography (HPLC) afforded the title compound as a white solid (0.15 g, 53%).

### Example 21: Preparation of methyl 3,4-dichloro-6-(4-chloro-2-fluoro-3-methoxyphenyl)picolinate (F76)

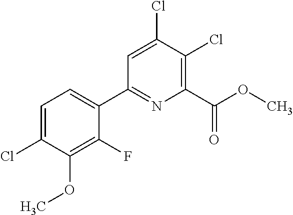 [SMILES: COC(=O)c1nc(-c2ccc(Cl)c(OC)c2F)cc(Cl)c1Cl]

Sodium nitrite (210 mg, 3.0 mmol) was dissolved in a minimum amount (˜1 mL) of water and added dropwise to a stirred suspension of 4-amino-3-chloro-6-(4-chloro-2-fluoro-3-methoxyphenyl)picolinic acid (prepared as in Balko, et al., U.S. Pat. No. 7,314,849 B2; 200 mg, 0.60 mmol) in concentrated HCl (2.0 mL) at 0° C. The resulting thick heterogeneous yellow mixture was immediately allowed to warm to room temperature and stirred for 20 h. The reaction mixture was vacuum filtered and rinsed repeatedly with water to afford the title compound as a tan powder (170 mg, 81%).

### Example 22: Preparation of methyl 3,4-dichloro-6-(2-chloro-4-(trifluoromethyl)phenyl)-5-fluoropicolinate (F43)

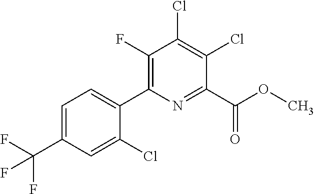 [SMILES: COC(=O)c1nc(-c2ccc(C(F)(F)F)cc2Cl)c(F)c(Cl)c1Cl]

To a solution of copper(II) chloride (0.12 g, 0.94 mmol) in CH₃CN (5 mL) was added tert-butyl nitrite (0.12 g, 1.17 mmol) at room temperature. The mixture was stirred for 15 min and cooled to 0° C. A solution of methyl 4-amino-3-chloro-6-(2-chloro-4-(trifluoromethyl)phenyl)-5-fluoropicolinate (C6; 0.3 g, 0.78 mmol) in CH₃CN was added slowly, and the reaction mixture was stirred at 0° C. for 1 h and at room temperature for 12 h. The reaction mixture was diluted with water and extracted with EtOAc. The organic layer was washed with brine, dried over Na₂SO₄, and concentrated. Purification by flash chromatography with 10% EtOAc in hexane as eluent yielded the title compound as a white solid (0.25 g, 80%): mp 86-88° C.; ¹H NMR (300 MHz, CDCl₃) δ 7.78 (s, 1H), 7.69-7.64 (m, 1H), 7.61 (d, J=8.05 Hz, 1H), 4.01 (s, 3H); ESIMS m/z 402 ([M+H]⁺).

### Example 23: Preparation of methyl 4-amino-3-chloro-6-(2-chloro-4-(trifluoromethyl)phenyl)-5-(methylthio)picolinate (C4)

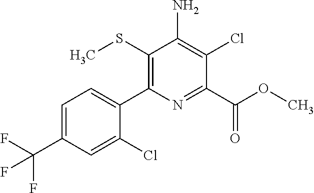 [SMILES: COC(=O)c1nc(-c2ccc(C(F)(F)F)cc2Cl)c(SC)c(N)c1Cl]

To a solution of methyl 4-amino-3-chloro-6-(2-chloro-4-(trifluoromethyl)phenyl)-5-fluoropicolinate (C6; 0.2 g, 0.52 mmol) in dry DMF (2 mL) was added sodium thiomethoxide (0.036 g, 0.52 mmol) at room temperature. The mixture was heated to 55° C. for 2 h, cooled to room temperature and partitioned between water and EtOAc. The organic layer was washed with saturated (satd) ammonium chloride (NH₄Cl) solution and brine, dried over Na₂SO₄, and concentrated. Purification by flash chromatography with 10% EtOAc in hexane as eluent provided the title compound as a colorless oil (0.12 g, 57%): ¹H NMR (300 MHz, CDCl₃) δ 7.72 (d, J=1.65 Hz, 1H), 7.62-7.56 (m, 1H), 7.46 (d, J=8.01 Hz, 1H), 5.74 (s, 2H), 3.96 (s, 3H), 2.07 (s, 3H).

### Example 24: Preparation of methyl 4-amino-3-chloro-6-(2-chloro-4-(trifluoromethyl)phenyl)-5-iodopicolinate (C5)

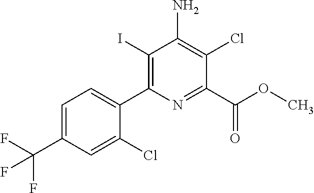 [SMILES: COC(=O)c1nc(-c2ccc(C(F)(F)F)cc2Cl)c(I)c(N)c1Cl]

To a solution of methyl 4-amino-3-chloro-6-(2-chloro-4-(trifluoromethyl)phenyl)picolinate (C7; 0.2 g, 0.55 mmol) in dry MeOH (5 mL) were added periodic acid (0.05 g, 0.22 mmol) and iodine (0.07 g, 0.55 mmol) at room temperature. The resulting brown-colored mixture was stirred at reflux (65° C.) for 12 h. A 5% sodium thiosulfate solution was added to the reaction mixture. The solid was removed by filtration and dried. The title compound was isolated as a colorless solid (0.23 g, 85%): mp 128-130° C.; ¹H NMR (300 MHz, CDCl₃) δ 7.73 (s, 1H), 7.62 (d, J=7.99 Hz, 1H), 7.43 (d, J=7.95 Hz, 1H), 5.52 (s, 2H), 3.95 (s, 3H); ESIMS m/z 491 ([M+H]⁺).

### Example 25: Preparation of benzyl 3-chloro-6-(2-chloro-4-(trifluoromethyl)phenyl)picolinate (F44)

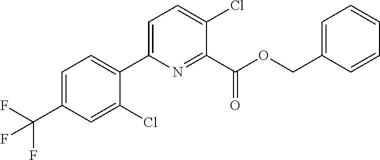 [SMILES: O=C(OCc1ccccc1)c1nc(-c2ccc(C(F)(F)F)cc2Cl)ccc1Cl]

A mixture of 3-chloro-6-(2-chloro-4-(trifluoromethyl)phenyl)picolinic acid (F61; 0.205 g, 0.610 mmol), potassium carbonate (0.126 g, 0.915 mmol) and benzyl bromide (0.087 mL, 0.732 mmol) in DMF (3.1 mL) was stirred at room temperature overnight. The reaction mixture was poured into a satd aqueous (aq) NaHCO₃ solution and extracted with EtOAc (2×). The combined organic layers were dried over magnesium sulfate (MgSO₄), filtered and concentrated. Purification by column chromatography with a hexane-EtOAc gradient afforded an oil that crystallized upon drying in vacuo. The title compound was isolated as a white solid (237 mg, 91%).

### Example 26: Preparation of prop-2-yn-1-yl 3-chloro-6-(2-chloro-4-(trifluoromethyl)phenyl)picolinate (F45)

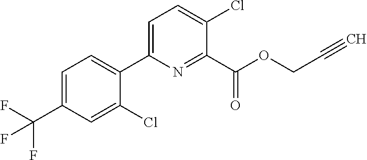 [SMILES: C#CCOC(=O)c1nc(-c2ccc(C(F)(F)F)cc2Cl)ccc1Cl]

A mixture of 3-chloro-6-(2-chloro-4-(trifluoromethyl)phenyl)picolinic acid (F61; 0.205 g, 0.610 mmol), potassium carbonate (0.126 g, 0.915 mmol) and propargyl bromide (0.082 mL, 0.732 mmol) in DMF (3.1 mL) was stirred at room temperature overnight. The reaction mixture was poured into a satd aq NaHCO₃ solution and extracted with EtOAc (2×). The combined organic layers were dried over MgSO₄, filtered and concentrated. Purification by column chromatography with a hexane-EtOAc gradient yielded an oil that crystallized using a minimum of diethyl ether (Et₂O) and drying in vacuo. The title compound was isolated as a white solid (220 mg, 96%).

### Example 27: Preparation of 3-chloro-6-(2-chloro-4-(trifluoromethyl)phenyl)picolinic acid (F61)

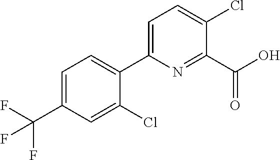 [SMILES: O=C(O)c1nc(-c2ccc(C(F)(F)F)cc2Cl)ccc1Cl]

To a solution of methyl 3-chloro-6-(2-chloro-4-(trifluoromethyl)phenyl)picolinate (F66; 1.22 g, 3.48 mmol) in THF (8.7 mL) and MeOH (8.7 mL) was added a 2 N solution of sodium hydroxide (NaOH; 5.23 mL, 10.5 mmol). The reaction mixture was stirred at room temperature overnight. The mixture was poured into a 1 N HCl solution and extracted with EtOAc (2×). The combined organic layers were dried over MgSO₄, filtered, concentrated and dried in vacuo. The title compound was isolated as a white solid (1.13 g, 96%).

### Example 28: Preparation of 3-chloro-6-(4-chloro-2-fluoro-3-methoxyphenyl)picolinic acid (F78)

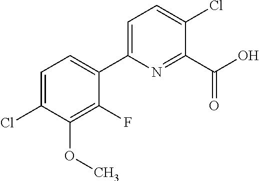 [SMILES: COc1c(Cl)ccc(-c2ccc(Cl)c(C(=O)O)n2)c1F]

To a solution of methyl 3-chloro-6-(4-chloro-2-fluoro-3-methoxyphenyl)picolinate (prepared as in Epp, J. B. et al. Bioorg. Med. Chem. 2016, 24, 362-371; 275 mg, 3.48 mmol) in MeOH (30 mL) was added a 1 N solution of NaOH (30 mL, 10.5 mmol). The reaction mixture was stirred at reflux for 1 h, was cooled, and was made acidic. The solid was collected and dried in vacuo. The title compound was isolated as a white solid (200 mg, 76%).

### Example 29: Preparation of 3,5-dichloro-6′-(trifluoromethyl)-[2,3′-bipyridine]-6-carboxylic acid (F84)

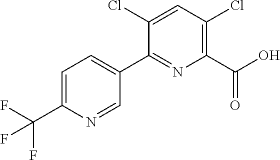 [SMILES: O=C(O)c1nc(-c2ccc(C(F)(F)F)nc2)c(Cl)cc1Cl]

To a solution of methyl 3,5-dichloro-6′-(trifluoromethyl)-[2,3′-bipyridine]-6-carboxylate (F99; 0.119 g, 0.339 mmol) in MeOH (0.7 mL) was added sodium hydroxide (1 M solution; 1.0 mL, 1.0 mmol). The solution was heated at reflux for 1 h and cooled to room temperature. The reaction mixture was made acidic with HCl and diluted with water and DCM. The phases were separated, and the organic layer was concentrated to afford the title compound as a white solid (0.093 g, 81%).

### Example 30: Preparation of methyl 5-chloro-3-hydroxy-6′-(trifluoromethyl)-[2,3′-bipyridine]-6-carboxylate (F87)

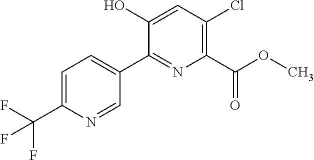 [SMILES: COC(=O)c1nc(-c2ccc(C(F)(F)F)nc2)c(O)cc1Cl]

To a solution of methyl 5-chloro-3-fluoro-6′-(trifluoromethyl)-[2,3′-bipyridine]-6-carboxylate (F92; 0.211 g, 0.671 mmol) in MeOH (1.3 mL) was added NaOH (1 M solution; 1.4 mL, 1.4 mmol). The solution was heated at reflux for 30 min and cooled to room temperature. The reaction mixture was made acidic with HCl and diluted with DCM. The biphasic mixture was passed through a phase separator, and the filtrate was concentrated to afford the title compound as a white solid (0.052 g, 90%).

### Example 31: Preparation of methyl 5-amino-3-chloro-6-(4-chlorophenyl)picolinate (F72)

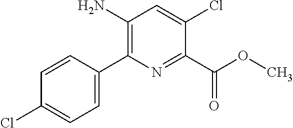 [SMILES: COC(=O)c1nc(-c2ccc(Cl)cc2)c(N)cc1Cl]

Methyl 5-acetamido-3-chloro-6-(4-chlorophenyl)picolinate (C8; 0.415 g, 1.224 mmol) was suspended in MeOH (20 mL) and acetyl chloride (1.740 mL, 24.47 mmol) was added dropwise. The reaction mixture was stirred overnight at room temperature and concentrated under vacuum. The residue was partitioned between EtOAc and 5% aq NaHCO₃ solution. The organic phase was concentrated onto silica gel. Purification by flash chromatography with an EtOAc-hexanes gradient, followed by reverse-phase HPLC, provided the title compound as a white solid (0.080 g, 22%).

### Example 32: Preparation of methyl 3-chloro-6-(2-chloro-4-(trifluoromethyl)phenyl)picolinate (F66)

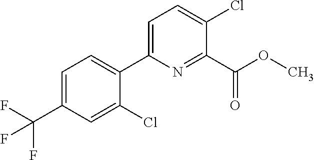 [SMILES: COC(=O)c1nc(-c2ccc(C(F)(F)F)cc2Cl)ccc1Cl]

A mixture of (2-chloro-4-(trifluoromethyl)phenyl)boronic acid (2.40 g, 10.7 mmol), methyl 3,6-dichloropicolinate (2 g, 9.71 mmol), Pd(PPh₃)₂Cl₂ (0.681 g, 0.971 mmol) and potassium fluoride (1.69 g, 29.1 mmol) in CH₃CN (29 mL) and water (9.7 mL) was stirred at reflux (˜85° C.) for 4 h. The reaction mixture was poured into a satd aq NaHCO₃ solution and extracted with EtOAc (2×). The combined organic layers were dried over MgSO₄, filtered and concentrated. Purification by flash chromatography with a hexane-EtOAc gradient provided a solid, which was triturated with Et₂O-hexane (1:9). The solid was filtered, washed with hexane and dried in vacuo. The filtrate was concentrated and the resulting solid was triturated with hexane, filtered and washed with hexane. The two solid batches were combined and dried in vacuo to afford the title compound as a white solid (1.97 g, 58%).

The following compounds were prepared in like manner to the procedure outlined in Example 32:

### Methyl 4-amino-3-chloro-6-(2-chloro-4-(trifluoromethyl)phenyl)-5-fluoropicolinate (C6)

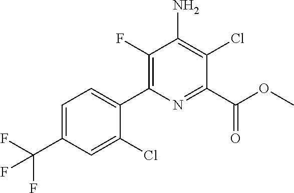 [SMILES: COC(=O)c1nc(-c2ccc(C(F)(F)F)cc2Cl)c(F)c(N)c1Cl]

Using the appropriate starting materials with heating in a sealed pressure tube at 80° C. for 12 h, the title compound was synthesized and isolated as an off-white solid (300 mg, 37%): mp 138-140° C.; ¹H NMR (300 MHz, CDCl₃) δ 7.74 (d, J=1.64 Hz, 1H), 7.67-7.55 (m, 2H), 4.99 (s, 2H), 3.97 (s, 3H); LCMS (M+1)=382.9.

### Methyl 4-amino-3-chloro-6-(2-chloro-4-(trifluoromethyl)phenyl)picolinate (C7)

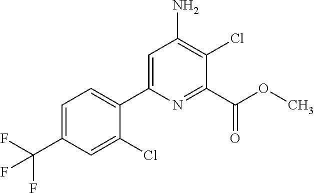 [SMILES: COC(=O)c1nc(-c2ccc(C(F)(F)F)cc2Cl)cc(N)c1Cl]

Using the appropriate starting materials with heating in a sealed pressure tube at 80° C. for 12 h, the title compound was synthesized and isolated as white foam (2 g, 30%): ¹H NMR (300 MHz, CDCl₃) δ 7.72 (d, J=8.37 Hz, 2H), 7.63-7.59 (m, 1H), 7.59-7.53 (m, 1H), 4.90 (s, 2H), 3.99 (s, 3H).

### Methyl 5-acetamido-3-chloro-6-(4-chlorophenyl)picolinate (C8)

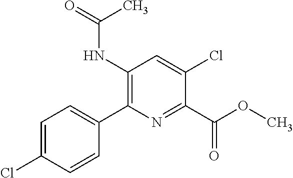 [SMILES: COC(=O)c1nc(-c2ccc(Cl)cc2)c(NC(C)=O)cc1Cl]

Using the appropriate starting materials in a 1:1 mixture of CH₃CN-water with heating in a microwave reactor at 110° C. for 20 min, the title compound was synthesized and isolated as a white solid (0.415 g, 80%): mp 146-150° C.; ¹H NMR (400 MHz, CDCl₃) δ 8.97 (s, 1H), 7.53 (d, J=1.6 Hz, 4H), 3.97 (s, 3H), 2.13 (s, 3H); ESIMS m/z 338 ([M−H]⁻).

### Example 33: Preparation of 2,4-dichlorobenzyl 3-chloro-6-(2-chloro-4-(trifluoromethyl)phenyl)picolinate (F116)

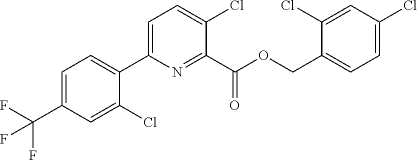 [SMILES: O=C(OCc1ccc(Cl)cc1Cl)c1nc(-c2ccc(C(F)(F)F)cc2Cl)ccc1Cl]

To a solution of 3-chloro-6-(2-chloro-4-(trifluoromethyl)phenyl)picolinic acid (F61; 0.077 g, 0.229 mmol) in DCM (2.3 mL) were added (2,4-dichlorophenyl)methanol (0.041 g, 0.229 mmol), N-(3-dimethylaminopropyl)-N′-ethylcarbodiimide hydrochloride (EDC; 0.036 g, 0.229 mmol) and 4-dimethylaminopyridine (DMAP; 0.028 g, 0.229 mmol). The reaction mixture was stirred overnight and then loaded directly onto Celite©. Purification over silica gel (0 to 10% EtOAc-hexane gradient) afforded the title compound as a clear oil (0.032 g, 28%).

### Example 34: Preparation of methyl 3-chloro-6-(4-chlorophenyl)picolinate (F82)

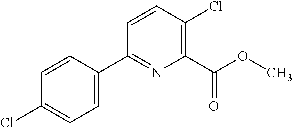 [SMILES: COC(=O)c1nc(-c2ccc(Cl)cc2)ccc1Cl]

A mixture of 4-chlorophenylboronic acid (7.0 g, 44.8 mmol), methyl 6-bromo-3-chloropicolinate (7.5 g, 29.9 mmol), dppb (2.0 g, 4.7 mmol), Et₃N (20 mL, 143 mmol). and Pd(OAc)₂ (1.0 g, 4.5 mmol) in CH₃CN was stirred at reflux overnight. The reaction mixture was poured into water and extracted with EtOAc. The combined organic extracts were washed with brine, dried over (Na₂SO₄), filtered and concentrated. Purification by flash chromatography with 4:1 hexanes-EtOAc as eluent yielded a mixture of the starting material and title compound. Purification by flash chromatography with 2:1 hexanes-DCM as eluent (2×) afforded the title compound. (1.9 g, 23%).

### Example 35: Preparation of methyl 3-chloro-6-(2-chloro-4-((hydroxyimino)methyl)phenyl)picolinate (F145)

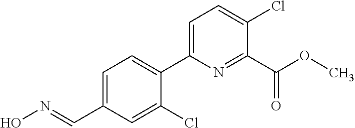 [SMILES: COC(=O)c1nc(-c2ccc(/C=N/O)cc2Cl)ccc1Cl]

To a solution of methyl 3-chloro-6-(2-chloro-4-formylphenyl)picolinate (F22; 40 mg, 0.129 mmol) in a mixture of H₂O/MeOH (5 mL, 1:2) were added in succession hydroxylamine hydrochloride (8.96 mg, 0.129 mmol) and sodium carbonate (7.52 mg, 0.071 mmol), and the mixture was stirred overnight at 25° C. The reaction mixture diluted with water (50 mL) and was extracted with Et₂O (3×100 mL). The combined organic extracts were dried over anhydrous magnesium sulfate, filtered, and concentrated. The solid was dried under vacuum. The title compound was isolated as a white solid (24 mg, 56%) as a 5:1 E/Z mixture.

### Example 36: Preparation of ethyl 3-amino-6-(4-chloro-2-fluoro-3-methoxyphenyl)-4-ethoxypicolinate (F77)

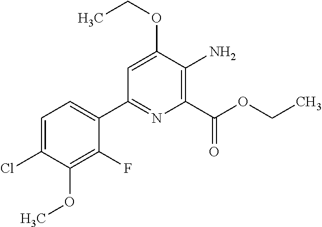 [SMILES: CCOC(=O)c1nc(-c2ccc(Cl)c(OC)c2F)cc(OCC)c1N]

To a solution of ethyl (E)-3-chloro-6-(4-chloro-2-fluoro-3-methoxyphenyl)-4-(((methylsulfonyl)oxy)imino)-1,4,5,6-tetrahydropyridine-2-carboxylate (prepared as in Renga, et al., U.S. Pat. No. 8,598,086; 200 mg, 0.440 mmol) in EtOH (1.5 mL) were added sequentially potassium carbonate (182 mg, 1.32 mmol) and sodium ethoxide (21% in EtOH; 427 mg, 1.32 mmol). The reaction mixture turned dark in color. After 10 min, the reaction mixture was quenched with 1 molar (M) HCl solution and extracted with Et₂O. The combined organic extracts were washed with brine, dried with Na₂SO₄, filtered, and concentrated. Purification by silica gel chromatography with 30% Et₂O-pentane as eluent yielded the title compound as an off-white solid (56 mg, 35%).

### Example 37: Preparation of methyl 5-chloro-2-(4-chloro-2-fluoro-3-methoxyphenyl)pyrimidine-4-carboxylate (F115)

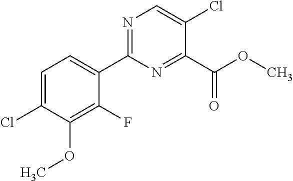 [SMILES: COC(=O)c1nc(-c2ccc(Cl)c(OC)c2F)ncc1Cl]

Methyl 5-chloro-2-(4-chloro-2-fluoro-3-methoxyphenyl)-6-(methylsulfonyl)pyrimidine-4-carboxylate (205 mg, 0.5 mmol) was dissolved in DMF (3 ML) and NaBH₄ (34 mg, 0.9 mmol) was added in 0.3 mmol portions until starting material was consumed. The reaction mixture was concentrated under vacuum and partitioned between EtOAc and water. The organic phase was dried and concentrated. Purification by flash chromatography with an EtOAc-hexanes gradient provided the title compound as a white solid (0.100 g, 60%).

### Example 38: Preparation of methyl 5-chloro-2-(4-chloro-2-fluoro-3-methoxyphenyl)-6-(methylsulfonyl)pyrimidine-4-carboxylate (C9)

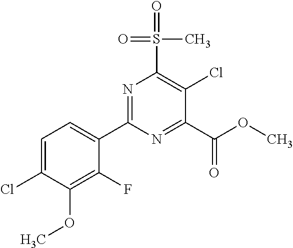 [SMILES: COC(=O)c1nc(-c2ccc(Cl)c(OC)c2F)nc(S(C)(=O)=O)c1Cl]

Methyl 5-chloro-2-(4-chloro-2-fluoro-3-methoxyphenyl)-6-(methylthio)pyrimidine-4-carboxylate (C10; 590 mg, 1.62 mmol) and meta-chloroperoxybenzoic acid (m-CPBA; 933 mg, 3.25 mmol) were combined in DCM (20 mL) and stirred at room temperature overnight. The reaction mixture was then concentrated and partitioned between EtOAc and water. The organic phase was washed with water, dried, and concentrated. Purification by flash chromatography with an EtOAc-DCM gradient provided the title compound as an impure white solid which was carried on to the next step (400 mg, 60%): ¹H NMR (300 MHz, CDCl₃) δ 7.90 (dd, J=8.8, 7.4 Hz, 1H), 7.31 (dd, J=8.6, 1.8 Hz, 1H), 4.09 (s, 3H), 4.02 (d, J=1.1 Hz, 3H), 3.52 (s, 3H); ESIMS m/z 410 ([M+H]⁺).

### Example 39: Preparation of methyl 5-chloro-2-(4-chloro-2-fluoro-3-methoxyphenyl)-6-(methylthio)pyrimidine-4-carboxylate (C10)

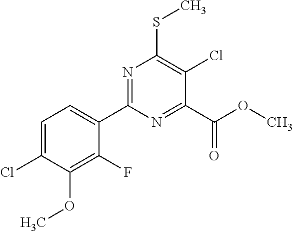 [SMILES: COC(=O)c1nc(-c2ccc(Cl)c(OC)c2F)nc(SC)c1Cl]

Methyl 2-bromo-5-chloro-6-(methylthio)pyrimidine-4-carboxylate (prepared as in Epp et al., WO2007082076A1; 3 g, 10 mmol), (4-chloro-2-fluoro-3-methoxyphenyl)boronic acid (2.46 g, 12 mmol), Pd(PPh₃)₂Cl₂ (702 mg, 1 mmol), and triethylamine (4.2 mL, 30 mmol) were combined in dioxane (20 mL) and heated at reflux for 4 h. The cooled reaction mixture was concentrated under vacuum. Purification by flash chromatography with an EtOAc-hexanes gradient provided the title compound as an impure solid which was carried on to the next step (590 mg, 16%): ¹H NMR (300 MHz, CDCl₃) δ 7.85 (dd, J=8.8, 7.4 Hz, 1H), 7.31-7.23 (m, 1H), 4.05 (s, 3H), 4.03 (d, J=1.1 Hz, 3H), 2.69 (s, 3H); ESIMS m/z 378 ([M+H]⁺).

### Example 40: Preparation of methyl 3-chloro-6-(2-chloro-4-(trifluoromethyl)phenyl)-4-fluoropicolinate (F109)

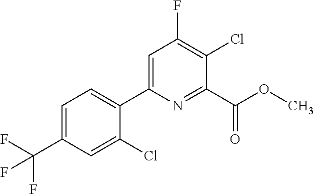 [SMILES: COC(=O)c1nc(-c2ccc(C(F)(F)F)cc2Cl)cc(F)c1Cl]

Cesium fluoride (160 mg, 1.0 mmol) was stirred in anhydrous dimethyl sulfoxide (DMSO; 10 mL) and heated to 100° C. for 90 min. The mixture was heated under vacuum (20-50 mmHg) until 1-2 mL distillate was taken overhead. After cooling, methyl 3,4-dichloro-6-(2-chloro-4-(trifluoromethyl)phenyl)picolinate (F108; 200 mg, 0.52 mmol) was added and the mixture was heated at 100° C. for 18 h. The mixture was partitioned between water (10 mL) and EtOAc (30 mL). The organic phase was washed with water (5 mL) and brine (5 mL), dried and evaporated. Purification via silica gel chromatography using a 0-30% EtOAc-hexane gradient provided the title compound as a white solid (24 mg, 12%).

### Example 41: Preparation of methyl 3′,5-dichloro-5′-(trifluoromethyl)-[2,2′-bipyridine]-6-carboxylate (F101)

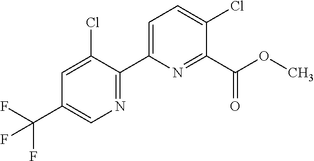 [SMILES: COC(=O)c1nc(-c2ncc(C(F)(F)F)cc2Cl)ccc1Cl]

A degassed (argon) mixture of methyl 3-chloro-6-(trimethylstannyl)picolinate (C16; 0.1 g, 0.299 mmol), 3-chloro-2-iodo-5-(trifluoromethyl)pyridine (0.11 g, 0.359 mmol), Pd(PPh₃)₂Cl₂ (0.031 g, 0.045 mmol) in DCE (1.5 mL) was stirred under microwave irradiation at 120° C. for 30 min. The reaction mixture was concentrated to a residue. Purification by silica gel column chromatography with a hexane-EtOAc gradient afforded the title compound as a yellow oil (0.069 g, 66%).

### Example 42: Preparation of 2-(2,4-dichloro-3-methylphenyl)-4,4,5,5-tetramethyl-1,3,2-dioxaborolane (C11)

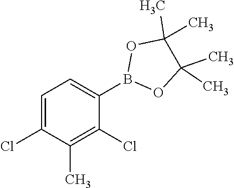 [SMILES: Cc1c(Cl)ccc(B2OC(C)(C)C(C)(C)O2)c1Cl]

To a stirred solution of 1-bromo-2,4-dichloro-3-methylbenzene (500 mg, 2.09 mmol) and 4,4,4′,4′,5,5,5′,5′-octamethyl-2,2′-bi(1,3,2-dioxaborolane) (637 mg, 2.51 mmol) in 1,4-dioxane was added potassium acetate (304 mg, 3.10 mmol), and the mixture was degassed for 10 min. [1,1′-Bis(diphenylphosphino)ferrocene]dichloropalladium(II) (Pd(dppf)Cl₂; 152 mg, 0.2 mmol) was added, and the mixture was degassed for 10 min. The mixture was heated at 90° C. for 5 h, was cooled and was diluted with EtOAc. The organic layer was washed with water and brine, dried over Na₂SO₄, and concentrated. Purification by MPLC with 5% EtOAc-Hexane as eluent afforded the title compound as a colorless liquid (430 mg): ¹H NMR (300 MHz, CDCl₃) δ 7.41 (dd, J=2.07, 8.10 Hz, 1H), 7.26-7.23 (m, 1H), 2.47 (s, 3H), 1.37 (s, 12H). Note: The title compound obtained was impure (containing diborane) and used in next step without further purification.

The following compound was prepared in like manner to the procedure outlined in Example 42:

### 2,5-Dichloro-4-(4,4,5,5-tetramethyl-1,3,2-dioxaborolan-2-yl)benzonitrile (C12)

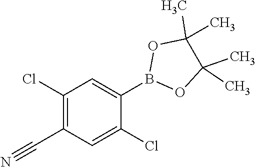 [SMILES: CC1(C)OB(c2cc(Cl)c(C#N)cc2Cl)OC1(C)C]

Using the appropriate starting materials, the title compound was synthesized and isolated: ¹H NMR (300 MHz, CDCl₃) δ 7.81 (d, J=10.5 Hz, 1H), 7.63 (d, J=6.24 Hz, 1H), 1.37 (s, 12H).

### Example 43: Preparation of 2-(2,6-dichloro-4-(trifluoromethyl)phenyl)-4,4,5,5-tetramethyl-1,3,2-dioxaborolane (C13)

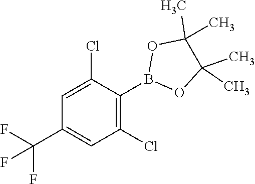 [SMILES: CC1(C)OB(c2c(Cl)cc(C(F)(F)F)cc2Cl)OC1(C)C]

In an oven-dried, nitrogen-flushed 100 mL round-bottomed flask, 1,3-dichloro-2-iodo-5-(trifluoromethyl)benzene (1.78 g, 5.22 mmol) was dissolved in THF (10 mL), and the resulting solution was cooled on an ice bath under nitrogen. Isopropylmagnesium(II) lithium chloride (1.3 M in THF; 4.42 mL, 5.74 mmol) was added dropwise with stirring over 5 min. After 1 h, 2-isopropoxy-4,4,5,5-tetramethyl-1,3,2-dioxaborolane (1.17 mL, 5.74 mmol) was added, and the reaction mixture was allowed to warm to room temperature and stir overnight. The reaction mixture was partitioned between EtOAc and satd aq NH₄Cl solution; the layers were separated; and the organic layer was dried over Na₂SO₄. Filtration and concentration under reduced pressure gave the title compound as an orange oil (1.74 g, 98%, as a 5:1 mixture with 1,3-dichloro-5-(trifluoromethyl)benzene: ¹H NMR (400 MHz, CDCl₃) δ 7.48 (s, 2H), 1.43 (s, 12H); ¹³C NMR (101 MHz, CDCl₃) δ 138.83, 133.70 (q, J=33.8 Hz), 123.71 (q, J=3.8 Hz), 122.54 (q, J=274.72 Hz), 85.45, 24.70, boron substituted carbon is too broad to be seen; ¹⁹F NMR (376 MHz, CDCl₃) δ−63.23; EIMS m/z 340.

### Example 44: Preparation of 2-(2-chloro-4-(1,1-difluoroethyl)phenyl)-4,4,5,5-tetramethyl-1,3,2-dioxaborolane (C14)

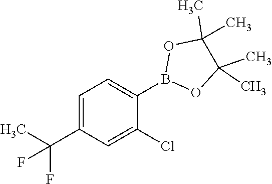 [SMILES: CC(F)(F)c1ccc(B2OC(C)(C)C(C)(C)O2)c(Cl)c1]

Step 1—Preparation of 1-bromo-2-chloro-4-(1,1-difluoroethyl)benzene: In a sealed vessel, a mixture of 1-(4-bromo-3-chlorophenyl)ethan-1-one (1 g, 4.28 mmol) and deoxofluor (3.16 mL, 17.1 mmol) was stirred at 85° C. for 2 h. The reaction mixture was poured into satd NaHCO₃ and extracted with EtOAc (2×). The combined organic layers were dried over MgSO₄, filtered, and concentrated. Purification of the residue by reverse-phase column chromatography (C₁₈) with a water-acetonitrile gradient afforded the title compounds as a brown oil (0.802 g, 62%, 85% purity): ¹H NMR (400 MHz, CDCl₃) δ 7.68 (dd, J=8.3, 1.0 Hz, 1H), 7.60 (dd, J=2.1, 1.0 Hz, 1H), 7.29-7.20 (m, 1H), 1.90 (t, J=18.1 Hz, 3H); ¹⁹F NMR (376 MHz, CDCl₃) δ−88.19; EIMS m/z 256.

Step 2—Preparation of 2-(2-chloro-4-(1,1-difluoroethyl)phenyl)-4,4,5,5-tetramethyl-1,3,2-dioxaborolane: To a solution of 1-bromo-2-chloro-4-(1,1-difluoroethyl)benzene (0.4 g, 1.33 mmol) in THF (5.3 mL) at 0° C. was added dropwise isopropylmagnesium(II) lithium chloride (1.3 M solution in THF; 1.23 mL, 1.60 mmol). The reaction mixture was stirred at 0° C. for 1 h, then 2-isopropoxy-4,4,5,5-tetramethyl-1,3,2-dioxaborolane (0.407 mL, 1.99 mmol) was added, and the reaction mixture was stirred at room temperature for 2 h. The mixture was poured into half saturated NH₄Cl and extracted with EtOAc (2×). The combined organic layers were dried over MgSO₄, filtered and dried in vacuo. The title compound was isolated as a brown oil and used without further purification in the next step (361 mg, 90%). EIMS m/z 302.

### Example 45: Preparation of 2-(2-chloro-4-((trifluoromethyl)thio)phenyl)-4,4,5,5-tetramethyl-1,3,2-dioxaborolane (C15)

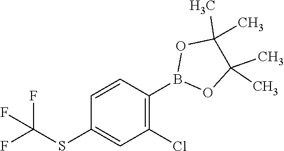 [SMILES: CC1(C)OB(c2ccc(SC(F)(F)F)cc2Cl)OC1(C)C]

Step 1—Preparation of 2-chloro-4-((trifluoromethyl)thio)phenol: 4-((Trifluoromethyl)thio)phenol (0.971 g, 5 mmol), toluene (12.5 mL), and diisobutylamine (87 μL, 0.5 mmol) were added sequentially to a 100-mL round-bottomed flask. Sulfuryl dichloride (0.405 mL, 5 mmol) was added dropwise, and the reaction mixture was stirred at room temperature for 1 h. The reaction mixture was concentrated. Purification of the residue by silica gel chromatography with a 0-10% EtOAc-hexane gradient afforded the title compound as a clear oil (0.888 g 78%): ¹H NMR (400 MHz, CDCl₃) δ 7.65 (d, J=2.2 Hz, 1H), 7.48 (dd, J=8.5, 2.2 Hz, 1H), 7.06 (d, J=8.5 Hz, 1H); ¹³C NMR (101 MHz, CDCl₃) δ 154.0, 137.1 (d, J=10.5 Hz), 130.9, 127.8, 120.6, 117.2, 115.9 (d, J=2.3 Hz); EIMS m/z 228.

Step 2—Preparation of 2-chloro-4-((trifluoromethyl)thio)phenyl trifluoromethanesulfonate: 2-Chloro-4-((trifluoromethyl)thio)phenol (0.492 g, 2.15 mmol) was placed in a scintillation vial and dissolved in dry DCM (4.3 mL). The solution was cooled to 0° C., and pyridine (0.348 mL, 4.3 mmol) and trifluoromethanesulfonic anhydride (0.434 mL, 2.58 mmol) were added sequentially. The reaction mixture was allowed to warm to room temperature. After 2 h the reaction mixture was quenched with satd NaHCO₃, diluted with water and DCM, and passed through a phase separator. The filtrate was concentrated. Purification by silica gel chromatography with a 0-10% EtOAc-hexane gradient afforded the title compound as a clear oil (0.633 g, 82%): ¹H NMR (400 MHz, CDCl₃) δ 7.85 (d, J=2.2 Hz, 1H), 7.65 (dd, J=8.6, 2.2 Hz, 1H), 7.43 (d, J=8.6 Hz, 1H); ¹³C NMR (101 MHz, CDCl₃) δ 147.4, 138.3, 135.8, 132.1, 130.9, 130.5, 129.9, 128.3, 127.4, 126.2, 123.8, 120.2, 116.9; EIMS m/z 360.

Step 3—Preparation of 2-(2-chloro-4-((trifluoromethyl)thio)phenyl)-4,4,5,5-tetramethyl-1,3,2-dioxaborolane: 2-Chloro-4-((trifluoromethyl)thio)phenyl trifluoromethanesulfonate (0.614 g, 1.702 mmol), 4,4,4′,4′,5,5,5′,5′-octamethyl-2,2′-bi(1,3,2-dioxaborolane) (0.562 g, 2.21 mmol), potassium acetate (0.334 g, 3.4 mmol) and Pd(dppf)Cl₂ (0.124 g, 0.170 mmol) were placed in a scintillation vial and dioxane (8.5 mL) was added. The reaction mixture was heated at 90° C. overnight. The reaction was cooled to room temperature and diluted with water and EtOAc. The layers were separated, and the aqueous layer was extracted with EtOAc (2×). The combined organic extracts were washed with brine, dried over Na₂SO₄, filtered and concentrated. Purification by silica gel chromatography with a 0-25% EtOAc-hexane gradient afforded the title compound as a clear oil that solidifies over time (0.303 g 53%): ¹H NMR (400 MHz, CDCl₃) δ 7.73 (d, J=7.8 Hz, 1H), 7.65 (d, J=1.7 Hz, 1H), 7.51 (dd, J=7.8, 1.6 Hz, 1H), 1.37 (s, 12H); ¹³C NMR (126 MHz, CDCl₃) δ 140.1, 137.0, 136.0, 133.0, 130.5, 129.2-126.5 (m), 84.6, 24.8; EIMS m/z 338.

### Example 46: Preparation of methyl 3-chloro-6-(trimethylstannyl)picolinate (C16)

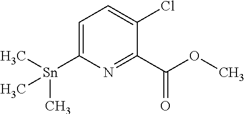

A degassed (nitrogen) mixture of methyl 3,6-dichloropicolinate (0.577 g, 2.80 mmol), Pd(PPh₃)₂Cl₂ (0.197 g, 0.28 mmol), and 1,1,1,2,2,2-hexamethyldistannane (0.917 g, 2.80 mmol) in dry dioxane (10 mL) was stirred at 80° C. for 2 h and then cooled to 20° C. The brown solution was adsorbed onto neutral alumina. Purification by column chromatography with 0-20% ethyl acetate-hexane afforded the title compound as a clear liquid (870 mg, 79%): ¹H NMR (400 MHz, CDCl₃) δ 7.60 (d, J=8.0 Hz, 1H), 7.48 (d, J=8.0 Hz, 1H), 4.00 (s, 3H), 0.36 (s, 9H); EIMS m/z 334.

The following compounds were prepared in like manner to the procedure outlined in Example 42:

### (E)-3-Chloro-2-(4,4,5,5-tetramethyl-1,3,2-dioxaborolan-2-yl)-5-(trifluoromethyl)benzaldehyde O-methyl oxime (C17)

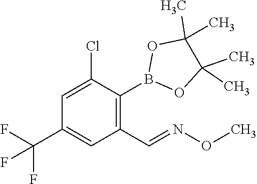 [SMILES: CO/N=C/c1cc(C(F)(F)F)cc(Cl)c1B1OC(C)(C)C(C)(C)O1]

Using the appropriate starting materials, the title compound was synthesized and isolated as a white solid (150 mg, 52%): ¹H NMR (400 MHz, CDCl₃) δ 8.15 (s, 1H), 7.86 (d, J=1.2 Hz, 1H), 7.56 (d, J=1.5 Hz, 1H), 4.00 (s, 3H), 1.43 (s, 12H).

### (E)-2-Chloro-3-(4,4,5,5-tetramethyl-1,3,2-dioxaborolan-2-yl)-6-(trifluoromethyl) benzaldehyde O-methyl oxime (C18)

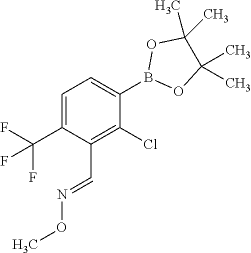 [SMILES: CO/N=C/c1c(C(F)(F)F)ccc(B2OC(C)(C)C(C)(C)O2)c1Cl]

Using the appropriate starting materials, the title compound was synthesized and isolated as white solid (25 mg, 44%): ¹H NMR (300 MHz, CDCl₃) δ 8.25 (d, J=2.1 Hz, 1H), 7.76 (d, J=7.8 Hz, 1H), 7.60 (d, J=8.0 Hz, 1H), 3.99 (s, 3H), 1.37 (s, 12H).

### (E)-2-(2-Chloro-3-(4,4,5,5-tetramethyl-1,3,2-dioxaborolan-2-yl)-6-(trifluoromethyl)benzylidene)-1,1-dimethylhydrazine (C19)

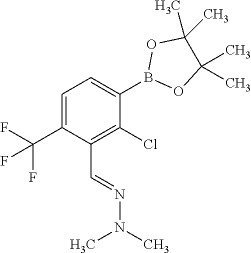 [SMILES: CN(C)/N=C/c1c(C(F)(F)F)ccc(B2OC(C)(C)C(C)(C)O2)c1Cl]

Using the appropriate starting materials, the title compound was synthesized and isolated as white solid (180 mg, 63%): ¹H NMR (300 MHz, CDCl₃) δ 7.58 (m, 2H), 7.30 (s, 1H), 2.99 (s, 6H), 1.37 (s, 12H).

The following compound was prepared in like manner to the procedure outlined in Example 46:

### Methyl 4-acetamido-3-chloro-6-(trimethylstannyl)picolinate (C20)

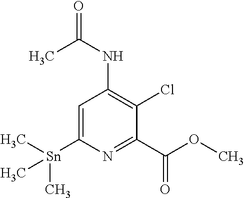

Using the appropriate starting materials and following the procedure outlined in WO 2003011853, the title compound was synthesized and isolated as a light yellow solid (3.05 g, 84%).

### Example 47: Preparation of 3-chloro-6-(2-chloro-4-(1,1-difluoro-2-methoxyethyl)phenyl)picolinic acid (F179)

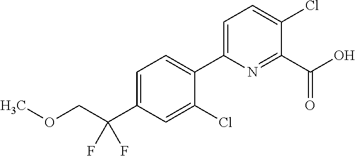 [SMILES: COCC(F)(F)c1ccc(-c2ccc(Cl)c(C(=O)O)n2)c(Cl)c1]

To a solution of methyl 3-chloro-6-(2-chloro-4-(1,1-difluoro-2-methoxyethyl)phenyl)picolinate (F177; 0.07 g, 0.186 mmol) in THF:MeOH:H₂O (2:1:1 ratio; 10 mL) was added lithium hydroxide hydrate (LiOH.H₂O; 0.02 g, 0.37 mmol) at 0° C., and the reaction mixture was stirred at room temperature for 16 h. The reaction mixture was concentrated under reduced pressure. The residue was taken up in water; the mixture was made acidic with 1 N HCl (pH ˜ 2); and the solution was extracted with DCM (2×30 mL). The organic layer was washed with brine, dried over anhydrous Na₂SO₄, filtered and concentrated under reduced pressure to afford the title compound as a brown liquid (0.04 g, 70%).

### Example 48: Preparation of 3-chloro-6-(2-chloro-4-(trifluoromethyl)phenyl)-5-fluoropicolinic acid (F352)

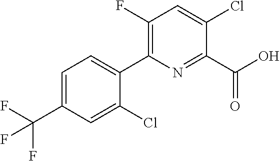 [SMILES: O=C(O)c1nc(-c2ccc(C(F)(F)F)cc2Cl)c(F)cc1Cl]

Lithium bromide (0.236 g, 2.72 mmol) was added to a solution of methyl 3-chloro-6-(2-chloro-4-(trifluoromethyl)phenyl)-5-fluoropicolinate (F107; 0.100 g, 0.272 mmol) and triethylamine (0.082 g, 0.815 mmol), and the reaction mixture was heated at 60° C. After 5 min, a white precipitate formed, and the reaction mixture was diluted with water and DCM and passed through a phase separator. The filtrate was concentrated, and the residue was dissolved in DCM, washed with 2 M HCl, and passed through a phase separator. The filtrate was concentrated. The title compound was recovered as a white solid (81 mg, 84%).

### Example 49: Preparation of 5-chloro-3-fluoro-2′-methoxy-6′-(trifluoromethyl)-[2,3′-bipyridine]-6-carboxylic acid (F386)

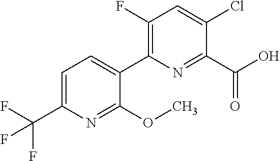 [SMILES: COc1nc(C(F)(F)F)ccc1-c1nc(C(=O)O)c(Cl)cc1F]

To a reaction vessel containing methyl 5-chloro-2′,3-difluoro-6′-(trifluoromethyl)-[2,3′-bipyridine]-6-carboxylate (F354; 0.100 g, 0.284 mmol) were added THF (2.8 mL) and sodium hydroxide (0.851 mL, 0.851 mmol). After 1 h, hydrolysis of the methyl ester was complete. Methanol (1.15 mL, 28.4 mmol) was added, and the resulting reaction mixture was stirred overnight at room temperature. The mixture was made acidic by adding a slight excess of 2 N HCl. The mixture was filtered and concentrated under vacuum but not to dryness. The precipitate that formed was washed with water and dried under vacuum. The title compound was isolated as a white solid (80 mg, 77%).

### Example 50: Preparation of 3-chloro-6-(4-(difluoromethyl)-3-fluoro-2-methoxyphenyl)-5-fluoropicolinic acid (F391)

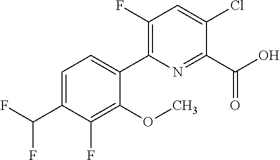 [SMILES: COc1c(-c2nc(C(=O)O)c(Cl)cc2F)ccc(C(F)F)c1F]

To a reaction vessel containing methyl 3-chloro-6-(4-(difluoromethyl)-3-fluoro-2-methoxyphenyl)-5-fluoropicolinate (F253; 0.130 g, 0.357 mmol) were added THF (3.57 mL) and sodium hydroxide (0.715 mL, 0.715 mmol). The reaction mixture was stirred overnight at room temperature and was made acidic by adding a slight excess of 2 N HCl. The mixture was concentrated, and the precipitate that formed was washed with water and dried under vacuum. The title compound was isolated as a white solid (100 mg, 77%).

### Example 51: Preparation of methyl 2′,3′,5-trichloro-[2,4′-bipyridine]-6-carboxylate (F86)

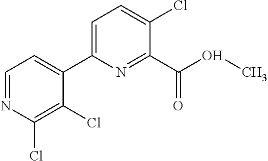 [SMILES: COC(=O)c1nc(-c2ccnc(Cl)c2Cl)ccc1Cl]

A solution of (2,3-dichloropyridin-4-yl)boronic acid (200 mg, 1.04 mmol), cesium fluoride (475 mg, 3.13 mmol), and methyl 3,6-dichloropicolinate (215 mg, 1.04 mmol) in acetonitrile (3910 μL) and water (1303 μL) was degassed (nitrogen) for 20 min before adding Pd(PPh₃)₂Cl₂ (73 mg, 0.104 mmol) and heating to 60-65° C. After heating for 2 h, the reaction mixture was cooled and loaded directly onto silica gel. Purification via reverse phase chromatography afforded the title compound as a white solid (62 mg, 18%).

### Example 52: Preparation of methyl 6-(2-chloro-4-(trifluoromethyl)phenyl)-3-fluoropicolinate (F113)

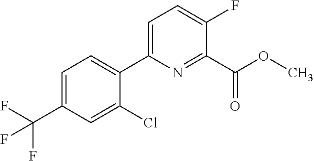 [SMILES: COC(=O)c1nc(-c2ccc(C(F)(F)F)cc2Cl)ccc1F]

A solution of methyl 6-chloro-3-fluoropicolinate (300 mg, 1.58 mmol), (2-chloro-4-(trifluoromethyl)phenyl)boronic acid (533 mg, 2.37 mmol), cesium fluoride (1.242 g, 8.18 mmol) in acetonitrile (6 mL) and water (2 mL) was degassed (nitrogen) for 20 min before the adding Pd(PPh₃)₂Cl₂ (0.111 g, 0.158 mmol) and heating to 60-65° C. for 6 h. After the first 30 min, the reaction turned clear red/orange that persisted throughout the reaction. The reaction mixture was loaded directly onto silica gel. Purification by automated column chromatography eluting with a hexanes-ethyl acetate gradient mobile phase (0-20% over 12 column volumes; 40% ethyl acetate over 4 column volumes) provided the title compound as a white solid (174 mg, 31%).

### Example 53: Preparation of methyl 5-chloro-2-(2-methoxy-4-(trifluoromethyl)phenyl)pyrimidine-4-carboxylate (F313)

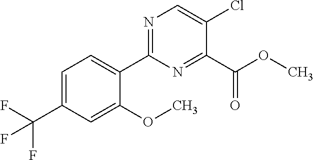 [SMILES: COC(=O)c1nc(-c2ccc(C(F)(F)F)cc2OC)ncc1Cl]

Potassium fluoride (0.146 g, 2.51 mmol) was dissolved in water (2.4 mL) in a microwave reaction vessel. Methyl 2,5-dichloropyrimidine-4-carboxylate (0.4 g, 1.9 mmol), (2-methoxy-4-(trifluoromethyl)phenyl)boronic acid (0.446 g, 2.03 mmol), and bis(triphenylphosphine)palladium chloride (0.068 g, 0.097 mmol) were added followed by 1,4-dioxane (7.25 mL). The resulting reaction mixture was heated in a Biotage microwave reactor at 110° C. for 20 min. The cooled reaction mixture was partitioned between DCM and brine. The organic phase was dried and concentrated. Purification by flash chromatography (1-10% ethyl acetate in hexanes) provided the title compound as a white solid (255 mg, 37%).

### Example 54: Preparation of methyl 6-(5-amino-2-chloro-4-(trifluoromethyl)phenyl)-3-chloropicolinate (F211)

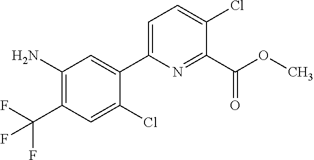 [SMILES: COC(=O)c1nc(-c2cc(N)c(C(F)(F)F)cc2Cl)ccc1Cl]

1-Butyl-3-methylimidazolium trifluoromethanesulfonate (0.33 mL, 0.150 mmol) and iron(III) chloride (8.1 mg, 0.050 mmol) were placed in a 1-dram vial and stirred at room temperature for 30 min. N-Chlorosucciminide (0.140 g, 1.05 mmol) and a solution of methyl 6-(3-amino-4-(trifluoromethyl)phenyl)-3-chloropicolinate (F218; 0.331 g, 1.0 mmol) in THF (0.63 mL) were added sequentially. The reaction mixture was heated at 60° C. overnight. The reaction mixture was cooled to room temperature, diluted with ethyl acetate, and washed with satd sodium thiosulfate and brine. ¹H NMR spectral analysis showed a mixture of the 2- and 6-chlorination isomers. Purification by silica gel chromatography afforded the title compound as a beige solid (102 mg, 28%).

### Example 55: Preparation of Methyl 6-(2-amino-6-chloro-4-(trifluoromethyl)phenyl)-3-chloropicolinate (F213)

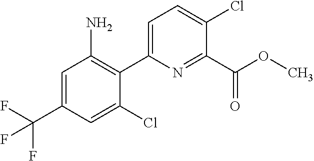 [SMILES: COC(=O)c1nc(-c2c(N)cc(C(F)(F)F)cc2Cl)ccc1Cl]

Methyl 3-chloro-6-(2-chloro-6-nitro-4-(trifluoromethyl)phenyl)picolinate (F206; 309 mg, 0.782 mmol) was diluted with ethanol (1738 μL), water (869 μL,) and acetic acid (470 mg, 7.82 mmol) in a 5 mL vial. Iron powder (87 mg, 1.56 mmol) was added, and the reaction mixture was stirred at room temperature. The reaction progress slowed after 1 h, and four portions of purified iron were used to fully consume the starting material. TLC analysis showed 3 spots—starting material, the intermediate hydroxylamine, and the product. The reaction was allowed to progress until a single spot appeared. The reaction mixture was diluted with ethyl acetate and water and neutralized with satd aq NaHCO₃. The layers were partioned, and the organic phase was extracted with ethyl acetate (3×). Purification by chromatography of the residue with a gradient of 0-50% ethyl acetate-hexanes. The title compound was isolated as a white solid (108 mg, 38%).

### Example 56: Preparation of methyl 3-chloro-6-(2-chloro-4-(trifluoromethyl)-6-vinylphenyl)picolinate (F216)

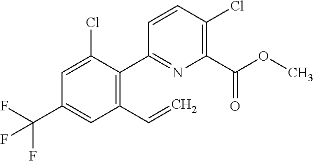 [SMILES: C=Cc1cc(C(F)(F)F)cc(Cl)c1-c1ccc(Cl)c(C(=O)OC)n1]

Methyl 3-chloro-6-(2-chloro-6-iodo-4-(trifluoromethyl)phenyl)picolinate (F214; 95 mg, 0.200 mmol), tributyl(vinyl)stannane (79 mg, 0.249 mmol), and toluene were added to a 25 mL vial. The mixture was degassed with nitrogen for 10 min before adding Pd(dppf)Cl₂ as a complex with dichloromethane (1:1; 15 mg, 0.020 mmol) and was heated at 100° C. for 16 h. The mixture was concentrated under reduced pressure, taken up in ethyl acetate (100 mL) and treated with a satd solution of potassium fluoride (50 mL) overnight. The mixture was filtered through a plug of Celite®, and the organic layer was separated and dried over sodium sulfate, filtered and concentrated under reduced pressure to give a residue. Purification by chromatography (silica gel, heptane-ethyl acetate) as a white solid (46 mg, 55%).

### Example 57: Preparation of methyl 3-chloro-6-(2-chloro-3-methyl-4-(trifluoromethyl)phenyl)picolinate (F136)

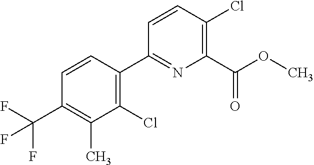 [SMILES: COC(=O)c1nc(-c2ccc(C(F)(F)F)c(C)c2Cl)ccc1Cl]

A solution of methyl 6-(3-bromo-2-chloro-4-(trifluoromethyl)phenyl)-3-chloropicolinate (F140; 150 mg, 0.349 mmol), 2,4,6-trimethyl-1,3,5,2,4,6-trioxatriborinane (0.06 mL, 0.419 mmol), and cesium carbonate (228 mg, 0.699 mmol) in 1,4-dioxane (10 mL) and water (1 mL) was purged with argon for 10 min. [1,1′-Bis(diphenylphosphino)ferrocene]dichloropalladium(II) complex (26 mg, 0.034 mmol) was added to the above mixture. The mixture was purged with argon for 10 min and heated to 100° C. for 3 h. The reaction mixture was cooled to room temperature, filtered through a Celite® bed, and washed with EtOAc. The filtrate layer was washed with water and brine and was concentrated under vacuum. Purification of the residue by column chromatography with 15% EtOAc in hexane as the eluent afforded the title compound as white solid (80 mg, 31%).

### Example 58: Preparation of methyl 3-chloro-6-(2-chloro-4-(trifluoromethyl)-3-vinylphenyl)picolinate (F137)

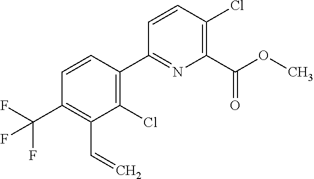 [SMILES: C=Cc1c(C(F)(F)F)ccc(-c2ccc(Cl)c(C(=O)OC)n2)c1Cl]

A solution of methyl 6-(3-bromo-2-chloro-4-(trifluoromethyl)phenyl)-3-chloropicolinate (F140; 150 mg, 0.349 mmol), 4,4,5,5-tetramethyl-2-vinyl-1,3,2-dioxaborolane (0.09 mL, 0.524 mmol) and sodium carbonate (110 mg, 1.048 mmol) in 1,4-dioxane (10 mL) and water (1 mL) was purged with argon for 10 min. Tetrakis(triphenylphosphine)palladium(0) (40 mg, 0.034 mmol) was added to the above reaction mixture, again purged with argon for 10 min, and heated to 100° C. for 2 h. The reaction mixture was cooled to room temperature, filtered through a Celite® bed and washed with EtOAc. The filtrate was washed with water and brine solution and then concentrated under vacuum. Purification by column chromatography with 15% EtOAc in hexane as eluent afforded the title compound as a white solid (25 mg, 19%).

### Example 59: Preparation of methyl 3-chloro-6-(2-chloro-3-(methylsulfonyl)-4-(trifluoromethyl)phenyl)picolinate (F139) and methyl 3-chloro-6-(2-chloro-6-(methylsulfonyl)-4-(trifluoromethyl)phenyl)picolinate (F138)

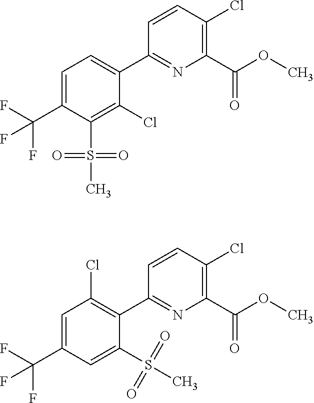 [SMILES: COC(=O)c1nc(-c2ccc(C(F)(F)F)c(S(C)(=O)=O)c2Cl)ccc1Cl ; COC(=O)c1nc(-c2c(Cl)cc(C(F)(F)F)cc2S(C)(=O)=O)ccc1Cl]

To a solution of methyl 3-chloro-6-(2-chloro-3-(methylsulfinyl)-4-(trifluoromethyl)phenyl)picolinate and methyl 3-chloro-6-(2-chloro-6-(methylsulfinyl)-4-(trifluoromethyl)phenyl)picolinate (F119 and F120; 250 mg, 0.630 mmol) in acetic acid (2 mL) was added sodium perborate tetrahydrate (194 mg, 1.261 mmol) at room temperature. The reaction mixture was heated to 100° C. for 16 h, was cooled to room temperature, and was poured slowly into satd sodium bicarbonate solution. The mixture was extracted with EtOAc. The organic layer was washed with water and brine and was concentrated under vacuum. Purification of the resulting material by column chromatography using 30% EtOAc in hexane as eluent, followed by a preparative HPLC purification, afforded the title compounds as a white solid (40 mg, 15%) and as a white solid (15 mg, 6%), respectively.

### Example 60: Preparation of methyl 3-chloro-6-(2-chloro-3-(methylsulfinyl)-4-(trifluoromethyl)phenyl)picolinate (F119) and methyl 3-chloro-6-(2-chloro-6-(methylsulfinyl)-4-(trifluoromethyl)phenyl)picolinate (F120)

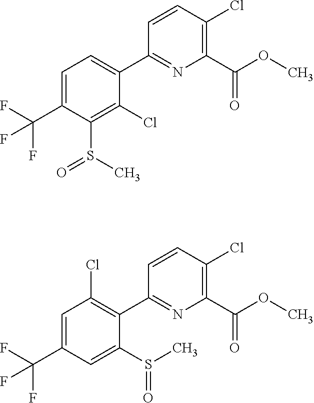 [SMILES: COC(=O)c1nc(-c2ccc(C(F)(F)F)c(S(C)=O)c2Cl)ccc1Cl ; COC(=O)c1nc(-c2c(Cl)cc(C(F)(F)F)cc2S(C)=O)ccc1Cl]

To a solution of methyl 3-chloro-6-(2-chloro-3-(methylthio)-4-(trifluoromethyl)phenyl)picolinate and methyl 3-chloro-6-(2-chloro-6-(methylthio)-4-(trifluoromethyl)phenyl)picolinate (F121 and F122; 200 mg, 0.504 mmol) in acetic acid (2 mL) was added sodium perborate tetrahydrate (78 mg, 0.504 mmol) at room temperature. The reaction mixture was heated to 100° C. for 2 h, was cooled to room temperature, was poured slowly into a satd sodium bicarbonate solution. The mixture was extracted with EtOAc. The organic layer was washed with water and brine and was concentrated under vacuum. Purification of the resulting material by column chromatography using 30% EtOAc in hexane as eluent, followed by a preparative HPLC purification, provided the title compounds as a colorless liquid (33 mg, 16%) and as a brown solid (70 mg, 34%), respectively.

### Example 61: Preparation of methyl 3-chloro-6-(2-chloro-3-(methylthio)-4-(trifluoromethyl)phenyl)picolinate (F121) and methyl 3-chloro-6-(2-chloro-6-(methylthio)-4-(trifluoromethyl)phenyl)picolinate (F122)

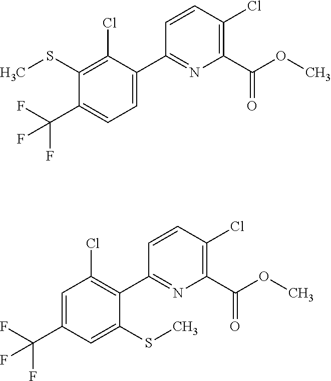 [SMILES: COC(=O)c1nc(-c2ccc(C(F)(F)F)c(SC)c2Cl)ccc1Cl ; COC(=O)c1nc(-c2c(Cl)cc(C(F)(F)F)cc2SC)ccc1Cl]

A solution of methyl 3,6-dichloropicolinate (245 mg, 1.189 mmol), 2-(2-chloro-3-(methylthio)-4-(trifluoromethyl)phenyl)-4,4,5,5-tetramethyl-1,3,2-dioxaborolane, 2-(2-chloro-6-(methylthio)-4-(trifluoromethyl)phenyl)-4,4,5,5-tetramethyl-1,3,2-dioxaborolane (500 mg, 1.427 mmol) and potassium fluoride (207 mg, 3.567 mmol) in acetonitrile (15 mL) and water (5 mL) was purged with argon for 10 min. Bis(triphenylphosphine)palladium(II) dichloride (83 mg, 0.118 mmol) was added to the above reaction mixture, and the mixture was purged with argon for 10 min and heated to 90° C. for 5 h. The reaction mixture was cooled to room temperature, filtered through a bed of Celite® and washed with EtOAc. The filtrate was washed with water and brine solution and was concentrated under vacuum. Purification of the resulting material by column chromatography using 15% EtOAc in hexane, followed by purification by preparative HPLC, furnished the title compounds as a colorless liquid (50 mg, 9%) and as a white solid (200 mg, 36%), respectively.

### Example 62: Preparation of methyl 3-chloro-6-(2-chloro-3-cyano-4-(trifluoromethyl)phenyl)picolinate (F143)

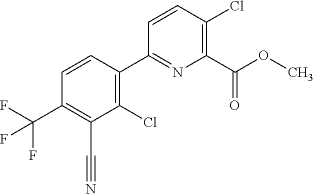 [SMILES: COC(=O)c1nc(-c2ccc(C(F)(F)F)c(C#N)c2Cl)ccc1Cl]

A solution of methyl 6-(3-bromo-2-chloro-4-(trifluoromethyl)phenyl)-3-chloropicolinate (F140; 150 mg, 0.349 mmol), zinc cyanide (62 mg, 0.52 mmol) and tetrakis(triphenylphosphine) palladium(0) (40 mg, 0.034 mmol) in DMF (5 mL) was purged with argon for 10 min. The reaction mixture was heated to 150° C. for 6 h and cooled to room temperature. The reaction mixture was poured into ice water and extracted with EtOAc. The organic layer was washed with water and brine and was concentrated under vacuum. Purification of the resulting material by column chromatography using 20% EtOAc in hexane afforded the title compound as an off-white solid (40 mg, 30%).

### Example 63: Preparation of methyl 3-chloro-5-fluoro-6-(2-fluoro-4-(trifluoromethyl)phenyl)picolinate (F158)

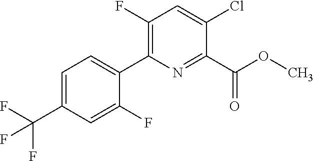 [SMILES: COC(=O)c1nc(-c2ccc(C(F)(F)F)cc2F)c(F)cc1Cl]

Potassium carbonate (0.241 g, 1.74 mmol) was dissolved in water (1.67 mL) in a microwave reaction vessel. (2-Fluoro-4-(trifluoromethyl)phenyl)boronic acid (0.292 g, 1.406 mmol), methyl 3,6-dichloro-5-fluoropicolinate (0.300 g, 1.339 mmol), and bis(triphenylphosphine)palladium dichloride (0.094 g, 0.134 mmol) were added followed by 1,4-dioxane (5.02 mL). The resulting reaction mixture was heated in a Biotage microwave reactor at 110° C. for 20 min. The cooled reaction mixture was partitioned between DCM and water. The organic phase was dried and concentrated. Purification by reverse-phase chromatography followed by flash chromatography (1-10% EtOAc in hexanes) provided the title compound as a white solid (353 mg, 71%).

The following compound was prepared in like manner to the procedure outlined in Example 63:

### Methyl 4-amino-3-chloro-6-(4-cyano-2-methoxyphenyl)-5-fluoropicolinate (C21)

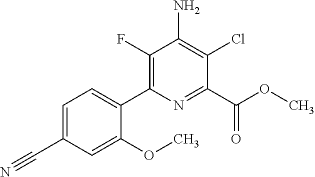 [SMILES: COC(=O)c1nc(-c2ccc(C#N)cc2OC)c(F)c(N)c1Cl]

Using the appropriate starting materials, the title compound was synthesized and isolated as an off-white solid (118 mg, 13%).

### Example 64: Preparation of methyl 3-chloro-6-(4-(difluoromethyl)-3-fluoro-2-methoxyphenyl)-5-fluoropicolinate (F253)

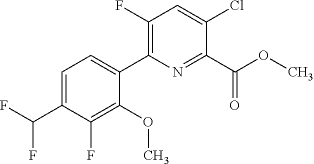 [SMILES: COC(=O)c1nc(-c2ccc(C(F)F)c(F)c2OC)c(F)cc1Cl]

Bis(2-methoxyethyl)aminosulfur trifluoride (0.370 g, 1.674 mmol) was added to a solution of methyl 3-chloro-5-fluoro-6-(3-fluoro-4-formyl-2-methoxyphenyl)picolinate (F357; 0.260 g, 0.761 mmol) in DCM (7.61 mL) cooled in an ice bath. The ice bath was removed, and the reaction mixture was stirred at room temperature for 2 h. Methanol was added, and the reaction mixture was stirred for 10 min and then concentrated onto silica gel. Purification of the resulting material by flash chromatography (0-30% EtOAc in hexanes gradient) provided the title compound as a white solid (271 mg, 93%).

### Example 65: Preparation of methyl 3-chloro-6-(2-chloro-4-(1,1-difluoro-2-methoxyethyl)phenyl)picolinate (F177)

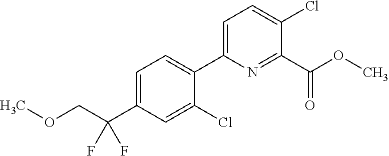

To a solution of 1-bromo-2-chloro-4-(1,1-difluoro-2-methoxyethyl)benzene (C89; 0.5 g, 1.75 mmol) and methyl 3-chloro-6-(trimethylstannyl)picolinate (C16; 0.5 g, 1.50 mmol) in toluene (10 mL) was added Pd(PPh₃)₄ (0.28 g, 0.24 mmol) and the reaction mixture was stirred at 110° C. for 16 h. The reaction mixture was cooled to room temperature, and water was added. The mixture was extracted with EtOAc. The organic layer was washed with brine, dried over anhydrous Na₂SO₄, filtered and concentrated under reduced pressure. Purification of the resulting material by column chromatography (silica gel 100-200 mesh) eluting with 40-50% EtOAc in petroleum ether afforded the title compound as a pale yellow liquid (0.15 g, 30%).

### Example 66: Preparation of methyl 3-chloro-6-(2-chloro-6-iodo-4-(trifluoromethyl)phenyl)picolinate (F214)

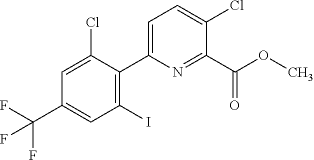 [SMILES: COC(=O)c1nc(-c2c(Cl)cc(C(F)(F)F)cc2I)ccc1Cl]

To a 5 mL vial were added methyl 6-(2-amino-6-chloro-4-(trifluoromethyl)phenyl)-3-chloropicolinate (F213; 96 mg, 0.263 mmol), tert-butyl nitrite (67.8 mg, 0.657 mmol), and diiodomethane (704 mg, 2.63 mmol). The vial was sealed, and the reaction mixture was heated to 65° C. for 2 h. The reaction mixture was loaded directly onto a silica gel column. Purification by column chromatography eluting with hexanes-EtOAc afforded the title compound as a yellow solid (92 mg, 74%).

### Example 67: Preparation of methyl 3-chloro-6-(2-chloro-6-ethynyl-4-(trifluoromethyl)phenyl)picolinate (F239)

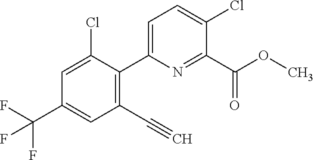 [SMILES: C#Cc1cc(C(F)(F)F)cc(Cl)c1-c1ccc(Cl)c(C(=O)OC)n1]

To 25 mL vial were added ethynyltrimethylsilane (0.028 g, 0.284 mmol), Pd(PPh₃)₂Cl₂ (0.013 g, 0.019 mmol) and CuI (0.004 g, 0.019 mmol) to a solution of methyl 3-chloro-6-(2-chloro-6-iodo-4-(trifluoromethyl)phenyl)picolinate (F214; 0.090 g, 0.189 mmol) in DMF (1 mL) and Et₃N (4 mL). The reaction mixture was stirred at 50° C. for 8 h. The reaction mixture was applied to silica gel. Purification by column chromatography eluting with a linear gradient of hexane-EtOAc (0% to 100% EtOAc). The title compound was isolated as a brown oil (35 mg, 50%).

### Example 68: Preparation of methyl 3-chloro-6-(2-chloro-4-(difluoromethoxy)phenyl)picolinate (F157)

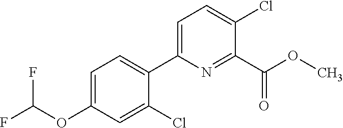 [SMILES: COC(=O)c1nc(-c2ccc(OC(F)F)cc2Cl)ccc1Cl]

Methyl 3,6-dichloropicolinate (0.290 g, 1.408 mmol), (2-chloro-4-(difluoromethoxy)phenyl)trimethylstannane (C93; 0.481 g, 1.408 mmol), Pd(PPh₃)₂Cl₂ (0.296 g, 0.422 mmol), and copper(I) iodide (0.080 g, 0.422 mmol) were combined with DMF (5.63 mL) in a microwave vessel and heated at 130° C. in a Biotage microwave reactor for 30 min. The reaction mixture was filtered and concentrated. Purification of the resulting product by reverse phase HPLC provided the title compound as an off-white solid (125 mg, 24%).

### Example 69: Preparation of methyl 3-chloro-6-(2-chloro-4-(((trifluoromethyl)sulfonyl)oxy)phenyl)picolinate (F364)

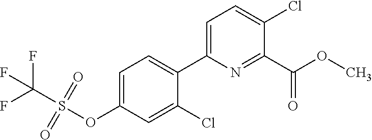 [SMILES: COC(=O)c1nc(-c2ccc(OS(=O)(=O)C(F)(F)F)cc2Cl)ccc1Cl]

Trifluoromethanesulfonic anhydride (0.298 mL, 1.76 mmol) was added to a solution of methyl 3-chloro-6-(2-chloro-4-hydroxyphenyl)picolinate (F355; 0.350 g, 1.17 mmol) and pyridine (0.190 mL, 2.35 mmol) in DCM (11.7 mL) cooled in an ice bath. The reaction mixture was allowed to warm to room temperature, stirred for 2 h, and partitioned between DCM and water. The organic phase was passed through a Biotage phase separator and concentrated under vacuum onto silica gel. Purification of the resulting product by flash chromatography (0-20% EtOAc in hexanes) provided the title compound as an off-white solid (243 mg, 47%).

### Example 70: Preparation of methyl 3-chloro-6-(2-chloro-4-((methylsulfonyl)oxy)phenyl)picolinate (F288)

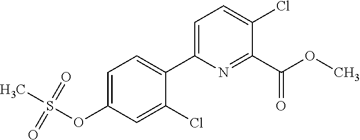

Methanesulfonyl chloride (0.131 mL, 1.68 mmol) was added dropwise to a solution of methyl 3-chloro-6-(2-chloro-4-hydroxyphenyl)picolinate (F355; 0.250 g, 0.839 mmol) and Et₃N (0.234 mL, 1.68 mmol) in DCM (8.39 mL) cooled in an ice bath. The reaction mixture was stirred for 1 h at room temperature and partitioned between DCM and water. The organic phase was dried by passing through a phase separator and concentrated onto silica gel. Purification of the resulting product by flash chromatography (0-40% EtOAc in hexanes gradient) provided the title compound as a white solid (293 mg, 90%).

### Example 71: Preparation of methyl 3-chloro-5-fluoro-6-(2-fluoro-4-iodophenyl)picolinate (F328)

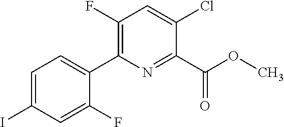 [SMILES: COC(=O)c1nc(-c2ccc(I)cc2F)c(F)cc1Cl]

Methyl 6-(4-amino-2-fluorophenyl)-3-chloro-5-fluoropicolinate (prepared as in U.S. Pat. No. 9,113,629 B2; 0.500 g, 1.674 mmol) was combined with diiodomethane (1.35 mL, 16.7 mmol), and tert-butyl nitrite (0.398 mL, 3.35 mmol) was added dropwise. The reaction mixture was heated to 100° C. for 30 min. The cooled reaction mixture was diluted with DCM and washed with an aqueous solution of sodium thiosulfate (1.323 g, 8.37 mmol). The organic phase was dried by passing through a phase separator and was concentrated onto silica gel. Purification by flash chromatography (0-50% EtOAc in hexanes) provided the title compound as a white solid (320 mg, 46%).

### Example 72: Preparation of methyl 4-amino-3-chloro-6-(2-fluoro-3-methyl-4-(trifluoromethyl)phenyl)picolinate (C22)

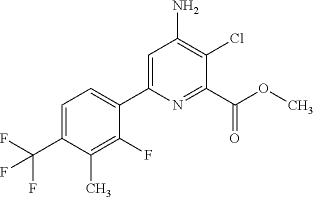 [SMILES: COC(=O)c1nc(-c2ccc(C(F)(F)F)c(C)c2F)cc(N)c1Cl]

To a 5 mL microwave vial were added methyl 4-amino-3,6-dichloropicolinate (400 mg, 1.81 mmol), 2-(2-fluoro-3-methyl-4-(trifluoromethyl)phenyl)-4,4,5,5-tetramethyl-1,3,2-dioxaborolane (C75; 660 mg, 2.17 mmol), potassium fluoride (273 mg, 4.71 mmol), and Pd(PPh₃)₂Cl₂ (127 mg, 0.181 mmol). A 1:1 mixture of acetonitrile-water (5.58 mL (2.79 mL each)) was added. The reaction vial was then sealed and heated in a Biotage microwave reactor to 115° C. for 20 min. The reaction mixture was cooled to room temperature, diluted with EtOAc, and washed with water. The organic phase was dried over Na₂SO₄, filtered and concentrated. Purification by flash chromatography (silica gel, hexane-EtOAc gradient) gave the title compound as a white solid (567 mg, 86%).

### Example 73: Cyanomethyl 3-chloro-6-(2-chloro-4-(trifluoromethyl)phenyl)picolinate (F382)

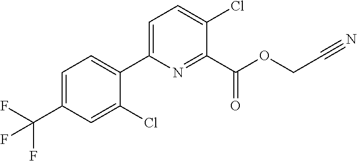 [SMILES: N#CCOC(=O)c1nc(-c2ccc(C(F)(F)F)cc2Cl)ccc1Cl]

A mixture of 3-chloro-6-(2-chloro-4-(trifluoromethyl)phenyl)picolinic acid (F61; 1 equiv), potassium carbonate (1.5 equiv) and benzyl bromide (1.2 equiv) in DMF (0.2 M) is stirred at room temperature overnight. The reaction mixture is then poured into a satd aq NaHCO₃ solution and extracted with EtOAc (2×). The combined organic layers are dried over magnesium sulfate, filtered and concentrated. Purification by column chromatography with a hexane-EtOAc gradient affords the title compound.

### Example 74: Preparation of methyl 4-amino-3-chloro-5-fluoro-6-(4-(trifluoromethyl)benzofuran-7-yl)picolinate (C23)

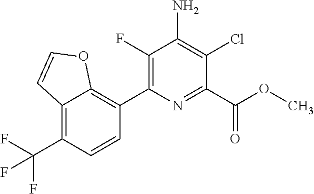 [SMILES: COC(=O)c1nc(-c2ccc(C(F)(F)F)c3ccoc23)c(F)c(N)c1Cl]

7-Bromo-4-(trifluoromethyl)benzofuran (150 mg, 0.566 mmol), methyl 4-amino-3-chloro-5-fluoro-6-(trimethylstannyl)picolinate (prepared as in U.S. Pat. No. 9,113,629 B2; 270 mg, 0.736 mmol) and cesium fluoride (215 mg, 1.415 mmol) were dissolved in DMF (5 mL) and the mixture was purged with nitrogen for 10 min. Copper(I) iodide (21.6 mg, 0.113 mmol) and Pd(PPh₃)₄ (65.4 mg, 0.057 mmol) were added, and the mixture was purged for an additional 5 min, capped and heated under microwave irradiation (Biotage) at 85° C. for 30 min. The mixture was diluted with EtOAc, washed with brine (×3), dried with MgSO₄, filtered and concentrated under vacuum to give a brown gum. Purification on silica gel eluting with DCM and methanol gave the title compound as a light brown solid (183 mg, 79%): ¹H NMR (400 MHz, CDCl) δ 7.89-7.74 (m, 1H), 7.73-7.61 (m, 2H), 7.11-7.00 (m, 1H), 5.00 (s, 2H), 3.99 (s, 3H); ¹⁹F NMR (376 MHz, CDCl₃) δ−61.42 (d, J=31.4 Hz), −137.04; ESIMS m/z 389.0 ([M+H]⁺).

### Example 75: Preparation of methyl 4-amino-3-chloro-6-(2-chloro-6-methoxyphenyl)-5-fluoropicolinate (C24)

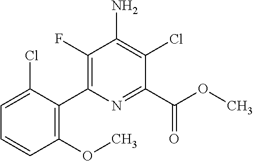 [SMILES: COC(=O)c1nc(-c2c(Cl)cccc2OC)c(F)c(N)c1Cl]

To a microwave vial were added methyl 4-amino-3,6-dichloro-5-fluoropicolinate (800 mg, 3.35 mmol), (2-chloro-6-methoxyphenyl)boronic acid (811 mg, 4.35 mmol), cesium fluoride (1.02 g, 6.69 mmol), and Pd(PPh₃)₂Cl₂ (235 mg, 0.335 mmol). A 1:1 mixture of acetonitrile-water (10 mL) was added. The reaction vial was then sealed and heated in a Biotage microwave reactor to 115° C. for 30 min. The mixture was shaken with EtOAc (35 mL) and satd aq sodium chloride solution (10 mL). The organic phase was washed with satd aq sodium chloride solution (10 mL), dried, and concentrated. Purification by silica gel chromatography with 0-50% EtOAc-hexane as eluent provided the title compound as a white solid (400 mg, 35%): ¹H NMR (400 MHz, CDCl₃) δ 7.31 (t, J=8.3 Hz, 1H), 7.08 (dd, J=8.1, 0.9 Hz, 1H), 6.87 (dd, J=8.4, 0.7 Hz, 1H), 4.88 (s, 2H), 3.95 (s, 3H), 3.74 (s, 3H); ¹⁹F NMR (376 MHz, CDCl₃) δ−137.86; ESIMS m/z 343.4 ([M+H]⁺).

### Example 76: Preparation of 4-amino-3-chloro-6-(3-(difluoromethyl)-2-fluoro-4-(trifluoromethyl)phenyl)-5-fluoropicolinic acid (C25)

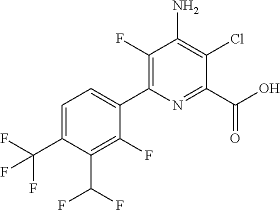 [SMILES: Nc1c(F)c(-c2ccc(C(F)(F)F)c(C(F)F)c2F)nc(C(=O)O)c1Cl]

To a solution of methyl 4-amino-3-chloro-6-(3-(difluoromethyl)-2-fluoro-4-(trifluoromethyl)phenyl)-5-fluoropicolinate (C26; 250 mg, 0.600 mmol) in MeOH (1 mL) was added 2 M NaOH (0.300 mL, 0.600 mmol). The reaction mixture was stirred at 25° C. for 15 h. The reaction mixture was then concentrated, and made acidic with 2 M HCl. The product precipitated out of solution and was collected in a Buchner Funnel. The title compound was isolated as an off-white solid (197 mg, 82%): ¹H NMR (400 MHz, DMSO-d₆) δ 8.03 (t, J=7.4 Hz, 1H), 7.92 (d, J=8.3 Hz, 1H), 7.61-7.24 (m, 1H); ¹⁹F NMR (376 MHz, DMSO-d₆) δ−55.95-−57.22 (m), −111.58-−112.73 (m), −113.96 (ddd, J=28.3, 14.1, 6.9 Hz), −138.51 (d, J=26.3 Hz); ESIMS m/z 403.08 ([M+H]⁺).

### Example 77: Preparation of methyl 4-amino-3-chloro-6-(3-(difluoromethyl)-2-fluoro-4-(trifluoromethyl)phenyl)-5-fluoropicolinate (C26)

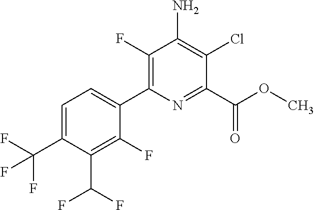 [SMILES: COC(=O)c1nc(-c2ccc(C(F)(F)F)c(C(F)F)c2F)c(F)c(N)c1Cl]

Step 1—Preparation of 2-(3-(difluoromethyl)-2-fluoro-4-(trifluoromethyl)phenyl)-4,4,5,5-tetramethyl-1,3,2-dioxaborolane: 2-(Difluoromethyl)-1-fluoro-3-(trifluoromethyl)benzene (1.756 g, 8.20 mmol) was added dropwise to a solution of butyllithium (3.61 mL, 9.02 mmol) in THF (20.5 mL) that was cooled to −78° C. under nitrogen. The reaction mixture was stirred at −70-−75° C. for 10 min, and 2-isopropoxy-4,4,5,5-tetramethyl-1,3,2-dioxaborolane (1.02 mL, 5.00 mmol) was added dropwise, keeping the temperature below −65° C. The reaction mixture was then allowed to warm to 0° C. Water was added, and the resulting mixture was extracted with Et2O. The aqueous phase was carefully acidified with 2M HCl, and extracted with Et₂O. The organic phase was dried and concentrated to give the title compound as an orange oil (1.2 g) that was used without purification in the Suzuki step reported below.

Step 2—Preparation of methyl 4-amino-3-chloro-6-(3-(difluoromethyl)-2-fluoro-4-(trifluoromethyl)phenyl)-5-fluoropicolinate: To a 5 mL microwave vial were added methyl 4-amino-3,6-dichloro-5-fluoropicolinate (224 mg, 0.937 mmol), 2-(3-(difluoromethyl)-2-fluoro-4-(trifluoromethyl)phenyl)-4,4,5,5-tetramethyl-1,3,2-dioxaborolane (382 mg, 1.13 mmol), potassium fluoride (142 mg, 2.44 mmol) and Pd(PPh₃)₂Cl₂ (65.8 mg, 0.094 mmol). A mixture of 1:1 acetonitrile-water (5.58 mL (2.79 mL each)) was added. The reaction vial was then sealed and heated in a Biotage microwave reactor to 115° C. for 20 min. The reaction mixture was cooled to room temperature, diluted with EtOAc, and washed with H₂O. The organic layer was dried over Na₂SO₄, filtered, and concentrated. Purification of the resulting material by flash chromatography (silica gel, EtOAc-hexane) provided the title compound as a (296 mg, 76%): ¹⁹F NMR (376 MHz, DMSO-d₆) δ−56.63 (t, J=8.2 Hz), −111.34-−113.42 (m), −113.42-−115.06 (m), −135.94 (s), −137.47 (d, J=27.0 Hz); IR (CH₂Cl₂) 3334, 3190, 1735, 1622, 1318, 1236, 1126 cm⁻¹; ESIMS m/z 417.1 ([M+H]⁺).

### Example 78: Preparation of benzyl 4-amino-3-chloro-6-(4,6-dichloro-2-fluoro-3-methoxyphenyl)-5-fluoropicolinate (C27)

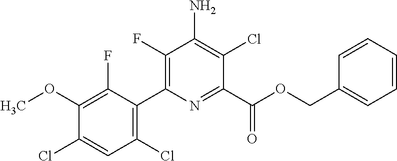 [SMILES: COc1c(Cl)cc(Cl)c(-c2nc(C(=O)OCc3ccccc3)c(Cl)c(N)c2F)c1F]

Step 1—Preparation of methyl 4-amino-3-chloro-6-(4-chloro-2-fluoro-3-methoxyphenyl)-5-fluoropicolinate. Methyl 4-amino-3,6-dichloro-5-fluoropicolinate (1.5 g, 6.28 mmol), 2-(4-chloro-2-fluoro-3-methoxyphenyl)-1,3,2-dioxaborinane (1.99 g, 8.16 mmol), potassium fluoride (0.948 g, 16.3 mmol), and Pd(PPh₃)₂Cl₂ (0.440 g, 0.628 mmol) were combined in acetonitrile (13.5 mL) and water (4.48 mL). The reaction mixture was then heated in a microwave at 115° C. in a sealed vial for 20 min. The cooled reaction mixture was partitioned between EtOAc and water. The organic phase was washed with water (2×) and concentrated onto silica gel (7 g). Purification by automated flash silica gel chromatography eluting with 2-20% EtOAc in DCM provided methyl 4-amino-3-chloro-6-(4-chloro-2-fluoro-3-methoxyphenyl)-5-fluoropicolinate as a white solid (1.5 g, 66%): ¹H NMR (400 MHz, DMSO-d₆) δ 7.47 (dd, J=8.6, 1.6 Hz, 1H), 7.30 (dd, J=8.5, 7.0 Hz, 1H), 7.13 (s, 2H), 3.93 (d, J=1.1 Hz, 3H), 3.87 (s, 3H); ¹⁹F NMR (376 MHz, DMSO-d₆) δ−137.67 (d, J=26.9 Hz), −129.19 (d, J=27.2 Hz); EIMS m/z 362.1

Step 2—Preparation of methyl 4-amino-3-chloro-6-(4,6-dichloro-2-fluoro-3-methoxyphenyl)-5-fluoropicolinate: To the solution of methyl 4-amino-3-chloro-6-(4-chloro-2-fluoro-3-methoxyphenyl)-5-fluoropicolinate (10.0 g, 27.6 mmol) and aluminum chloride (AlCl₃; 0.37 g, 2.76 mmol) in sulfuryl chloride (SO₂Cl₂; 12 mL) was added diphenyl sulfide (0.51 g, 2.76 mmol) at 20° C. After addition, the reaction mixture was stirred at 75° C. for 10 h. The reaction was quenched with water (100 mL), and the mixture was extracted with DCM (100 mL). The organic phase was washed with water (2×100 mL) and brine (100 mL), dried over Na₂SO₄ and concentrated in vacuo. Purification of the residue by silica gel column chromatography (4:1 hexanes-EtOAc) furnished the title compound as a white powder (5.5 g, 50%): mp 121-122° C.; ¹H NMR (400 MHz, DMSO-d₆) 7.78 (d, 1H), 7.22 (s, 1H), 3.94 (d, 3H), 3.86 (s, 3H); ¹³C NMR (100 MHz, DMSO-d₆) 165.09, 155.08, 152.57, 143.75, 142.14, 134.76, 129.56, 126.50, 122.70, 114.05, 62.24-62.29, 53.23; ESIMS m/z ([M+H]⁺) 397.

Step 3—Preparation of benzyl 4-amino-3-chloro-6-(4,6-dichloro-2-fluoro-3-methoxyphenyl)-5-fluoropicolinate: To a solution of the compound in Step 2 (5.5 g, 13.8 mmol) in benzyl alcohol (3 mL, 27.7 mmol) was added titanium isopropoxide (Ti(OiPr)₄; 0.39 g, 1.38 mmol) at 100° C. After addition, the reaction mixture was stirred at 100° C. for 16 h. The reaction was quenched with water (100 mL), and the mixture was extracted with DCM (100 mL). The organic phase was washed with water (2×100 mL) and brine (100 mL), dried over Na₂SO₄, and concentrated in vacuo. Purification of the residue by silica gel column chromatography with DCM as eluent afforded the title compound as a colorless solid (3.0 g, 50%): mp 54.1-55.2° C.; ¹H NMR (400 MHz, DMSO-d₆): 7.78-7.77 (d, J=4.0 Hz, 1H), 7.45-7.36 (m, 5H), 7.22 (s, 2H), 5.38 (s, 2H), 3.94-3.93 (d, J=4.0 Hz, 3H); ¹³C NMR (100 MHz, DMSO-d₆) 169.30, 149.92, 149.17, 148.37, 146.89, 139.56, 133.67, 132.73, 131.28, 118.70-118.67, 72.39, 67.02-66.98; ESIMS m/z ([M+H]⁺) 473.

### Example 79: Preparation of methyl 4-amino-3-chloro-6-(4-chloro-3-(dimethylamino)phenyl)picolinate (C28)

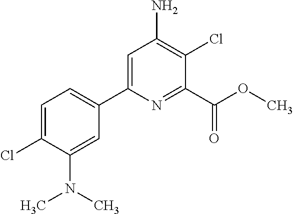 [SMILES: COC(=O)c1nc(-c2ccc(Cl)c(N(C)C)c2)cc(N)c1Cl]

Methyl 4-amino-6-(3-amino-4-chlorophenyl)-3-chloropicolinate (C29; 250 mg, 0.80 mmol) was dissolved in THF (1.6 mL) and treated sequentially with formaldehyde (48 mg, 1.6 mmol, 0.12 mL of a 37% aq solution), dibutyldichlorostannane (5 mg, 0.016 mmol) and phenylsilane (95 mg, 0.88 mmol, 0.109 mL) with stirring at room temperature. After 26 h, the reaction mixture was concentrated. Purification by silica gel chromatography with 2:1 hexane-EtOAc as the eluent gave the title compound as a white foam (225 mg, 83%): ¹H NMR (300 MHz, CDCl₃) δ 7.62 (br s, 1H), 7.36 (ad, J=1.2 Hz, 2H), 7.03 (s, 1H), 4.88 (br s, 2H), 3.99 (s, 3H), 2.85 (s, 6H); IR (thin film) 3472, 3368, 2947, 1734, 1621, 1578, 1443, 1227, 1030 cm⁻¹; ESIMS m/z 340 ([M+H]⁺).

### Example 80: Preparation of methyl 4-amino-6-(3-amino-4-chlorophenyl)-3-chloropicolinate (C29)

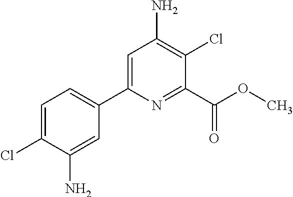 [SMILES: COC(=O)c1nc(-c2ccc(Cl)c(N)c2)cc(N)c1Cl]

To a slurry of methyl 4-amino-3-chloro-6-(4-chloro-3-nitrophenyl)picolinate (C35; 125 mg, 0.365 mmol) in acetic acid (4 mL) was added iron powder (204 mg, 36.5 mmol), and the mixture was heated to 85° C. with stirring for 0.25 h. The reaction mixture was cooled to room temperature and filtered through Celite© with EtOH and concentrated in vacuo. The reaction mixture was partitioned between EtOAc and satd NaHCO₃, the layers separated and the organic layer dried over Na₂SO₄, filtered and concentrated. The title compound was isolated as an orange solid (111 mg, 97%): mp 130-131° C.; ¹H NMR (300 MHz, CDCl₃) δ 7.36 (d, J=2.1 Hz, 1H), 7.25 (d, J=8.1 Hz, 1H), 7.09 (dd, J=8.1, 2.1 Hz, 1H), 6.98 (s, 1H), 4.85 (br s, 2H), 4.15 (br s, 2H), 3.98 (s, 3H); ESIMS m/z 312 ([M+H]⁺).

### Example 81: Preparation of methyl 4-amino-3′,5,5′-trichloro-4′-(difluoromethyl)-[2,2′-bipyridine]-6-carboxylate (C30)

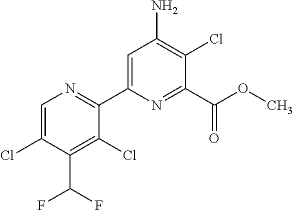 [SMILES: COC(=O)c1nc(-c2ncc(Cl)c(C(F)F)c2Cl)cc(N)c1Cl]

Acetyl chloride (97 mg, 1.24 mmol, 0.088 mL) was added dropwise to a slurry of methyl 4-acetamido-3′,5,5′-trichloro-4′-(difluoromethyl)-[2,2′-bipyridine]-6-carboxylate (C36; 105 mg, 0.247 mmol) in MeOH (3 mL) with stirring at room temperature. After 16 h, most of the MeOH was removed in vacuo and the remaining reaction mixture was added to ice cold satd aq NaHCO₃ with stirring. The resulting precipitate was collected by filtration, washed with water and dried. The title compound was isolated as an off white solid (93 mg, 98%): ¹H NMR (400 MHz, CDCl₃) δ 8.63 (s, 1H), 7.25 (t, J=52.6 Hz, 1H), 7.06 (s, 1H), 5.02 (s, 2H), 3.98 (s, 3H); ¹³C NMR (101 MHz, CDCl₃) δ 165.15, 154.17, 152.87, 150.67, 148.31, 147.66, 137.03 (t, J=23.1 Hz), 130.99 (t, J=2.4 Hz), 130.13 (t, J=3.5 Hz), 115.23, 111.56, 111.18 (t, J=242.7 Hz), 53.05; ¹⁹F NMR (376 MHz, CDCl₃) δ−117.85; ESIMS m/z 382 ([M+H]⁺).

The following compounds were prepared in like manner to the procedure outlined in Example 81:

### Methyl 4-amino-3′,5,5′-trichloro-[2,2′-bipyridine]-6-carboxylate (C31)

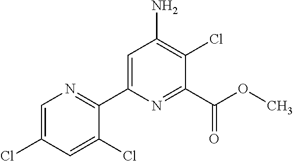 [SMILES: COC(=O)c1nc(-c2ncc(Cl)cc2Cl)cc(N)c1Cl]

Using the appropriate starting materials, the title compound was synthesized and isolated as a white foam (126 mg, 62%): ¹H NMR (400 MHz, CDCl₃) δ 8.52 (d, J=2.1 Hz, 1H), 7.82 (d, J=2.1 Hz, 1H), 7.11 (s, 1H), 5.09 (s, 2H), 3.97 (s, 3H); ¹³C NMR (101 MHz, CDCl₃) δ 165.31, 152.89, 151.98, 150.62, 147.54, 146.32, 137.85, 131.90, 130.91, 114.95, 111.55, 52.95; ESIMS m/z 332 ([M+H]⁺).

### Methyl 4-amino-4′,5-dichloro-6′-(trifluoromethyl)-[2,3′-bipyridine]-6-carboxylate (C32)

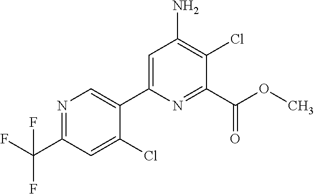 [SMILES: COC(=O)c1nc(-c2cnc(C(F)(F)F)cc2Cl)cc(N)c1Cl]

Using the appropriate starting materials, the title compound was synthesized and isolated as a white solid (158 mg, 93%): ¹H NMR (400 MHz, CDCl₃) δ 8.87 (s, 1H), 7.77 (s, 1H), 7.06 (s, 1H), 5.00 (br s, 2H), 4.00 (s, 3H); ¹³C NMR (101 MHz, CDCl₃) δ 165.14, 152.23, 150.38, 150.28, 148.80 (q, J=35.6 Hz), 148.75, 143.44, 136.43, 121.99 (q, J=3.1 Hz), 120.84 (q, J=274.6 Hz), 115.01, 111.77, 53.10; ¹⁹F NMR (376 MHz, CDCl₃ 6-68.07; ESIMS m/z 366 ([M+H]⁺).

### Methyl 4-amino-5-chloro-3′-fluoro-5′-methyl-[2,2′-bipyridine]-6-carboxylate (C33)

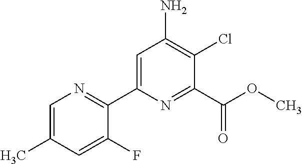

Using the appropriate starting materials, the title compound was synthesized and isolated as a white solid (37 mg, 77%): ¹H NMR (400 MHz, CDCl₃) δ 8.36 (s, 1H), 7.37 (d, J=1.3 Hz, 1H), 7.32 (d, J=11.4 Hz, 1H), 4.94 (s, 2H), 3.99 (s, 3H), 2.40 (s, 3H); ¹³C NMR (101 MHz, CDCl₃) δ 165.44, 157.78 (d, J=264.6 Hz), 151.98 (d, J=6.4 Hz), 150.54, 147.99, 145.90 (d, J=4.5 Hz), 141.18 (d, J=9.2 Hz), 136.13 (d, J=4.3 Hz), 125.11 (d, J=19.7 Hz), 114.81, 110.97 (d, J=4.5 Hz), 52.89, 17.97; ¹⁹F NMR (376 MHz, CDCl₃) δ−122.64; ESIMS m/z 296 ([M+H]⁺).

### Methyl 4-amino-3′,5,6′-trichloro-5′-(trifluoromethyl)-[2,2′-bipyridine]-6-carboxylate (C34)

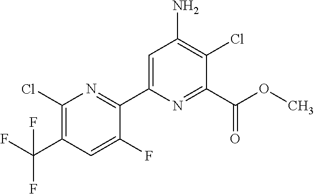 [SMILES: COC(=O)c1nc(-c2nc(Cl)c(C(F)(F)F)cc2F)cc(N)c1Cl]

Using the appropriate starting materials, the title compound was synthesized and isolated as a white foam (30 mg, 66%): ¹H NMR (400 MHz, CDCl₃) δ 8.10 (s, 1H), 7.22 (s, 1H), 5.04 (s, 2H), 3.99 (s, 3H); ¹³C NMR (101 MHz, CDCl₃) δ 165.09, 155.99, 151.52, 150.75, 147.58, 145.77, 139.30 (q, J=5.0 Hz), 129.52, 125.81 (q, J=34.1 Hz), 121.31 (q, J=273.4 Hz), 115.65, 111.54, 53.02; ¹⁹F NMR (376 MHz, CDCl₃) δ−63.73; ESIMS m/z 400 ([M+H]⁺).

### Example 82: Preparation of methyl 4-amino-3-chloro-6-(4-chloro-3-nitrophenyl)picolinate (C35)

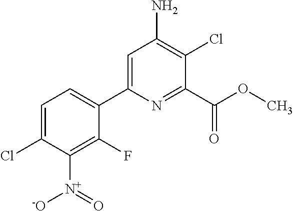

Methyl 4-amino-3-chloro-6-(4-chlorophenyl)picolinate (prepared as in Balko et al., WO2003011853A1; 1.58 g, 5.32 mmol) was added as a fine powder to ice cold con. sulfuric acid (26 mL) with stirring. Sodium nitrite (474 mg, 5.58 mmol) was added, and the mixture was allowed to warm slowly to room temperature. A large excess of ice was added to the reaction mixture, and the resulting solid was collected by filtration, washed with water and dried. The title compound was isolated as a yellow solid (1.74 g, 96%): mp 199-200° C.; ¹H NMR (300 MHz, CDCl₃) δ 8.44 (d, J=2.1 Hz, 1H), 8.11 (dd, J=2.1, 8.1 Hz, 1H), 7.61 (d, J=8.1 Hz, 1H), 7.14 (s, 1H), 4.92 (br s, 2H), 4.02, (s, 3H); ESIMS m/z 342 ([M+H]⁺).

### Example 83: Preparation of methyl 4-acetamido-3′,5,5′-trichloro-4′-(difluoromethyl)-[2,2′-bipyridine]-6-carboxylate (C36)

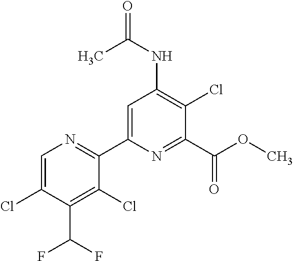 [SMILES: COC(=O)c1nc(-c2ncc(Cl)c(C(F)F)c2Cl)cc(NC(C)=O)c1Cl]

A solution of 2-bromo-3,5-dichloro-4-(difluoromethyl)pyridine (C99; 253 mg, 0.914 mmol) in DMF (2 mL) was purged with nitrogen through a needle for 30 min, followed by the sequential addition of methyl 4-acetamido-3-chloro-6-(trimethylstannyl)picolinate (C20; 250 mg, 0.64 mmol), Pd(PPh₃)₂Cl₂ (45 mg, 0.064 mmol), CuI (24 mg, 128 mmol), and cesium fluoride (194 mg, 1.28 mmol). The resulting mixture was heated to 45-55° C. under a nitrogen purge for 4 h. The cooled reaction mixture was partitioned between brine and EtOAc, the layers separated, and the organic layer dried over Na₂SO₄, filtered and concentrated. Purification by silica gel chromatography with 2:1 hexane-EtOAc as the eluent gave the title compound as an off-white solid (105 mg, 39%): ¹H NMR (400 MHz, CDCl₃) δ 8.95 (s, 1H), 8.68 (s, 1H), 8.07 (s, 1H), 7.26 (t, J=52.6 Hz, 1H), 4.01 (s, 3H), 2.33 (s, 3H); ¹⁹F NMR (376 MHz, CDCl₃) δ−117.83; ESIMS m/z 424 ([M+H]⁺).

The following compounds were prepared in like manner to the procedure outlined in Example 83:

### Methyl 4-acetamido-3′,5,5′-trichloro-[2,2′-bipyridine]-6-carboxylate (C37)

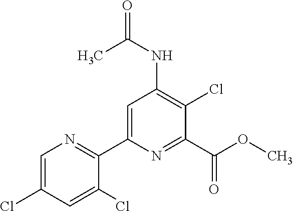 [SMILES: COC(=O)c1nc(-c2ncc(Cl)cc2Cl)cc(NC(C)=O)c1Cl]

Using the appropriate starting materials, the title compound was synthesized and isolated as a white powder (251 mg, 61%): mp 212-217° C.; ¹H NMR (400 MHz, DMSO-d₆) δ 10.02 (s, 1H), 8.75 (d, J=2.1 Hz, 1H), 8.61 (s, 1H), 8.41 (d, J=2.1 Hz, 1H), 3.93 (s, 3H), 2.25 (s, 3H); ¹³C NMR (101 MHz, DMSO-d₆) δ 170.12, 164.65, 153.26, 151.02, 148.72, 146.55, 144.04, 138.16, 131.58, 129.96, 118.27, 117.93, 52.98, 24.10; ESIMS m/z 374 ([M+H]⁺).

### Methyl 4-acetamido-4′,5-dichloro-6′-(trifluoromethyl)-[2,3′-bipyridine]-6-carboxylate (C38)

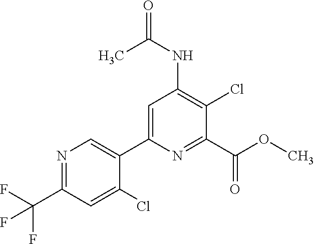 [SMILES: COC(=O)c1nc(-c2cnc(C(F)(F)F)cc2Cl)cc(NC(C)=O)c1Cl]

Using the appropriate starting materials, the title compound was synthesized and isolated as a white solid (189 mg, 40%): mp 192-198° C.; ¹H NMR (400 MHz, CDCl₃) δ 9.02 (s, 1H), 8.91 (s, 1H), 8.10 (s, 1H), 7.82 (s, 1H), 4.03 (s, 3H), 2.35 (s, 3H); ¹³C NMR (101 MHz, CDCl₃) δ 168.88, 164.44, 152.17, 151.36, 149.03 (q, J=35.5 Hz), 148.55, 143.75, 143.01, 136.03, 122.10 (q, J=3.1 Hz), 120.79 (q, J=275.7 Hz), 117.85, 116.75, 53.30, 25.12; ¹⁹F NMR (376 MHz, CDCl₃) δ−68.09; ESIMS m/z 408 ([M+H]⁺).

### Methyl 4-acetamido-5-chloro-3′-fluoro-5′-methyl-[2,2′-bipyridine]-6-carboxylate (C39)

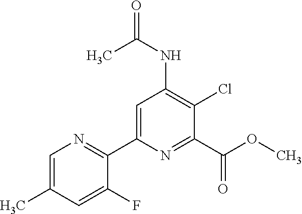

Using the appropriate starting materials, the title compound was synthesized and isolated as a white powder (55 mg, 32%): ¹H NMR (400 MHz, CDCl₃) δ 9.15 (d, J=1.3 Hz, 1H), 8.40 (s, 1H), 8.09 (s, 1H), 7.35 (d, J=11.2 Hz, 1H), 4.00 (s, 3H), 2.42 (s, 3H), 2.33 (s, 3H); ¹³C NMR (101 MHz, CDCl₃) δ 168.68, 164.75, 157.86 (d, J=264.8 Hz), 153.04 (d, J=6.3 Hz), 147.89, 146.20 (d, J=4.6 Hz), 142.96, 140.79 (d, J=9.5 Hz), 136.55 (d, J=4.2 Hz), 125.04 (d, J=19.7 Hz), 117.57, 116.14 (d, J=5.5 Hz), 53.09, 25.10, 18.00; ¹⁹F NMR (376 MHz, CDCl₃) δ−122.9; ESIMS m/z 338 ([M+H]⁺).

### Methyl 4-acetamido-3′,5,6′-trichloro-5′-(trifluoromethyl)-[2,2′-bipyridine]-6-carboxylate (C40)

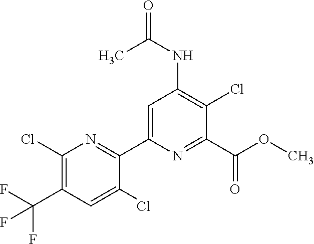 [SMILES: COC(=O)c1nc(-c2nc(Cl)c(C(F)(F)F)cc2Cl)cc(NC(C)=O)c1Cl]

Using the appropriate starting materials, the title compound was synthesized and isolated as a white powder (49 mg, 22%): ¹H NMR (400 MHz, CDCl₃) δ 9.02 (s, 1H), 8.13 (s, 1H), 8.09 (br s, 1H), 4.01 (s, 3H), 2.34 (s, 3H); ¹³C NMR (101 MHz, CDCl₃) δ 168.73, 164.33, 155.75, 152.53, 147.60, 146.19, 143.28, 139.11 (q, J=5.0 Hz), 129.53, 126.21 (q, J=34.2 Hz), 121.29 (q, J=273.4 Hz), 118.58, 116.64, 53.26, 25.12; ¹⁹F NMR (376 MHz, CDCl₃) δ−63.79; ESIMS m/z 442 ([M+H]⁺).

### Example 84: Preparation of 1-chloro-2-iodo-3-nitro-5-(trifluoromethyl)benzene (C41)

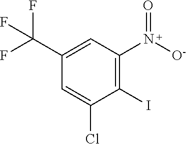

To a 5 mL vial were added 2-chloro-6-nitro-4-(trifluoromethyl)aniline (100 mg, 0.416 mmol), tert-butyl nitrite (124 μL, 1.039 mmol), and diiodomethane (1113 mg, 4.16 mmol). The vial was sealed and the reaction mixture was heated to 65° C. for 2 h. The reaction mixture was loaded directly onto silica gel. Purification by chromatography eluting with hexanes-EtOAc afforded the title compound as a yellow solid (66 mg, 45%): ¹H NMR (500 MHz, CDCl₃) δ 7.90 (d, J=1.9 Hz, 1H), 7.76 (d, J=1.9 Hz, 1H); ¹⁹F NMR (471 MHz, CDCl₃) δ−63.27; ¹⁹F NMR (471 MHz, CDCl3) δ−63.27; EIMS m/z 350.9.

### Example 85: Preparation of 4,4,5,5-tetramethyl-2-(2,2,6-trifluorobenzo[d][1,3]dioxol-5-yl)-1,3,2-dioxaborolane (C42)

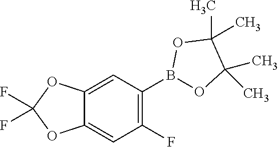 [SMILES: CC1(C)OB(c2cc3c(cc2F)OC(F)(F)O3)OC1(C)C]

The title compound was prepared according to Preparation 22 in Eckelbarger, et al., U.S. Patent Application Publication 2014/0274701 A1. 2,2,5-Trifluoro-6-iodobenzo[d][1,3]dioxole (C43; 1.0 g, 3.31 mmol) was dissolved in dry THF (10 mL). The mixture was cooled to 5° C., treated with isopropylmagnesium chloride lithium chloride complex (2.67 mL of a 1.3 M solution in THF, 3.48 mmol), and stirred for 1 h. 2-Isopropoxy-4,4,5,5-tetramethyl-1,3,2-dioxaborolane (0.723 mL, 3.31 mmol) was added and stirring was continued for 20 min. The reaction was quenched by addition of satd NH₄Cl (5 mL) solution. The reaction mixture was diluted with EtOAc (20 mL) and satd NaCl (10 mL). The organic phase was washed with satd NaCl (10 mL), dried, and evaporated. The residue was dried under vacuum. The title compound was isolated as a white solid (1.0 g, 100%): ¹H NMR (400 MHz, acetone-d₆) δ 7.52 (d, J=5.6 Hz, 1H), 7.42 (d, J=8.8 Hz, 1H), 6.49 (s, 1H), 3.93 (s, 3H); ¹⁹F NMR (376 MHz, CDCl₃) δ−49.96 (s), −104.21 (s); ¹⁹F NMR (376 MHz, CDCl₃) δ−49.96 (s), −104.21 (s); EIMS m/z 302.0.

### Example 86: Preparation of 2,2,5-trifluoro-6-iodobenzo[d][1,3]dioxole (C43)

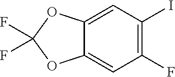 [SMILES: Fc1cc2c(cc1I)OC(F)(F)O2]

The title compound was prepared according to Preparation 21 in Eckelbarger, et al., U.S. Patent Application Publication 2014/0274701 A1. 2,2,6-Trifluorobenzo[d][1,3]dioxol-5-amine (8.0 g, 41.9 mmol) was added to concentrated HCl (200 mL). The mixture was cooled to 5° C. and treated dropwise with sodium nitrite (4.33 g, 62.8 mmol) in water (10 mL) over ca 10 min. The mixture was stirred for 30 min at 5-10° C. and then poured into a rapidly stirred two-phase mixture of sodium iodide in water (200 mL) and DCM (100 mL). After 20 min the mixture was stirred with 10% sodium bisulfite solution for 20 min. The separated aqueous phase was extracted with DCM (75 mL), and the combined extracts were washed with satd NaCl (30 mL), dried, and evaporated. Purification of the residue by silica gel chromatography eluting with hexane gave the title compound as a white solid (6.4 g, 51%): ¹H NMR (400 MHz, CDCl₃) δ 7.41 (d, J=5.0 Hz, 1H), 6.90 (d, J=6.6 Hz, 1H); ¹⁹F NMR (376 MHz, CDCl₃) δ−49.63 (s), −95.24 (s); ¹⁹F NMR (376 MHz, CDCl₃) δ−49.63 (s), −95.24 (s); EIMS m/z 302.0.

### Example 87: Preparation of 4,4,5,5-tetramethyl-2-(2,2,4 trifluorobenzo[d][1,3]dioxol-5-yl)-1,3,2-dioxaborolane (C44)

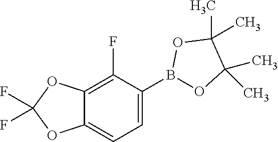 [SMILES: CC1(C)OB(c2ccc3c(c2F)OC(F)(F)O3)OC1(C)C]

The title compound was prepared according to Preparation 28 in Eckelbarger, et al., U.S. Patent Application Publication 2014/0274701 A1. 5-Bromo-2,2,4-trifluorobenzo[d][1,3]dioxole (C45; 4.0 g, 15.7 mmol) was dissolved in dry THF (20 mL). The mixture was cooled to −20° C. and treated with isopropylmagnesium chloride lithium chloride complex (12.7 mL of a 1.3 M solution in THF, 16.5 mmol) in portions over ca 10 min. The mixture was stirred for 30 min during which time the temperature rose to 0° C. 2-Isopropoxy-4,4,5,5-tetramethyl-1,3,2-dioxaborolane (3.42 mL, 16.8 mmol) was added, and the mixture was stirred for 30 min at 10-15° C. The mixture was treated with satd NH₄Cl solution (10 mL) and diluted with EtOAc (50 mL). The organic phase was washed with satd NaCl (15 mL), dried, and evaporated. The title compound was isolated as a white solid (3.5 g, 74%): ¹H NMR (400 MHz, CDCl₃) δ 7.46 (d, J=26.5 Hz, 1H), 6.90 (dd, J=18.5, 4.5 Hz, 1H), 1.35 (s, 12H); ¹⁹F NMR (376 MHz, CDCl₃) δ−49.70 (s), −126.00 (s); ¹⁹F NMR (376 MHz, CDCl₃) δ−49.70 (s), −126.00 (s); EIMS m/z 302.0.

### Example 88: Preparation of 5-bromo-2,2,4-trifluorobenzo[d][1,3]dioxole (C45)

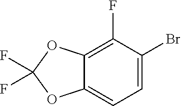 [SMILES: Fc1c(Br)ccc2c1OC(F)(F)O2]

The title compound was prepared according to Preparation 27 in Eckelbarger, et al., U.S. Patent Application Publication 2014/0274701 A1. 4-Fluorobenzo[d][1,3]dioxole-2-thione (C46; 4.8 g, 28.2 mmol) was dissolved in DCM (75 mL). The mixture was cooled to −30° C., was treated with HF-pyridine solution (70%, 18.15 mL, 141 mmol), and in portions with 1,3-dibromo-5,5-dimethylimidazolidine-2,4-dione (9.68 g, 33.9 mmol) over 30 min. The mixture was stirred for 2 h at −20 to −30° C. and then stirred with 5% sodium bisulfite solution. The separated organic phase was washed with satd NaCl (20 mL), dried and the bulk of the DCM was removed by distillation through a Vigreux column. More volatiles were removed by distillation (with no column) at 150 mm. The flask was put under vacuum (4-6 mmHg) and 2.5 g of distillate was taken overhead at 48-55° C. head temperature. This consisted of a 90:10 mixture of a single brominated isomer and the unbrominated product. ¹H NMR spectral analysis showed the bromo product to be the 4-bromo isomer. The product was used without further purification for conversion to the boronate as a brown oil (3.2 g, 45%): ¹H NMR (400 MHz, CDCl₃) δ 7.28 (dd, J=8.6, 6.2 Hz, 1H), 6.81 (dd, J=8.6, 1.3 Hz, 1H); ¹⁹F NMR (376 MHz, CDCl₃) δ−49.25 (s), −126.72 (s); ¹⁹F NMR (376 MHz, CDCl₃) δ−49.25 (s), −126.72 (s); EIMS m/z 254.0.

### Example 89: Preparation of 4-fluorobenzo[d][1,3]dioxole-2-thione (C46)

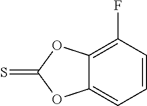 [SMILES: Fc1cccc2oc(=S)oc12]

The title compound was prepared according to Preparation 26 in Eckelbarger, et al., U.S. Patent Application Publication 2014/0274701 A1. 3-Fluorobenzene-1,2-diol (5.0 g, 39.0 mmol) and thiophosgene (3.29 mL, 42.9 mmol) were combined in CHCl₃ (50 mL). The mixture was cooled to 10° C. and treated dropwise with a 10% NaOH solution (36 g, 90 mmol) over ca 30 min. The reaction mixture was stirred for 2 h at 20° C. The solvent was removed under vacuum, and the solid was collected by filtration and washed with water. The solid was dissolved in EtOAc (100 mL), and the solution was washed with water (30 mL), satd NaCl (30 mL), dried, and concentrated. Purification of the residue by silica gel chromatography with 0-30% EtOAc-hexane afforded the title compound as a brown solid (5.1 g, 77%): mp 58-59° C.; ¹H NMR (400 MHz, CDCl₃) δ 7.28 (m, 1H), 7.12 (m, 1H); ¹⁹F NMR (376 MHz, CDCl₃) δ−131.32; EIMS m/z 170.

### Example 90: Preparation of 2-(6-chloro-2,2-difluorobenzo[d][1,3]dioxol-5-yl)-4,4,5,5-tetramethyl-1,3,2-dioxaborolane (C47)

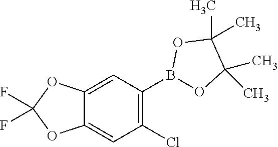 [SMILES: CC1(C)OB(c2cc3c(cc2Cl)OC(F)(F)O3)OC1(C)C]

The title compound was prepared according to Preparation 47 in Eckelbarger, et al., U.S. Patent Application Publication 2014/0274701 A1. 5-Bromo-6-chloro-2,2-difluorobenzo[d][1,3]dioxole (1.0 g, 3.68 mmol) was dissolved in dry THF (47.9 mL). The mixture was cooled to 0-5° C. and was treated with the isopropylmagnesium chloride lithium chloride complex (2.9 mL of a 1.3 M solution in THF, 3.87 mmol) over 10 min. After 30 min, a solution of 2-isopropoxy-4,4,5,5-tetramethyl-1,3,2-dioxaborolane (1.91 g, 10.3 mmol) in THF (15 mL) was added over 5 min. Stirring was continued at 10-15° C. for 30 min. After 45 min after the addition of the borolane, satd NH₄Cl solution (10 mL) was added. The mixture was shaken with EtOAc (20 mL) and satd NaCl (10 mL). The organic phase was washed with satd NaCl (10 mL), dried, and concentrated. The title compound was isolated as a white solid (1.2 g, 100%) that was used without further purification: ¹H NMR (400 MHz, CDCl₃) δ 7.40 (s, 1H), 7.08 (s, 1H), 1.36 (s, 12H); EIMS m/z 318.

### Example 91: Preparation of 2,2-difluoro-5-iodobenzo[d][1,3]dioxol-4-ol (C48)

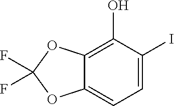 [SMILES: Oc1c(I)ccc2c1OC(F)(F)O2]

The title compound was prepared according to a method in Altenbach, R. J., et al., WO 2017/009804 A1. A solution of 2,2-difluorobenzo[d][1,3]dioxol-4-ol (2.00 g, 11.5 mmol) in MeOH (20.2 mL) was cooled to <0° C., and N-ethyl-N-isopropylpropan-2-amine (1.16 mL, 12.6 mmol) and iodine chloride (1.27 mL, 25.3 mmol) were added (drop wise at <5° C.). After 30 min, the reaction was quenched with satd aq Na₂S₂O₃ (10 mL), and the mixture was partitioned between water (10 mL) and Et₂O (30 mL). The organic layer was washed with brine (5 mL), dried over Na₂SO₄ filtered, and concentrated. Purification of the residue by silica gel column chromatography eluting with 0-20% EtOAc-hexane gave 2,2-difluoro-5,7-diiodobenzo[d][1,3]dioxol-4-ol (1.03 g, 21%), 2,2-difluoro-5-iodobenzo[d][1,3]dioxol-4-ol (0.487 g, 14%), and 2,2-difluoro-7-iodobenzo[d][1,3]dioxol-4-ol (1.19 g, 35%). ¹H NMR (400 MHz, CDCl₃) δ 7.39 (d, J=8.5 Hz, 1H), 6.52 (d, J=8.5 Hz, 1H), 6.24 (s, 1H); ¹⁹F NMR (376 MHz, CDCl₃) δ−49.42; ¹⁹F NMR (376 MHz, CDCl₃) δ−49.42; EIMS m/z 300.

### Example 92: Preparation of 2,2-difluoro-5-iodo-4-methoxybenzo[d][1,3]dioxole (C49)

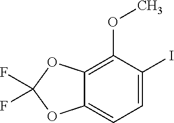

To a cooled 0° C. solution of 2,2-difluoro-5-iodobenzo[d][1,3]dioxol-4-ol (C48; 500 mg, 1.67 mmol) in anhydrous THF were added potassium carbonate (576 mg, 4.17 mmol) and dimethyl sulfate (0.788 mL, 8.33 mmol). The reaction mixture was warmed to room temperature and allowed to stir for 3 h. The reaction was quenched with satd aq NH₄Cl and extracted with EtOAc (3×25 mL). The organic extracts were combined, dried over Na₂SO₄, and concentrated. Purification of the resulting brown residue by silica gel chromatography afforded the title compound as a white solid (448 mg, 69%): ¹H NMR (500 MHz, CDCl₃) δ 7.47 (d, J=8.4 Hz, 1H), 6.54 (d, J=8.4 Hz, 1H), 4.12 (s, 3H); ¹⁹F NMR (471 MHz, CDCl₃) δ −49.78; EIMS m/z 314.0.

### Example 93: Preparation of 4,4,5,5-tetramethyl-2-(2,2,7-trifluorobenzo[d][1,3]dioxol-5-yl)-1,3,2-dioxaborolane (C50)

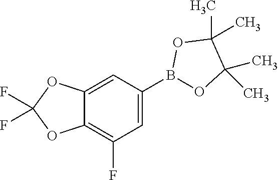 [SMILES: CC1(C)OB(c2cc(F)c3c(c2)OC(F)(F)O3)OC1(C)C]

The title compound was prepared according to Preparation 51 in Eckelbarger, et al., U.S. Patent Application Publication 2014/0274701 A1. 6-Bromo-2,2,4-trifluorobenzo[d][1,3]dioxole (C51; 2.00 g, 7.84 mmol) was dissolved in dry THF (10 mL), cooled to −5 to 0° C., and treated in portions with isopropylmagnesium chloride lithium chloride complex (6.34 mL of a 1.3 M solution in THF, 8.24 mmol) while keeping the temp. below 5° C. The cooling bath was removed, and the mixture was stirred for 30 min. 2-Isopropoxy-4,4,5,5-tetramethyl-1,3,2-dioxaborolane (1.56 g, 8.39 mmol) was added, and the mixture was stirred for 1 h. The mixture was treated with satd NH₄Cl (5 mL) and stirred for 5 min. The mixture was diluted with EtOAc (40 mL) and satd NaCl (10 mL). The pH was adjusted to ca 2 with HCl and after extraction, the organic phase was washed with satd NaCl (5 mL), dried and concentrated. The title compound was isolated as a brown oil (2.1 g, 89%), which was used without further purification: ¹H NMR (400 MHz, CDCl₃) δ 7.36 (d, J=9.8 Hz, 1H), 7.29 (d, J=6.5 Hz, 1H), 1.33 (s, 12H); ¹⁹F NMR (376 MHz, CDCl₃) δ−49.79, −136.26; EIMS m/z 302.0.

### Example 94: Preparation of 6-bromo-2,2,4-trifluorobenzo[d][1,3]dioxole (C51)

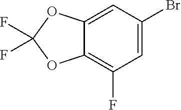 [SMILES: Fc1cc(Br)cc2c1OC(F)(F)O2]

The title compound was prepared according to Preparation 50 in Eckelbarger, et al., U.S. Patent Application Publication 2014/0274701 A1. 6-Bromo-4-fluorobenzo[d][1,3]dioxole-2-thione (C52; 6.5 g, 26.1 mmol) was dissolved in DCM (150 mL). The solution was cooled to −35° C., was treated with HF-pyridine solution (70%, 35.0 mL, 272 mmol), and in portions with 1-iodopyrrolidine-2,5-dione (19.0 g, 84.4 mmol). Over 30 min, the reaction was warmed from −35 to 0° C. The cooling bath was removed and allowed to warm to 25° C. over 30 min, at which point, the conversion was complete. The reaction mixture was treated in portions with external cooling below 15° C. with NaHSO₃ (8 g) in water (50 mL) with stirring for 15 min. The mixture was further diluted with water (200 mL) to dissolve solids. The organic phase was washed with satd NaCl (30 mL) and dried. The volatiles were removed by distillation through a 7 tray Oldershaw column and then through a 200 mm Vigreux column at 1 atm until the pot volume was ca. 50 mL. Distillation was stopped when the head temperature was maintained at 75° C. during removal of ca. 10 mL distillate and then dropped as heating was applied. After cooling, the product was distilled at ca. 50 mmHg at a temperature of 75-80° C. The title compound was isolated as a pink liquid (5.3 g, 74%): ¹H NMR (400 MHz, CDCl₃) δ 7.11 (dd, J=9.0, 1.7 Hz, 1H), 7.07 (m, 1H); ¹⁹F NMR (376 MHz, CDCl₃) δ−49.56, −132.65; EIMS m/z 254.0.

### Example 95: Preparation of 6-bromo-4-fluorobenzo[d][1,3]dioxole-2-thione (C52)

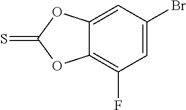 [SMILES: Fc1cc(Br)cc2oc(=S)oc12]

The title compound was prepared according to Preparation 49 in Eckelbarger, et al., U.S. Patent Application Publication 2014/0274701 A1. 5-Bromo-3-fluorobenzene-1,2-diol (2.0 g, 9.66 mmol) was dissolved in chloroform (25 mL) and was treated with thiophosgene (0.815 mL, 10.6 mmol). The reaction mixture was cooled to 0-5° C. A 10% aq NaOH solution (8.89 g, 22.2 mmol) was added dropwise with vigorous stirring over ca 30 min. The reaction was stirred for another 30 min after the addition was complete. After 1 h, the chloroform was removed under vacuum, and the pH was adjusted to ca 2 by addition of 6 M HCl. The solid product was taken up in EtOAc (120 mL). The organic phase was washed with satd NaCl (30 mL), dried, and concentrated. Purification by silica gel chromatography with 0-30% EtOAc-hexane provided the title compound as a brown solid (1.5 g, 59%): ¹H NMR (400 MHz, CDCl3) δ 7.35-7.30 (m, 1H), 7.29 (d, J=1.6 Hz, 1H); ¹⁹F NMR (376 MHz, CDCl₃) δ−128.93; EIMS m/z 248.0.

### Example 96: Preparation of 2-(7-fluorobenzo[b]thiophen-6-yl)-4,4,5,5-tetramethyl-1,3,2-dioxaborolane (C53)

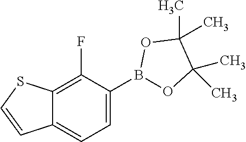 [SMILES: CC1(C)OB(c2ccc3ccsc3c2F)OC1(C)C]

6-Bromo-7-fluorobenzo[b]thiophene (1.00 g, 4.33 mmol), anhydrous potassium acetate (0.849 g, 8.65 mmol) and 4,4,4′,4′,5,5,5′,5′-octamethyl-2,2′-bi(1,3,2-dioxaborolane) (1.21 g, 4.76 mmol) were combined in dry dioxane (15 mL), and the mixture was sparged with nitrogen for 10 min and treated with Pd(dppf)Cl₂ (0.177 g, 0.216 mmol). The reaction mixture was heated to 90° C. for 20 h. The mixture was cooled, stirred with EtOAc (50 mL) and water (20 mL) and filtered through Celite©. The organic phase was washed with satd NaCl (10 mL), dried, and concentrated. Purification by silica gel chromatography with 0-30% EtOAc-hexane gave the title compound as a white solid (820 mg, 65%): mp 107-108° C.; ¹H NMR (400 MHz, DMSO-d₆) δ 7.98 (d, J=5.3 Hz, 1H), 7.74 (dd, J=7.9, 1.0 Hz, 1H), 7.64-7.57 (m, 1H), 1.33 (s, 12H); ¹⁹F NMR (376 MHz, DMSO-d₆) δ−104.03; EIMS m/z 278.

### Example 97: Preparation of 6-bromo-7-fluorobenzo[b]thiophene (C54)

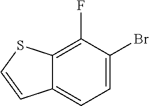 [SMILES: Fc1c(Br)ccc2ccsc12]

Ethyl 6-bromo-7-fluorobenzo[b]thiophene-2-carboxylate (4.1 g, 13.52 mmol) was added to EtOH (50 mL) and water (50 mL), treated with potassium hydroxide (4.17 g, 74.4 mmol), and heated to reflux for 3 h. After cooling, much of the EtOH was removed by evaporation under vacuum. The residue was taken up in water and made acidic with 1 M HCl. The precipitated acid was taken up in EtOAc (100 mL), and the solution was washed with satd NaCl (15 mL), dried, and concentrated to give 3.5 g of the acid. The acid (2.5 g, 12 mmol) and copper powder (260 mg, 4.0 mmol) were combined in quinoline (12 mL), and the mixture was heated to 185° C. Evolution of gases was observed. After 45 min of heating, the mixture was cooled, diluted with EtOAc (100 mL), and stirred with 1 M HCl (150 mL) for 10 min. The mixture was filtered through Celite© to remove solids. The organic phase was washed with water (20 mL) and satd NaCl (20 mL), dried, and concentrated. Purification by silica gel chromatography with 0-5% EtOAc-hexanes provided material that contained ca 80% of the title compound. The material was further purified by RP-HPLC with 70% acetonitrile buffered with 0.20% H₃PO₄ as the eluent. The title compound was isolated as a white crystalline solid: mp 45-46° C.; ¹H NMR (400 MHz, DMSO-d₆) δ 7.92 (dd, J=5.3, 0.5 Hz, 1H), 7.72 (d, J=8.4 Hz, 1H), 7.67 (dd, J=8.4, 6.3 Hz, 1H), 7.58 (dd, J=5.3, 3.9 Hz, 1H); ¹⁹F NMR (376 MHz, DMSO-d₆) δ−110.20; EIMS m/z 232.

### Example 98: Preparation of 6-(4,4,5,5-tetramethyl-1,3,2-dioxaborolan-2-yl)benzo[d]oxazole (C55)

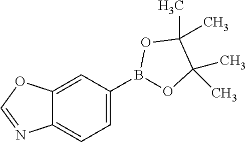 [SMILES: CC1(C)OB(c2ccc3ncoc3c2)OC1(C)C]

6-Bromobenzo[d]oxazole (0.600 g, 3.03 mmol), anhydrous potassium acetate (0.595 g, 6.06 mmol) and 4,4,4′,4′,5,5,5′,5′-octamethyl-2,2′-bi(1,3,2-dioxaborolane) (0.846 g, 3.33 mmol) were combined in dry dioxane (10 mL), and the mixture was sparged with nitrogen for 15 min. The reaction mixture was treated with Pd(dppf)Cl₂ (0.124 mg, 0.152 mmol) and was heated to 90° C. for 16 h After cooling, the mixture was shaken with EtOAc (45 mL) and satd NaCl (10 mL) and was filtered to remove dark solids. The organic phase was dried and concentrated. Purification by silica gel chromatography with 0-30% EtOAc-hexane afforded the title compound as white crystals (600 mg, 74%): mp 79-81° C.; ¹H NMR (400 MHz, CDCl₃) δ 8.13 (s, 1H), 8.04 (s, 1H), 7.80 (qt, J=3.3, 1.8 Hz, 2H), 1.37 (s, 12H); EIMS m/z 245.

### Example 99: Preparation of 1-fluoro-5-iodo-4-methoxy-2-(trifluoromethyl)benzene (C56)

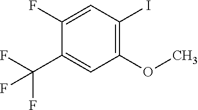

A flask was charged with DCM (95 mL) and 1-chloro-4-methoxy-2-(trifluoromethyl)benzene (5 g, 23.74 mmol) to form a clear solution. Iodine (6.63 g, 26.1 mmol) and silver trifluoromethanesulfonate (7.32 g, 28.5 mmol) were added sequentially. The reaction mixture was allowed to stir at room temperature under nitrogen atmosphere. After 1.5 h, he reaction was filtered through Celite®, eluting with DCM until the eluent was no longer purple. The purple filtrate was extracted with satd aq sodium thiosulfate (50 mL) until the mixture was all light yellow. The biphasic mixture was diluted with water (100 mL) and the layers were separated. The aqueous layer was extracted with DCM (2×50 mL). The combined organic extracts were dried with Na₂SO₄, filtered, and concentrated by rotary evaporation to give 9.0 g of brown liquid containing fine needle crystals. Purification of the mixture (which was loaded in minimal DCM directly onto a dry column) by silica gel flash column chromatography eluting with 0-3% EtOAc-heptane. The title compound was isolated as a white crystalline solid (1.677 g, 85%): ¹H NMR (500 MHz, CDCl₃) δ 7.69-7.54 (m, 1H), 6.93 (d, J=5.8 Hz, 1H), 3.90 (s, 3H); ¹⁹F NMR (471 MHz, CDCl₃) δ−61.54, −123.86; ¹⁹F NMR (471 MHz, CDCl₃) δ−61.54, −123.86; EIMS m/z 320.0.

### Example 100: Preparation of 2-(5-fluoro-2-methoxy-4-(trifluoromethyl)phenyl)-4,4,5,5-tetramethyl-1,3,2-dioxaborolane (C57)

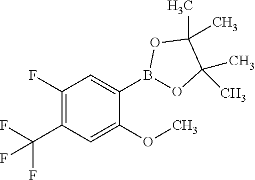 [SMILES: COc1cc(C(F)(F)F)c(F)cc1B1OC(C)(C)C(C)(C)O1]

To a degassed solution of potassium acetate (1.023 g, 10.43 mmol), 1-fluoro-5-iodo-4-methoxy-2-(trifluoromethyl)benzene (C56; 776 mg, 2.425 mmol) and 4,4,4′,4′,5,5,5′,5′-octamethyl-2,2′-bi(1,3,2-dioxaborolane) (739 mg, 2.91 mmol) in 1,4-dioxane (12.1 mL) was added Pd(PPh₃)₂Cl₂ (0.170 g, 0.242 mmol). The reaction was stirred at reflux overnight. Another 0.10 equiv of catalyst was added. After 3 h, the reaction mixture was cooled and concentrated. The residue was loaded onto silica. Purification by column chromatography (hexanes-EtOAc) afforded the title compound as a white solid (313 mg, 34%): ¹H NMR (300 MHz, CDCl₃) δ 7.47 (d, J=10.2 Hz, 1H), 7.00 (d, J=5.2 Hz, 1H), 3.85 (s, 3H), 1.36 (s, 12H); ¹⁹F NMR (471 MHz, CDCl₃) δ−61.67, −126.88; EIMS m/z 320.1.

### Example 101: Preparation of 2-(2-fluoro-5-methyl-4-(trifluoromethyl)phenyl)-4,4,5,5-tetramethyl-1,3,2-dioxaborolane (C58)

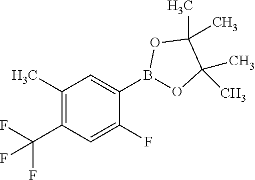 [SMILES: Cc1cc(B2OC(C)(C)C(C)(C)O2)c(F)cc1C(F)(F)F]

Benzoic peroxyanhydride (0.025 g, 0.104 mmol) and 4,4,4′,4′,5,5,5′,5′-octamethyl-2,2′-bi(1,3,2-dioxaborolane) (1.446 g, 5.70 mmol) were weighed in a 20 mL sealed tube. MeCN (15.7 mL), 2-fluoro-5-methyl-4-(trifluoromethyl)aniline (1.00 g, 5.18 mmol) and tert-butyl nitrite (0.924 mL, 7.77 mmol) were then added in succession. The resulting reaction solution was allowed to stir for 1-2 h at 60° C. (Nitrogen gas evolution was completed within 5 min.) The solution was concentrated under reduced pressure. Purification of the residue by flash chromatography afforded the title compound as an orange oil (0.745 g, 45%): ¹H NMR (500 MHz, CDCl₃) δ 7.63 (d, J=5.7 Hz, 1H), 7.32-7.24 (m, 1H), 2.44 (dd, J=2.3, 1.3 Hz, 3H), 1.37 (s, 12H); ¹⁹F NMR (471 MHz, CDCl₃) δ−62.71, −106.05; EIMS m/z 289.1 ([M-Me]).

### Example 102: Preparation of 1-chloro-2-iodo-4-methoxy-5-(trifluoromethyl)benzene (C59)

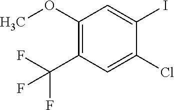

In a 5 mL vial were added 2-chloro-5-methoxy-4-(trifluoromethyl)aniline (429 mg, 1.90 mmol), tert-butyl nitrite (23.1 μL, 0.194 mmol), and diiodomethane (208 mg, 0.776 mmol). The vial was sealed and the reaction mixture was heated to 65° C. for 2 h. The reaction mixture was loaded directly onto a silica gel column. Purification by column chromatography eluting with hexanes-EtOAc afforded the title compound as a yellow solid (132 mg, 20.6%): ¹H NMR (500 MHz, CDCl₃) δ 7.58 (s, 1H), 7.44 (s, 1H), 3.89 (s, 3H); ¹⁹F NMR (471 MHz, CDCl₃) δ−63.01; EIMS m/z 336.0.

### Example 103: Preparation of 5-amino-4-chloro-2-(trifluoromethyl)benzonitrile (C60)

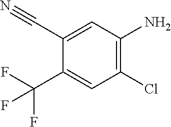 [SMILES: N#Cc1cc(N)c(Cl)cc1C(F)(F)F]

A 20 mL vial was charged with 5-bromo-2-chloro-4-(trifluoromethyl)aniline (159 mg, 0.579 mmol) and N,N-dimethylformamide (1158 μL). The mixture was heated to 140° C. for 12 h. The reaction mixture was cooled and poured into water (20 mL) containing concentrated ammonium hydroxide (2 mL). The mixture was diluted DCM (100 mL) and was filtered through Celite®. The layers were separated, and the aqueous layer was extracted with DCM. The organic extracts were combined, dried over Na₂SO₄, and concentrated under reduced pressure. The resulting brown residue was loaded onto silica gel. Purification by column chromatography eluting with a gradient of pure hexanes (300 mL) to hexanes-EtOAc (20:1, 300 mL; 10:1, 300 mL; and 5:1, 300 mL). An incremental gradient eluting with less polar solvent systems is critical to separate the cyanobromide byproduct from the desired product. The title compound was isolated as a white solid (41 mg, 32%): ¹H NMR (500 MHz, CDCl₃) δ 7.64 (s, 1H), 7.12 (s, 1H), 4.67 (s, 2H); ¹⁹F NMR (471 MHz, CDCl₃) δ−60.60; EIMS m/z 221.

### Example 104: Preparation of 4-chloro-5-iodo-2-(trifluoromethyl)benzonitrile (C61)

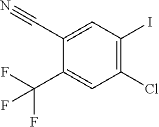 [SMILES: N#Cc1cc(I)c(Cl)cc1C(F)(F)F]

To a 20 mL vial was added 5-amino-4-chloro-2-(trifluoromethyl)benzonitrile (C60; 0.041 g, 0.186 mmol) and diiodomethane (0.299 mL, 3.72 mmol). The mixture was heated to 100° C. before adding tert-butyl nitrite (0.055 mL, 0.465 mmol). Vigorous gas evolution was observed upon addition. The reaction mixture was allowed to stir at elevated temperature for 2 hours before cooling and loading the reaction onto silica gel chromatographing with hexanes/EtOAc to afford 4-chloro-5-iodo-2-(trifluoromethyl)benzonitrile (37.5 mg, 0.107 mmol, 57.8% yield). as a clear oil (37.5 mg, 57.8%); ¹H NMR (400 MHz, CDCl₃) δ 8.35-8.27 (m, 1H), 7.82 (s, 1H).; ¹⁹F NMR (376 MHz, CDCl3) δ−62.37.; ¹⁹F NMR (376 MHz, CDCl3) δ−62.37; EIMS m/z 331.0.

### Example 105: Preparation of 5-fluoro-6-(trimethylstannyl)benzo[c][1,2,5]oxadiazole (C62)

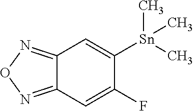

To a solution of 1,1,1,2,2,2-hexamethyldistannane (1.051 ml, 5.07 mmol) in toluene (9.22 mL) was added 5-bromo-6-fluorobenzo[c][1,2,5]oxadiazole (1.000 g, 4.61 mmol). For the preparation of 5-bromo-6-fluorobenzo[c][1,2,5]oxadiazole (1.000 g, 4.61 mmol) see U.S. Patent Application Publication 2014/0274702. After being de-gassed and backfilled with nitrogen, Pd(dppf)Cl₂ (0.376 g, 0.461 mmol, 0.1 equiv) was added. The reaction was stirred at reflux overnight under nitrogen, cooled and concentrated. The residue was purified by silica gel column chromatography eluting with pure hexanes followed by 10:1 hexanes-EtOAc. The product containing fractions were collected and concentrated to afford 5-fluoro-6-(trimethylstannyl)benzo[c][1,2,5]oxadiazole (0.953 g, 69%).

### Example 106: Preparation of 1-chloro-2-iodo-3-methyl-5-(trifluoromethyl)benzene (C63)

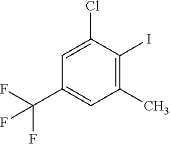

To a 50 mL sealed tube were added 2-chloro-6-methyl-4-(trifluoromethyl)aniline (C64; 0.895 g, 4.27 mmol) and diiodomethane (22.88 g, 85 mmol). The heterogeneous mixture was heated to 110° C. tert-Butyl nitrite (1.10 g, 10.7 mmol) was added in a single portion. Upon addition, the reaction mixture turned orange and became homogenous. Two additional 2.5-equiv portions (total of 5 equivs) of tert-butyl nitrite were added over 4 h. After 4 h, the reaction was cooled to room temperature and loaded onto silica gel Purification by column chromatography eluting with hexanes furnished the title compound as a clear oil (634 mg, 46%): ¹H NMR (400 MHz, CDCl₃) δ 7.52 (dt, J=2.3, 0.8 Hz, 1H), 7.34 (dt, J=2.2, 0.7 Hz, 1H), 2.59 (s, 3H); ¹⁹F NMR (376 MHz, CDCl₃) δ−63.05; EIMS m/z 320.0.

### Example 107: Preparation of 2-chloro-6-methyl-4-(trifluoromethyl)aniline (C64)

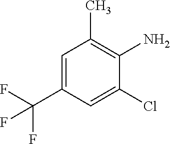

A 50 mL sealed vial was charged with 2-methyl-4-(trifluoromethyl)aniline (2.50 g, 14.3 mmol), 1-chloropyrrolidine-2,5-dione (2.10 g, 15.7 mmol) and MeCN (28.5 mL). The mixture was headed to 80° C. for 12 h. The reaction mixture was loaded directly onto silica gel. Purification by column chromatography eluting with a linear gradient of 0-100% EtOAc-hexanes afforded the title compound as a clear, viscous oil (0.895 g, 30%): ¹H NMR (400 MHz, CDCl₃) δ 7.44-7.34 (m, 1H), 7.23-7.13 (m, 1H), 4.32 (s, 2H), 2.23 (s, 3H); ¹⁹F NMR (376 MHz, CDCl₃) δ−61.32; ESIMS m/z 210.0 ([M+H]⁺).

### Example 108: Preparation of 2-(2-chloro-5-nitro-4-(trifluoromethyl)phenyl)-4,4,5,5-tetramethyl-1,3,2-dioxaborolane (C65)

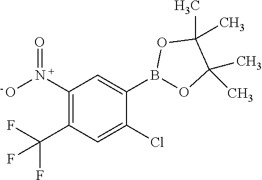

Benzoic peroxyanhydride (0.020 g, 0.083 mmol) and 4,4,4′,4′,5,5,5′,5′-octamethyl-2,2′-bi(1,3,2-dioxaborolane) (1.16 g, 4.58 mmol) were weighed in a 20 mL sealed tube. MeCN (12.6 mL), 2-chloro-5-nitro-4-(trifluoromethyl)aniline (C66; 1.00 g, 4.16 mmol) and tert-butyl nitrite (0.742 mL, 6.24 mmol) were then added in succession. The resulting reaction solution was allowed to stir for 2 h at 60° C. (Nitrogen gas evolution was complete within 5 min.) The solution was then concentrated under reduced pressure, and the brown oily residue was used without further purification: ¹H NMR (400 MHz, CDCl₃) δ 8.24 (s, 1H), 7.79 (s, 1H), 1.39 (s, 12H); ¹⁹F NMR (376 MHz, CDCl₃) δ−61.50.

### Example 109: Preparation of 2-chloro-5-nitro-4-(trifluoromethyl)aniline (C66)

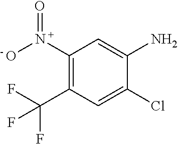

3-Nitro-4-(trifluoromethyl)aniline (1.5 g, 7.28 mmol) was weighed into a 25 mL round bottom flask equipped with a septa. Methanol (14.6 mL) was added and the mixture was stirred until complete dissolution was achieved. 1-Chloropyrrolidine-2,5-dione (0.972 g, 7.28 mmol) was added in a single portion with stirring. The reaction mixture was allowed to stir at room temperature until complete consumption of the starting material was observed. After 24 h, the reaction mixture was concentrated and loaded directly onto silica gel. Purification by column chromatography eluting with 90% hexanes and EtOAc furnished the title compound as an orange solid (1.00 g, 57%): ¹H NMR (400 MHz, CDCl₃) δ 7.66 (s, 1H), 7.25 (s, 1H), 4.78 (s, 3H); ¹⁹F NMR (376 MHz, CDCl₃) δ−58.75; EIMS m/z 240.

### Example 110: Preparation of 2,2-difluoro-5-iodo-4-methoxy-7-vinylbenzo[d][1,3]dioxole (C67)

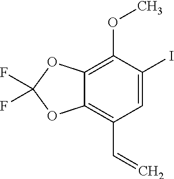 [SMILES: C=Cc1cc(I)c(OC)c2c1OC(F)(F)O2]

To a cooled 0° C. solution of 2,2-difluoro-5-iodo-7-vinylbenzo[d][1,3]dioxol-4-ol (C68; 36.0 mg, 0.110 mmol) in anhydrous THF was added potassium carbonate (76 mg 0.552 mmol). The reaction mixture was allowed to stir at 0° C. for 30 min before the addition of dimethyl sulfate (0.1 mL, 1.06 mmol) as a 2.0 M solution in tert-butyl methyl ether. The reaction mixture was warmed to room temperature and allowed to stir for 3 h before being concentrated and loaded directly onto silica. Purification by column chromatography eluting with hexanes and ethyl acetate provided the title compound as a clear oil (40 mg, 100%): ¹H NMR (500 MHz, CDCl₃) δ 7.50 (s, 1H), 6.55-6.48 (m, 1H), 5.83 (dd, J=17.7, 0.7 Hz, 1H), 5.42 (dd, J=11.2, 0.7 Hz, 1H), 4.11 (s, 3H); ¹⁹F NMR (471 MHz, CDCl₃) δ−49.39; ESIMS m/z 341.4 ([M+H]⁺).

### Example 111: Preparation of 2,2-difluoro-5-iodo-7-vinylbenzo[d][1,3]dioxol-4-ol (C68)

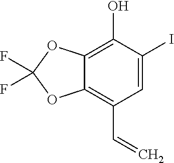

To a solution of ((2,2-difluoro-5-iodo-7-vinylbenzo[d][1,3]dioxol-4-yl)oxy)triisopropylsilane (C69; 140 mg, 0.290 mmol) in THF (2 mL) was added tetrabutylammonium fluoride hydrate (81 mg, 0.290 mmol) in a single portion at room temperature. The reaction mixture was allowed to stir overnight. The reaction mixture was concentrated and the residue was loaded onto silica gel. Purification by column chromatography with a linear gradient of hexanes-EtOAc afforded the title compound as a clear oil (36 mg, 36%): ¹H NMR (500 MHz, CDCl₃) δ 7.41 (s, 1H), 6.51 (dd, J=17.8, 11.3 Hz, 1H), 5.82 (d, J=17.8 Hz, 1H), 5.42 (d, J=11.3 Hz, 1H); ¹⁹F NMR (471 MHz, CDCl₃) δ−48.98; EIMS m/z 326.0.

### Example 112: Preparation of ((2,2-difluoro-5-iodo-7-vinylbenzo[d][1,3]dioxol-4-yl)oxy)triisopropylsilane (C69)

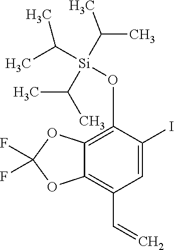 [SMILES: C=Cc1cc(I)c(O[Si](C(C)C)(C(C)C)C(C)C)c2c1OC(F)(F)O2]

Procedure adapted from: U.S. Pat. Appl. Publ., 20140080862, 20 Mar. 2014. To a 25 mL vial was added ((2,2-difluoro-5,7-diiodobenzo[d][1,3]dioxol-4-yl)oxy)triisopropylsilane (C70; 200 mg, 0.344 mmol), tributyl(vinyl)stannane (120 mg, 0.378 mmol) and toluene. The mixture was degassed with nitrogen for 10 min before adding Pd(dppf) as a complex with dichloromethane (1:1). The reaction mixture was heated at 100° C. for 16 h and was concentrated under reduced pressure. Purification of the residue by chromatography (silica gel, heptane-EtOAc) afforded the title compound as a clear oil (140 mg, 84%): ¹H NMR (500 MHz, CDCl₃) δ 7.50 (s, 1H), 6.51 (dd, J=17.7, 11.3 Hz, 1H), 5.81 (dd, J=17.8, 0.7 Hz, 1H), 5.39 (dd, J=11.3, 0.7 Hz, 1H), 1.44-1.36 (m, 3H), 1.14 (d, J=7.6 Hz, 18H); ¹⁹F NMR (471 MHz, CDCl₃) δ−49.40; EIMS m/z 482.2.

### Example 113: Preparation of ((2,2-difluoro-5,7-diiodobenzo[d][1,3]dioxol-4-yl)oxy)triisopropylsilane (C70)

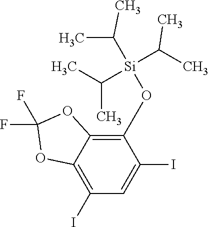 [SMILES: CC(C)[Si](Oc1c(I)cc(I)c2c1OC(F)(F)O2)(C(C)C)C(C)C]

A solution of 2,2-difluoro-5,7-diiodobenzo[d][1,3]dioxol-4-ol (C72; 491 mg, 1.15 mmol) in DCM (2023 μL) and 2,6-dimethylpyridine (267 μL, 2.306 mmol) in DCM (2023 μLl) was cooled to <0° C. Triisopropylsilyl trifluoromethanesulfonate (465 μL, 1.73 mmol) was added (drop wise at <5° C.), and the reaction mixture was allowed to stir at room temperature for 2 h before being loaded directly onto silica gel. Purification by silica gel chromatography eluting with hexanes-EtOAc (100% to 10%) afforded the title compound as a clear oil (510 mg, 76%): ¹H NMR (500 MHz, CDCl₃) δ 7.76 (s, 1H), 1.43-1.34 (m, 3H), 1.13 (d, J=7.5 Hz, 18H); ¹⁹F NMR (471 MHz, CDCl₃) δ−49.18; EIMS m/z 582.1.

### Example 114: Preparation of 2-(3-bromo-2-chloro-4-(trifluoromethyl)phenyl)-4,4,5,5-tetramethyl-1,3,2-dioxaborolane (C71)

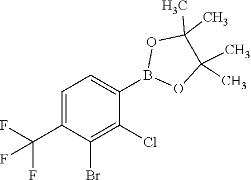 [SMILES: CC1(C)OB(c2ccc(C(F)(F)F)c(Br)c2Cl)OC1(C)C]

Lithium 2,2,6,6-tetramethylpiperidin-1-ide (2.84 g, 19.27 mmol) was placed in an oven-dried 250-mL round-bottomed flask in a glovebox and removed. Diethyl ether (75 mL) was added and the solution was cooled to −78° C. (reaction mixture is not homogeneous). 2-Bromo-1-chloro-3-(trifluoromethyl)benzene (5.00 g, 19.3 mmol, 1.0 equiv) was added as solution in ether (25 mL) dropwise over 10 minutes and the mixture was allowed to stir at −78° C. for 1 hour. 2-Isopropoxy-4,4,5,5-tetramethyl-1,3,2-dioxaborolane (3.9 mL, 19.27 mmol, 1.0 equiv) was then added to the non-homogeneous reaction mixture over 10 minutes and the reaction was allowed to warm slowly to room temperature overnight. The reaction was quenched with saturated NH4Cl at 0° C. and warmed to room temperature, layers separated. The aqueous was further extracted with diethyl ether (2) and the combined organics dried over sodium sulfate, filtered and concentrated. Purification over silica gel using a 0 to 5% ethyl acetate/hexane gradient afforded the title compound (3.87 g, 52% yield) as a viscous yellow oil.

### Example 115: Preparation of 2,2-difluoro-5,7-diiodobenzo[d][1,3]dioxol-4-ol (C72)

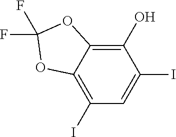 [SMILES: Oc1c(I)cc(I)c2c1OC(F)(F)O2]

The title compound was prepared according to a method in Altenbach, R. J., et al., WO 2017/009804 A1. A solution of 2,2-difluorobenzo[d][1,3]dioxol-4-ol (2.00 g, 11.5 mmol) in MeOH (20.2 mL) was cooled to <0° C., and N-ethyl-N-isopropylpropan-2-amine (1.16 mL, 12.6 mmol) and iodine chloride (1.27 mL, 25.3 mmol) were added (drop wise at <5° C.). After 30 min, the reaction was quenched with satd aq Na₂S₂O₃ (10 mL), and the mixture was partitioned between water (10 mL) and Et₂O (30 mL). The organic layer was washed with brine (5 mL), dried over Na₂SO₄ filtered, and concentrated. Purification of the residue by silica gel column chromatography eluting with 0-20% EtOAc-hexane gave 2,2-difluoro-5,7-diiodobenzo[d][1,3]dioxol-4-ol (1.03 g, 21%), 2,2-difluoro-5-iodobenzo[d][1,3]dioxol-4-ol (0.487 g, 14%), and 2,2-difluoro-7-iodobenzo[d][1,3]dioxol-4-ol (1.19 g, 35%). The structure of 2,2-difluoro-7-iodobenzo[d][1,3]dioxol-4-ol was confirmed using two-dimensional NMR experiments. The compound was isolated as a white solid (1.03 g, 21%): ¹H NMR (400 MHz, CDCl₃) δ 7.69 (s, 1H); ¹⁹F NMR (376 MHz, CDCl3) δ−49.42; EIMS m/z 425.9.

### Example 116: Preparation of 6-fluoro-5-(4,4,5,5-tetramethyl-1,3,2-dioxaborolan-2-yl)benzo[d]thiazole (C73)

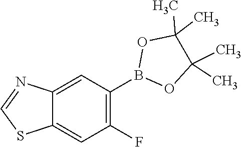 [SMILES: CC1(C)OB(c2cc3ncsc3cc2F)OC1(C)C]

5-Bromo-6-fluorobenzo[d]thiazole (2.5 g, 10.8 mmol) and potassium acetate (KOAc; 2.11 g, 21.5 mmol) were combined in dry dioxane (20 mL), sparged with a stream of nitrogen for 15 min, treated with the 4,4,4′,4′,5,5,5′,5′-octamethyl-2,2′-bi(1,3,2-dioxaborolane) (3.01 g, 11.9 mmol) and Pd(dppf)Cl₂ (0.440 g, 0.539 mmol) and heated to 95° C. for 6 h. Additional catalyst (240 mg) was added and heating was continued for 6 h more. The cooled reaction mixture was stirred with EtOAc (50 mL) and water (20 mL) for 20 min, and filtered to remove the dark solids. The organic phase was washed with satd NaCl (10 mL), dried, and concentrated. Purification by chromatography with 0-30% EtOAc-hexane as eluent gave the title compound as tan crystals (1.5 g, 45%): ¹H NMR (400 MHz, DMSO-d₆) δ 9.39 (s, 1H), 8.26 (d, J=5.1 Hz, 1H), 8.05 (d, J=9.0 Hz, 1H), 1.34 (s, 12H); ¹⁹F NMR (376 MHz, DMSO-d₆) δ−106.91; EIMS m/z 231/233.

### Example 117: Preparation of 5-bromo-6-fluorobenzo[d]thiazole (C74)

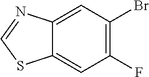 [SMILES: Fc1cc2scnc2cc1Br]

Potassium o-ethyl carbondithioate (5.36 g, 33.5 mmol), 5-bromo-2,4-difluoroaniline (5.8 g, 27.9 mmol) were combined in dry N-methyl-2-pyrrolidone (NMP; 40 mL) and heated to 100° C. for 18 h. After cooling, the mixture was partitioned between EtOAc (50 mL) and water (30 mL). The aqueous. phase was extracted with EtOAc (30 mL) and the combined organic phases were washed water (2×25 mL), satd NaCl (25 mL), dried and concentrated to give the intermediate thiol (5.4 g) as a tan solid. This material was combined in methanol (120 mL) with nickel chloride hexahydrate (3.3 g, 14 mmol)and zinc powder (3.7 g, 56 mmol), heated to reflux and treated dropwise with concentrated HCl (20 mL). The reaction mixture was heated for 2 h after the addition was complete. The cooled mixture was stirred with EtOAc (200 mL) and treated with concentrated aq ammonia until the pH was >10. The organic phase was washed with satd NaCl (40 mL), dried and concentrated. Purification by chromatography with 0-40% EtOAc-hexanes gave the title compound as a yellow solid (3.2 g, 47%): mp 86-88° C.; ¹H NMR (400 MHz, DMSO-d₆) δ 9.44 (s, 1H), 8.47 (d, J=6.3 Hz, 1H), 8.30 (d, J=8.6 Hz, 1H); ¹⁹F NMR (376 MHz, DMSO-d₆) δ−111.18; EIMS m/z 232.

### Example 118: Preparation of 2-(2-fluoro-3-methyl-4-(trifluoromethyl)phenyl)-4,4,5,5-tetramethyl-1,3,2-dioxaborolane (C75)

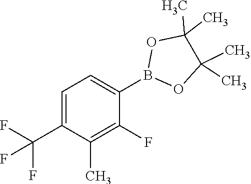 [SMILES: Cc1c(C(F)(F)F)ccc(B2OC(C)(C)C(C)(C)O2)c1F]

To a stirred solution of bis(isopropyl)amine (2.36 mL, 16.8 mmol) in THF (46.8 mL) at −78° C. was added butyllithium (6.18 mL, 15.44 mmol). The resulting pale yellow solution was stirred at −78° C. for 15 min, warmed to 0° C. for 15 min, then recooled to −78° C. for 15 min. 1-Fluoro-2-methyl-3-(trifluoromethyl)benzene (2.5 g, 14.0 mmol) was then added and the resulting solution was stirred at −78° C. for 2 h. 2-Isopropoxy-4,4,5,5-tetramethyl-1,3,2-dioxaborolane (2.86 mL, 14.02 mmol) was then added, and the solution was allowed to slowly warm to room temperature and stirred overnight. The reaction mixture was diluted with 0.1 M HCl and extracted with DCM. The combined organic extracts were dried over Mg₂SO₄, filtered, and concentrated. Purification of the residue by flash chromatography on silica (5-30% EtOAc-hexane) yielded the title compound as a clear oil (2.45 g, 57%): ¹H NMR (400 MHz, CDCl₃) δ 7.68-7.56 (m, 1H), 7.39 (d, J=7.8 Hz, 1H), 2.39-2.33 (m, 3H), 1.37 (s, 13H); ¹⁹F NMR (376 MHz, CDCl₃) δ−61.39 (s), −104.31 (s).

### Example 119: Preparation of 7-bromo-4-(trifluoromethyl)benzofuran (C76)

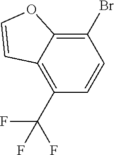 [SMILES: FC(F)(F)c1ccc(Br)c2occc12]

To 1-bromo-2-(2,2-diethoxyethoxy)-4-(trifluoromethyl)benzene (1.5 g, 4.20 mmol) in toluene (4.94 mL) was added Amberlyst© 15 hydrogen form (252 mg, 4.20 mmol). The reaction mixture was heated at 120° C. for ˜24 h and then at room temperature for ˜72 h. The reaction mixture was directly loaded onto a Celite© cartridge with a syringe to decant the solution from the resin beads. Purification by flash chromatography (0-30% EtOAc-hexanes) provided the title compound as a clear oil 450 mg, 40%): ¹H NMR (400 MHz, CDCl₃) δ 7.82 (d, J=2.2 Hz, 1H), 7.56 (d, J=8.1 Hz, 1H), 7.42 (d, J=8.1 Hz, 1H), 7.04 (t, J=1.9 Hz, 1H); ¹⁹F NMR (376 MHz, CDCl3) δ−61.37; EIMS m/z 262, 264.

### Example 120: Preparation of 2,2-difluoro-5-iodo-6-methoxybenzo[d][1,3]dioxole (C77)

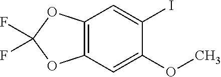

The title compound was prepared according to Preparation 45 in Eckelbarger, et al., U.S. Patent Application Publication 2014/0274701 A1. 2,2-Difluoro-6-methoxybenzo[d][1,3]dioxol-5-amine (Preparation 44 in U.S. Patent Application Publication 2014/0274701 A1; 1.40 g, 6.89 mmol) was dissolved in DCM (5 mL) and added in portions to concentrated HCl (75 mL) with rapid stirring to form a loose white slurry. The mixture was cooled to 3-5° C. and treated in portions with sodium nitrite (0.713 g, 10.3 mmol) dissolved in water (10 mL) over ca 5 min. The diazonium solution was poured into a solution of sodium iodide (3.10 g, 20.7 mmol) in water (75 mL) stirred with DCM (50 mL). After 30 min total the mixture was stirred with 15% NaHSO₃ (20 mL) for 10 min. The aqueous phase was further extracted with DCM (30 mL), and the combined organic phases were washed with satd NaCl (15 mL), dried, and concentrated. Purification of the residue on silica gel with 0-15% EtOAc-hexane gave the title compound as a white crystalline solid (1.8 g, 83%): 50-51° C.; ¹H NMR (400 MHz, CDCl₃) δ 7.45 (s, 1H), 6.69 (s, 1H), 3.86 (s, 3H); ¹⁹F NMR (376 MHz, CDCl₃) δ−49.81 (s).

### Example 121: Preparation of 1-chloro-2-iodo-3-methoxy-5-(trifluoromethyl)benzene (C78)

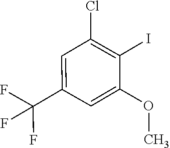

To a 5 mL vial were added 2-chloro-6-methoxy-4-(trifluoromethyl)aniline (106 mg, 0.470 mmol) and diiodomethane (208 mg, 0.776 mmol). The vial was sealed and the reaction mixture was heated to 100° C. before adding tert-butyl nitrite (121 mg, 1.175 mmol). The reaction was held at elevated temperatures for 2 h. The cooled reaction mixture was loaded directly onto silica gel. Purification by column chromatography with hexanes-EtOAc afforded the title compound as a brown oil (227 mg, 100%): ¹H NMR (400 MHz, CDCl₃) δ 7.38 (s, 1H), 6.30 (s, 1H), 3.78 (s, 3H); ¹⁹F NMR (376 MHz, CDCl₃) δ−60.73; EIMS m/z 336.0.

### Example 122: Preparation of (5-bromo-4-chloro-2-(trifluoromethyl)phenyl)(methyl)sulfane (C79)

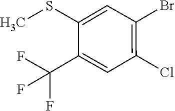

Step 1—Preparation of 2-bromo-4-(methylthio)-5-(trifluoromethyl)aniline: 4-(Methylthio)-3-(trifluoromethyl)aniline (500 mg, 2.41 mmol) was weighed into a 25 mL round bottom flask equipped with a septum. Methanol (12.1 mL) was added and the mixture was stirred until complete dissolution was achieved. 1-Bromopyrrolidine-2,5-dione (472 mg, 2.65 mmol) was added in a single portion with stirring. The reaction was allowed to stir at room temperature until complete consumption of the starting material was observed. The reaction mixture was concentrated, taken up in ether and washed with satd aq NaCl. The organic phase was separated, dried and concentrated to afford the title compound that was used without further purification in step 2.

Step 2—Preparation of (5-bromo-4-chloro-2-(trifluoromethyl)phenyl)(methyl)sulfane: To a 5 mL vial were added 2-bromo-4-(methylthio)-5-(trifluoromethyl)aniline (236 mg, 0.825 mmol), tert-butyl nitrite (196 μL, 1.65 mmol), and copper(II) chloride (222 mg, 1.65 mmol). The vial was sealed and the reaction mixture was heated to 65° C. for 2 h. The reaction mixture was loaded directly onto a silica gel column. Purification of the resultant product eluting with hexanes-EtOAc afforded the title compound as a white solid (82 mg, 33%): ¹H NMR (500 MHz, CDCl₃) δ 7.66 (s, 1H), 7.53 (s, 1H), 2.52 (s, 4H); ¹⁹F NMR (471 MHz, CDCl₃) δ−62.24; EIMS m/z 305.9.

### Example 123: Preparation of 2-(2-chloro-3-(methylthio)-4-(trifluoromethyl)phenyl)-4,4,5,5-tetramethyl-1,3,2-dioxaborolane (C80) and 2-(2-chloro-6-(methylthio)-4-(trifluoromethyl)phenyl)-4,4,5,5-tetramethyl-1,3,2-dioxaborolane (C81)

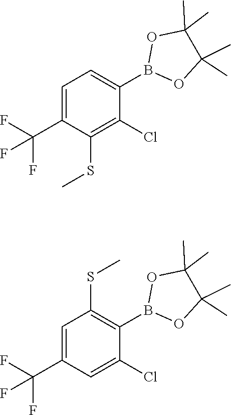 [SMILES: CSc1c(C(F)(F)F)ccc(B2OC(C)(C)C(C)(C)O2)c1Cl ; CSc1cc(C(F)(F)F)cc(Cl)c1B1OC(C)(C)C(C)(C)O1]

To a solution of tetramethylethylenediamine (2.92 mL, 13.1 mmol) in Et₂O (75 mL) was added n-butyllithium (2.5 M solution in hexane; 5.2 mL, 13.1 mmol) via syringe over 10 min at −78° C., and the mixture was stirred for 15 min. (3-Bromo-2-chloro-6-(trifluoromethyl)phenyl)(methyl)sulfane and (2-bromo-3-chloro-5-(trifluoromethyl)phenyl)(methyl)sulfane (C87 and C88; 4 g, 13.1 mmol) in Et₂O (70 mL) were added to the above mixture via syringe over 15 min. The mixture was stirred for 1 h at −78° C. 2-Isopropoxy-4,4,5,5-tetramethyl-1,3,2-dioxaborolane (3.21 mL, 15.7 mmol) was added to the above reaction mixture via syringe over 10 min. The reaction mixture was stirred for 1 h at −78° C., was warmed slowly to room temperature and was stirred for an additional 2 h. The reaction mixture was quenched with a satd NH₄Cl solution at −78° C., warmed to room temperature and extracted with Et₂O. The organic layer was washed with water and brine and concentrated under vacuum. Purification of the resultant compound mixture by column chromatography using 2% EtOAc in hexane as eluent furnished the title compound mixture as a colorless liquid (800 mg, 17%): ¹H NMR (300 MHz, CDCl₃) δ 7.67 (d, J=8.0 Hz, 1H), 7.59 (d, J=7.7 Hz, 1H), 7.36 (d, J=6.9 Hz, 2H), 2.50 (s, 3H), 2.40 (s, 3H), 1.43 (s, 12H), 1.39 (s, 12H).

### Example 124: Preparation of (E)-2-bromo-3-chloro-5-(trifluoromethyl)benzaldehyde O-methyl oxime (C82)

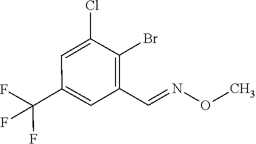 [SMILES: CO/N=C/c1cc(C(F)(F)F)cc(Cl)c1Br]

To a solution of 2-bromo-3-chloro-5-(trifluoromethyl)benzaldehyde and 3-bromo-2-chloro-6-(trifluoromethyl)benzaldehyde (C85 and C86; 1.8 g, 6.26 mmol) in ethanol was added sequentially methoxylamine hydrochloride (1.05 g, 12.5 mmol) and Et₃N (1.74 mL, 12.5 mmol) at room temperature. The reaction mixture was stirred at reflux for 16 h. The reaction mixture was cooled to room temperature and concentrated under vacuum. The residue was poured into water and extracted with EtOAc. The organic layer was washed with water and brine and concentrated under vacuum. Purification of the compound mixture by column chromatography using 5% EtOAc in hexane afforded the title compound as a white solid (250 mg, 13%): ¹H NMR (300 MHz, CDCl₃) δ 8.48 (s, 1H), 8.03 (d, J=2.5 Hz, 1H), 7.70 (d, J=2.1 Hz, 1H), 4.04 (s, 3H).

### Example 125: Preparation of (E)-3-bromo-2-chloro-6-(trifluoromethyl)benzaldehyde O-methyl oxime (C83)

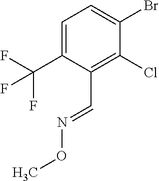 [SMILES: CO/N=C/c1c(C(F)(F)F)ccc(Br)c1Cl]

To a solution of 2-bromo-3-chloro-5-(trifluoromethyl)benzaldehyde and 3-bromo-2-chloro-6-(trifluoromethyl)benzaldehyde (C85 and C86; 1.8 g, 6.26 mmol) in ethanol was added sequentially methoxylamine hydrochloride (1.05 g, 12.5 mmol) and Et₃N (1.74 mL, 12.5 mmol) at room temperature. The reaction mixture was stirred at reflux for 16 h. The reaction mixture was cooled to room temperature and concentrated under vacuum. The residue was poured into water and extracted with EtOAc. The organic layer was washed with water and brine and concentrated under vacuum. Purification of the compound mixture by column chromatography using 5% EtOAc in hexane afforded the title compound as a white solid (50 mg, 5%): ¹H NMR (300 MHz, CDCl₃) δ 8.23 (d, J=1.8 Hz, 1H), 7.78 (d, J=8.3 Hz, 1H), 7.50 (d, J=8.6 Hz, 1H), 4.00 (s, 3H).

### Example 126: Preparation of (E)-2-(3-bromo-2-chloro-6-(trifluoromethyl)benzylidene)-1,1-dimethylhydrazine (C84)

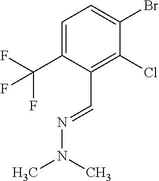 [SMILES: CN(C)/N=C/c1c(C(F)(F)F)ccc(Br)c1Cl]

To a solution of 2-bromo-3-chloro-5-(trifluoromethyl)benzaldehyde and 3-bromo-2-chloro-6-(trifluoromethyl)benzaldehyde (C85 and C86; 2.5 g, 8.696 mmol) in ethanol was added sequentially N,N-dimethylhydrazine (0.627 g, 10.436 mmol) Et3N (1.45 mL, 10.4 mmol) at room temperature and the mixture was heated to reflux for 16 h. The reaction mixture was cooled to room temperature and concentrated under vacuum. The residue was poured into water and extracted with EtOAc. The organic layer was washed with water and brine and concentrated under vacuum. Purification of the resulting mixture by column chromatography using 5% EtOAc in hexane furnished the title compound as a white solid (125 mg, 4%): ¹H NMR (300 MHz, CDCl₃) δ 7.63 (d, J=8.5 Hz, 1H), 7.45 (d, J=8.7 Hz, 1H), 7.23 (s, 1H), 3.03 (s, 6H).

### Example 127: Preparation of 2-bromo-3-chloro-5-(trifluoromethyl)benzaldehyde (C85) and 3-bromo-2-chloro-6-(trifluoromethyl)benzaldehyde (C86)

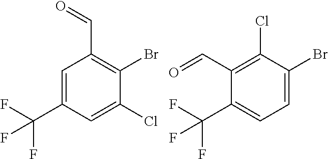

To a solution of diisopropylamine (5.7 mL, 40.5 mmol) in Et₂O (50 mL) was added n-butyllithium (2.5 M solution in hexane; 10.8 mL, 27.0 mmol) at 0° C. via syringe over 10 min and the mixture was stirred for 15 min. The reaction mixture was cooled to −78° C. and stirred for 1 h. 1-Bromo-2-chloro-4-(trifluoromethyl)benzene (7 g, 27.0 mmol) in Et₂O (75 mL) was added to the above mixture via syringe over 15 min, and the reaction mixture was stirred for 1 h at −78° C. Dimethylformamide (2.52 mL, 32.4 mmol) was added via syringe over 10 min, and the mixture was stirred for 1 h at −78° C. The reaction mixture was quenched with a satd NH₄Cl solution at −78° C., warmed to room temperature and extracted with Et₂O. The organic layer was washed with water and brine and concentrated under vacuum. The title compound mixture was isolated as an orange liquid (4.8 g), which was used in the next step without further purification.

### Example 128: Preparation of (3-bromo-2-chloro-6-(trifluoromethyl)phenyl)(methyl)sulfane (C87) and (2-bromo-3-chloro-5-(trifluoromethyl)phenyl)(methyl)sulfane (C88)

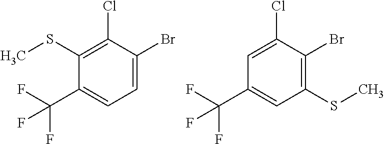

To a solution of diisopropylamine (10.8 mL, 77.1 mmol) in Et₂O (75 mL) was added n-butyllithium (2.5 M solution in hexane; 15.4 mL, 38.5 mmol) at 0° C. via syringe over 10 min and the mixture was stirred for 15 min. The reaction mixture was cooled to −78° C. and stirred for 1 h. 1-Bromo-2-chloro-4-(trifluoromethyl)benzene (10 g, 38.5 mmol) in Et₂O (75 mL) was added to the above reaction mixture via syringe over 15 min, and the reaction mixture was stirred for 1 h at −78° C. Dimethyl disulfide (4.11 mL, 46.3 mmol) was added via syringe over 10 min, and the mixture was stirred for 1 h at −78° C. The reaction mixture was slowly warmed to room temperature and stirred for 2 h. The reaction mixture was quenched with a satd NH₄Cl solution at −78° C. and was extracted with Et₂O. The organic layer was washed with water and brine and was concentrated under vacuum. Purification of the crude residue by column chromatography using 0.5% EtOAc in hexane as eluent afforded the mixture of title compounds as a colorless liquid (4 g, 34%): ¹H NMR (300 MHz, CDCl₃) δ 7.73 (d, J=8.5 Hz, 1H), 7.51-7.44 (m, 2H), 7.15 (d, J=2.0 Hz, 1H), 2.52 (s, 3H), 2.43 (s, 3H).

### Example 129: Preparation of 1-bromo-2-chloro-4-(1,1-difluoro-2-methoxyethyl)benzene (C89)

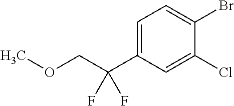

To a solution of 2-(4-bromo-3-chlorophenyl)-2,2-difluoroethan-1-ol (C90; 1 g, 3.69 mmol) in DMF (10 mL) were added sequentially sodium hydride (NaH, 60% suspension in mineral oil; 0.13 g, 5.53 mmol) at 0° C. and iodomethane (CH₃I; 0.62 g, 4.42 mmol). The reaction mixture was stirred at room temperature for 16 h and was quenched with ice water. The mixture was extracted with EtOAc. The organic layer was washed with brine, dried over anhydrous Na₂SO₄, filtered and concentrated under reduced pressure. Purification of the resulting product by column chromatography (silica gel 100-200 mesh) eluting with 10-30% EtOAc in petroleum ether afforded the title compound as a pale yellow liquid (0.4 g, 40%): ¹H NMR (300 MHz, CDCl₃) δ 7.68 (d, J=8.4 Hz, 1H), 7.61-7.60 (m, 1H), 7.28-7.26 (m, 1H), 3.78 (t, J=12.3 Hz, 2H), 3.42 (s, 3H); ESIMS m/z 284.00 ([M]⁺).

### Example 130: Preparation of 2-(4-bromo-3-chlorophenyl)-2,2-difluoroethan-1-ol (C90)

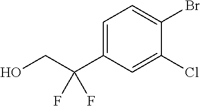 [SMILES: OCC(F)(F)c1ccc(Br)c(Cl)c1]

To a solution of ethyl 2-(4-bromo-3-chlorophenyl)-2,2-difluoroacetate (C91; 1.5 g, 4.80 mmol) in MeOH (15 mL) was added NaBH₄ (0.27 g, 7.21 mmol) at 0° C., and the reaction mixture was stirred at room temperature for 16 h. The reaction mixture was poured into water and was extracted with DCM. The organic layer was washed with brine, dried over anhydrous Na₂SO₄, filtered and concentrated under reduced pressure. Purification of the crude product by column chromatography (silica gel 100-200 mesh) eluting with 20-40% EtOAc in petroleum ether afforded the title compound as a brown liquid (0.7 g, 50%): ¹H NMR (400 MHz, DMSO-d₆) δ 7.91 (d, J=8.4 Hz, 1H), 7.75 (d, J=1.2 Hz, 1H), 7.42 (dd, J=1.6, 8.0 Hz, 1H), 5.67 (t, J=6.4 Hz, 1H), 3.92-3.83 (m, 2H); ESIMS m/z 270.00 ([M]⁺).

### Example 131: Preparation of ethyl 2-(4-bromo-3-chlorophenyl)-2,2-difluoroacetate (C91)

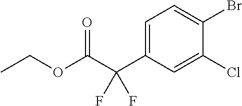 [SMILES: CCOC(=O)C(F)(F)c1ccc(Br)c(Cl)c1]

To a solution of ethyl 2-bromo-2,2-difluoroacetate (13 g, 126.18 mmol) in DMSO (60 mL) was added copper powder (4 g, 126.18 mmol) at room temperature, and the reaction mixture was stirred for 2 h. 1-Bromo-2-chloro-4-iodobenzene (10 g, 63.1 mmol) was added and the reaction mixture was stirred at 95° C. for 16 h. The reaction mixture was cooled to room temperature, EtOAc (150 mL) was added, and the reaction mixture was stirred for 1 h. The mixture was filtered through a pad of Celite®, which was washed with EtOAc (30 mL). The filtrate was washed with satd NH₄Cl (100 mL) and brine (50 mL), dried over anhydrous Na₂SO₄, filtered and concentrated under reduced pressure. Purification by column chromatography (silica gel 100-200 mesh) of the resulting product eluting with 10-20% EtOAc in petroleum ether afforded the title compound as a pale-brown liquid (4.5 g, 46%): ¹H NMR (300 MHz, CDCl₃) δ 7.73-7.68 (m, 1H), 7.40-7.34 (m, 1H), 7.20-7.16 (m, 1H), 4.31 (q, J=6.9 Hz, 2H), 1.32 (t, J=7.2 Hz, 3H); ESIMS m/z 312.00 ([M]⁺).

### Example 132: Preparation of 2-(4-bromo-3-chlorophenyl)-2,2-difluoroacetamide (C92)

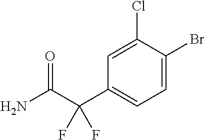 [SMILES: NC(=O)C(F)(F)c1ccc(Br)c(Cl)c1]

To a solution of ethyl 2-(4-bromo-3-chlorophenyl)-2,2-difluoroacetate (C91; 1 g, 3.20 mmol) in MeOH (20 mL) was added methanolic ammonia (7 M in Methanol; 10 mL) at 0° C., and the reaction mixture was stirred at room temperature for 4 h. The reaction mixture was concentrated under reduced pressure. The title compound was isolated as a white solid (0.85 g, 95%): ¹H NMR (300 MHz, CDCl₃) δ 7.74-7.71 (m, 2H), 7.41-7.37 (m, 1H), 6.38 (br s, 1H), 5.68 (br s, 1H); ESIMS m/z 282.31 ([M−H]⁺).

### Example 133: Preparation of (2-chloro-4-(difluoromethoxy)phenyl)trimethylstannane (C93)

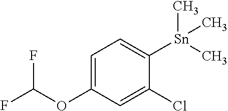

1-Bromo-2-chloro-4-(difluoromethoxy)benzene (1.440 g, 5.59 mmol), 1,1,1,2,2,2-hexamethyldistannane (3.66 g, 11.19 mmol), Pd(PPh₃)₂Cl₂ (0.393 g, 0.559 mmol) were combined in 1,4-dioxane (5.59 mL) and heated at 90° C. for 24 h. The cooled reaction mixture was filtered through silica gel with diethyl ether and concentrated under vacuum. Purification by flash chromatography (silica gel, hexanes) provided the title compound as a clear oil (0.7 g, 36%): ¹H NMR (500 MHz, CDCl₃) δ 7.37 (d, J=8.0 Hz, 1H), 7.14 (d, J=2.3 Hz, 1H), 7.00 (ddd, J=8.0, 1.9, 1.2 Hz, 1H), 6.49 (t, J=73.5 Hz, 1H), 0.37 (s, 8H); EIMS m/z 327 ([M-CH₃]).

The following compound was prepared in like manner to the procedure outlined in Example 133:

### (4-(Difluoromethoxy)-2-methoxyphenyl)trimethylstannane (C94)

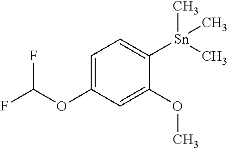 [SMILES: COc1cc(OC(F)F)cc[c]1[Sn]([CH3])([CH3])[CH3]]

Using the appropriate starting materials, the title compound was synthesized and isolated as a clear oil (550 mg, 39%): ¹H NMR (500 MHz, CDCl₃) δ 7.30 (d, J=7.8 Hz, 1H), 6.75-6.67 (m, 1H), 6.57 (dd, J=7.5, 2.1 Hz, 1H), 6.51 (t, J=74.3 Hz, 1H), 3.78 (s, 3H), 0.26 (s, 9H); EIMS m/z 323 ([M−CH₃]).

### Example 134: Preparation of 2-(4-bromo-3-chlorophenyl)-2,2-difluoroacetonitrile (C95)

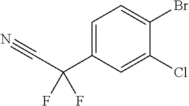 [SMILES: N#CC(F)(F)c1ccc(Br)c(Cl)c1]

To a solution of 2-(4-bromo-3-chlorophenyl)-2,2-difluoroacetamide (0.5 g, 1.76 mmol) in DCM (10 mL) was added triethylamine (0.27 g, 2.65 mmol) followed by the addition of trifluoroacetic anhydride (0.5 g, 2.65 mmol) at 0° C. The reaction mixture was stirred at room temperature for 4 h and was concentrated under reduced pressure. The title compound was isolated as a pale yellow liquid (0.3 g, 64%): ¹H NMR (300 MHz, CDCl₃) δ 7.82 (d, J=8.4 Hz, 1H), 7.76-7.75 (m, 1H), 7.44-7.41 (m, 1H); ESIMS m/z 265.00 ([M]⁺).

### Example 135: Preparation of 1-bromo-2-chloro-4-(difluoromethoxy)benzene (C96)

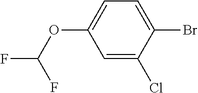 [SMILES: FC(F)Oc1ccc(Br)c(Cl)c1]

4-Bromo-3-chlorophenol (2.00 g, 9.64 mmol), tris(2-phenylpyridine)iridium(III) (0.032 g, 0.048 mmol), potassium bromodifluoroacetate (4.11 g, 19.3 mmol), and cesium carbonate (9.42 g, 28.9 mmol) were combined in DMF (16.1 mL) in a round bottom flask under nitrogen. The reaction mixture was vigorously stirred and irradiated with a blue LED light. The reaction was nearly complete after 30 min but was stirred and irradiated for another 15 min. The reaction mixture was partitioned between Et₂O and water. The organic phase was dried and concentrated onto silica gel. Purification by flash chromatography (0-10% EtOAc in hexanes gradient solvent system) provided the title compound as a clear oil (1.44 g, 56%): ¹H NMR (500 MHz, CDCl₃) δ 7.60 (d, J=8.8 Hz, 1H), 7.27 (d, J=2.8 Hz, 1H), 6.94 (dd, J=8.8, 2.6 Hz, 1H), 6.49 (t, J=72.8 Hz, 1H); EIMS m/z 258.

The following compound was prepared in like manner to the procedure outlined in Example 135:

### 1-Bromo-4-(difluoromethoxy)-2-methoxybenzene (C97)

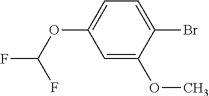

Using the appropriate starting materials, the title compound was synthesized and isolated as a clear oil (1.05 g, 41%): ¹H NMR (500 MHz, Chloroform-d) δ 7.50 (d, J=8.6 Hz, 1H), 6.68 (d, J=2.6 Hz, 1H), 6.64-6.60 (m, 1H), 6.65-6.33 (m, 1H), 3.89 (s, 3H); EIMS m/z 253.

### Example 136: Preparation of 2-bromo-3,5-dichloroisonicotinaldehyde (C98)

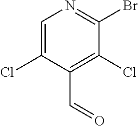 [SMILES: O=Cc1c(Cl)cnc(Br)c1Cl]

To a solution of diisopropylamine (2.45 g, 24.2 mmol, 3.39 mL) in THF (50 mL) cooled to −25° C. (internal temperature) under nitrogen was added n-butyllithium (1.55 g, 24.2 mmol, 9.68 mL of a 2.5 M solution) dropwise via syringe. The resulting solution of lithium diisopropylamide was cooled to −60° C. and treated with a solution of 2-bromo-3,5-dichloroisonicotinaldehyde (C98; 5.0 g, 22 mmol) in THF (8 mL) at a rate sufficient to keep the internal temperature below −50° C. After 1 h, methylformate (2.65 g, 44.1 mmol, 2.72 mL) was added at a rate sufficient to keep the internal temperature below −50° C. After 1 h, the reaction mixture was poured into satd NaHCO₃ and extracted with EtOAc (×2). The combined organic layers were dried over Na₂SO₄, filtered and concentrated. Purification by silica gel chromatography with 20:1 hexane-EtOAc as eluent gave the title compound as an off-white solid (3.42 g, 61%): mp 61-62° C.; ¹H NMR (300 MHz, CDCl₃) δ 10.34 (s, 1H), 8.43 (s, 1H); EIMS m/z 254 ([M+H]⁺).

### Example 137: Preparation of 2-bromo-3,5-dichloro-4 difluoromethyl)pyridine (C99)

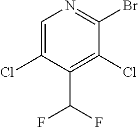 [SMILES: FC(F)c1c(Cl)cnc(Br)c1Cl]

A solution of 2-bromo-3,5-dichloroisonicotinaldehyde (C98; 1.5 g, 5.9 mmol) in DCM (25 mL) stirred at 0° C. under nitrogen was treated with DAST (3.0 g, 18.5 mmol, 2.5 mL) in three equal portions, allowing the reaction mixture to warm to room temperature between additions and re-cooling in an ice bath prior to adding the 2^{nd} and 3^{rd} aliquots. After stirring at room temperature for 3 days, the reaction was carefully quenched with satd NaHCO₃, transferred to a separatory funnel, and the layers separated. The aqueous phase was extracted with DCM, and the combined organic layers were dried over Na₂SO₄, filtered and concentrated. Purification by silica gel chromatography with 20:1 hexane-EtOAc as eluent gave the title compound as an off-white solid (1.49 g, 91%): mp 51-52° C.; ¹H NMR (300 MHz, CDCl₃) δ 8.39 (s, 1H), 7.16, (t, J=52.5 Hz, 1H); EIMS m/z 276 ([M+H]⁺).

Table 1 includes, inter alia, data for compounds F1 through F381, including synthesis data as described below and as in the above examples. The analytical data for the aforementioned compounds can be found in Table 2 also below.

TABLE 1
Structures and Preparation Data for F Compounds

| | | | | | Yield |
|---|---|---|---|---|---|
| No. | Structure | Chemical Name | Preparation | Appearance | (Amt, %) |
| F1 | 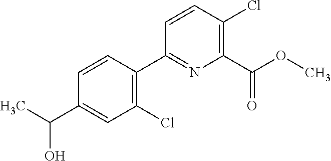 [SMILES: COC(=O)c1nc(-c2ccc(C(C)O)cc2Cl)ccc1Cl] | Methyl 3-chloro-6- (2-chloro-4-(1- hydroxyethyl)phenyl) picolinate | Example 2 using F26 | Off-White Solid | 60 mg, 50% |
| F2 | 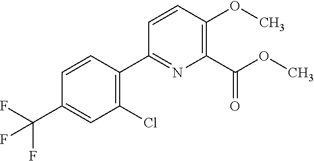 [SMILES: COC(=O)c1nc(-c2ccc(C(F)(F)F)cc2Cl)ccc1OC] | Methyl 6-(2-chloro-4- 4- (trifluoromethyl) phenyl)-3- methoxypicolinate | Example 32 with heating in a sealed tube at 80° C. for 12 h | White Solid | 0.08 g, 56% |
| F3 | 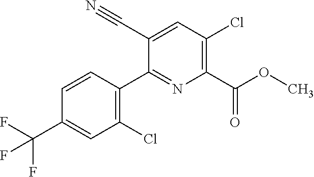 [SMILES: COC(=O)c1nc(-c2ccc(C(F)(F)F)cc2Cl)c(C#N)cc1Cl] | Methyl 3-chloro-6- (2-chloro-4- (trifluoromethyl) phenyl)-5- cyanopicolinate | Example 15 using Pd(PPh₃)₄ as the catalyst and with heating in a Biotage microwave reactor for 30 min at 150° C. | Yellow Solid | 0.012 g, 15% |
| F4 | 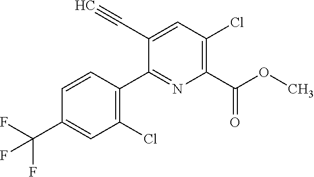 [SMILES: C#Cc1cc(Cl)c(C(=O)OC)nc1-c1ccc(C(F)(F)F)cc1Cl] | Methyl 3-chloro-6- (2-chloro-4- (trifluoromethyl) phenyl)-5- ethynylpicolinate | Example 6 | White Solid | 0.04 g, 51% |
| F5 | 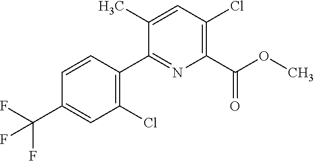 [SMILES: COC(=O)c1nc(-c2ccc(C(F)(F)F)cc2Cl)c(C)cc1Cl] | Methyl 3-chloro-6- (2-chloro-4- (trifluoromethyl) phenyl)- methylpicolinate | Example 16 | Colorless Solid | 0.017 g, 23% |
| F6 | 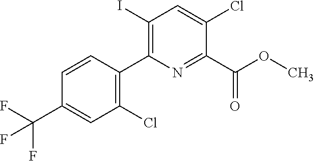 [SMILES: COC(=O)c1nc(-c2ccc(C(F)(F)F)cc2Cl)c(I)cc1Cl] | Methyl 3-chloro-6- (2-chloro-4- (trifluoromethyl) phenyl)-5- iodopicolinate | Example 19 | White Solid | 0.3 g, 77% |
| F7 | 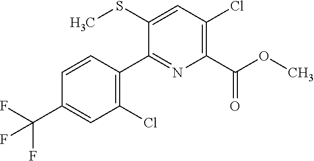 [SMILES: COC(=O)c1nc(-c2ccc(C(F)(F)F)cc2Cl)c(SC)cc1Cl] | Methyl 3-chloro-6- (2-chloro-4- (trifluoromethyl) phenyl)-5- (methylthio)picolinate | Example 19 | White Solid | 0.08 g, 67% |
| F8 | 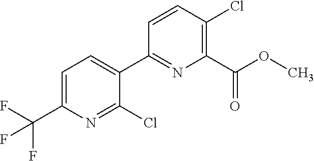 [SMILES: COC(=O)c1nc(-c2ccc(C(F)(F)F)nc2Cl)ccc1Cl] | Methyl 2′,5-dichloro- 6′-(trifluoromethyl)- [2,3′-bipyridine]-6- carboxylate | Example 32 with heating in a sealed tube at 80° C. for 12 h | Colorless Solid | 0.04 g, 26% |
| F9 | 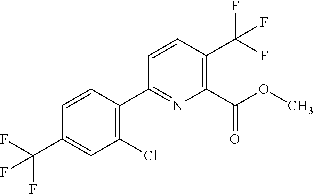 [SMILES: COC(=O)c1nc(-c2ccc(C(F)(F)F)cc2Cl)ccc1C(F)(F)F] | Methyl 6-(2-chloro-4- (trifluoromethyl) phenyl)-3- (trifluoromethyl) picolinate | Example 18 | White Solid | 0.05 g, 50% |
| F10 | 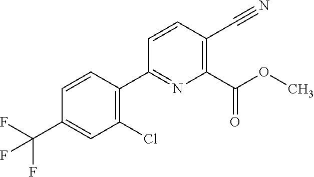 [SMILES: COC(=O)c1nc(-c2ccc(C(F)(F)F)cc2Cl)ccc1C#N] | Methyl 6-(2-chloro- 4- (trifluoromethyl) phenyl)-3- cyanopicolinate | Example 15 using Pd(PPh₃)₄ as the catalyst and with heating in a Biotage microwave reactor for 30 min at 150° C. | Off-White Solid | 0.03 g, 34% |
| F11 | 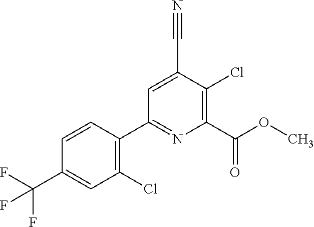 [SMILES: COC(=O)c1nc(-c2ccc(C(F)(F)F)cc2Cl)cc(C#N)c1Cl] | Methyl 3-chloro-6- (2-chloro-4- (trifluoromethyl) phenyl)-4- cyanopicolinate | Example 15 using Pd(PPh₃)₄ as the catalyst and with heating in a Biotage microwave reactor for 30 min at 150 ° C. | Off White Solid | 0.015 g, 34% |
| F12 | 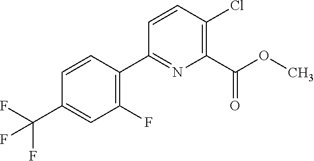 [SMILES: COC(=O)c1nc(-c2ccc(C(F)(F)F)cc2F)ccc1Cl] | Methyl 3-chloro-6- (2-fluoro-4- (trifluoromethyl) phenyl)picolinate | Example 32 with heating at 120° C. for 6 h | White Solid | 90 mg |
| F13 | 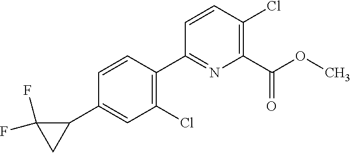 [SMILES: COC(=O)c1nc(-c2ccc(C3CC3(F)F)cc2Cl)ccc1Cl] | Methyl 3-chloro-6- (2-chloro-4-(2,2 difluorocyclopropyl) phenyl)picolinate | Example 13 using F23 | Off-White Solid | 110 mg, 55% |
| F14 | 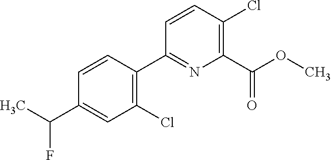 [SMILES: COC(=O)c1nc(-c2ccc(C(C)F)cc2Cl)ccc1Cl] | Methyl 3-chloro-6- (2-chloro-4-(1- fluoroethyl)phenyl)- picolinate | Example 1 using F1 | Off-White Solid | 55 mg, 70% |
| F15 | 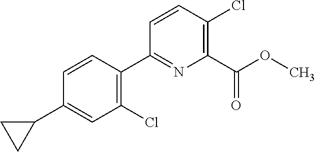 [SMILES: COC(=O)c1nc(-c2ccc(C3CC3)cc2Cl)ccc1Cl] | Methyl 3-chloro-6- (2-chloro-4- cyclopropylphenyl) picolinate | Example 4 using F27 | Pale Yellow Solid | 200 mg, 40% |
| F16 | 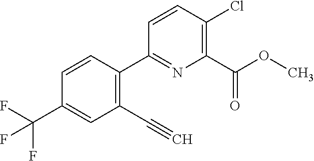 [SMILES: C#Cc1cc(C(F)(F)F)ccc1-c1ccc(Cl)c(C(=O)OC)n1] | Methyl 3-chloro-6- (2-ethynyl-4- (trifluoromethyl) phenyl)picolinate | Example 7 using C1 | Brown Solid | 30 mg, 30% |
| F17 | 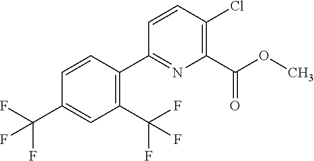 [SMILES: COC(=O)c1nc(-c2ccc(C(F)(F)F)cc2C(F)(F)F)ccc1Cl] | Methyl 6-(2,4- bis(trifluoromethyl) phenyl)-3- chloropicolinate | Example 32 with heating at 120° C. for 6 h | White Solid | 125 mg |
| F18 | 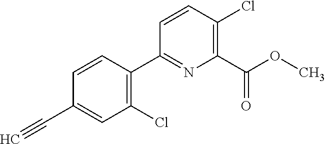 [SMILES: C#Cc1ccc(-c2ccc(Cl)c(C(=O)OC)n2)c(Cl)c1] | Methyl 3-chloro-6- (2-chloro-4- ethynylphenyl) picolinate | Example 7 | Off-White Solid | 0.035 g |
| F19 | 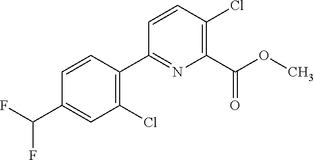 [SMILES: COC(=O)c1nc(-c2ccc(C(F)F)cc2Cl)ccc1Cl] | Methyl 3-chloro-6- (2-chloro-4- (difluoromethyl) phenyl)picolinate | Example 9 using F22 | Off-White Solid | 85 mg, 60% |
| F20 | 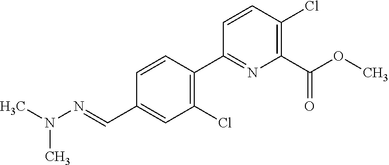 | Methyl 3-chloro-6- (2-chloro-4-((2,2- dimethylhydrazono) methyl)phenyl) picolinate | Example 10 using F22 | Off-White Solid | 55 mg, 50% |
| F21 | 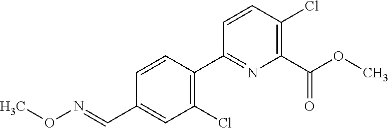 | Methyl 3-chloro-6- (2-chloro-4- ((methoxyimino) methyl)phenyl) picolinate | Example 11 using F22 | Off White Solid | 110 mg, 70% |
| F22 | 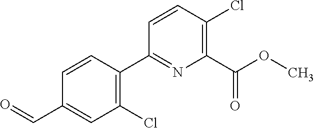 [SMILES: COC(=O)c1nc(-c2ccc(C=O)cc2Cl)ccc1Cl] | Methyl 3-chloro-6- (2-chloro-4- formylphenyl) picolinate | Example 12 using F23 | Off-White Solid | 600 mg, 65% |
| F23 | 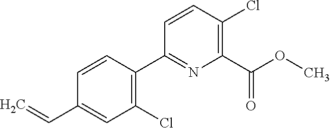 [SMILES: C=Cc1ccc(-c2ccc(Cl)c(C(=O)OC)n2)c(Cl)c1] | Methyl 3-chloro-6- (2-chloro-4- vinylphenyl) picolinate | Example 14 using F27 | Off-White Solid | 160 mg, 55% |
| F24 | 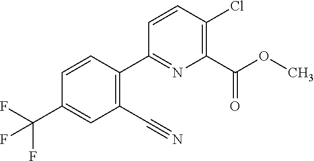 [SMILES: COC(=O)c1nc(-c2ccc(C(F)(F)F)cc2C#N)ccc1Cl] | Methyl 3-chloro-6- (2-cyano-4- (trifluoromethyl) phenyl) picolinate | Example 32 with heating at 120° C. for 6 h | White Solid | 45 mg |
| F25 | 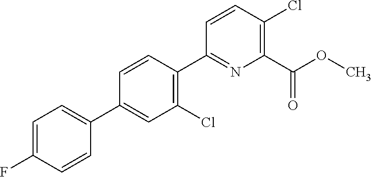 [SMILES: COC(=O)c1nc(-c2ccc(-c3ccc(F)cc3)cc2Cl)ccc1Cl] | Methyl 3-chloro-6- (3-chloro-4′-fluoro- [1,1′-biphenyl]-4-yl) picolinate | Example 5 using F27 | Off-White Solid | 90 mg, 40% |
| F26 | 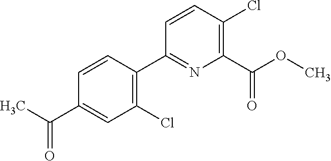 [SMILES: COC(=O)c1nc(-c2ccc(C(C)=O)cc2Cl)ccc1Cl] | Methyl 6-(4-acetyl-2- chlorophenyl)-3- chloropicolinate | Example 3 using F27 | Off-White Solid | 280 mg, 30% |
| F27 | 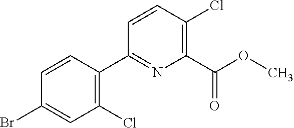 [SMILES: COC(=O)c1nc(-c2ccc(Br)cc2Cl)ccc1Cl] | Methyl 6-(4-bromo- 2-chlorophenyl)-3- chloropicolinate | Example 32 with heating at 120° C. for 6 h | Off-White Solid | 280 mg, 50% |
| F28 | 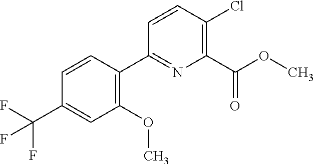 [SMILES: COC(=O)c1nc(-c2ccc(C(F)(F)F)cc2OC)ccc1Cl] | Methyl 3-chloro-6- (2-methoxy-4- (trifluoromethyl) phenyl)picolinate | Example 32 with heating at 120° C. for 6 h | White Solid | 100 mg |
| F29 | 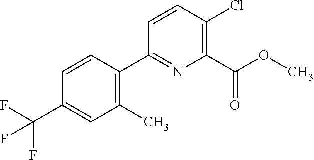 [SMILES: COC(=O)c1nc(-c2ccc(C(F)(F)F)cc2C)ccc1Cl] | Methyl 3-chloro-6- (2-methyl-4- (trifluoromethyl) phenyl)picolinate | Example 32 with heating at 120° C. for 6 h | White Solid | 136 mg |
| F30 | 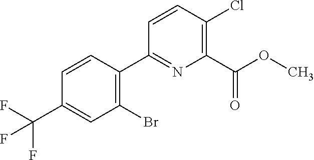 [SMILES: COC(=O)c1nc(-c2ccc(C(F)(F)F)cc2Br)ccc1Cl] | Methyl 6-(2-bromo-4- (trifluoromethyl) phenyl)-3- chloropicolinate | Example 32 with heating at 120° C. for 6 h | White Solid | 97 mg |
| F31 | 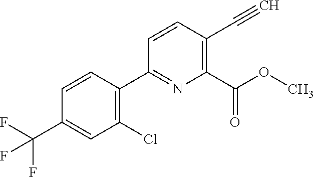 [SMILES: C#Cc1ccc(-c2ccc(C(F)(F)F)cc2Cl)nc1C(=O)OC] | Methyl 6-(2-chloro-4- (trifluoromethyl) phenyl)-3- ethynylpicolinate | Example 6 | Off-White Solid | 0.08 g, 47% |
| F32 | 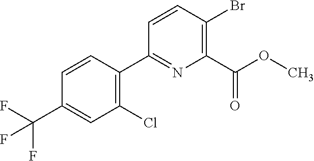 [SMILES: COC(=O)c1nc(-c2ccc(C(F)(F)F)cc2Cl)ccc1Br] | Methyl 3-bromo-6- (2-chloro-4- (trifluoromethyl) yl)picolinate | Example 32 with heating in a sealed tube at 80° C. for 12 h | Colorless Solid | 0.7 g, 53% |
| F33 | 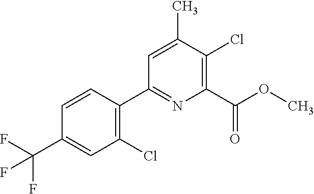 [SMILES: COC(=O)c1nc(-c2ccc(C(F)(F)F)cc2Cl)cc(C)c1Cl] | Methyl 3-chloro-6- (2-chloro-4- (trifluoromethyl) phenyl)-4- methylpicolinate | Example 16 using F41 | White Solid | 0.16 g, 47% |
| F34 | 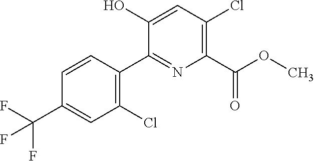 [SMILES: COC(=O)c1nc(-c2ccc(C(F)(F)F)cc2Cl)c(O)cc1Cl] | Methyl 3-chloro-6- (2-chloro-4- (trifluoromethyl) phenyl)-5- hydroxypicolinate | Example 20 using C6 | White Solid | 0.15 g, 53% |
| F35 | 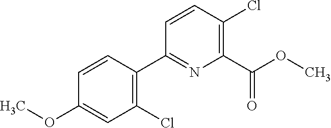 | Methyl 3-chloro-6- (2-chloro-4- methoxyphenyl) picolinate | Example 32 with heating at 120° C. for 6 h | White Solid | 245 mg |
| F36 | 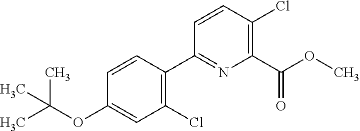 [SMILES: COC(=O)c1nc(-c2ccc(OC(C)(C)C)cc2Cl)ccc1Cl] | Methyl 6-(4-(tert- butoxy)-2- chlorophenyl)-3- chloropicolinate | Example 32 with heating at 120° C. for 6 h | White Solid | 65 mg |
| F37 | 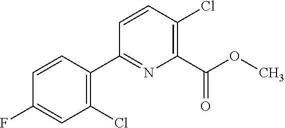 [SMILES: COC(=O)c1nc(-c2ccc(F)cc2Cl)ccc1Cl] | Methyl 3-chloro-6- (2-chloro-4- fluorophenyl) picolinate | Example 32 with heating at 120° C. for 6 h | White Solid | 180 mg, 50% |
| F38 | 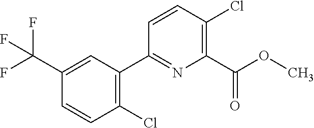 [SMILES: COC(=O)c1nc(-c2cc(C(F)(F)F)ccc2Cl)ccc1Cl] | Methyl 3-chloro-6- (2-chloro-5- (trifluoromethyl) phenyl)picolinate | Example 32 with heating at 120° C. for 6 h | White Solid | 170 mg |
| F39 | 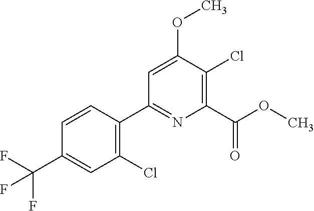 [SMILES: COC(=O)c1nc(-c2ccc(C(F)(F)F)cc2Cl)cc(OC)c1Cl] | Methyl 3-chloro-6- (trifluoromethyl) phenyl)-4- methoxypicolinate | Example 17 | White Solid | 0.03 g, 34% |
| F40 | 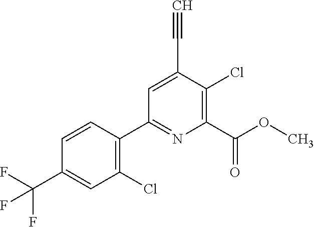 [SMILES: C#Cc1cc(-c2ccc(C(F)(F)F)cc2Cl)nc(C(=O)OC)c1Cl] | Methyl 3-chloro-6- (2-chloro-4- (trifluoromethyl) phenyl)-4- ethynylpicolinate | Example 6 using F41 | White Solid | 0.04 g, 23% |
| F41 | 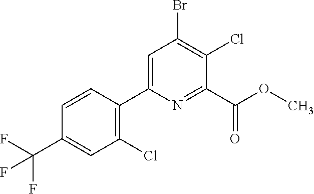 [SMILES: COC(=O)c1nc(-c2ccc(C(F)(F)F)cc2Cl)cc(Br)c1Cl] | Methyl 4-bromo-3- chloro-6-(2-chloro-4- (trifluoromethyl) phenyl)picolinate | Example 22 using copper(II) bromide as the bromine source | White Solid | 1 g, 42% |
| F42 | 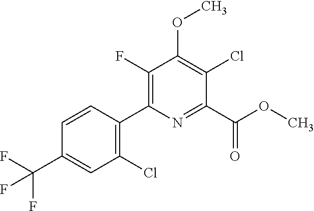 [SMILES: COC(=O)c1nc(-c2ccc(C(F)(F)F)cc2Cl)c(F)c(OC)c1Cl] | Methyl 3-chloro-6- (2-chloro-4- (trifluoromethyl) phenyl)-5-fluoro-4- methoxypicolinate | Example 17 using F43 | White Solid | 0.06 g, 33% |
| F43 | 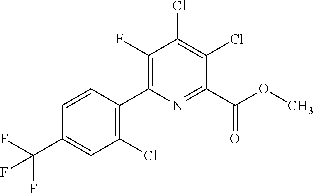 [SMILES: COC(=O)c1nc(-c2ccc(C(F)(F)F)cc2Cl)c(F)c(Cl)c1Cl] | Methyl 3,4-dichloro- 6-(2-chloro-4- (trifluoromethyl) phenyl)-5- fluoropicolinate | Example 22 using | White Solid | 0.25 g, 80% |
| F44 | 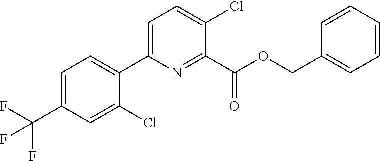 [SMILES: O=C(OCc1ccccc1)c1nc(-c2ccc(C(F)(F)F)cc2Cl)ccc1Cl] | Benzyl 3-chloro-6- (2-chloro-4- (trifluoromethyl) phenyl)picolinate | Example 25 using F61 | White Solid | 237 mg, 91% |
| F45 | 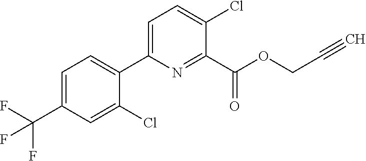 [SMILES: C#CCOC(=O)c1nc(-c2ccc(C(F)(F)F)cc2Cl)ccc1Cl] | Prop-2-yn-1-yl 3- chloro-6-(2-chloro-4- (trifluoromethyl) phenyl)picolinate | Example 26 using F61 | White Solid | 220 mg, 96% |
| F46 | 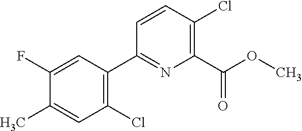 | Methyl 3-chloro-6- (2-chloro-5-fluoro-4- methylphenyl) picolinate | Example 32 with heating at 90° C. for 4 h | White Solid | 70 mg |
| F47 | 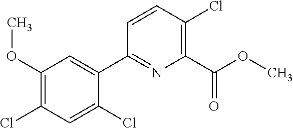 [SMILES: COC(=O)c1nc(-c2cc(OC)c(Cl)cc2Cl)ccc1Cl] | Methyl 3-chloro-6- (2,4-dichloro-5- methoxyphenyl) picolinate | Example 32 with heating at 90° C. for 4 h | White Solid | 70 mg |
| F48 | 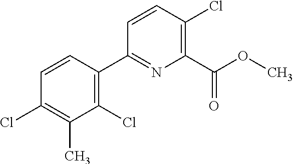 [SMILES: COC(=O)c1nc(-c2ccc(Cl)c(C)c2Cl)ccc1Cl] | Methyl 3-chloro-6- (2,4-dichloro-3- methylphenyl) picolinate | Example 32 with heating at 90° C. for 4 h | White Solid | 150 mg, 33% |
| F49 | 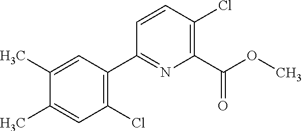 | Methyl 3-chloro-6- (2-chloro-4,5- dimethylphenyl) picolinate | Example 32 with heating at 90° C. for 4 h | White Solid | 30 mg |
| F50 | 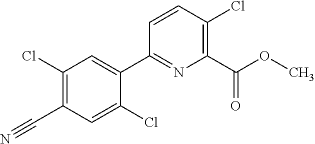 [SMILES: COC(=O)c1nc(-c2cc(Cl)c(C#N)cc2Cl)ccc1Cl] | Methyl 3-chloro-6- (2,5-dichloro-4- cyanophenyl) picolinate | Example 32 with heating at 90° C. for 4 h | White Solid | 20 mg |
| F51 | 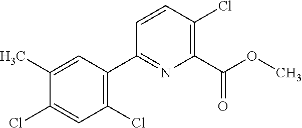 [SMILES: COC(=O)c1nc(-c2cc(C)c(Cl)cc2Cl)ccc1Cl] | Methyl 3-chloro-6- (2,4-dichloro-5- methylphenyl) picolinate | Example 32 with heating at 90° C. for 4 h | White Solid | 80 mg |
| F52 | 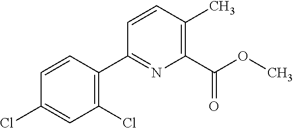 [SMILES: COC(=O)c1nc(-c2ccc(Cl)cc2Cl)ccc1C] | Methyl 6-(2,4- dichlorophenyl)-3- methylpicolinate | Example 32 with heating at 90° C. for 4 h | White Solid | 207 mg |
| F53 | 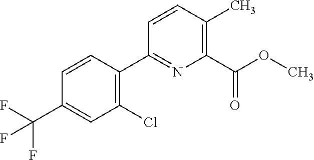 [SMILES: COC(=O)c1nc(-c2ccc(C(F)(F)F)cc2Cl)ccc1C] | Methyl 6-(2-chloro-4- (trifluoromethyl) phenyl)-3- methylpicolinate | Example 32 with heating at 90° C. for 4 h | White Solid | 185 mg |
| F54 | 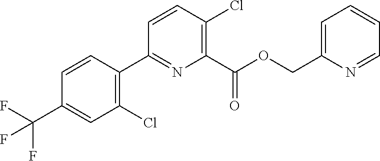 [SMILES: O=C(OCc1ccccn1)c1nc(-c2ccc(C(F)(F)F)cc2Cl)ccc1Cl] | Pyridin-2-ylmethyl 3- chloro-6-(2-chloro-4- (trifluoromethyl) phenyl)picolinate | Example 33 | White Solid | 65 mg, 49% |
| F55 | 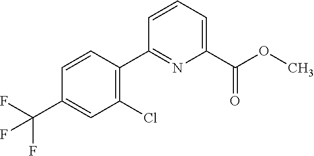 [SMILES: COC(=O)c1cccc(-c2ccc(C(F)(F)F)cc2Cl)n1] | Methyl 6-(2-chloro- 4-(trifluoromethyl) phenyl)picolinate | Example 32 with heating at 90° C. for 4 h | White Solid | 150 mg, 33% |
| F56 | 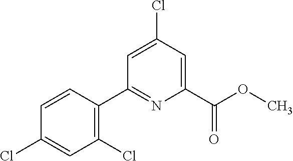 [SMILES: COC(=O)c1cc(Cl)cc(-c2ccc(Cl)cc2Cl)n1] | Methyl 4-chloro-6- (2,4- dichlorophenyl) picolinate | Example 32 with heating at 90° C. for 4 h | White Solid | 95 mg |
| F57 | 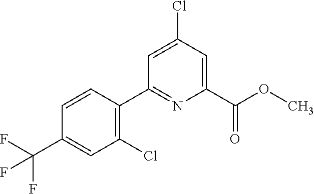 [SMILES: COC(=O)c1cc(Cl)cc(-c2ccc(C(F)(F)F)cc2Cl)n1] | Methyl 4-chloro-6- (2-chloro-4- (trifluoromethyl) phenyl)picolinate | Example 32 with heating at 90° C. for 4 h | White Solid | 150 mg, 35% |
| F58 | 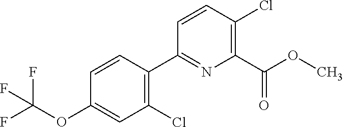 [SMILES: COC(=O)c1nc(-c2ccc(OC(F)(F)F)cc2Cl)ccc1Cl] | Methyl 3-chloro-6- (2-chloro-4- (trifluoromethoxy) phenyl)picolinate | Example 32 with heating at 90° C. for 4 h | White Solid | 235 mg |
| F59 | 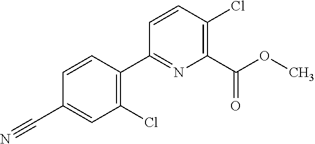 [SMILES: COC(=O)c1nc(-c2ccc(C#N)cc2Cl)ccc1Cl] | Methyl 3-chloro-6- (2-chloro-4- cyanophenyl) picolinate | Example 15 using F27 | Off-White Solid | 70 mg, 40% |
| F60 | 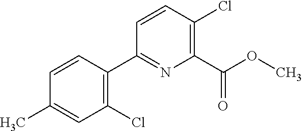 | Methyl 3-chloro-6- (2-chloro-4- methylphenyl) picolinate | Example 32 with heating at 90° C. for 4 h | White Solid | 240 mg |
| F61 | 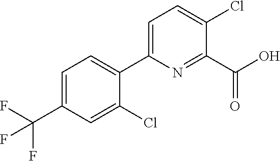 [SMILES: O=C(O)c1nc(-c2ccc(C(F)(F)F)cc2Cl)ccc1Cl] | 3-Chloro-6-(2-chloro- 4- (trifluoromethyl) phenyl) picolinic acid | Example 27 using F66 | White Solid | 1.13 g, 96% |
| F62 | 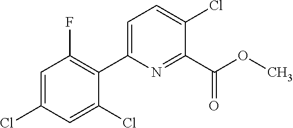 [SMILES: COC(=O)c1nc(-c2c(F)cc(Cl)cc2Cl)ccc1Cl] | Methyl 3-chloro-6- (2,4-dichloro-6- fluorophenyl) picolinate | Example 32 | White Solid | 142 mg, 44% |
| F63 | 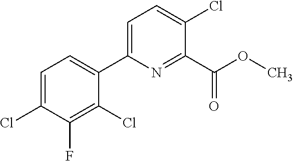 [SMILES: COC(=O)c1nc(-c2ccc(Cl)c(F)c2Cl)ccc1Cl] | Methyl 3-chloro-6- (2,4-dichloro-3- fluorophenyl) picolinate | Example 32 | White Solid | 55 mg, 34% |
| F64 | 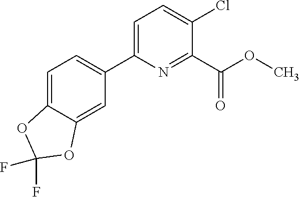 [SMILES: COC(=O)c1nc(-c2ccc3c(c2)OC(F)(F)O3)ccc1Cl] | Methyl 3-chloro-6- (2,2- difluorobenzo[d] [1,3]dioxol-5-yl) picolinate | Example 32 | Colorless Oil | 70 mg, 43% |
| F65 | 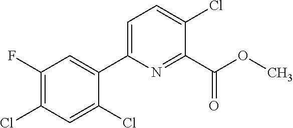 [SMILES: COC(=O)c1nc(-c2cc(F)c(Cl)cc2Cl)ccc1Cl] | Methyl 3-chloro-6- (2,4-dichloro-5- fluorophenyl) picolinate | Example 32 | White Solid | 55 mg, 34% |
| F66 | 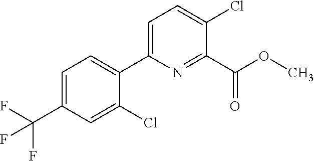 [SMILES: COC(=O)c1nc(-c2ccc(C(F)(F)F)cc2Cl)ccc1Cl] | Methyl 3-chloro-6- (2-chloro-4- (trifluoromethyl) phenyl)picolinate | Example 32 | White Solid | 1.97 g, 58% |
| F67 | 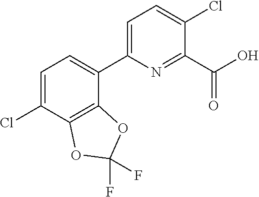 [SMILES: O=C(O)c1nc(-c2ccc(Cl)c3c2OC(F)(F)O3)ccc1Cl] | 3-Chloro-6-(7-chloro- 2,2- difluorobenzo[d] [1,3]dioxol-4-yl) picolinic acid | Example 27 using MeOH only with 8 equivalents of NaOH | Matte White/Off- White Residual Solid Film | 0.099 g, 55% |
| F68 | 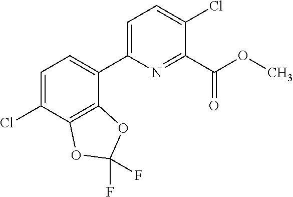 [SMILES: COC(=O)c1nc(-c2ccc(Cl)c3c2OC(F)(F)O3)ccc1Cl] | Methyl 3-chloro-6- (7-chloro-2,2- difluorobenzo[d] [1,3]dioxol- 4-yl)picolinate | Example 32 in a 5:1 mixture of CH₃CN-water with heating in a microwave reactor at 120° C. for 40 min | Light Yellow Semi-Solid | 216 mg, 30% |
| F69 | 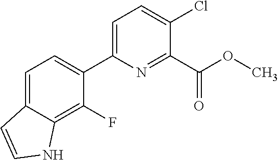 [SMILES: COC(=O)c1nc(-c2ccc3cc[nH]c3c2F)ccc1Cl] | Methyl 3-chloro-6- (7-fluoro-1H-indol-6- yl)picolinate | Example 32 in a 1:1 mixture of CH₃CN-water with heating in a microwave reactor at 110° C. for 20 min | Off-White Solid | 180 mg, 29% |
| F70 | 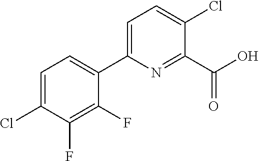 [SMILES: O=C(O)c1nc(-c2ccc(Cl)c(F)c2F)ccc1Cl] | 3-Chloro-6-(4-chloro- 2,3- difluorophenyl) picolinic acid | Example 27 | White Solid | 0.088 g, 60% |
| F71 | 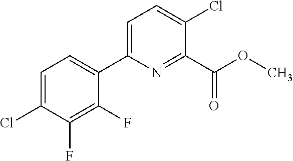 [SMILES: COC(=O)c1nc(-c2ccc(Cl)c(F)c2F)ccc1Cl] | Methyl 3-chloro-6- (4-chloro-2,3- difluorophenyl) picolinate | Example 32 in a 1:1 mixture of CH₃CN-water with heating in a microwave reactor at 110° C. for 20 min | White Solid | 333 mg, 72% |
| F72 | 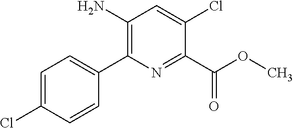 [SMILES: COC(=O)c1nc(-c2ccc(Cl)cc2)c(N)cc1Cl] | Methyl 5-amino-3- chloro-6-(4- chlorophenyl) picolinate | Example 31 using C8 | White Solid | 0.080 g, 22% |
| F73 | 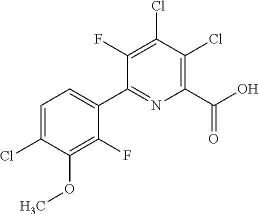 [SMILES: COc1c(Cl)ccc(-c2nc(C(=O)O)c(Cl)c(Cl)c2F)c1F] | 3,4-Dichloro-6-(4- chloro-2-fluoro-3- methoxyphenyl)-5- fluoropicolinic acid | Example 22 with heating at 65° C. for 10 min reaction time | Cream Powder | 92 mg, 44% |
| F74 | 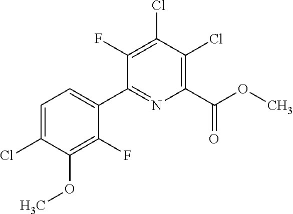 [SMILES: COC(=O)c1nc(-c2ccc(Cl)c(OC)c2F)c(F)c(Cl)c1Cl] | Methyl 3,4-dichloro- 6-(4-chloro-2-fluoro- 3-methoxyphenyl)-5- fluoropicolinate | Example 22 with heating at 65° C. for 10 min reaction time | White Powder | 180 mg, 57% |
| F75 | 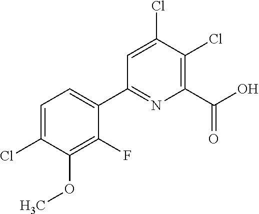 [SMILES: COc1c(Cl)ccc(-c2cc(Cl)c(Cl)c(C(=O)O)n2)c1F] | 3,4-Dichloro-6-(4- chloro-2-fluoro-3- methoxyphenyl) picolinic acid | Example 21 | Tan Powder | 170 mg, 81% |
| F76 | 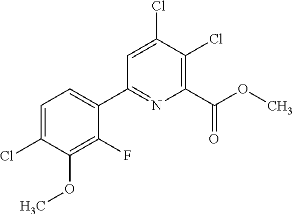 [SMILES: COC(=O)c1nc(-c2ccc(Cl)c(OC)c2F)cc(Cl)c1Cl] | Methyl 3,4-dichloro- 6-(4-chloro-2-fluoro-3- methoxyphenyl) picolinate | Example 21 | White Powder | 140 mg, 67% |
| F77 | 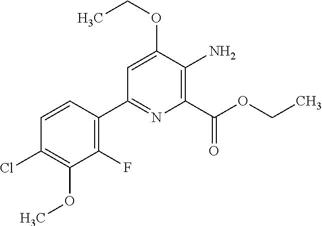 [SMILES: CCOC(=O)c1nc(-c2ccc(Cl)c(OC)c2F)cc(OCC)c1N] | Ethyl 3-amino-6-(4- chloro-2-fluoro-3- methoxyphenyl)-4- ethoxypicolinate | Example 36 | Off-White Solid | 56 mg, 35% |
| F78 | 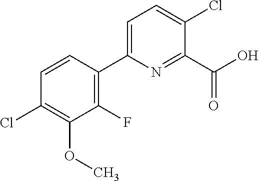 [SMILES: COc1c(Cl)ccc(-c2ccc(Cl)c(C(=O)O)n2)c1F] | 3-Chloro-6-(4-chloro- 2-fluoro-3- methoxyphenyl) picolinic acid | Example 28 | White Solid | 200 mg, 76% |
| F79 | 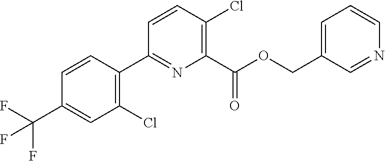 [SMILES: O=C(OCc1cccnc1)c1nc(-c2ccc(C(F)(F)F)cc2Cl)ccc1Cl] | Pyridin-3-ylmethyl 3- chloro-6-(2-chloro-4- (trifluoromethyl) phenyl)picolinate | Example 33 | White Solid | 24 mg, 24% |
| F80 | 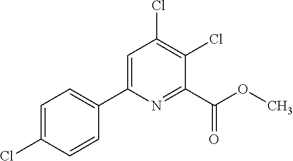 [SMILES: COC(=O)c1nc(-c2ccc(Cl)cc2)cc(Cl)c1Cl] | Methyl 3,4-dichloro- 6-(4- chlorophenyl) picolinate | Example 51 | White Solid | 820 mg, 63% |
| F81 | 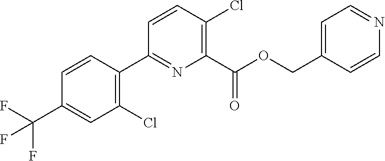 [SMILES: O=C(OCc1ccncc1)c1nc(-c2ccc(C(F)(F)F)cc2Cl)ccc1Cl] | Pyridin-4-ylmethyl 3- chloro-6-(2-chloro-4- (trifluoromethyl) phenyl)picolinate | Example 33 | White Solid | 118 mg, 78% |
| F82 | 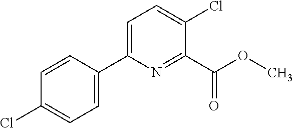 [SMILES: COC(=O)c1nc(-c2ccc(Cl)cc2)ccc1Cl] | Methyl 3-chloro-6- (4- chlorophenyl) picolinate | Example 34 | Yellow Oil | 1.9 g, 23% |
| F83 | 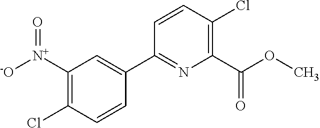 | Methyl 3-chloro-6- (4-chloro-3- nitrophenyl) picolinate | Example 19 using tert-butylnitrite and DMF as solvent, as described in J. Org. Chem. 1977, 42, 3494 | Off-White Powder | 0.077 g, 81% |
| F84 | 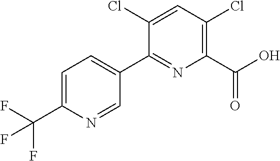 [SMILES: O=C(O)c1nc(-c2ccc(C(F)(F)F)nc2)c(Cl)cc1Cl] | 3,5-Dichloro-6′- (trifluoromethyl)- [2,3′-bipyridine]-6- carboxylic acid | Example 29 | White Solid | 0.093 g, 81% |
| F85 | 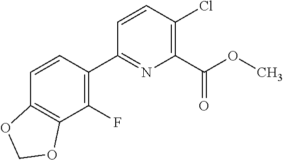 [SMILES: COC(=O)c1nc(-c2ccc3c(c2F)OCO3)ccc1Cl] | Methyl 3-chloro-6- (4- fluorobenzo[d][1,3] dioxol-5-yl)picolinate | Example 19 using tert-butylnitrite and DMF as solvent, as described in J. Org. Chem. 1977, 42, 3494 | Light Yellow Powder | 0.088 g, 92% |
| F86 | 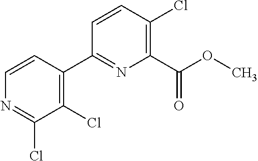 [SMILES: COC(=O)c1nc(-c2ccnc(Cl)c2Cl)ccc1Cl] | Methyl 2′,3′,5- trichloro-[2,4′- bipyridine]-6- carboxylate | Example 51 | White Solid | 62 mg, 18% |
| F87 | 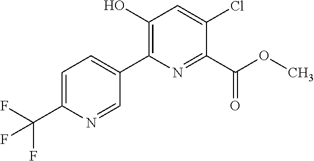 [SMILES: COC(=O)c1nc(-c2ccc(C(F)(F)F)nc2)c(O)cc1Cl] | Methyl 5-chloro-3- hydroxy-6′- (trifluoromethyl)- [2,3′-bipyridine]-6- carboxylate | Example 30 using F92 | White Solid | 0.052 g, 90% |
| F88 | 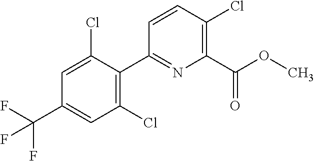 [SMILES: COC(=O)c1nc(-c2c(Cl)cc(C(F)(F)F)cc2Cl)ccc1Cl] | Methyl 3-chloro-6- (2,6-dichloro-4- (trifluoromethyl) phenyl)picolinate | Example 32 | Light Yellow Oil | 24 mg, 10% |
| F89 | 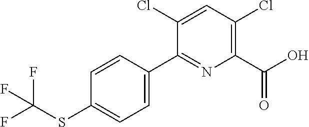 [SMILES: O=C(O)c1nc(-c2ccc(SC(F)(F)F)cc2)c(Cl)cc1Cl] | 3,5-Dichloro-6-(4- ((trifluoromethyl) thio)phenyl) picolinic acid | Example 29 | White Solid | 0.129 g, 82% |
| F90 | 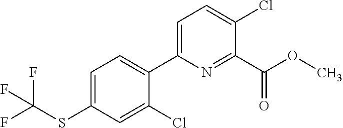 [SMILES: COC(=O)c1nc(-c2ccc(SC(F)(F)F)cc2Cl)ccc1Cl] | Methyl 3-chloro-6- (2-chloro-4- ((trifluoromethyl) thio)phenyl) picolinate | Example 32 | Colorless Oil | 133 mg, 57% |
| F91 | 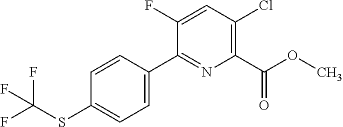 [SMILES: COC(=O)c1nc(-c2ccc(SC(F)(F)F)cc2)c(F)cc1Cl] | Methyl 3-chloro-5- fluoro-6-(4- ((trifluoromethyl) thio)phenyl) picolinate | Example 32 | White Solid | 200 mg, 66% |
| F92 | 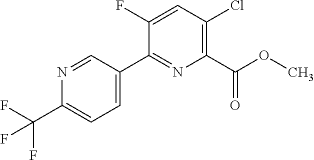 [SMILES: COC(=O)c1nc(-c2ccc(C(F)(F)F)nc2)c(F)cc1Cl] | Methyl 5-chloro-3- fluoro-6′- (trifluoromethyl)- [2,3′-bipyridine]-6- carboxylate | Example 32 | White Solid | 144 mg, 41% |
| F93 | 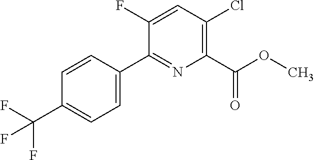 [SMILES: COC(=O)c1nc(-c2ccc(C(F)(F)F)cc2)c(F)cc1Cl] | Methyl 3-chloro-5- fluoro-6-(4- (trifluoromethyl) phenyl)picolinate | Example 32 | Colorless Oil | 277 mg, 70% |
| F94 | 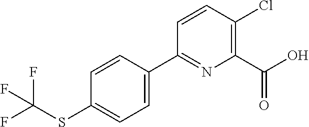 [SMILES: O=C(O)c1nc(-c2ccc(SC(F)(F)F)cc2)ccc1Cl] | 3-Chloro-6-(4- ((trifluoromethyl)thio) phenyl)picolinic acid | Example 29 | White Solid | 0.286 g, 96% |
| F95 | 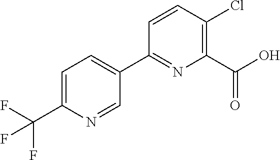 [SMILES: O=C(O)c1nc(-c2ccc(C(F)(F)F)nc2)ccc1Cl] | 5-Chloro-6′- (trifluoromethyl)- [2,3′-bipyridine]-6- carboxylic acid | Example 29 | White Solid | 0.154 g, 47% |
| F96 | 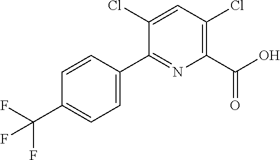 [SMILES: O=C(O)c1nc(-c2ccc(C(F)(F)F)cc2)c(Cl)cc1Cl] | 3,5-Dichloro-6-(4- (trifluoromethyl) phenyl)picolinic acid | Example 29 | White Solid | 0.115 g, 92% |
| F97 | 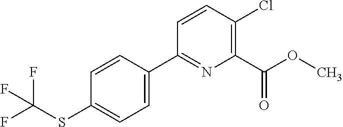 [SMILES: COC(=O)c1nc(-c2ccc(SC(F)(F)F)cc2)ccc1Cl] | Methyl 3-chloro-6- (4- ((trifluoromethyl)thio) phenyl)picolinate | Example 32 | Colorless Oil | 76 mg, 45% |
| F98 | 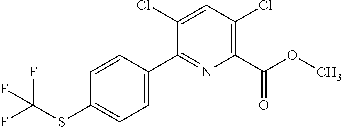 [SMILES: COC(=O)c1nc(-c2ccc(SC(F)(F)F)cc2)c(Cl)cc1Cl] | Methyl 3,5-dichloro- 6-(4- ((trifluoromethyl)thio) phenyl)picolinate | Example 32 | Colorless Oil | 312 mg, 66% |
| F99 | 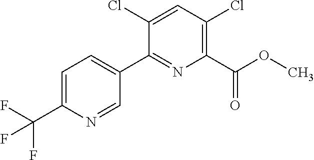 [SMILES: COC(=O)c1nc(-c2ccc(C(F)(F)F)nc2)c(Cl)cc1Cl] | Methyl 3,5-dichloro- 6′-(trifluoromethyl)- [2,3′-bipyridine]-6- carboxylate | Example 32 | White Solid | 297 mg, 68% |
| F100 | 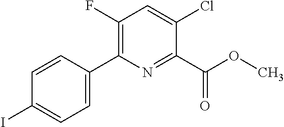 [SMILES: COC(=O)c1nc(-c2ccc(I)cc2)c(F)cc1Cl] | Methyl 3-chloro-5- fluoro-6-(4- iodophenyl) picolinate | Example 19 | White Solid | 200 mg, 65% |
| F101 | 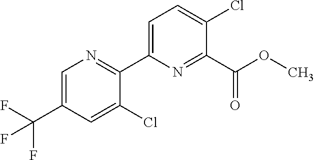 [SMILES: COC(=O)c1nc(-c2ncc(C(F)(F)F)cc2Cl)ccc1Cl] | Methyl 3′,5-dichloro- 5′-(trifluoromethyl)- [2,2′-bipyridine]-6- carboxylate | Example 41 using C16 | Yellow Oil | 0.069 g, 66% |
| F102 | 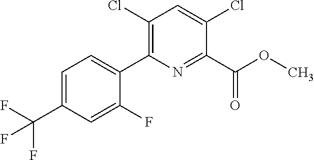 [SMILES: COC(=O)c1nc(-c2ccc(C(F)(F)F)cc2F)c(Cl)cc1Cl] | Methyl 3,5-dichloro- 6-(2-fluoro-4- (trifluoromethyl) phenyl)picolinate | Example 32 | White Solid | 240 mg, 48% |
| F103 | 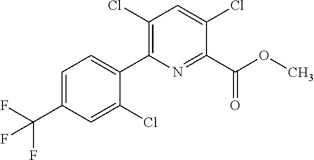 [SMILES: COC(=O)c1nc(-c2ccc(C(F)(F)F)cc2Cl)c(Cl)cc1Cl] | Methyl 3,5-dichloro- 6-(2-chloro-4- (trifluoromethyl) phenyl)picolinate | Example 32 | White Solid | 290 mg, 57% |
| F104 | 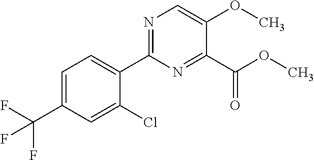 [SMILES: COC(=O)c1nc(-c2ccc(C(F)(F)F)cc2Cl)ncc1OC] | Methyl 2-(2-chloro- 4- (trifluoromethyl) phenyl)-5- methoxypyrimidine- 4-carboxylate | Example 32 | Clear Gum | 40 mg, 15% |
| F105 | 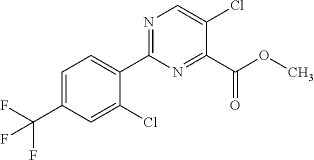 [SMILES: COC(=O)c1nc(-c2ccc(C(F)(F)F)cc2Cl)ncc1Cl] | Methyl 5-chloro-2- (2-chloro-4- (trifluoromethyl) phenyl)pyrimidine-4- carboxylate | Example 32 | Clear Gum | 70 mg, 40% |
| F106 | 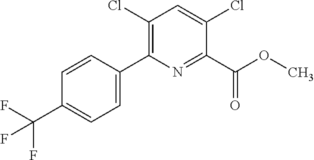 [SMILES: COC(=O)c1nc(-c2ccc(C(F)(F)F)cc2)c(Cl)cc1Cl] | Methyl 3,5-dichloro- 6-(4- (trifluoromethyl) phenyl)picolinate | Example 32 | White Solid | 155 mg, 33% |
| F107 | 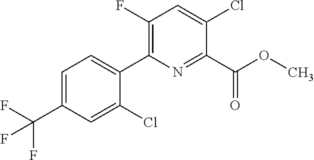 [SMILES: COC(=O)c1nc(-c2ccc(C(F)(F)F)cc2Cl)c(F)cc1Cl] | Methyl 3-chloro-6- (2-chloro-4- (trifluoromethyl) phenyl)-5- fluoropicolinate | Example 32 | White Solid | 144 mg, 72% |
| F108 | 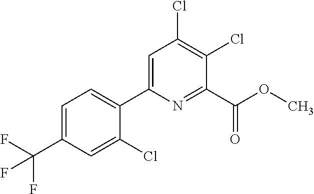 [SMILES: COC(=O)c1nc(-c2ccc(C(F)(F)F)cc2Cl)cc(Cl)c1Cl] | Methyl 3,4-dichloro- 6-(2-chloro-4- (trifluoromethyl) phenyl)picolinate | Example 32 | White Solid | 110 mg, 44% |
| F109 | 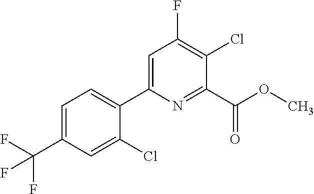 [SMILES: COC(=O)c1nc(-c2ccc(C(F)(F)F)cc2Cl)cc(F)c1Cl] | Methyl 3-chloro-6- (2-chloro-4- (trifluoromethyl) phenyl)-4- fluoropicolinate | Example 40 using F108 | White Solid | 24 mg, 12% |
| F110 | 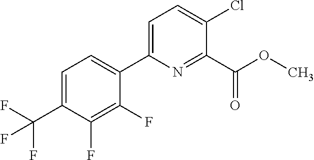 [SMILES: COC(=O)c1nc(-c2ccc(C(F)(F)F)c(F)c2F)ccc1Cl] | Methyl 3-chloro-6- (2,3-difluoro-4- (trifluoromethyl) phenyl)picolinate | Example 32 | White Solid | 264 mg, 52% |
| F111 | 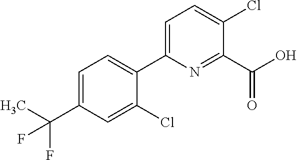 [SMILES: CC(F)(F)c1ccc(-c2ccc(Cl)c(C(=O)O)n2)c(Cl)c1] | 3-Chloro-6-(2-chloro- 4-(1,1- difluoroethyl)phenyl) picolinic acid | Example 27 | Yellow Oil | 65 mg, 65% |
| F112 | 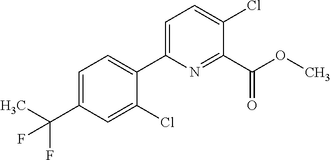 [SMILES: COC(=O)c1nc(-c2ccc(C(C)(F)F)cc2Cl)ccc1Cl] | Methyl 3-chloro-6- (2-chloro-4-(1,1- difluoroethyl)phenyl) picolinate | Example 32 | Brown Oil | 138 mg, 33% |
| F113 | 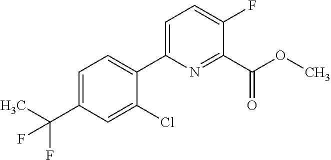 [SMILES: COC(=O)c1nc(-c2ccc(C(C)(F)F)cc2Cl)ccc1F] | Methyl 6-(2-chloro- 4- (trifluoromethyl) phenyl)-3- fluoropicolinate | Example 52 | White Solid | 174 mg, 31% |
| F114 | 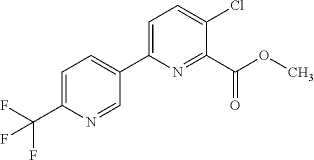 [SMILES: COC(=O)c1nc(-c2ccc(C(F)(F)F)nc2)ccc1Cl] | Methyl 5-chloro-6′- (trifluoromethyl)- [2,3′-bipyridine]-6- carboxylate | Example 32 | White Solid | 94 mg, 61% |
| F115 | 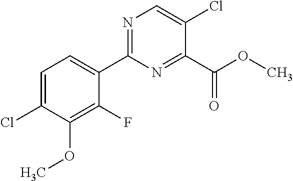 [SMILES: COC(=O)c1nc(-c2ccc(Cl)c(OC)c2F)ncc1Cl] | Methyl 5-chloro-2- (4-chloro-2-fluoro-3 - methoxyphenyl) pyrimidine-4- carboxylate | Example 37 | White Solid | 0.100 g, 60% |
| F116 | 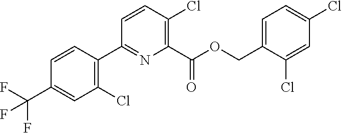 [SMILES: O=C(OCc1ccc(Cl)cc1Cl)c1nc(-c2ccc(C(F)(F)F)cc2Cl)ccc1Cl] | 2,4-Dichlorobenzyl 3-chloro-6-(2-chloro- 4- (trifluoromethyl) phenyl)picolinate | Example 33 | Clear Oil | 32 mg, 27% |
| F117 | 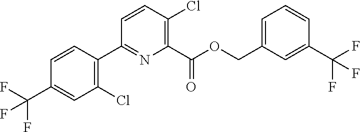 [SMILES: O=C(OCc1cccc(C(F)(F)F)c1)c1nc(-c2ccc(C(F)(F)F)cc2Cl)ccc1Cl] | 3- (Trifluoromethyl) benzyl 3-chloro-6-(2- chloro-4- (trifluoromethyl) phenyl)picolinate | Example 33 | White Solid | 87 mg, 57% |
| F118 | 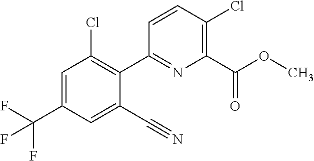 [SMILES: COC(=O)c1nc(-c2c(Cl)cc(C(F)(F)F)cc2C#N)ccc1Cl] | Methyl 3-chloro-6- (2-chloro-6-cyano-4- (trifluoromethyl) phenyl)picolinate | Example 32 with heating at 90° C. | Colorless Liquid for 5 h | 31% |
| F119 | 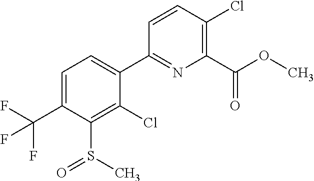 [SMILES: COC(=O)c1nc(-c2ccc(C(F)(F)F)c(S(C)=O)c2Cl)ccc1Cl] | Methyl 3-chloro-6- (2-chloro-3- (methylsulfinyl)-4- (trifluoromethyl) phenyl)picolinate | Example 60 | Colorless Liquid | 33 mg, 16% |
| F120 | 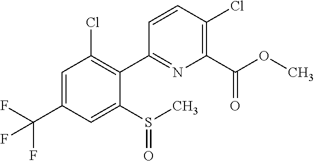 [SMILES: COC(=O)c1nc(-c2c(Cl)cc(C(F)(F)F)cc2S(C)=O)ccc1Cl] | Methyl 3-chloro-6- (2-chloro-6- (methylsulfinyl)-4- (trifluoromethyl) phenyl)picolinate | Example 60 | Brown Solid | 70 mg, 34% |
| F121 | 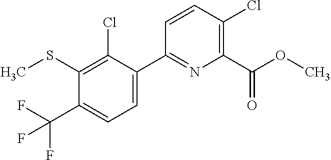 [SMILES: COC(=O)c1nc(-c2ccc(C(F)(F)F)c(SC)c2Cl)ccc1Cl] | Methyl 3-chloro-6- (2-chloro-3- (methylthio)-4- (trifluoromethyl) phenyl)picolinate | Example 61 | Colorless Liquid | 50 mg, 9% |
| F122 | 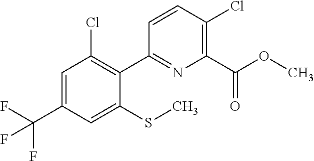 [SMILES: COC(=O)c1nc(-c2c(Cl)cc(C(F)(F)F)cc2SC)ccc1Cl] | Methyl 3-chloro-6- (2-chloro-6- (methylthio)-4- (trifluoromethyl) phenyl)picolinate | Example 61 | White Solid | 200 mg, 36% |
| F123 | 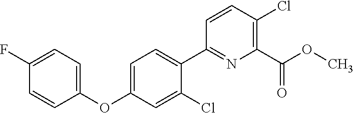 [SMILES: COC(=O)c1nc(-c2ccc(Oc3ccc(F)cc3)cc2Cl)ccc1Cl] | Methyl 3-chloro-6- (2-chloro-4-(4- fluorophe)phenyl) picolinate | Example 32 | Colorless Oil | 66 mg, 28% |
| F124 | 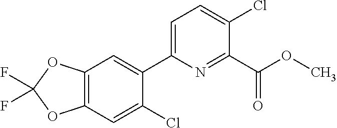 [SMILES: COC(=O)c1nc(-c2cc3c(cc2Cl)OC(F)(F)O3)ccc1Cl] | Methyl 3-chloro-6- (6-chloro-2,2- difluorobenzo[d] [1,3]dioxol-5- yl)picolinate | Example 51 | White Solid | 148 mg, 42% |
| F125 | 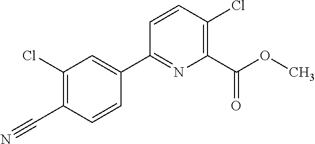 [SMILES: COC(=O)c1nc(-c2ccc(C#N)c(Cl)c2)ccc1Cl] | Methyl 3-chloro-6- (3-chloro-4- cyanophenyl) picolinate | Example 32 | White Solid | 46 mg, 31% |
| F126 | 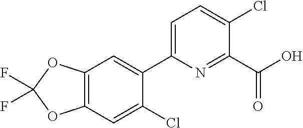 [SMILES: O=C(O)c1nc(-c2cc3c(cc2Cl)OC(F)(F)O3)ccc1Cl] | 3-Chloro-6-(6-chloro- difluorobenzo[d] [1,3]dioxol-5- yl)picolinic acid | Example 27 | White Solid | 86 mg, 90% |
| F127 | 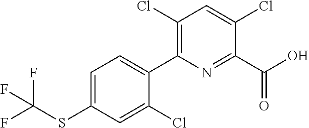 [SMILES: O=C(O)c1nc(-c2ccc(SC(F)(F)F)cc2Cl)c(Cl)cc1Cl] | 3,5-Dichloro-6-(2- chloro-4- ((trifluoromethyl) thio)phenyl) picolinic acid | Example 27 | Clear Sticky Solid | 49 mg, 91% |
| F128 | 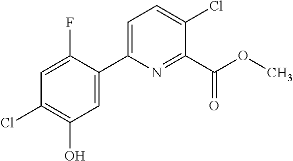 [SMILES: COC(=O)c1nc(-c2cc(O)c(Cl)cc2F)ccc1Cl] | Methyl 3-chloro-6- (4-chloro-2-fluoro-5- hydroxyphenyl) picolinate | Example 51 | White Solid | 161 mg, 38% |
| F129 | 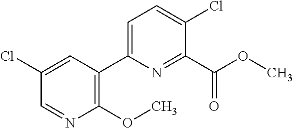 [SMILES: COC(=O)c1nc(-c2cc(Cl)cnc2OC)ccc1Cl] | Methyl 5,5′-dichloro- 2′-methoxy-[2,3′- bipyridine-6- carboxylate | Example 51 | White Solid | 191 mg, 56% |
| F130 | 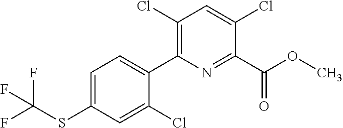 [SMILES: COC(=O)c1nc(-c2ccc(SC(F)(F)F)cc2Cl)c(Cl)cc1Cl] | Methyl 3,5-dichloro- 6-(2-chloro-4- ((trifluoromethyl) thio)phenyl) picolinate | Example 32 | Clear | 85 mg, 49% Viscous Oil |
| F131 | 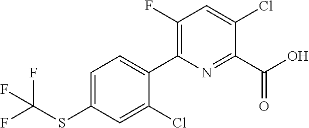 [SMILES: O=C(O)c1nc(-c2ccc(SC(F)(F)F)cc2Cl)c(F)cc1Cl] | 3-Chloro-6-(2-chloro- 4- ((trifluoromethyl) thio)phenyl)-5- fluoropicolinic acid | Example 27 | Clear Viscous Oil | 82 mg, 85% |
| F132 | 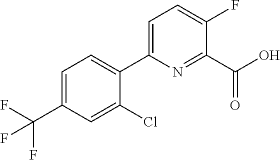 [SMILES: O=C(O)c1nc(-c2ccc(C(F)(F)F)cc2Cl)ccc1F] | 6-(2-Chloro-4- (trifluoromethyl) phenyl)-3- fluoropicolinic acid | Example 27 | White solid | 87 mg, 83% |
| F133 | 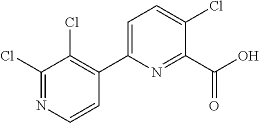 [SMILES: O=C(O)c1nc(-c2ccnc(Cl)c2Cl)ccc1Cl] | 2′,3′,5-Trichloro- [2,4′-bipyridine]-6- carboxylic acid | Example 27 | White Solid | 35 mg, 56% |
| F134 | 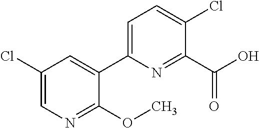 [SMILES: COc1ncc(Cl)cc1-c1ccc(Cl)c(C(=O)O)n1] | 5,5′-Dichloro-2′- methoxy-[2,3′- bipyridine]-6- carboxylic acid | Example 27 | White Solid | 90 mg, 70% |
| F135 | 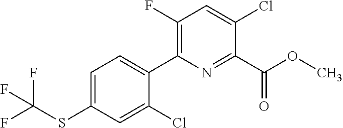 [SMILES: COC(=O)c1nc(-c2ccc(SC(F)(F)F)cc2Cl)c(F)cc1Cl] | Methyl 3-chloro-6- (2-chloro-4- ((trifluoromethyl) thio)phenyl)-5 fluoropicolinate | Example 32 | White Solid | 273 mg, 76% |
| F136 | 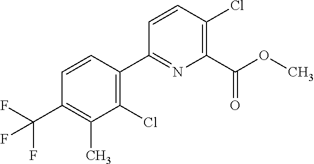 [SMILES: COC(=O)c1nc(-c2ccc(C(F)(F)F)c(C)c2Cl)ccc1Cl] | Methyl 3-chloro-6- (2-chloro-3- methyl-4- (trifluoromethyl) phenyl)picolinate | Example 57 | White Solid | 80 mg, 31% |
| F137 | 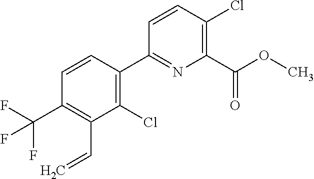 [SMILES: C=Cc1c(C(F)(F)F)ccc(-c2ccc(Cl)c(C(=O)OC)n2)c1Cl] | Methyl 3-chloro-6- (2-chloro-4- (trifluoromethyl)-3- vinylphenyl) picolinate | JKY | White Solid | 25 mg, 19% |
| F138 | 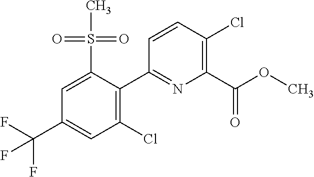 [SMILES: COC(=O)c1nc(-c2c(Cl)cc(C(F)(F)F)cc2S(C)(=O)=O)ccc1Cl] | Methyl 3-chloro-6- (2-chloro-6- (methylsulfonyl)-4- (trifluoromethyl) phenyl)picolinate | Example 59 | White Solid | 15 mg, 6% |
| F139 | 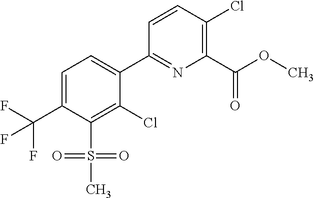 [SMILES: COC(=O)c1nc(-c2ccc(C(F)(F)F)c(S(C)(=O)=O)c2Cl)ccc1Cl] | Methyl 3-chloro-6- (2-chloro-3- (methylsulfonyl)-4- (trifluoromethyl) phenyl)picolinate | Example 59 | White Solid | 40 mg, 15% |
| F140 | 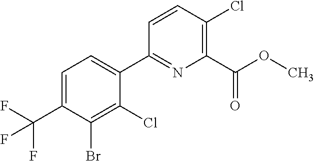 [SMILES: COC(=O)c1nc(-c2ccc(C(F)(F)F)c(Br)c2Cl)ccc1Cl] | Methyl 6-(3-bromo- 2-chloro-4- (trifluoromethyl) phenyl)-3- chloropicolinate | Example 32 with heating at 90° C. for 5 h | White Solid | 22% |
| F141 | 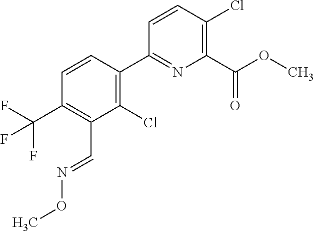 [SMILES: CO/N=C/c1c(C(F)(F)F)ccc(-c2ccc(Cl)c(C(=O)OC)n2)c1Cl] | Methyl (E)-3-chloro- 6-(2-chloro-3- ((methoxyimino) methyl)-4- (trifluoromethyl) phenyl)picolinate | Example 32 with heating at 90° C. for 5 h | White Solid | 16% |
| F142 | 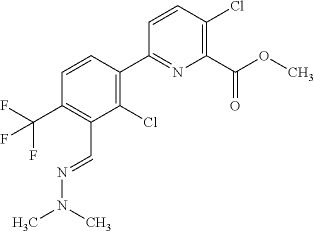 [SMILES: COC(=O)c1nc(-c2ccc(C(F)(F)F)c(/C=N/N(C)C)c2Cl)ccc1Cl] | Methyl (E)-3-chloro- 6-(2-chloro-3-((2,2- dimethylhydrazono) methyl)-4- (trifluoromethyl) phenyl)picolinate | Example 32 with heating at 90° C. for 5 h | White Solid | 38% |
| F143 | 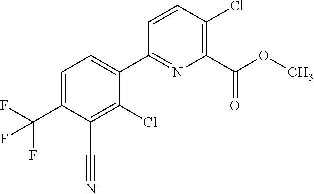 [SMILES: COC(=O)c1nc(-c2ccc(C(F)(F)F)c(C#N)c2Cl)ccc1Cl] | Methyl 3-chloro-6- (2-chloro-3-cyano-4- (trifluoromethyl) phenyl)picolinate | Example 62 | Off-White Solid | 40 mg, 30% |
| F144 | 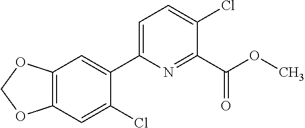 [SMILES: COC(=O)c1nc(-c2cc3c(cc2Cl)OCO3)ccc1Cl] | Methyl 3-chloro-6- (6- chlorobenzo[d] [1,3]dioxol-5-yl) picolinate | Example 51 | White Solid | 181 mg, 51% |
| F145 | 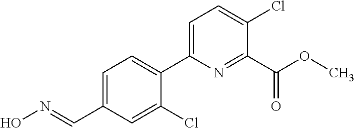 [SMILES: COC(=O)c1nc(-c2ccc(/C=N/O)cc2Cl)ccc1Cl] | Methyl 3-chloro-6- (2-chloro-4- ((hydroxyimino) methyl) phenyl)picolinate | Example 35 using F22 | White Solid | 24 mg, 56% |
| F146 | 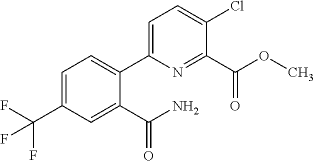 [SMILES: COC(=O)c1nc(-c2ccc(C(F)(F)F)cc2C(N)=O)ccc1Cl] | Methyl 6-(2- carbamoyl-4- (trifluoromethyl) phenyl)- 3-chloropicolinate | Example 32 using (2-(4,4,5,5- tetramethyl-1,3,2- dioxaborolan- 2-yl)-5- (trifluoromethyl) benzonitrile (the nitrile hydrolyzes to the primary amide under the | Off-White Solid | 263 mg, 60% |
| | | | reaction | | |
| | | | conditions) | | |
| F147 | 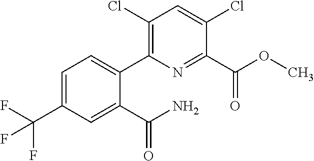 [SMILES: COC(=O)c1nc(-c2ccc(C(F)(F)F)cc2C(N)=O)c(Cl)cc1Cl] | Methyl 6-(2- carbamoyl-4- (trifluoromethyl) phenyl)-3,5- dichloropicolinate | Example 32 using (2-(4,4,5,5- tetramethyl-1,3,2- dioxaborolan-2-yl)-5- (trifluoromethyl) benzonitrile (the nitrile hydrolyzes to the primary amide under the reaction | Off-White Soild | 220 mg, 54% |
| | | | conditions) | | |
| F148 | 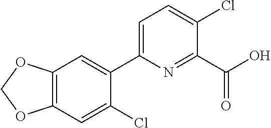 [SMILES: O=C(O)c1nc(-c2cc3c(cc2Cl)OCO3)ccc1Cl] | 3-Chloro-6-(6- chlorobenzo[d][1,3] dioxol-5-yl)picolinic acid | Example 27 | White Solid | 40 mg, 78% |
| F149 | 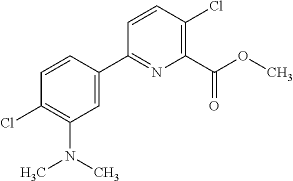 [SMILES: COC(=O)c1nc(-c2ccc(Cl)c(N(C)C)c2)ccc1Cl] | Methyl 3-chloro-6- (4-chloro-3- (dimethylamino) phenyl)picolinate | Example 19 using tert-butylnitrite and DMF as solvent, as described in J. Org. Chem. 1977, 42, 3494 | Yellow Oil | 11 mg, 15% |
| F150 | 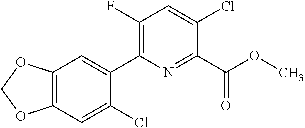 [SMILES: COC(=O)c1nc(-c2cc3c(cc2Cl)OCO3)c(F)cc1Cl] | Methyl 3-chloro-6- (6- chlorobenzo[d] [1,3]oxol-5-yl)-5- fluoropicolinate | Example 51 | White Solid | 115 mg, 31% |
| F151 | 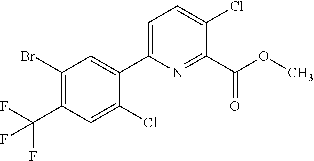 [SMILES: COC(=O)c1nc(-c2cc(Br)c(C(F)(F)F)cc2Cl)ccc1Cl] | Methyl 6-(5-bromo- 2-chloro-4- (trifluoromethyl) phenyl)-3- chloropicolinate | Example 51 | White Solid | 53 mg, 29% |
| F152 | 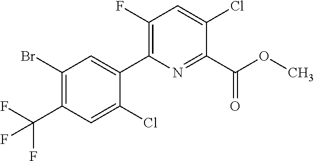 [SMILES: COC(=O)c1nc(-c2cc(Br)c(C(F)(F)F)cc2Cl)c(F)cc1Cl] | Methyl 6-(5-bromo- 2-chloro-4- (trifluoromethyl) phenyl)-3-chloro-5- fluoropicolinate | Example 51 | White Solid | 67 mg, 36% |
| F153 | 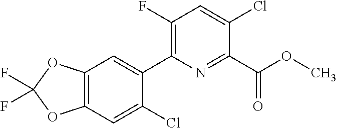 [SMILES: COC(=O)c1nc(-c2cc3c(cc2Cl)OC(F)(F)O3)c(F)cc1Cl] | Methyl 3-chloro-6- (6-chloro-2,2- difluorobenzo[d] [1,3]dioxol-5-yl)- 5- fluoropicolinate | Example 51 | White Solid | 58 mg, 29% |
| F154 | 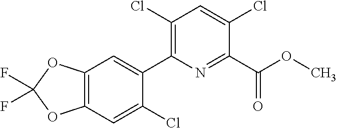 [SMILES: COC(=O)c1nc(-c2cc3c(cc2Cl)OC(F)(F)O3)c(Cl)cc1Cl] | Methyl 3,5-dichloro- 6-(6-chloro-2,2- difluorobenzo[d] [1,3]dioxol- 5-yl)picolinate | Example 51 | Orange Solid | 377 mg, 45% |
| F155 | 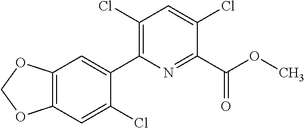 [SMILES: COC(=O)c1nc(-c2cc3c(cc2Cl)OCO3)c(Cl)cc1Cl] | Methyl 3,5-dichloro- 6-(6- chlorobenzo[d] [1,3]dioxol- 5-yl)picolinate | Example 51 | Yellow Oil | 126 mg, 35% |
| F156 | 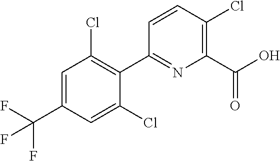 [SMILES: O=C(O)c1nc(-c2c(Cl)cc(C(F)(F)F)cc2Cl)ccc1Cl] | 3-Chloro-6-(2,6- dichloro-4- (trifluoromethyl) phenyl)picolinic acid | Example 28 | Tan Solid | 29 mg, 100% |
| F157 | 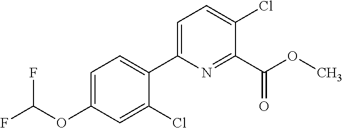 [SMILES: COC(=O)c1nc(-c2ccc(OC(F)F)cc2Cl)ccc1Cl] | Methyl 3-chloro-6- (2-chloro-4- (difluoromethoxy) phenyl)picolinate | Example 68 using C93 | Off-White Solid | 125 mg, 24% |
| F158 | 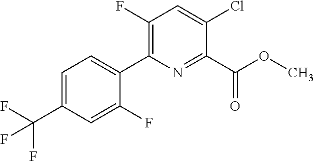 [SMILES: COC(=O)c1nc(-c2ccc(C(F)(F)F)cc2F)c(F)cc1Cl] | Methyl 3-chloro-5- fluoro-6-(2-fluoro-4- (trifluoromethyl) phenyl)picolinate | Example 63 | White Solid | 353 mg, 71% |
| F159 | 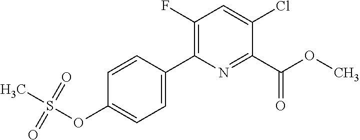 | Methyl 3-chloro-5- fluoro-6-(4- ((methylsulfonyl)oxy) phenyl)picolinate | Example 63 | Light Yellow Solid | 248 mg, 50% |
| F160 | 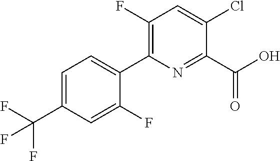 [SMILES: O=C(O)c1nc(-c2ccc(C(F)(F)F)cc2F)c(F)cc1Cl] | 3-Chloro-5-fluoro-6- (2-fluoro-4- (trifluoromethyl) phenyl) picolinic acid | Example 50 | White Solid | 197 mg, 77% |
| F161 | 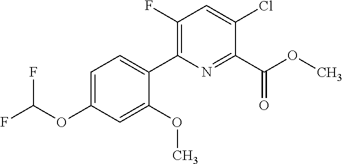 [SMILES: COC(=O)c1nc(-c2ccc(OC(F)F)cc2OC)c(F)cc1Cl] | Methyl 3-chloro-6- 4- (difluoromethoxy)-2- methoxyphenyl)-5- fluoropicolinate | Example 68 using C94 | Yellow Solid | 163 mg, 25% |
| F162 | 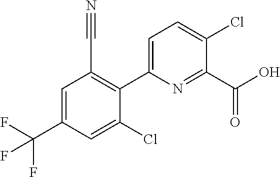 [SMILES: N#Cc1cc(C(F)(F)F)cc(Cl)c1-c1ccc(Cl)c(C(=O)O)n1] | 3-Chloro-6-(2-chloro- 6-cyano-4- (trifluoromethyl) phenyl)picolinic acid | Example 27 | White Solid | 160 mg, 84% |
| F163 | 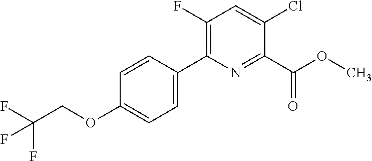 [SMILES: COC(=O)c1nc(-c2ccc(OCC(F)(F)F)cc2)c(F)cc1Cl] | Methyl 3-chloro-5- fluoro-6-(4-(2,2,2- trifluoroethoxy) phenyl) picolinate | Example 63 | White Solid | 261 mg, 52% |
| F164 | 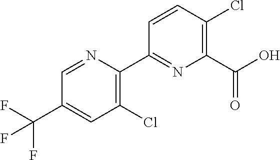 [SMILES: O=C(O)c1nc(-c2ncc(C(F)(F)F)cc2Cl)ccc1Cl] | 3′,5-Dichloro-5′- (trifluoromethyl)- [2,2′-bipyridine]-6- carboxylic acid | Example 27 | Orange Oil | 110 mg, 97% |
| F165 | 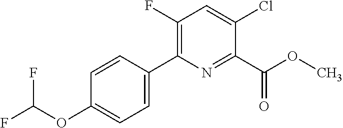 [SMILES: COC(=O)c1nc(-c2ccc(OC(F)F)cc2)c(F)cc1Cl] | Methyl 3-chloro-6- (4- (difluoromethoxy) phenyl)-5- fluoropicolinate | Example 63 | White Solid | 225 mg, 49% |
| F166 | 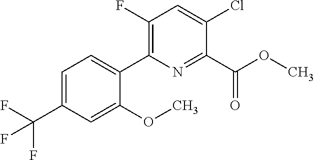 [SMILES: COC(=O)c1nc(-c2ccc(C(F)(F)F)cc2OC)c(F)cc1Cl] | Methyl 3-chloro-5- fluoro-6-(2-methoxy- 4- (trifluoromethyl) phenyl)picolinate | Example 63 | White Solid | 479 mg, 30% |
| F167 | 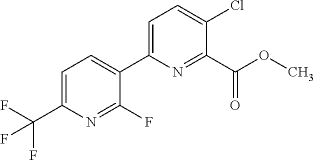 [SMILES: COC(=O)c1nc(-c2ccc(C(F)(F)F)nc2F)ccc1Cl] | Methyl 5-chloro-2′- fluoro-6′- (trifluoromethyl)- [2,3′-bipyridine]-6- carboxylate | Example 63 | White Solid | 77 mg, 29% |
| F168 | 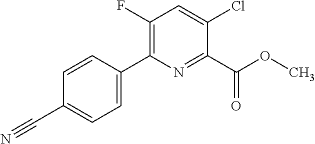 [SMILES: COC(=O)c1nc(-c2ccc(C#N)cc2)c(F)cc1Cl] | Methyl 3-chloro-6- (4-cyanophenyl)-5- fluoropicolinate | Example 63 | White Solid | 196 mg, 49% |
| F169 | 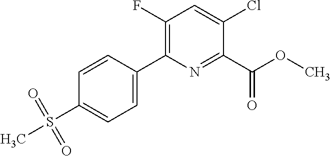 | Methyl 3-chloro-5- fluoro-6-(4- (methylsulfonyl) phenyl)picolinate | Example 63 | Off-White Solid | 193 mg, 38% |
| F170 | 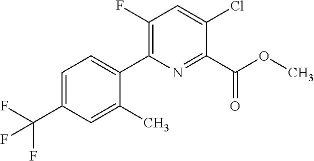 [SMILES: COC(=O)c1nc(-c2ccc(C(F)(F)F)cc2C)c(F)cc1Cl] | Methyl 3-chloro-5- fluoro-6-(2-methyl-4- (trifluoromethyl) phenyl)picolinate | Example 63 | White Solid | 223 mg, 47% |
| F171 | 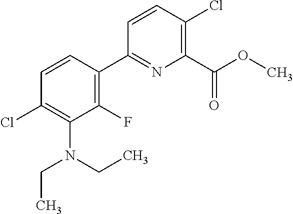 [SMILES: CCN(CC)c1c(Cl)ccc(-c2ccc(Cl)c(C(=O)OC)n2)c1F] | Methyl 4-amino-3- chloro-6-(4-chloro-3- (diethylamino)-2- fluorophenyl) picolinate | Example 19 using tert-butylnitrite and DMF as solvent, as described in J. Org. Chem. 1977, 42, 3494 | Colorless Oil | 16 mg, 22% |
| F172 | 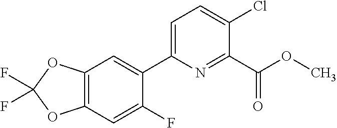 [SMILES: COC(=O)c1nc(-c2cc3c(cc2F)OC(F)(F)O3)ccc1Cl] | Methyl 3-chloro-6- (2,2,6- trifluorobenzo[d] [1,3]dioxol- 5-yl)picolinate | Example 51 | White Solid | 293 mg, 51% |
| F173 | 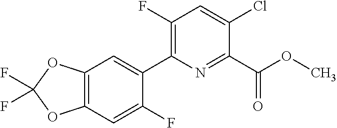 [SMILES: COC(=O)c1nc(-c2cc3c(cc2F)OC(F)(F)O3)c(F)cc1Cl] | Methyl 3-chloro-5- fluoro-6-(2,2,6- trifluorobenzo[d] [1,3]dioxol- 5-yl)picolinate | Example 51 | White Solid | 231 mg, 38% |
| F174 | 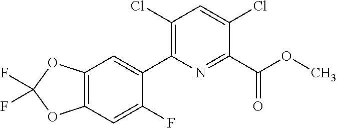 [SMILES: COC(=O)c1nc(-c2cc3c(cc2F)OC(F)(F)O3)c(Cl)cc1Cl] | Methyl 3,5-dichloro- 6-(2,2,6- trifluorobenzo[d] [1,3]dioxol-5- yl)picolinate | Example 51 | White Solid | 341 mg, 54% |
| F175 | 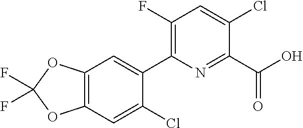 [SMILES: O=C(O)c1nc(-c2cc3c(cc2Cl)OC(F)(F)O3)c(F)cc1Cl] | 3-Chloro-6-(6-chloro- 2,2- difluorobenzo[d] [1,3]dioxol-5- yl)-5- fluoropicolinic acid | Example 27 | Yellow Oil | 27 mg, 92% |
| F176 | 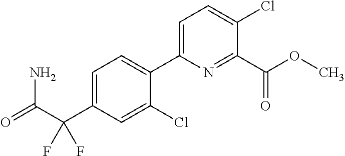 [SMILES: COC(=O)c1nc(-c2ccc(C(F)(F)C(N)=O)cc2Cl)ccc1Cl] | Methyl 6-(4-(2- amino-1,1-difluoro-2- oxoethyl)-2- chlorophenyl)-3- chloropicolinate | Example 65 | White Solid | 0.1 g, 25% |
| F177 | 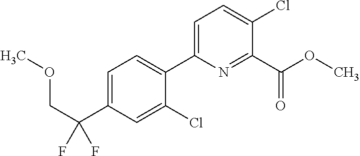 | Methyl 3-chloro-6- (2-chloro-4-(1,1- difluoro-2- methoxyethyl) phenyl)picolinate | Example 65 | Pale Yellow Liquid | 0.15 g, 30% |
| F178 | 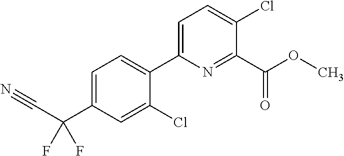 [SMILES: COC(=O)c1nc(-c2ccc(C(F)(F)C#N)cc2Cl)ccc1Cl] | Methyl 3-chloro-6- (2-chloro-4- (cyanodifluoromethyl) phenyl)picolinate | Example 65 | Off-White Solid | 0.6 g, 20% |
| F179 | 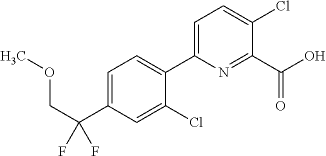 [SMILES: COCC(F)(F)c1ccc(-c2ccc(Cl)c(C(=O)O)n2)c(Cl)c1] | 3-Chloro-6-(2-chloro- 4-(1,1-difluoro-2- methoxyethyl)phenyl) picolinic acid | Example 47 | Brown Liquid | 0.04 g, 70% |
| F180 | 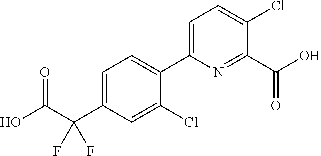 [SMILES: O=C(O)c1nc(-c2ccc(C(F)(F)C(=O)O)cc2Cl)ccc1Cl] | 6-(4- (Carboxy- difluoromethyl)- 2-chlorophenyl)- 3-chloropicolinic acid | Example 47 | White Solid | 0.025 g, 20% |
| F181 | 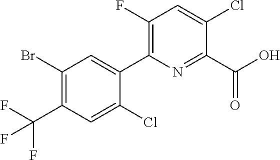 [SMILES: O=C(O)c1nc(-c2cc(Br)c(C(F)(F)F)cc2Cl)c(F)cc1Cl] | 6-(5-Bromo-2-chloro- 4- (trifluoromethyl) phenyl)-3-chloro-5- fluoropicolinic acid | Example 27 | White Solid | 31 mg, 52% |
| F182 | 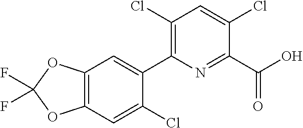 [SMILES: O=C(O)c1nc(-c2cc3c(cc2Cl)OC(F)(F)O3)c(Cl)cc1Cl] | 3,5-Dichloro-6-(6- chloro-2,2- difluorobenzo[d][1,3] dioxol-5-yl)picolinic acid | Example 27 | Clear Oil | 48 mg, 98% |
| F183 | 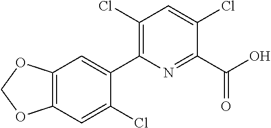 [SMILES: O=C(O)c1nc(-c2cc3c(cc2Cl)OCO3)c(Cl)cc1Cl] | 3,5-Dichloro-6-(6- chlorobenzo[d][1,3] dioxol-5-yl)picolinic acid | Example 27 | Yellow Solid | 84 mg, 68% |
| F184 | 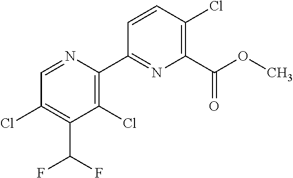 [SMILES: COC(=O)c1nc(-c2ncc(Cl)c(C(F)F)c2Cl)ccc1Cl] | Methyl 3,5,5′- trichloro-4′- (difluoromethyl)- [2,2′-bipyridine]-6- carboxylate | Example 19 using tert-butylnitrite and DMF as solvent, as described in J. Org. Chem. 1977, 42, 3494 | Colorless Oil | 82 mg, 92% |
| F185 | 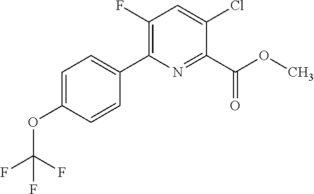 [SMILES: COC(=O)c1nc(-c2ccc(OC(F)(F)F)cc2)c(F)cc1Cl] | Methyl 3-chloro-5- fluoro-6-(4- (trifluoromethoxy) phenyl)picolinate | Example 63 | Low Melting, Glass Solid | 125 mg, 26% |
| F186 | 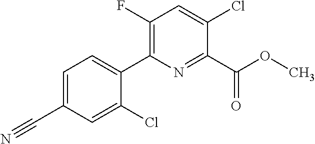 [SMILES: COC(=O)c1nc(-c2ccc(C#N)cc2Cl)c(F)cc1Cl] | Methyl 3-chloro-6- (2-chloro-4- cyanophenyl)-5- fluoropicolinate | Example 63 | White Solid | 57 mg, 9.3% |
| F187 | 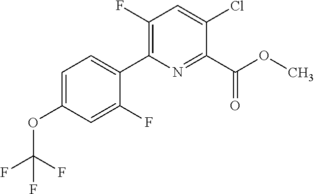 [SMILES: COC(=O)c1nc(-c2ccc(OC(F)(F)F)cc2F)c(F)cc1Cl] | Methyl 3-chloro-5- fluoro-6-(2-fluoro-4- (trifluoromethoxy) phenyl)picolinate | Example 63 | White Solid | 162 mg, 24% |
| F188 | 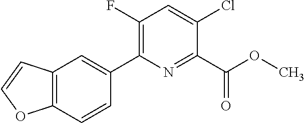 [SMILES: COC(=O)c1nc(-c2ccc3occc3c2)c(F)cc1Cl] | Methyl 6- (benzofuran-5-yl)-3- chloro-5- fluoropicolinate | Example 63 | White Solid | 380 mg, 68% |
| F189 | 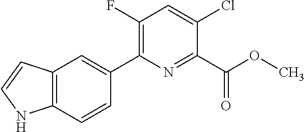 [SMILES: COC(=O)c1nc(-c2ccc3[nH]ccc3c2)c(F)cc1Cl] | Methyl 3-chloro-5- fluoro-6-(1H-indol-5- yl)picolinate | Example 63 | White Solid | 250 mg, 45% |
| F190 | 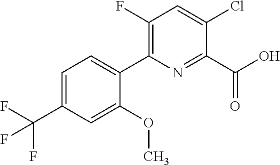 [SMILES: COc1cc(C(F)(F)F)ccc1-c1nc(C(=O)O)c(Cl)cc1F] | 3-Chloro-5-fluoro-6- (2-methoxy-4- (trifluoromethyl) phenyl) picolinic acid | Example 50 | White Solid | 150 mg, 83% |
| F191 | 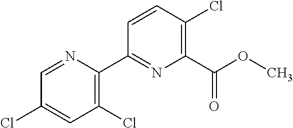 [SMILES: COC(=O)c1nc(-c2ncc(Cl)cc2Cl)ccc1Cl] | Methyl 3′,5,5′- trichloro-[2,2′- bipyridine]-6- carboxylate | Example 19 using tert-butylnitrite and DMF as solvent, as described in J. Org. Chem. 1977, 42, 3494 | Orange Oil | 94 mg, 100% |
| F192 | 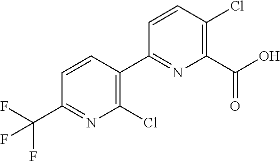 [SMILES: O=C(O)c1nc(-c2ccc(C(F)(F)F)nc2Cl)ccc1Cl] | 2′,5-Dichloro-6′- (trifluoromethyl)- [2,3′-bipyridine]-6- carboxylic acid | Example 28 | Tan Solid | 59 mg, 79% |
| F193 | 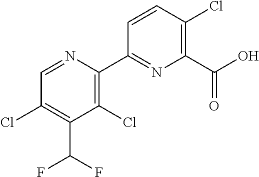 [SMILES: O=C(O)c1nc(-c2ncc(Cl)c(C(F)F)c2Cl)ccc1Cl] | 3′,5,5′-Trichloro-4′- (difluoromethyl)- [2,2′-bipyridine]-6- carboxylic acid | Example 27 | Orange Oil | 30 mg, 74% |
| F194 | 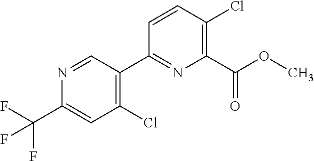 [SMILES: COC(=O)c1nc(-c2cnc(C(F)(F)F)cc2Cl)ccc1Cl] | Methyl 4′,5-dichloro- 6′-(trifluoromethyl)- [2,3′-bipyridine]-6- carboxylate | Example 19 using tert-butylnitrite and DMF as solvent, as described in J. Org. Chem. 1977, 42, 3494 | Off-White Solid | 138 mg, 91% |
| F195 | 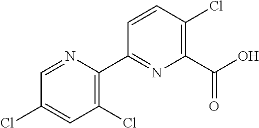 [SMILES: O=C(O)c1nc(-c2ncc(Cl)cc2Cl)ccc1Cl] | 3′,5,5′-Trichloro- [2,2′-bipyridine]-6- carboxylic acid | Example 27 | Yellow Oil | 44 mg, 96% |
| F196 | 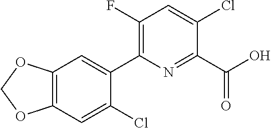 [SMILES: O=C(O)c1nc(-c2cc3c(cc2Cl)OCO3)c(F)cc1Cl] | 3-Chloro-6-(6- chlorobenzo[d][1,3] dioxol-5-yl)-5- fluoropicolinic acid | Example 27 | White Solid | 48 mg, 98% |
| F197 | 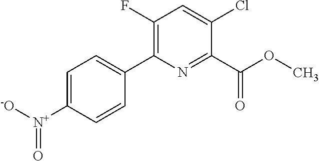 | Methyl 3-chloro-5- fluoro-6-(4- nitrophenyl) picolinate | Example 63 | White Solid | 290 mg, 50% |
| F198 | 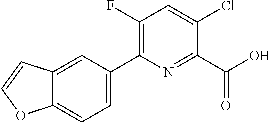 [SMILES: O=C(O)c1nc(-c2ccc3occc3c2)c(F)cc1Cl] | 6-(Benzofuran-5-yl)- 3-chloro-5- fluoropicolinic acid | Example 50 | White Solid | 130 mg, 67% |
| F199 | 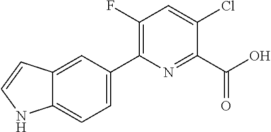 [SMILES: O=C(O)c1nc(-c2ccc3[nH]ccc3c2)c(F)cc1Cl] | 3-Chloro-5-fluoro-6- (1H-indol-5- yl)picolinic acid | Example 50 | Off-White Solid | 113 mg, 77% |
| F200 | 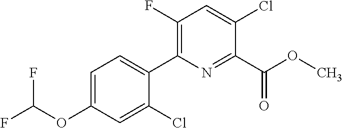 [SMILES: COC(=O)c1nc(-c2ccc(OC(F)F)cc2Cl)c(F)cc1Cl] | Methyl 3-chloro-6- (2-chloro-4- (difluoromethoxy) phenyl)-5- fluoropicolinate | Example 68 using C93 | Yellow Solid | 250 mg, 33% |
| F201 | 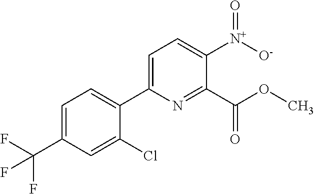 [SMILES: COC(=O)c1nc(-c2ccc(C(F)(F)F)cc2Cl)ccc1[N+](=O)[O-]] | Methyl 6-(2-chloro-4- (trifluoromethyl) phenyl)-3- nitropicolinate | Example 51 | White Solid | 239 mg, 75% |
| F202 | 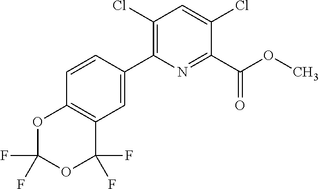 [SMILES: COC(=O)c1nc(-c2ccc3c(c2)C(F)(F)OC(F)(F)O3)c(Cl)cc1Cl] | Methyl 3,5-dichloro- 6-(2,2,4,4-tetrafluoro- 4H- benzo[d][1,3]dioxin- 6-yl)picolinate | Example 51 | White Solid | 61 mg, 25% |
| F203 | 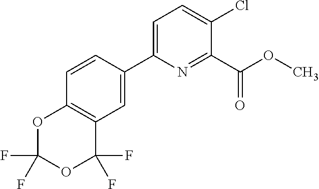 [SMILES: COC(=O)c1nc(-c2ccc3c(c2)C(F)(F)OC(F)(F)O3)ccc1Cl] | Methyl 3-chloro-6- (2,2,4,4-tetrafluoro- 4H- benzo[d][1,3] dioxin-6-yl) picolinate | Example 51 | White Solid | 198 mg, 56% |
| F204 | 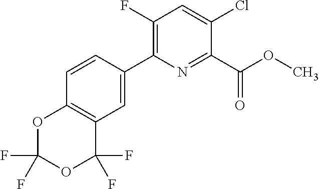 [SMILES: COC(=O)c1nc(-c2ccc3c(c2)C(F)(F)OC(F)(F)O3)c(F)cc1Cl] | Methyl 3-chloro-5- fluoro-6-(2,2,4,4- tetrafluoro-4H- benzo[d][1,3]dioxin- 6-yl)picolinate | Example 51 | Brown Oil | 199 mg, 55% |
| F205 | 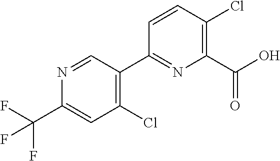 [SMILES: O=C(O)c1nc(-c2cnc(C(F)(F)F)cc2Cl)ccc1Cl] | 4′,5-Dichloro-6′- (trifluoromethyl)- [2,3′-bipyridine]-6- carboxylic acid | Example 28 | White Solid | 65 mg, 88% |
| F206 | 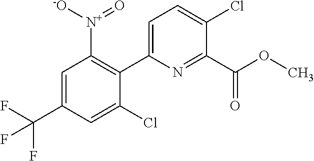 | Methyl 3-chloro-6- (2-chloro-6-nitro-4- (trifluoromethyl) phenyl)picolinate | Example 41 | Yellow Oil | 334 mg, 20% |
| F207 | 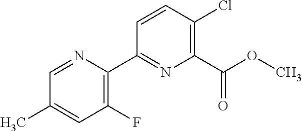 | Methyl 5-chloro-3′- fluoro-5′-methyl- [2,2′-bipyridine]-6- carboxylate | Example 19 using tert-butylnitrite and DMF as solvent, as described in J. Org. Chem. 1977, 42, 3494 | Colorless Oil | 25 mg, 68% |
| F208 | 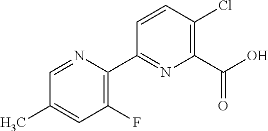 [SMILES: Cc1cnc(-c2ccc(Cl)c(C(=O)O)n2)c(F)c1] | 5-Chloro-3′-fluoro-5′- methyl-[2,2′- bipyridine]-6- carboxylic acid | Example 28 | Light Orange Solid | 19 mg, 80% |
| F209 | 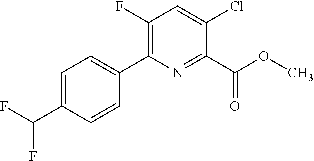 [SMILES: COC(=O)c1nc(-c2ccc(C(F)F)cc2)c(F)cc1Cl] | Methyl 3-chloro-6-(4- (difluoromethyl) phenyl)- 5-fluoropicolinate | Example 63 | White Solid | 208 mg, 48% |
| F210 | 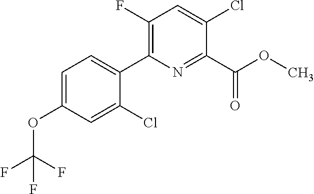 [SMILES: COC(=O)c1nc(-c2ccc(OC(F)(F)F)cc2Cl)c(F)cc1Cl] | Methyl 3-chloro-6- (2-chloro-4- (trifluoromethoxy) phenyl)-5- fluoropicolinate | Example 63 | White Solid | 158 mg, 23% |
| F211 | 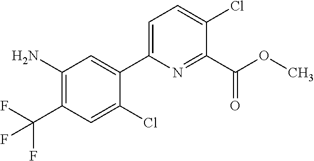 [SMILES: COC(=O)c1nc(-c2cc(N)c(C(F)(F)F)cc2Cl)ccc1Cl] | Methyl 6-(5-amino-2- chloro-4- (trifluoromethyl) phenyl)- yl)-3- chloropicolinate | Example 54 | Beige Solid | 102 mg, 28% |
| F212 | 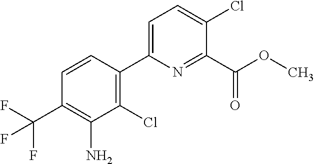 [SMILES: COC(=O)c1nc(-c2ccc(C(F)(F)F)c(N)c2Cl)ccc1Cl] | Methyl 6-(3-amino-2- chloro-4- (trifluoromethyl) phenyl)- 3-chloropicolinate | Example 54 | White Solid | 49 mg, 13% |
| F213 | 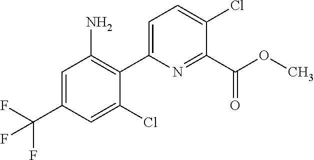 [SMILES: COC(=O)c1nc(-c2c(N)cc(C(F)(F)F)cc2Cl)ccc1Cl] | Methyl 6-(2-amino-6- chloro-4- (trifluoromethyl) phenyl)- 3-chloropicolinate | Example 55 | White Solid | 108 mg, 38% |
| F214 | 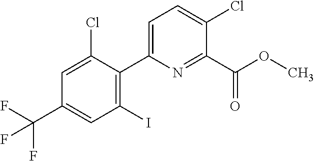 [SMILES: COC(=O)c1nc(-c2c(Cl)cc(C(F)(F)F)cc2I)ccc1Cl] | Methyl 3-chloro-6- (2-chloro-6-iodo-4- (trifluoromethyl) phenyl)picolinate | Example 66 | Yellow Solid | 92 mg, 74% |
| F215 | 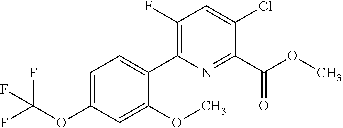 [SMILES: COC(=O)c1nc(-c2ccc(OC(F)(F)F)cc2OC)c(F)cc1Cl] | Methyl 3-chloro-5- fluoro-6-(2-methoxy- 4- (trifluoromethoxy) phenyl)picolinate | Example 63 | White Solid | 397 mg, 57% |
| F216 | 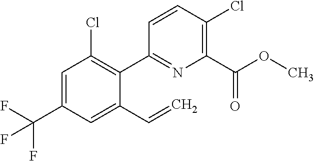 [SMILES: C=Cc1cc(C(F)(F)F)cc(Cl)c1-c1ccc(Cl)c(C(=O)OC)n1] | Methyl 3-chloro-6- (2-chloro-4- (trifluoromethyl)-6- vinylphenyl) picolinate | Example 56 | White Solid | 46 mg, 55% |
| F217 | 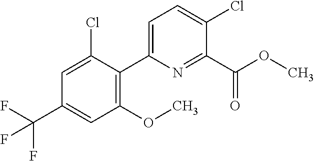 [SMILES: COC(=O)c1nc(-c2c(Cl)cc(C(F)(F)F)cc2OC)ccc1Cl] | Methyl 3-chloro-6- (2-chloro-6-methoxy- 4- (trifluoromethyl) phenyl)picolinate | Example 41 | White Solid | 35 mg, 13% |
| F218 | 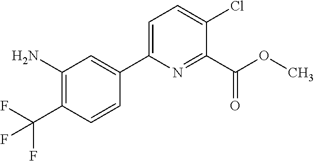 [SMILES: COC(=O)c1nc(-c2ccc(C(F)(F)F)c(N)c2)ccc1Cl] | Methyl 6-(3-amino-4- (trifluoromethyl) phenyl)- 3-chloropicolinate | Example 32 | Viscous Clear Oil | 1.13 g, 70% |
| F219 | 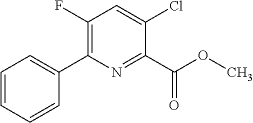 [SMILES: COC(=O)c1nc(-c2ccccc2)c(F)cc1Cl] | Methyl 3-chloro-5- fluoro-6- phenylpicolinate | Example 63 | Clear Oil | 208 mg, 57% |
| F220 | 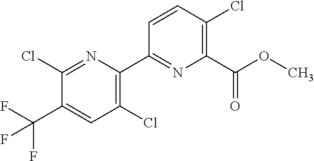 [SMILES: COC(=O)c1nc(-c2nc(Cl)c(C(F)(F)F)cc2Cl)ccc1Cl] | Methyl 3′,5,6′- trichloro-5′- (trifluoromethyl)- [2,2′-bipyridine]-6- carboxylate | Example 19 using tert-butylnitrite and DMF as solvent, as described in J. Org. Chem. 1977, 42, 3494 | Colorless Oil | 29 mg, 100% |
| F221 | 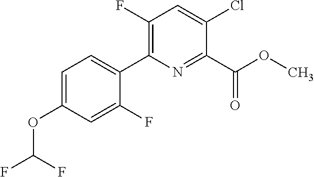 [SMILES: COC(=O)c1nc(-c2ccc(OC(F)F)cc2F)c(F)cc1Cl] | Methyl 3-chloro-6- (4- (difluoromethoxy)-2- fluorophenyl)-5- fluoropicolinate | Example 63 | White Solid | 91 mg, 14% |
| F222 | 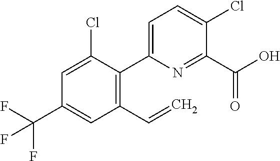 [SMILES: C=Cc1cc(C(F)(F)F)cc(Cl)c1-c1ccc(Cl)c(C(=O)O)n1] | 3-Chloro-6-(2-chloro- 4-(trifluoromethyl)-6- vinylphenyl) picolinic acid | Example 27 | Yellow Oil | 36 mg, 100% |
| F223 | 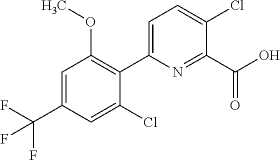 [SMILES: COc1cc(C(F)(F)F)cc(Cl)c1-c1ccc(Cl)c(C(=O)O)n1] | 3-Chloro-6-(2-chloro- 6-methoxy-4- (trifluoromethyl) phenyl) picolinic acid | Example 27 | Yellow Oil | 33 mg, 99% |
| F224 | 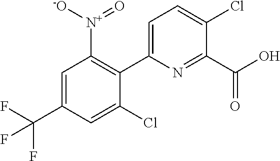 | 3-Chloro-6-(2-chloro- 6-nitro-4- (trifluoromethyl) phenyl)picolinic acid | Example 27 | White Solid | 39 mg, 100% |
| F225 | 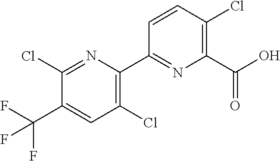 [SMILES: O=C(O)c1nc(-c2nc(Cl)c(C(F)(F)F)cc2Cl)ccc1Cl] | 3′,5,6′-Trichloro-5'- (trifluoromethyl)- [2,2′-bipyridine]-6- carboxylic acid | Example 27 | Yellow Foam | 23 mg, 80% |
| F226 | 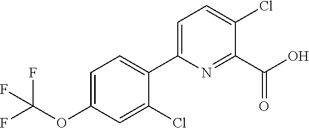 [SMILES: O=C(O)c1nc(-c2ccc(OC(F)(F)F)cc2Cl)ccc1Cl] | 3-Chloro-6-(2- chloro-4- 4- (trifluoromethoxy) phenyl)picolinic acid | Example 50 | White Solid | 104 mg, 71% |
| F227 | 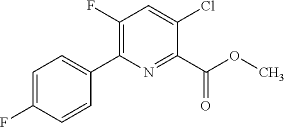 [SMILES: COC(=O)c1nc(-c2ccc(F)cc2)c(F)cc1Cl] | Methyl 3-chloro-5- fluoro-6-(4- fluorophenyl) picolinate | Example 63 | Clear Oil | 226 mg, 58% |
| F228 | 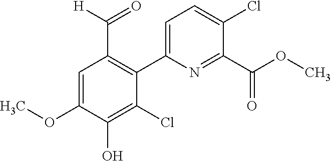 | Methyl 3-chloro-6- (2-chloro-6-formyl-3- hydroxy-4- methoxyphenyl) picolinate | Example 41 | Brown Solid | 39 mg, 14% |
| F229 | 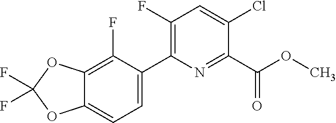 [SMILES: COC(=O)c1nc(-c2ccc3c(c2F)OC(F)(F)O3)c(F)cc1Cl] | Methyl 3-chloro-5- fluoro-6-(2,2,4- trifluorobenzo[d][1,3] dioxol-5-yl)picolinate | Example 51 | Brown Solid | 345 mg, 64% |
| F230 | 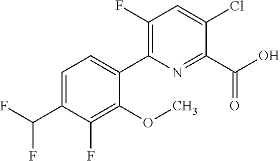 [SMILES: COc1c(-c2nc(C(=O)O)c(Cl)cc2F)ccc(C(F)F)c1F] | 3-Chloro-6-(4- (difluoromethyl)-3- fluoro-2- methoxyphenyl)-5- fluoropicolinic acid | Example 50 | White Solid | 100 mg, 77% |
| F231 | 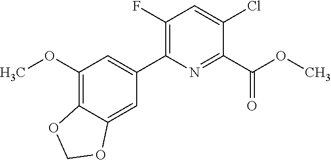 | Methyl 3-chloro-5- fluoro-6-(7- methoxybenzo[d] [1,3]dioxol-5- yl)picolinate | Example 19 using starting materials described in US 20140274701 and tert-butylnitrite and DMF as solvent, as described in J. Org. Chem. 1977, 42, 3494 | Brown Solid | 85 mg, 97% |
| F232 | 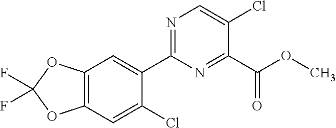 [SMILES: COC(=O)c1nc(-c2cc3c(cc2Cl)OC(F)(F)O3)ncc1Cl] | Methyl 5-chloro-2- (6-chloro-2,2- difluorobenzo[d][1,3] dioxol-5- yl)pyrimidine-4- carboxylate | Example 51 | Yellow Solid | 155 mg, 46% |
| F233 | 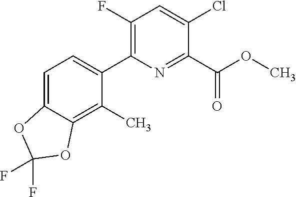 [SMILES: COC(=O)c1nc(-c2ccc3c(c2C)OC(F)(F)O3)c(F)cc1Cl] | Methyl 3-chloro-6- (2,2-difluoro-4- methylbenzo[d][1,3] dioxol-5-yl)-5- fluoropicolinate | Example 51 | White Solid | 51 mg, 58% |
| F234 | 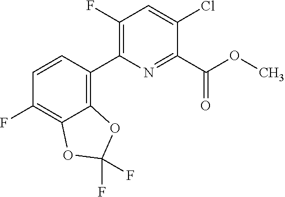 [SMILES: COC(=O)c1nc(-c2ccc(F)c3c2OC(F)(F)O3)c(F)cc1Cl] | Methyl 3-chloro-5- fluoro-6-(2,2,7- trifluorobenzo[d][1,3] dioxol-4-yl)picolinate | Example 51 | White Solid | 235 mg, 65% |
| F235 | 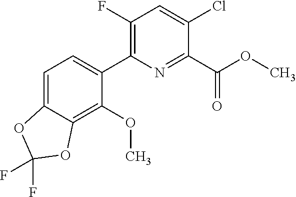 [SMILES: COC(=O)c1nc(-c2ccc3c(c2OC)OC(F)(F)O3)c(F)cc1Cl] | Methyl 3-chloro-6- (2,2-difluoro-4- methoxybenzo[d][1,3] dioxol-5-yl)-5- fluoropicolinate | Example 41 | White Solid | 148 mg, 51% |
| F236 | 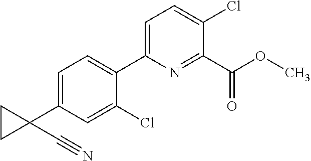 [SMILES: COC(=O)c1nc(-c2ccc(C3(C#N)CC3)cc2Cl)ccc1Cl] | Methyl 3-chloro-6- (2-chloro-4-(1- cyanocyclopropyl) phenyl)picolinate | Example 51 | Yellow Oil | 212 mg, 93% |
| F237 | 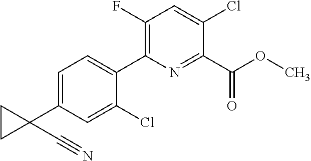 [SMILES: COC(=O)c1nc(-c2ccc(C3(C#N)CC3)cc2Cl)c(F)cc1Cl] | Methyl 3-chloro-6- (2-chloro-4-(1- cyanocyclopropyl) phenyl)-5- fluoropicolinate | Example 51 | White Solid | 218 mg, 91% |
| F238 | 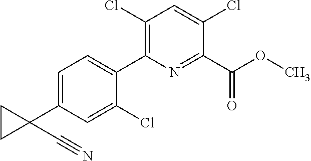 [SMILES: COC(=O)c1nc(-c2ccc(C3(C#N)CC3)cc2Cl)c(Cl)cc1Cl] | Methyl 3,5-dichloro- 6-(2-chloro-4-(1- cyanocyclopropyl) phenyl)picolinate | Example 51 | Yellow Oil | 221 mg, 88% |
| F239 | 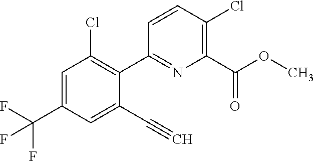 [SMILES: C#Cc1cc(C(F)(F)F)cc(Cl)c1-c1ccc(Cl)c(C(=O)OC)n1] | Methyl 3-chloro-6- (2-chloro-6-ethynyl- 4- (trifluoromethyl) phenyl)picolinate | Example 67 | Brown Oil | 35 mg, 50% |
| F240 | 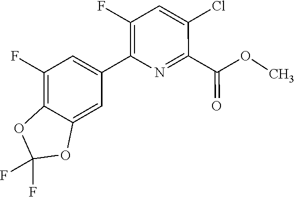 [SMILES: COC(=O)c1nc(-c2cc(F)c3c(c2)OC(F)(F)O3)c(F)cc1Cl] | Methyl 3-chloro-5- fluoro-6-(2,2,7- trifluorobenzo[d][1,3] dioxol-5-yl)picolinate | Example 51 | White Solid | 125 mg, 24% |
| F241 | 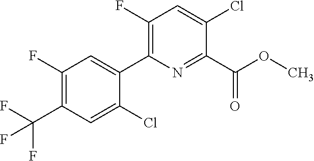 [SMILES: COC(=O)c1nc(-c2cc(F)c(C(F)(F)F)cc2Cl)c(F)cc1Cl] | Methyl 3-chloro-6- (2-chloro-5-fluoro-4- (trifluoromethyl) phenyl)- 5-fluoropicolinate | Example 51 | White Solid | 84 mg, 21% |
| F242 | 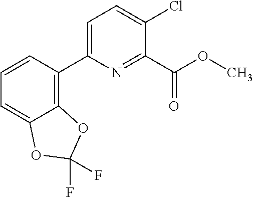 [SMILES: COC(=O)c1nc(-c2cccc3c2OC(F)(F)O3)ccc1Cl] | Methyl 3-chloro-6- (2,2- difluorobenzo[d][1,3] dioxol-4-yl)picolinate | Example 51 | White Solid | 202 mg, 89% |
| F243 | 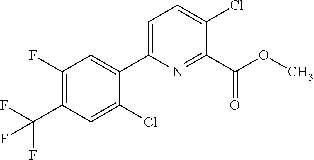 [SMILES: COC(=O)c1nc(-c2cc(F)c(C(F)(F)F)cc2Cl)ccc1Cl] | Methyl 3-chloro-6- (2-chloro-5-fluoro-4- (trifluoromethyl) phenyl)picolinate | Example 51 | Brown Solid | 34 mg, 9% |
| F244 | 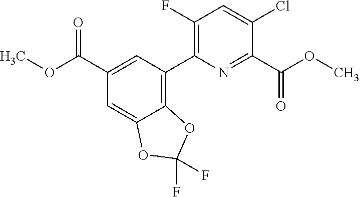 | Methyl 3-chloro-6- (2,2-difluoro-6- (methoxycarbonyl) benzo[d][1,3]dioxol-4- yl)-5-fluoropicolinate | Example 51 | White Solid | 67 mg, 26% |
| F245 | 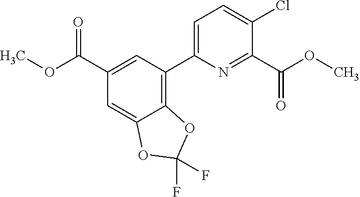 | Methyl 3-chloro-6- (2,2-difluoro-6- (methoxycarbonyl) benzo[d][1,3]dioxol- 4-yl)picolinate | Example 51 | White Solid | 234 mg, 88% |
| F246 | 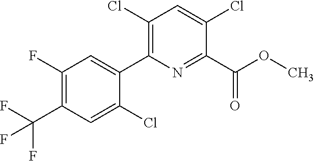 [SMILES: COC(=O)c1nc(-c2cc(F)c(C(F)(F)F)cc2Cl)c(Cl)cc1Cl] | Methyl 3,5-dichloro- 6-(2-chloro-5-fluoro- 4- (trifluoromethyl) phenyl)picolinate | Example 51 | Brown Solid | 69 mg, 17% |
| F247 | 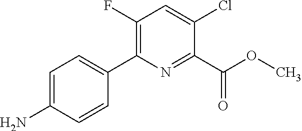 [SMILES: COC(=O)c1nc(-c2ccc(N)cc2)c(F)cc1Cl] | Methyl 6-(4- aminophenyl)-3- chloro-5- fluoropicolinate | Example 63 | Yellow Solid | 376 mg, 49% |
| F248 | 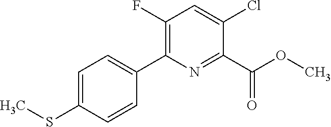 | Methyl 3-chloro-5- fluoro-6-(4- (methylthio)phenyl) picolinate | Example 63 | White Solid | 297 mg, 69% |
| F249 | 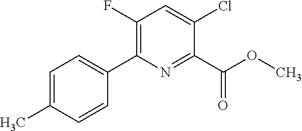 | Methyl 3-chloro-5- fluoro-6-(p- tolyl)picolinate | Example 63 | White Solid | 226 mg, 58% |
| F250 | 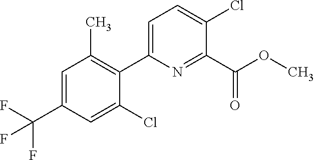 [SMILES: COC(=O)c1nc(-c2c(C)cc(C(F)(F)F)cc2Cl)ccc1Cl] | Methyl 3-chloro-6- (2-chloro-6-methyl-4- (trifluoromethyl) phenyl) picolinate | Example 41 | Yellow Oil | 195 mg, 27% |
| F251 | 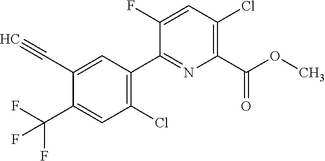 [SMILES: C#Cc1cc(-c2nc(C(=O)OC)c(Cl)cc2F)c(Cl)cc1C(F)(F)F] | Methyl 3-chloro-6- (2-chloro-5-ethynyl- 4- (trifluoromethyl) phenyl)-5-yl) picolinate | Example 6 | Brown Oil | 57 mg, 16% |
| F252 | 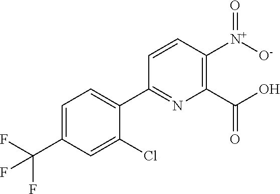 [SMILES: O=C(O)c1nc(-c2ccc(C(F)(F)F)cc2Cl)ccc1[N+](=O)[O-]] | 6-(2-Chloro-4- (trifluoromethyl) phenyl)- 3-nitropicolinic acid | Example 27 | White Solid | 50 mg, 22% |
| F253 | 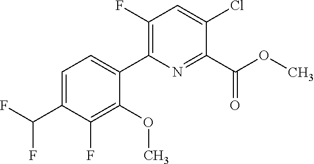 [SMILES: COC(=O)c1nc(-c2ccc(C(F)F)c(F)c2OC)c(F)cc1Cl] | Methyl 3-chloro-6- (4-(difluoromethyl)- 3-fluoro-2- methoxyphenyl)-5- fluoropicolinate | Example 64 using F357 | White Solid | 271 mg, 93% |
| F254 | 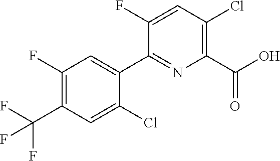 [SMILES: O=C(O)c1nc(-c2cc(F)c(C(F)(F)F)cc2Cl)c(F)cc1Cl] | 3-Chloro-6-(2-chloro- 5-fluoro-4- (trifluoromethyl) phenyl)-5- fluoropicolinic acid | Example 27 | White Solid | 48 mg, 59% |
| F255 | 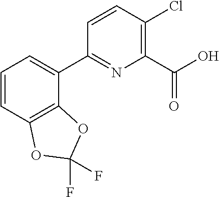 [SMILES: O=C(O)c1nc(-c2cccc3c2OC(F)(F)O3)ccc1Cl] | 3-Chloro-6-(2,2- difluorobenzo[d][1,3] dioxol-4-yl)picolinic acid | Example 27 | White Solid | 127 mg, 66% |
| F256 | 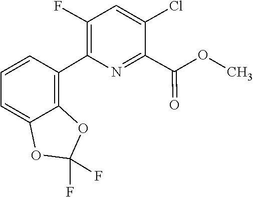 [SMILES: COC(=O)c1nc(-c2cccc3c2OC(F)(F)O3)c(F)cc1Cl] | Methyl 3-chloro-6- (2,2- difluorobenzo[d][1,3] dioxol-4-yl)-5- fluoropicolinate | Example 51 | White Solid | 137 mg, 62% |
| F257 | 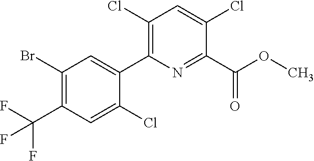 [SMILES: COC(=O)c1nc(-c2cc(Br)c(C(F)(F)F)cc2Cl)c(Cl)cc1Cl] | Methyl 6-(5-bromo- 2-chloro-4- (trifluoromethyl) phenyl)-3,5- dichloropicolinate | Example 51 | White Solid | 69 mg, 25% |
| F258 | 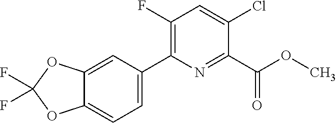 [SMILES: COC(=O)c1nc(-c2ccc3c(c2)OC(F)(F)O3)c(F)cc1Cl] | Methyl 3-chloro-6- (2,2- difluorobenzo[d][1,3] dioxol-5-yl)-5- fluoropicolinate | Example 51 | White Solid | 95 mg, 43% |
| F259 | 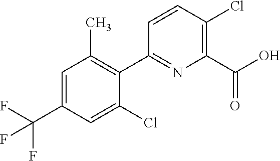 [SMILES: Cc1cc(C(F)(F)F)cc(Cl)c1-c1ccc(Cl)c(C(=O)O)n1] | 3-Chloro-6-(2-chloro- 6-methyl-4- (trifluoromethyl) phenyl)picolinic acid | Example 27 | Clear Oil | 125 mg, 93% |
| F260 | 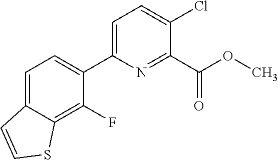 [SMILES: COC(=O)c1nc(-c2ccc3ccsc3c2F)ccc1Cl] | Methyl 3-chloro-6- (7- fluorobenzo[b] thiophen-6-yl) picolinate | Example 51 | White Solid | 95 mg, 71% |
| F261 | 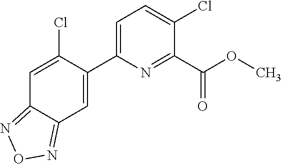 [SMILES: COC(=O)c1nc(-c2cc3nonc3cc2Cl)ccc1Cl] | Methyl 3-chloro-6- (6- chlorobenzo[c][1,2,5] oxadiazol-5- yl)picolinate | Example 41 using starting materials described in US 20140274702 | White Solid | 27 mg, 15% |
| F262 | 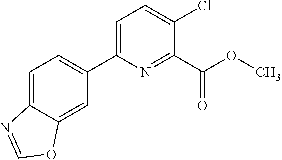 [SMILES: COC(=O)c1nc(-c2ccc3ncoc3c2)ccc1Cl] | Methyl 6- (benzo[d]oxazol-6- yl)-3-chloropicolinate | Example 51 | White Solid | 29 mg, 24% |
| F263 | 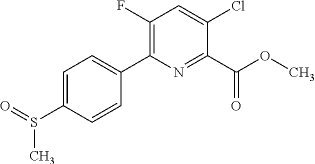 [SMILES: COC(=O)c1nc(-c2ccc(S(C)=O)cc2)c(F)cc1Cl] | Methyl 3-chloro-5- fluoro-6-(4- (methylsulfinyl) phenyl)picolinate | Example 63 | Viscous Yellow Oil | 251 mg, 55% |
| F264 | 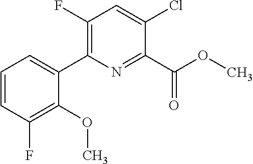 [SMILES: COC(=O)c1nc(-c2cccc(F)c2OC)c(F)cc1Cl] | Methyl 3-chloro-5- fluoro-6-(3-fluoro-2- methoxyphenyl) picolinate | Example 63 | White Solid | 295 mg, 49% |
| F265 | 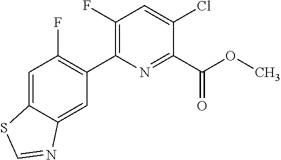 [SMILES: COC(=O)c1nc(-c2cc3ncsc3cc2F)c(F)cc1Cl] | Methyl 3-chloro-5- fluoro-6-(6- fluorobenzo[d]thiazol- 5-yl)picolinate | Example 51 | White Solid | 79 mg, 33% |
| F266 | 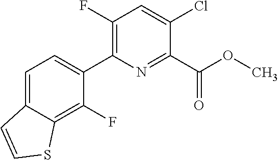 [SMILES: COC(=O)c1nc(-c2ccc3ccsc3c2F)c(F)cc1Cl] | Methyl 3-chloro-5- fluoro-6-(7- fluorobenzo[b] thiophen- 6-yl)picolinate | Example 51 | White Solid | 35 mg, 19% |
| F267 | 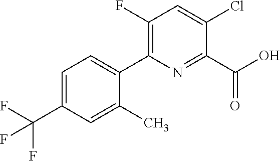 [SMILES: Cc1cc(C(F)(F)F)ccc1-c1nc(C(=O)O)c(Cl)cc1F] | 3-Chloro-5-fluoro-6- (2-methyl-4- (trifluoromethyl) phenyl) picolinic acid | Example 50 | Off-White Solid | 78 mg, 34% |
| F268 | 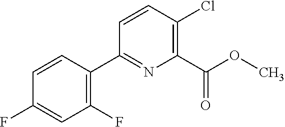 [SMILES: COC(=O)c1nc(-c2ccc(F)cc2F)ccc1Cl] | Methyl 3-chloro-6- (2,4- difluorophenyl) picolinate | Example 19 using starting materials described in WO 2003011853 and tert-butylnitrite and DMF as solvent, as described in J. | White Solid | 34 mg, 46% |
| | | | Org. Chem. 1977, | | |
| | | | 42, 3494 | | |
| F269 | 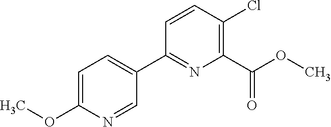 | Methyl 5-chloro-6′- methoxy-[2,3′- bipyridine]-6- carboxylate | Example 41 | White Solid | 41 mg, 55% |
| F270 | 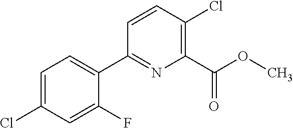 [SMILES: COC(=O)c1nc(-c2ccc(Cl)cc2F)ccc1Cl] | Methyl 3-chloro-6- (4-chloro-2- fluorophenyl) picolinate | Example 19 using starting materials described in WO 2003011853 and tert-butylnitrite and DMF as solvent, as described in J. | White Solid | 42 mg, 56% |
| | | | Org. Chem. 1977, | | |
| | | | 42, 3494 | | |
| F271 | 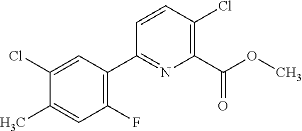 | Methyl 3-chloro-6- (5-chloro-2-fluoro-4- methylphenyl) picolinate | Example 19 using starting materials described in WO 2003011853 and tert-butylnitrite and DMF as solvent, as described in J. | White Solid | 38 mg, 51% |
| | | | Org. Chem. 1977, | | |
| | | | 42, 3494 | | |
| F272 | 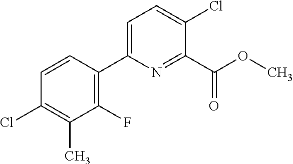 [SMILES: COC(=O)c1nc(-c2ccc(Cl)c(C)c2F)ccc1Cl] | Methyl 3-chloro-6- (4-chloro-2-fluoro-3- methylphenyl) picolinate | Example 19 using starting materials described in WO 2007082098 and tert-butylnitrite and DMF as solvent, as described in J. Org. Chem. 1977, 42, 3494 | White Solid | 41 mg, 54% |
| F273 | 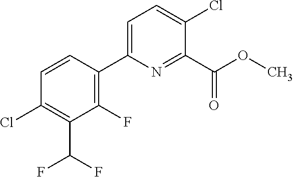 [SMILES: COC(=O)c1nc(-c2ccc(Cl)c(C(F)F)c2F)ccc1Cl] | Methyl 3-chloro-6- (4-chloro-3- (difluoromethyl)-2- fluorophenyl) picolinate | Example 19 using starting materials described in WO 2007082098 and tert-butylnitrite and DMF as solvent, as described in J. Org. Chem. 1977, 42, 3494 | White Solid | 44 mg, 58% |
| F274 | 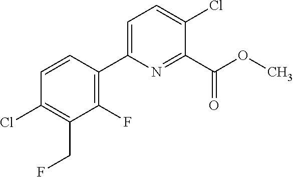 [SMILES: COC(=O)c1nc(-c2ccc(Cl)c(CF)c2F)ccc1Cl] | Methyl 3-chloro-6- (4-chloro-2-fluoro-3- (fluoromethyl) phenyl) picolinate | Example 19 using starting materials described in U.S. Pat. No. 7,314,849 and tert-butylnitrite and DMF as solvent, as described in J. Org. Chem. 1977, 42, 3494 | White Solid | 42 mg, 55% |
| F275 | 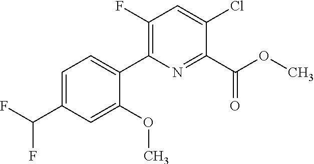 [SMILES: COC(=O)c1nc(-c2ccc(C(F)F)cc2OC)c(F)cc1Cl] | Methyl 3-chloro-6- (4-(difluoromethyl)- 2-methoxyphenyl)- 5- fluoropicolinate | Example 64 | White Solid | 179 mg, 54% |
| F276 | 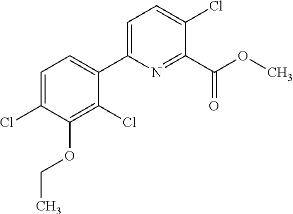 [SMILES: CCOc1c(Cl)ccc(-c2ccc(Cl)c(C(=O)OC)n2)c1Cl] | Methyl 3-chloro-6- (2,4-dichloro-3- ethoxyphenyl) picolinate | Example 19 using starting materials described in WO 2007082098 and tert-butylnitrite and DMF as solvent, as described in J. Org. Chem. 1977, 42, 3494 | White Solid | 37 mg, 49% |
| F277 | 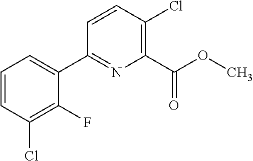 [SMILES: COC(=O)c1nc(-c2cccc(Cl)c2F)ccc1Cl] | Methyl 3-chloro-6- (3-chloro-2- fluorophenyl) picolinate | Example 41 | White Solid | 39 mg, 52% |
| F278 | 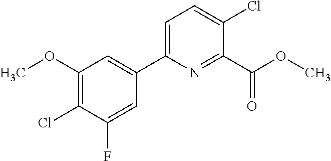 | Methyl 3-chloro-6- (4-chloro-3-fluoro-5- methoxyphenyl) picolinate | Example 19 using starting materials described in WO 2012080187 and tert-butylnitrite and DMF as solvent, as described in J. Org. Chem. 1977, 42, 3494 | White Solid | 36 mg, 47% |
| F279 | 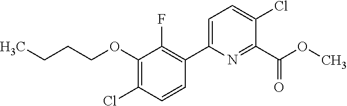 [SMILES: CCCCOc1c(Cl)ccc(-c2ccc(Cl)c(C(=O)OC)n2)c1F] | Methyl 6-(3-butoxy- 4-chloro-2- fluorophenyl)-3- chloropicolinate | Example 19 using starting materials described in WO 2007082098 and tert-butylnitrite and DMF as solvent, as described in J. | White Solid | 29 mg, 38% |
| | | | Org. Chem. 1977, | | |
| | | | 42, 3494 | | |
| F280 | 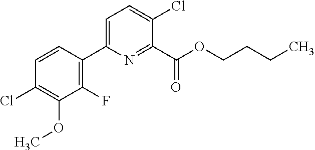 [SMILES: CCCCOC(=O)c1nc(-c2ccc(Cl)c(OC)c2F)ccc1Cl] | Butyl 3-chloro-6-(4- chloro-2-fluoro-3- methoxyphenyl) picolinate | Example 19 using starting materials described in WO 2009029518 and tert-butylnitrite and DMF as solvent, as described in J. Org. Chem. 1977, 42, 3494 | White Solid | 44 mg, 58% |
| F281 | 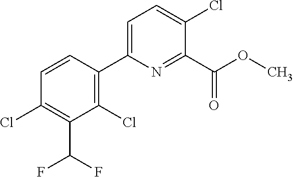 [SMILES: COC(=O)c1nc(-c2ccc(Cl)c(C(F)F)c2Cl)ccc1Cl] | Methyl 3-chloro-6- (2,4-dichloro-3- (difluoromethyl) phenyl)picolinate | Example 19 using starting materials described in WO 2007082098 and tert-butylnitrite and DMF as solvent, as described in J. Org. Chem. 1977, 42, 3494 | White Solid | 27 mg, 35% |
| F282 | 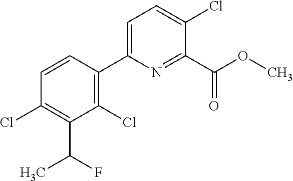 [SMILES: COC(=O)c1nc(-c2ccc(Cl)c(C(C)F)c2Cl)ccc1Cl] | Methyl 3-chloro-6- (2,4-dichloro-3-(1- fluoroethyl)phenyl) picolinate | Example 19 using starting materials described in WO 2007082098 and tert-butylnitrite and DMF as solvent, as described in J. Org. Chem. 1977, 42, 3494 | White Solid | 40 mg, 53% |
| F283 | 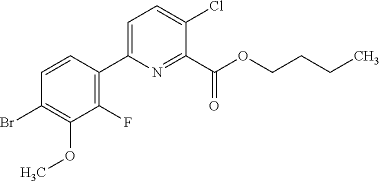 [SMILES: CCCCOC(=O)c1nc(-c2ccc(Br)c(OC)c2F)ccc1Cl] | Butyl 6-(4-bromo-2- fluoro-3- methoxyphenyl)-3- chloropicolinate | Example 19 using starting materials described in WO 2007082098 and tert-butylnitrite and DMF as solvent, as described in J. Org. Chem. 1977, 42, 3494 | White Solid | 38 mg, 49% |
| F284 | 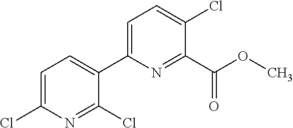 [SMILES: COC(=O)c1nc(-c2ccc(Cl)nc2Cl)ccc1Cl] | Methyl 2′,5,6′- trichloro-[2,3′- bipyridine]-6- carboxylate | Example 19 using starting materials described in US 20140274703 and tert-butylnitrite and DMF as solvent, as described in J. | White Solid | 28 mg, 37% |
| | | | Org. Chem. 1977, | | |
| | | | 42, 3494 | | |
| F285 | 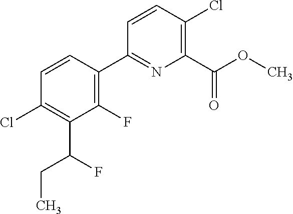 [SMILES: CCC(F)c1c(Cl)ccc(-c2ccc(Cl)c(C(=O)OC)n2)c1F] | Methyl 3-chloro-6- (4-chloro-2-fluoro-3- (1- fluoropropyl)phenyl) picolinate | Example 19 using starting materials described in WO 2007082098 and tert-butylnitrite and DMF as solvent, as described in J. Org. Chem. 1977, 42, 3494 | White Solid | 42 mg, 55% |
| F286 | 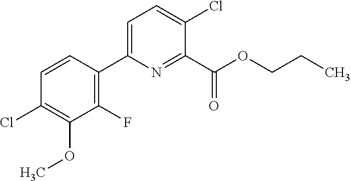 [SMILES: CCCOC(=O)c1nc(-c2ccc(Cl)c(OC)c2F)ccc1Cl] | Propyl 3-chloro-6-(4- chloro-2-fluoro-3- methoxyphenyl) picolinate | Example 19 using tert-butylnitrite and DMF as solvent, as described in J. Org. Chem. 1977, 42, 3494 | White Solid | 42 mg, 55% |
| F287 | 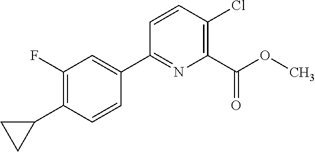 [SMILES: COC(=O)c1nc(-c2ccc(C3CC3)c(F)c2)ccc1Cl] | Methyl 3-chloro-6- (4-cyclopropyl-3- fluorophenyl) picolinate | Example 41 | White Solid | 39 mg, 52% |
| F288 | 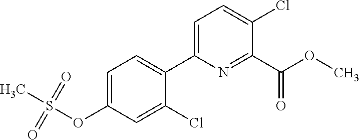 | Methyl 3-chloro-6- (2-chloro-4- ((methylsulfonyl)oxy) phenyl)picolinate | Example 70 | White Solid | 293 mg, 90% |
| F289 | 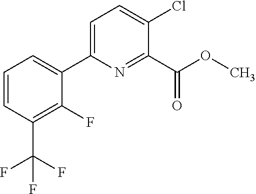 [SMILES: COC(=O)c1nc(-c2cccc(C(F)(F)F)c2F)ccc1Cl] | Methyl 3-chloro-6- (2-fluoro-3- (trifluoromethyl) phenyl)picolinate | Example 41 | White Solid | 40 mg, 53% |
| F290 | 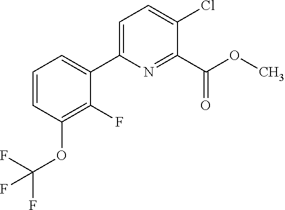 [SMILES: COC(=O)c1nc(-c2cccc(OC(F)(F)F)c2F)ccc1Cl] | Methyl 3-chloro-6- (2-fluoro-3- (trifluoromethoxy) phenyl)picolinate | Example 41 | White Solid | 43 mg, 56% |
| F291 | 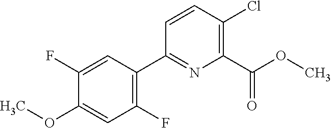 | Methyl 3-chloro-6- (2,5-difluoro-4- methoxyphenyl) picolinate | Example 41 | White Solid | 37 mg, 49% |
| F292 | 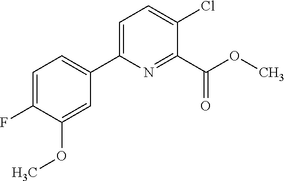 [SMILES: COC(=O)c1nc(-c2ccc(F)c(OC)c2)ccc1Cl] | Methyl 3-chloro-6- (4-fluoro-3- methoxyphenyl) picolinate | Example 41 | White Solid | 34 mg, 45% |
| F293 | 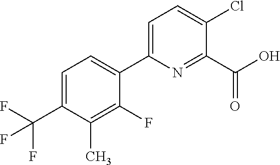 [SMILES: Cc1c(C(F)(F)F)ccc(-c2ccc(Cl)c(C(=O)O)n2)c1F] | 3-Chloro-6-(2-fluoro- 3-methyl-4- (trifluoromethyl) phenyl)picolinic acid | Example 19 using tert-butylnitrite and DMF following the hydrolysis of the intermediate methyl ester C22 (Example 27) | White Solid | 28 mg, 37% |
| F294 | 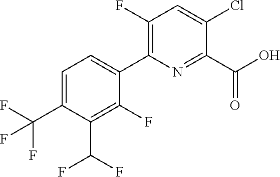 [SMILES: O=C(O)c1nc(-c2ccc(C(F)(F)F)c(C(F)F)c2F)c(F)cc1Cl] | 3-Chloro-6-(3- (difluoromethyl)-2- fluoro-4- (trifluoromethyl) phenyl)-5- fluoropicolinic acid | Example 19 using C25 tert- butylnitrite and DMF as solvent, as described in J. Org. Chem. 1977, 42, 3494 | White Solid | 12 mg, 16% |
| F295 | 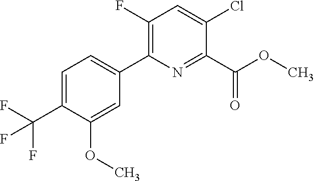 [SMILES: COC(=O)c1nc(-c2ccc(C(F)(F)F)c(OC)c2)c(F)cc1Cl] | Methyl 3-chloro-5- fluoro-6-(3-methoxy- 4- (trifluoromethyl) phenyl)picolinate | Example 41 | White Solid | 40 mg, 53% |
| F296 | 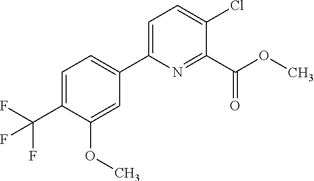 [SMILES: COC(=O)c1nc(-c2ccc(C(F)(F)F)c(OC)c2)ccc1Cl] | Methyl 3-chloro-6- (3-methoxy-4- (trifluoromethyl) phenyl)picolinate | Example 41 | White Solid | 42 mg, 55% |
| F297 | 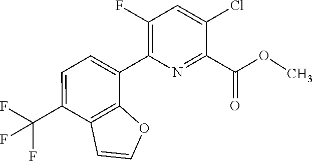 [SMILES: COC(=O)c1nc(-c2ccc(C(F)(F)F)c3ccoc23)c(F)cc1Cl] | Methyl 3-chloro-5- fluoro-6-(4- (trifluoromethyl) benzofuran-7-yl) picolinate | Example 19 using C23, tert- butylnitrite and DMF as solvent, as described in J. Org. Chem. 1977, 42, 349 | White S | 17 mg, 22% |
| F298 | 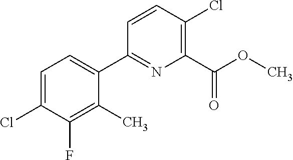 [SMILES: COC(=O)c1nc(-c2ccc(Cl)c(F)c2C)ccc1Cl] | Methyl 3-chloro-6- (4-chloro-3-fluoro-2- methylphenyl) picolinate | Example 19 using starting materials described in US 20140274703 and tert-butylnitrite and DMF as solvent, as described in J. Org. Chem. 1977, 42, 3494 | White Solid | 46 mg, 62% |
| F299 | 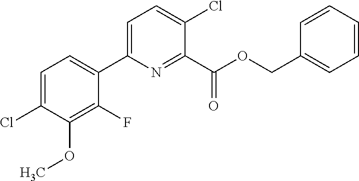 [SMILES: COc1c(Cl)ccc(-c2ccc(Cl)c(C(=O)OCc3ccccc3)n2)c1F] | Benzyl 3-chloro-6-(4- chloro-2-fluoro-3- methoxyphenyl) picolinate | Example 19 using starting materials described in US 20120190551 and tert-butylnitrite and DMF as solvent, as described in J. Org. Chem. 1977, 42, 3494 | White Solid | 44 mg, 57% |
| F300 | 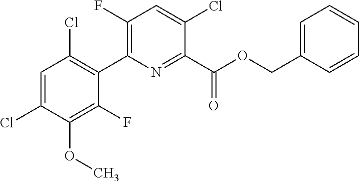 [SMILES: COc1c(Cl)cc(Cl)c(-c2nc(C(=O)OCc3ccccc3)c(Cl)cc2F)c1F] | Benzyl 3-chloro-6- (4,6-dichloro-2- fluoro-3- methoxyphenyl)-5- fluoropicolinate | Example 19 using tert-butylnitrite and DMF as solvent, as described in J. Org. Chem. 1977, 42, 3494 | White Solid | |
| F301 | 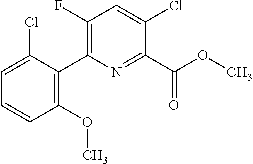 [SMILES: COC(=O)c1nc(-c2c(Cl)cccc2OC)c(F)cc1Cl] | Methyl 3-chloro-6- (2-chloro-6- methoxyphenyl)-5- fluoropicolinate | Example 19 using C24, tert- butylnitrite and DMF as solvent, as described in J. Org. Chem. 1977, 42, 3494 | White Solid | 20 mg, 27% |
| F302 | 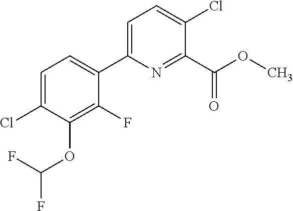 [SMILES: COC(=O)c1nc(-c2ccc(Cl)c(OC(F)F)c2F)ccc1Cl] | Methyl 3-chloro-6- (4-chloro-3- (difluoromethoxy)-2- fluorophenyl) picolinate | Example 19 using starting materials described in WO 2007082098 and tert-butylnitrite and DMF as solvent, as described in J. Org. Chem. 1977, 42, 3494 | White Solid | 1.9 mg, 2.6% |
| F303 | 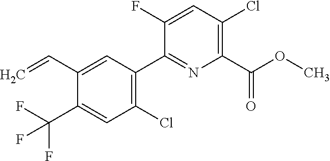 [SMILES: C=Cc1cc(-c2nc(C(=O)OC)c(Cl)cc2F)c(Cl)cc1C(F)(F)F] | Methyl 3-chloro-6- (2-chloro-4- (trifluoromethyl)-5- vinylphenyl)-5- fluoropicolinate | Example 41 | White Solid | 35 mg, 7.9% |
| F304 | 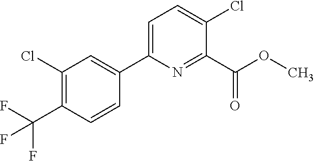 [SMILES: COC(=O)c1nc(-c2ccc(C(F)(F)F)c(Cl)c2)ccc1Cl] | Methyl 3-chloro-6- (3-chloro-4- (trifluoromethyl) phenyl)picolinate | Example 32 | White Solid | 267 mg, 76% |
| F305 | 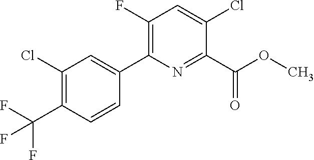 [SMILES: COC(=O)c1nc(-c2ccc(C(F)(F)F)c(Cl)c2)c(F)cc1Cl] | Methyl 3-chloro-6- (3-chloro-4- (trifluoromethyl) phenyl)- 5-fluoropicolinate | Example 32 | White Solid | 233 mg, 63% |
| F306 | 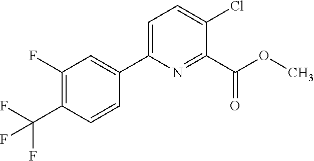 [SMILES: COC(=O)c1nc(-c2ccc(C(F)(F)F)c(F)c2)ccc1Cl] | Methyl 3-chloro-6- (3-fluoro-4- (trifluoromethyl) phenyl)picolinate | Example 32 | Viscous Clear Oil | 304 mg, 91% |
| F307 | 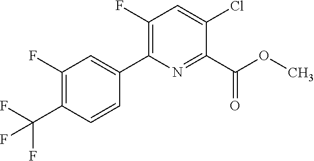 [SMILES: COC(=O)c1nc(-c2ccc(C(F)(F)F)c(F)c2)c(F)cc1Cl] | Methyl 3-chloro-5- fluoro-6-(3-fluoro-4- (trifluoromethyl) phenyl) picolinate | Example 32 | White Solid | 242 mg, 69% |
| F308 | 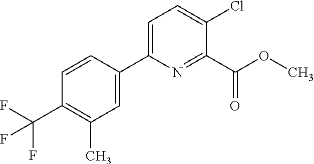 [SMILES: COC(=O)c1nc(-c2ccc(C(F)(F)F)c(C)c2)ccc1Cl] | Methyl 3-chloro-6- (3-methyl-4- (trifluoromethyl) phenyl)picolinate | Example 32 | White Solid | 188 mg, 57% |
| F309 | 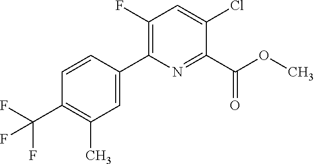 [SMILES: COC(=O)c1nc(-c2ccc(C(F)(F)F)c(C)c2)c(F)cc1Cl] | Methyl 3-chloro-5- fluoro-6-(3-methyl-4- (trifluoromethyl) phenyl)picolinate | Example 32 | Viscous Clear Oil | 225 mg, 65% |
| F310 | 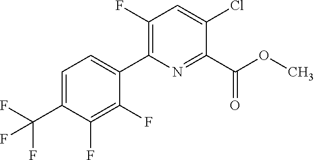 [SMILES: COC(=O)c1nc(-c2ccc(C(F)(F)F)c(F)c2F)c(F)cc1Cl] | Methyl 3-chloro-6- (2,3-difluoro-4- (trifluoromethyl) phenyl)- 5-fluoropicolinate | Example 63 | White Solid | 275 mg, 40% |
| F311 | 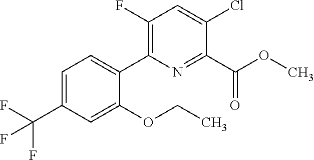 [SMILES: CCOc1cc(C(F)(F)F)ccc1-c1nc(C(=O)OC)c(Cl)cc1F] | Methyl 3-chloro-6- (2-ethoxy-4- (trifluoromethyl) phenyl)- 5-fluoropicolinate | Example 63 | White Solid | 435 mg, 62% |
| F312 | 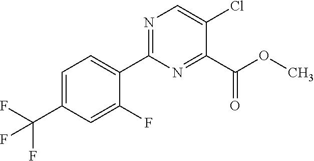 [SMILES: COC(=O)c1nc(-c2ccc(C(F)(F)F)cc2F)ncc1Cl] | Methyl 5-chloro-2- (2-fluoro-4- (trifluoromethyl) phenyl) pyrimidine-4- carboxylate | Example 63 | Orange Oil | 130 mg, 20% |
| F313 | 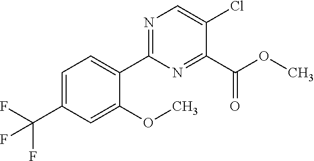 [SMILES: COC(=O)c1nc(-c2ccc(C(F)(F)F)cc2OC)ncc1Cl] | Methyl 5-chloro-2- (2-methoxy-4- (trifluoromethyl) phenyl) pyrimidine-4- carboxylate | Example 53 | White Solid | 255 mg, 37% |
| F314 | 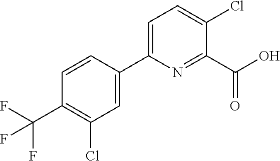 [SMILES: O=C(O)c1nc(-c2ccc(C(F)(F)F)c(Cl)c2)ccc1Cl] | 3-Chloro-6-(3-chloro- 4- (trifluoromethyl) phenyl) picolinic acid | Example 27 | White Solid | 90 mg, 94% |
| F315 | 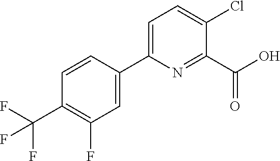 [SMILES: O=C(O)c1nc(-c2ccc(C(F)(F)F)c(F)c2)ccc1Cl] | 3-Chloro-6-(3-fluoro- 4- (trifluoromethyl) phenyl) picolinic acid | Example 27 | White Solid | 95 mg, 99% |
| F316 | 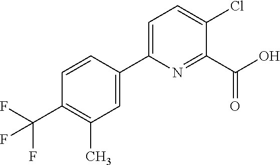 [SMILES: Cc1cc(-c2ccc(Cl)c(C(=O)O)n2)ccc1C(F)(F)F] | 3-Chloro-6-(3- methyl-4- (trifluoromethyl) phenyl) picolinic acid | Example 27 | White Solid | 93 mg, 97% |
| F317 | 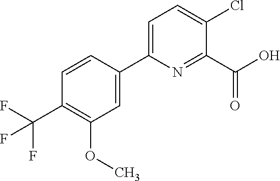 [SMILES: COc1cc(-c2ccc(Cl)c(C(=O)O)n2)ccc1C(F)(F)F] | 3-Chloro-6-(3- methoxy-4- (trifluoromethyl) phenyl)picolinic acid | Example 27 | White Solid | 90 mg, 94% |
| F318 | 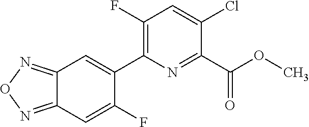 [SMILES: COC(=O)c1nc(-c2cc3nonc3cc2F)c(F)cc1Cl] | Methyl 3-chloro-5- fluoro-6-(6- fluorobenzo[c][1,2,5] oxadiazol-5- yl)picolinate | Example 41 | White Solid | 164 mg, 46% |
| F319 | 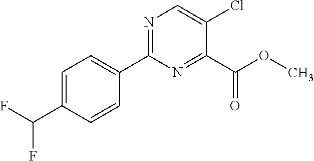 [SMILES: COC(=O)c1nc(-c2ccc(C(F)F)cc2)ncc1Cl] | Methyl 5-chloro-2- (4- (difluoromethyl) phenyl)pyrimidine-4- carboxylate | Example 53 | White Solid | 211 mg, 36% |
| F320 | 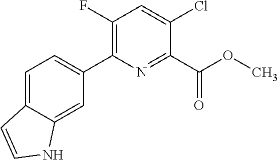 [SMILES: COC(=O)c1nc(-c2ccc3cc[nH]c3c2)c(F)cc1Cl] | Methyl 3-chloro-5- fluoro-6-(1H-indol-6- yl)picolinate | Example 63 | Off-White Solid | 33 mg, 7.6% |
| F321 | 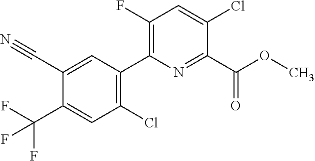 [SMILES: COC(=O)c1nc(-c2cc(C#N)c(C(F)(F)F)cc2Cl)c(F)cc1Cl] | Methyl 3-chloro-6- (2-chloro-5-cyano-4- (trifluoromethyl) phenyl)- 5-fluoropicolinate | Example 41 | White Solid | 72 mg, 40% |
| F322 | 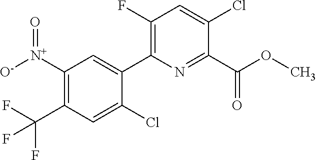 [SMILES: COC(=O)c1nc(-c2cc([N+](=O)[O-])c(C(F)(F)F)cc2Cl)c(F)cc1Cl] | Methyl 3-chloro-6- (2-chloro-5-nitro-4- (trifluoromethyl) phenyl)- 5-fluoropicolinate | Example 51 | White Solid | 36 mg, 6.1% |
| F323 | 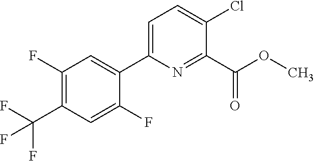 [SMILES: COC(=O)c1nc(-c2cc(F)c(C(F)(F)F)cc2F)ccc1Cl] | Methyl 3-chloro-6- (2,5-difluoro-4- (trifluoromethyl) phenyl)picolinate | Example 51 | Amber Oil | 57 mg, 28% |
| F324 | 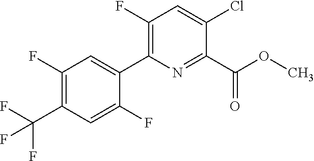 [SMILES: COC(=O)c1nc(-c2cc(F)c(C(F)(F)F)cc2F)c(F)cc1Cl] | Methyl 3-chloro-6- (2,5-difluoro-4- (trifluoromethyl) phenyl)- 5-fluoropicolinate | Example 51 | Amber Solid | 101 mg, 43% |
| F325 | 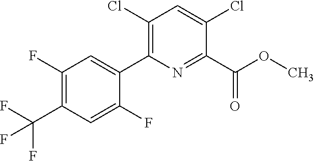 [SMILES: COC(=O)c1nc(-c2cc(F)c(C(F)(F)F)cc2F)c(Cl)cc1Cl] | Methyl 3,5-dichloro- 6-(2,5-difluoro-4- (trifluoromethyl) phenyl)picolinate | Example 51 | White Solid | 62 mg, 26% |
| F326 | 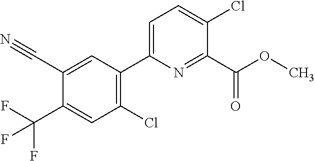 [SMILES: COC(=O)c1nc(-c2cc(C#N)c(C(F)(F)F)cc2Cl)ccc1Cl] | Methyl 3-chloro-6- (2-chloro-5-cyano-4- (trifluoromethyl) phenyl)picolinate | Example 41 | White Solid | 53 mg, 39% |
| F327 | 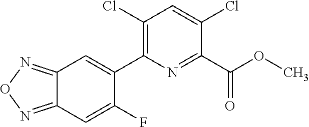 [SMILES: COC(=O)c1nc(-c2cc3nonc3cc2F)c(Cl)cc1Cl] | Methyl 3,5-dichloro- 6-(6- fluorobenzo[c][1,2,5] oxadiazol-5- yl)picolinate | Example 41 | White Solid | 25 mg, 7.3% |
| F328 | 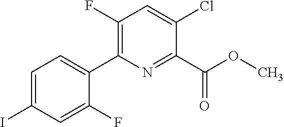 [SMILES: COC(=O)c1nc(-c2ccc(I)cc2F)c(F)cc1Cl] | Methyl 3-chloro-5- fluoro-6-(2-fluoro-4- iodophenyl) picolinate | Example 71 | White Solid | 320 mg, 46% |
| F329 | 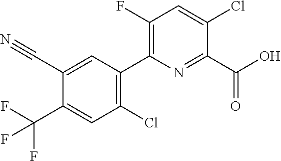 [SMILES: N#Cc1cc(-c2nc(C(=O)O)c(Cl)cc2F)c(Cl)cc1C(F)(F)F] | 3-Chloro-6-(2-chloro- 5-cyano-4- (trifluoromethyl) phenyl)- 5-fluoropicolinic acid | Example 27 | White Solid | 38 mg, 94% |
| F330 | 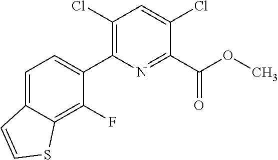 [SMILES: COC(=O)c1nc(-c2ccc3ccsc3c2F)c(Cl)cc1Cl] | Methyl 3,5-dichloro- 6-(7- fluorobenzo[b] thiophen- 6-yl)picolinate | Example 51 | White Solid | 46 mg, 31% |
| F331 | 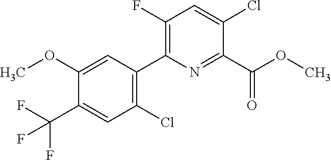 [SMILES: COC(=O)c1nc(-c2cc(OC)c(C(F)(F)F)cc2Cl)c(F)cc1Cl] | Methyl 3-chloro-6- (2-chloro-5-methoxy- 4- (trifluoromethyl) phenyl)-5- fluoropicolinate | Example 41 | White Solid | 46 mg, 29% |
| F332 | 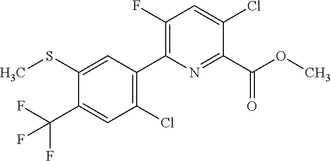 | Methyl 3-chloro-6- (2-chloro-5- (methylthio)-4- (trifluoromethyl) phenyl) 5-fluoropicolinate | Example 41 | White Solid | 25 mg, 22% |
| F333 | 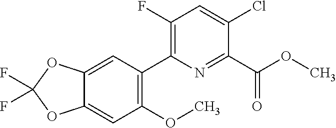 [SMILES: COC(=O)c1nc(-c2cc3c(cc2OC)OC(F)(F)O3)c(F)cc1Cl] | Methyl 3-chloro-6- (2,2-difluoro-6- methoxybenzo[d][1,3] dioxol-5-yl)-5- fluoropicolinate | Example 41 | White Solid | 20 mg, 37% |
| F334 | 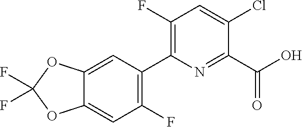 [SMILES: O=C(O)c1nc(-c2cc3c(cc2F)OC(F)(F)O3)c(F)cc1Cl] | 3-Chloro-5-fluoro-6- (2,2,6- trifluorobenzo[d][1,3] dioxol-5-yl)picolinic acid | Example 27 | White Solid | 66 mg, 69% |
| F335 | 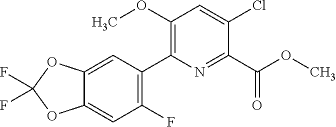 [SMILES: COC(=O)c1nc(-c2cc3c(cc2F)OC(F)(F)O3)c(OC)cc1Cl] | Methyl 3-chloro-5- methoxy-6-(2,2,6- trifluorobenzo[d][1,3] dioxol-5-yl)picolinate | Example 17 | White Solid | 59 mg, 44% |
| F336 | 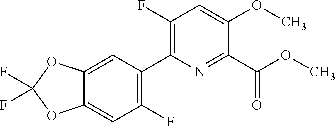 [SMILES: COC(=O)c1nc(-c2cc3c(cc2F)OC(F)(F)O3)c(F)cc1OC] | Methyl 5-fluoro-3- methoxy-6-(2,2,6- trifluorobenzo[d][1,3] dioxol-5-yl)picolinate | Example 17 | White Solid | 9 mg, 7% |
| F337 | 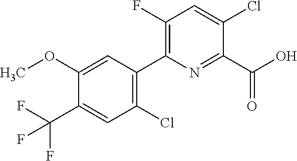 [SMILES: COc1cc(-c2nc(C(=O)O)c(Cl)cc2F)c(Cl)cc1C(F)(F)F] | 3-Chloro-6-(2-chloro- 5-methoxy-4- (trifluoromethyl) phenyl)-5- fluoropicolinic acid | Example 27 | White Solid | 13 mg, 80% |
| F338 | 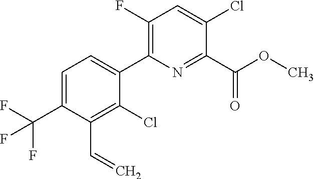 [SMILES: C=Cc1c(C(F)(F)F)ccc(-c2nc(C(=O)OC)c(Cl)cc2F)c1Cl] | Methyl 3-chloro-6- (2-chloro-4- (trifluoromethyl)-3- vinylphenyl)-5- fluoropicolinate | Example 58 | White Solid | 55 mg, 25% |
| F339 | 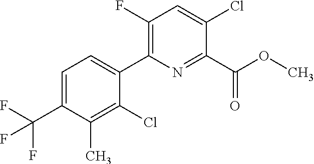 [SMILES: COC(=O)c1nc(-c2ccc(C(F)(F)F)c(C)c2Cl)c(F)cc1Cl] | Methyl 3-chloro-6- (2-chloro-3-methyl-4- (trifluoromethyl) phenyl)- 5-fluoropicolinate | Example 57 | Viscous Clear Oil | 46 mg, 22% |
| F340 | 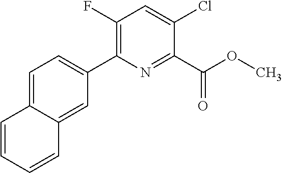 [SMILES: COC(=O)c1nc(-c2ccc3ccccc3c2)c(F)cc1Cl] | Methyl 3-chloro-5- fluoro-6-(naphthalen- 2-yl)picolinate | Example 63 | White Solid | 292 mg, 67% |
| F341 | 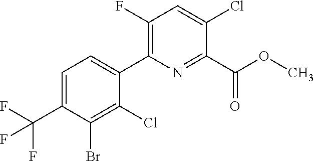 [SMILES: COC(=O)c1nc(-c2ccc(C(F)(F)F)c(Br)c2Cl)c(F)cc1Cl] | Methyl 6-(3-bromo- 2-chloro-4- (trifluoromethyl) phenyl)- 3-chloro-5 -fluoropicolinate | Example 32 | White Solid | 722 mg, 72% |
| F342 | 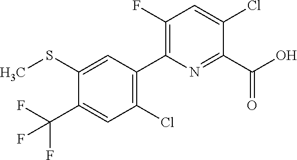 [SMILES: CSc1cc(-c2nc(C(=O)O)c(Cl)cc2F)c(Cl)cc1C(F)(F)F] | 3-Chloro-6-(2-chloro- 5-(methylthio)-4- (trifluoromethyl) phenyl)- 5-fluoropicolinic acid | Example 27 | White Solid | 14 mg, 92% |
| F343 | 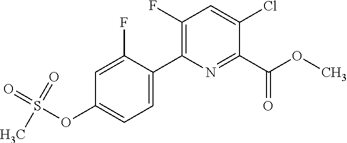 [SMILES: COC(=O)c1nc(-c2ccc(OS(C)(=O)=O)cc2F)c(F)cc1Cl] | Methyl 3-chloro-5- ((methylsulfonyl) oxy) phenyl)picolinate | Example 70 | White Solid | 144 mg, 74% |
| F344 | 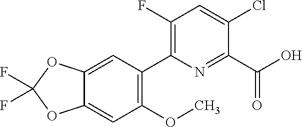 [SMILES: COc1cc2c(cc1-c1nc(C(=O)O)c(Cl)cc1F)OC(F)(F)O2] | 3-Chloro-6-(2,2- difluoro-6- methoxybenzo[d][1,3] dioxol-5-yl)-5- fluoropicolinic acid | Example 27 | White Solid | 11 mg, 99% |
| F345 | 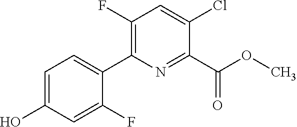 [SMILES: COC(=O)c1nc(-c2ccc(O)cc2F)c(F)cc1Cl] | Methyl 3-chloro-5- fluoro-6-(2-fluoro-4- hydroxyphenyl) picolinate | Example 63 | Off-White Solid | 227 mg, 22% |
| F346 | 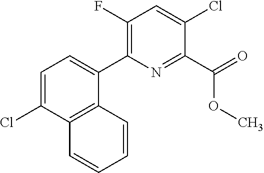 [SMILES: COC(=O)c1nc(-c2ccc(Cl)c3ccccc23)c(F)cc1Cl] | Methyl 3-chloro-6- (4-chloronaphthalen- 1-yl)-5- fluoropicolinate | Example 63 | Off-White Solid | 128 mg, 27% |
| F347 | 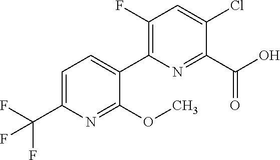 [SMILES: COc1nc(C(F)(F)F)ccc1-c1nc(C(=O)O)c(Cl)cc1F] | 5-Chloro-3-fluoro-2′- methoxy-6′- (trifluoromethyl)- [2,3′-bipyridine]-6- carboxylic acid | Example 49 | White Solid | 80 mg, 77% |
| F348 | 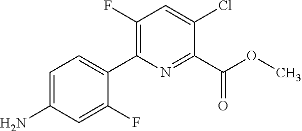 [SMILES: COC(=O)c1nc(-c2ccc(N)cc2F)c(F)cc1Cl] | Methyl 6-(4-amino-2- fluorophenyl)-3- chloro-5- fluoropicolinate | Example 63 | Yellow Solid | 555 mg, 43% |
| F349 | 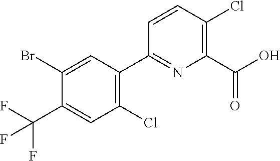 [SMILES: O=C(O)c1nc(-c2cc(Br)c(C(F)(F)F)cc2Cl)ccc1Cl] | 6-(5-Bromo-2-chloro- 4- (trifluoromethyl) phenyl)- 3-chloropicolinic acid | Example 27 | White Solid | 13 mg, 95% |
| F350 | 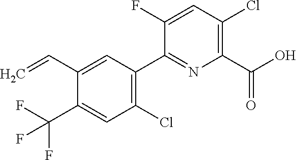 [SMILES: C=Cc1cc(-c2nc(C(=O)O)c(Cl)cc2F)c(Cl)cc1C(F)(F)F] | 3-Chloro-6-(2-chloro- 4-(trifluoromethyl)-5- vinylphenyl)-5- fluoropicolinic acid | Example 27 | White Solid | 31 mg, 86% |
| F351 | 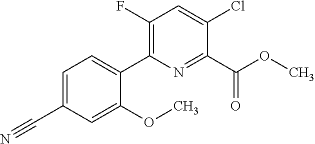 [SMILES: COC(=O)c1nc(-c2ccc(C#N)cc2OC)c(F)cc1Cl] | Methyl 3-chloro-6- (4-cyano-2- methoxyphenyl)-5- fluoropicolinate | Example 63 | White Solid | 88 mg, 12% |
| F352 | 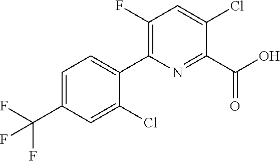 [SMILES: O=C(O)c1nc(-c2ccc(C(F)(F)F)cc2Cl)c(F)cc1Cl] | 3-Chloro-6-(2-chloro- 4- (trifluoromethyl) phenyl)- 5-fluoropicolinic acid | Example 48 | White Solid | 81 mg, 84% |
| F353 | 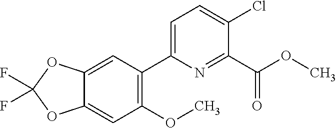 [SMILES: COC(=O)c1nc(-c2cc3c(cc2OC)OC(F)(F)O3)ccc1Cl] | Methyl 3-chloro-6- (2,2-difluoro-6- methoxybenzo[d][1,3] dioxol-5-yl)picolinate | Example 41 | Yellow Solid | 166 mg, 57% |
| F354 | 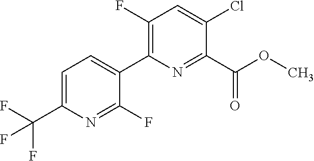 [SMILES: COC(=O)c1nc(-c2ccc(C(F)(F)F)nc2F)c(F)cc1Cl] | Methyl 5-chloro-2′,3- difluoro-6′- (trifluoromethyl)- [2,3′-bipyridine]-6- carboxylate | Example 63 | White Solid | 276 mg, 14% |
| F355 | 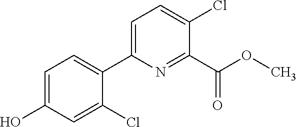 [SMILES: COC(=O)c1nc(-c2ccc(O)cc2Cl)ccc1Cl] | Methyl 3-chloro-6- (2-chloro-4- hydroxyphenyl) picolinate | Example 63 | White Solid | 795 mg, 48% |
| F356 | 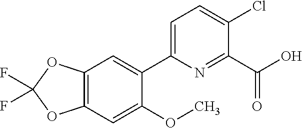 [SMILES: COc1cc2c(cc1-c1ccc(Cl)c(C(=O)O)n1)OC(F)(F)O2] | 3-Chloro-6-(2,2- difluoro-6- methoxybenzo[d][1,3] dioxol-5-yl)picolinic acid | Example 27 | White Solid | 54 mg, 86% |
| F357 | 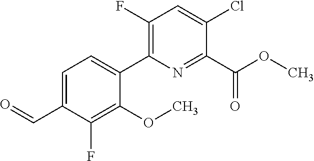 [SMILES: COC(=O)c1nc(-c2ccc(C=O)c(F)c2OC)c(F)cc1Cl] | Methyl 3-chloro-5- fluoro-6-(3-fluoro-4- formyl-2- methoxyphenyl) picolinate | Example 63 | White Solid | 314 mg, 23% |
| F358 | 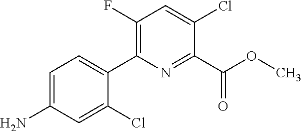 [SMILES: COC(=O)c1nc(-c2ccc(N)cc2Cl)c(F)cc1Cl] | Methyl 6-(4-amino-2- chlorophenyl)-3- chloro-5- fluoropicolinate | Example 63 | Off-White Solid | 234 mg, 18% |
| F359 | 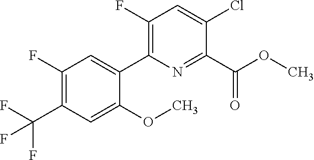 [SMILES: COC(=O)c1nc(-c2cc(F)c(C(F)(F)F)cc2OC)c(F)cc1Cl] | Methyl 3-chloro-5- fluoro-6-(5-fluoro-2- methoxy-4- (trifluoromethyl) phenyl)picolinate | Example 51 | White Solid | 118 mg, 50% |
| F360 | 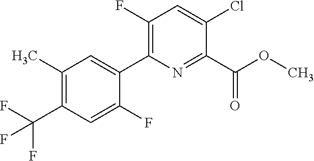 [SMILES: COC(=O)c1nc(-c2cc(C)c(C(F)(F)F)cc2F)c(F)cc1Cl] | Methyl 3-chloro-5- fluoro-6-(2-fluoro-5- methyl-4- (trifluoromethyl) phenyl)picolinate | Example 51 | Brown Liquid | 118 mg, 33% |
| F361 | 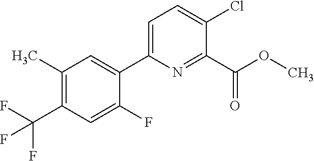 [SMILES: COC(=O)c1nc(-c2cc(C)c(C(F)(F)F)cc2F)ccc1Cl] | Methyl 3-chloro-6- (2-fluoro-5-methyl-4- (trifluoromethyl) phenyl)picolinate | Example 51 | Brown Oil | 90 mg, 26% |
| F362 | 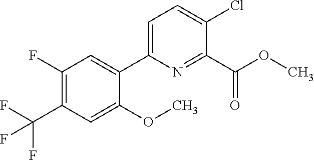 [SMILES: COC(=O)c1nc(-c2cc(F)c(C(F)(F)F)cc2OC)ccc1Cl] | Methyl 3-chloro-6- (5-fluoro-2-methoxy- 4- (trifluoromethyl) phenyl)picolinate | Example 51 | White Solid | 62 mg, 52% |
| F363 | 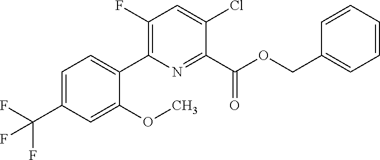 [SMILES: COc1cc(C(F)(F)F)ccc1-c1nc(C(=O)OCc2ccccc2)c(Cl)cc1F] | Benzyl 3-chloro-5- fluoro-6-(2-methoxy-4- (trifluoromethyl) phenyl)picolinate | Example 25 | White Solid | 215 mg, 70% |
| F364 | 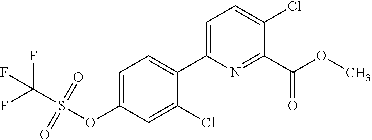 [SMILES: COC(=O)c1nc(-c2ccc(OS(=O)(=O)C(F)(F)F)cc2Cl)ccc1Cl] | Methyl 3-chloro-6- (2-chloro-4- (((trifluoromethyl) sulfonyl)oxy)phenyl)- picolinate | Example 69 | Off-White | 243 mg, 47% |
| F365 | 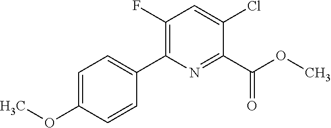 | Methyl 3-chloro-5- fluoro-6-(4- methoxyphenyl) picolinate | Example 63 | Off-White Solid | 152 mg, 35% |
| F366 | 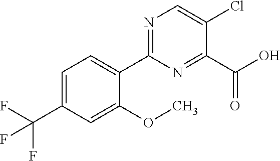 [SMILES: COc1cc(C(F)(F)F)ccc1-c1ncc(Cl)c(C(=O)O)n1] | 5-Chloro-2-(2- methoxy-4- (trifluoromethyl) phenyl)pyrimidine- 4-carboxylic acid | Example 50 | White Solid | 123 mg, 75% |
| F367 | 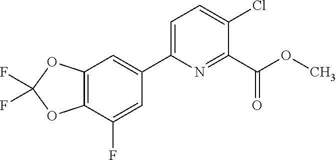 [SMILES: COC(=O)c1nc(-c2cc(F)c3c(c2)OC(F)(F)O3)ccc1Cl] | Methyl 3-chloro-6- (2,2,7- trifluorobenzo[d][1,3] dioxol-5-yl)picolinate | Example 19 using starting materials described in US 20140274701 | White Solid | 64 mg, 68% |
| F368 | 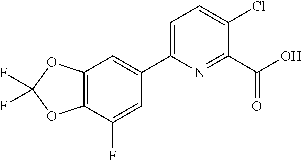 [SMILES: O=C(O)c1nc(-c2cc(F)c3c(c2)OC(F)(F)O3)ccc1Cl] | 3-Chloro-6-(2,2,7- trifluorobenzo[d][1,3] dioxol-5-yl)picolinic acid | Example 27 | White Solid | 31 mg, 44% |
| F369 | 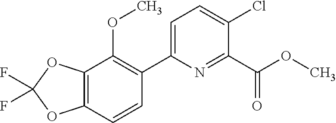 [SMILES: COC(=O)c1nc(-c2ccc3c(c2OC)OC(F)(F)O3)ccc1Cl] | Methyl 3-chloro-6- (2,2-difluoro-4- methoxybenzo[d][1,3] dioxol-5- yl)picolinate | Example 19 using starting materials described in US 20140274701 | White Solid | 125 mg, 66% |
| F370 | 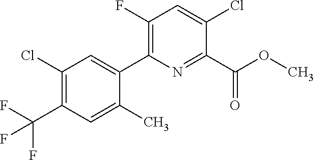 [SMILES: COC(=O)c1nc(-c2cc(Cl)c(C(F)(F)F)cc2C)c(F)cc1Cl] | Methyl 3-chloro-6- (5-chloro-2-methyl-4- (trifluoromethyl) phenyl)- 5-fluoropicolinate | Example 51 | White Solid | 41 mg, 69% |
| F371 | 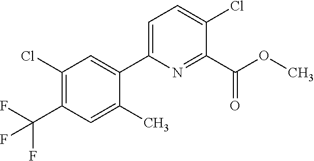 [SMILES: COC(=O)c1nc(-c2cc(Cl)c(C(F)(F)F)cc2C)ccc1Cl] | Methyl 3-chloro-6- (5-chloro-2-methyl-4- (trifluoromethyl) phenyl)picolinate | Example 51 | White Solid | 31 mg, 72% |
| F372 | 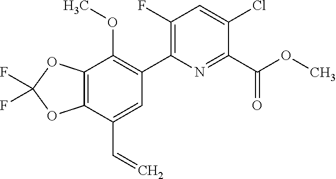 [SMILES: C=Cc1cc(-c2nc(C(=O)OC)c(Cl)cc2F)c(OC)c2c1OC(F)(F)O2] | Methyl 3-chloro-6- (2,2-difluoro-4- methoxy-7- vinylbenzo[d][1,3] dioxol-5-yl)-5- fluoropicolinate | Example 41 | White Solid | 20 mg, 41% |
| F373 | 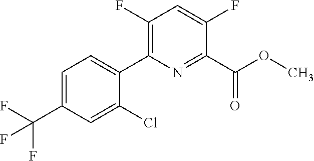 [SMILES: COC(=O)c1nc(-c2ccc(C(F)(F)F)cc2Cl)c(F)cc1F] | Methyl 6-(2-chloro- 4-(trifluoromethyl) phenyl)-3,5- difluoropicolinate | Example 32 | White Solid | 179 mg, 64% |
| F374 | 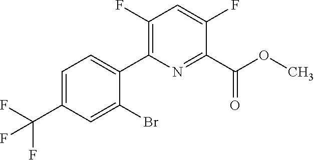 [SMILES: COC(=O)c1nc(-c2ccc(C(F)(F)F)cc2Br)c(F)cc1F] | Methyl 6-(2-bromo- 4- (trifluoromethyl) phenyl)-3,5- difluoropicolinate | Example 32 | White Solid | 166 mg, 53% |
| F375 | 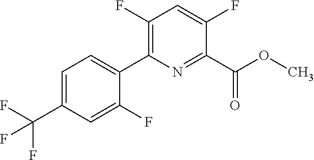 [SMILES: COC(=O)c1nc(-c2ccc(C(F)(F)F)cc2F)c(F)cc1F] | Methyl 3,5-difluoro- 6-(2-fluoro-4- (trifluoromethyl) phenyl)picolinate | Example 32 | White Solid | 130 mg, 49% |
| F376 | 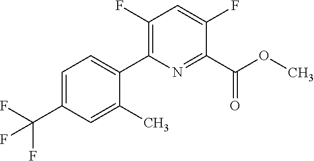 [SMILES: COC(=O)c1nc(-c2ccc(C(F)(F)F)cc2C)c(F)cc1F] | Methyl 3,5-difluoro- 6-(2-methyl-4- (trifluoromethyl) phenyl)picolinate | Example 32 | White Solid | 94 mg, 72% |
| F377 | 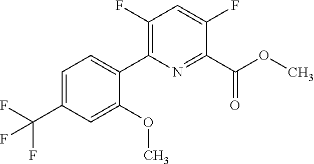 [SMILES: COC(=O)c1nc(-c2ccc(C(F)(F)F)cc2OC)c(F)cc1F] | Methyl 3,5-difluoro- 6-(2-methoxy-4- (trifluoromethyl) phenyl)picolinate | Example 32 | White Solid | 245 mg, 89% |
| F378 | 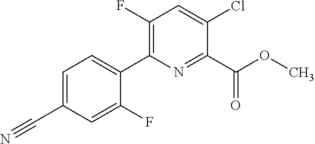 [SMILES: COC(=O)c1nc(-c2ccc(C#N)cc2F)c(F)cc1Cl] | Methyl 3-chloro-6- (4-cyano-2- fluorophenyl)-5- fluoropicolinate | Example 63 | White Solid | 131 mg, 22% |
| F379 | 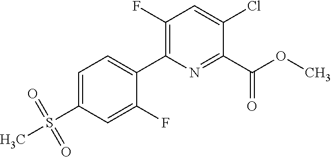 | Methyl 3-chloro-5- fluoro-6-(2-fluoro-4- (methylsulfonyl) phenyl)picolinate | Example 63 | Off-White Solid | 83 mg, 12% |
| F380 | 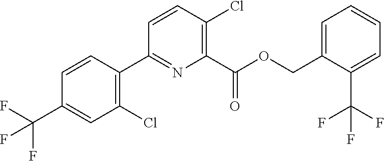 [SMILES: O=C(OCc1ccccc1C(F)(F)F)c1nc(-c2ccc(C(F)(F)F)cc2Cl)ccc1Cl] | 2- (Trifluoromethyl) benzyl 3-chloro-6-(2- chloro-4- (trifluoromethyl) phenyl)picolinate | Example 33 | Clear Oil | 87 mg, 56% |
| F381 | 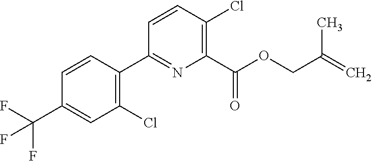 [SMILES: C=C(C)COC(=O)c1nc(-c2ccc(C(F)(F)F)cc2Cl)ccc1Cl] | 2-Methylallyl 3- chloro-6-(2-chloro-4- (trifluoromethyl) phenyl)picolinate | Example 33 | White Solid | 35 mg, 26% |

TABLE 2
Analytical Data for F Compounds

| | mp | | |
|---|---|---|---|
| No. | (° C.) | MASS SPEC | NMR |
| F1 | | ESIMS m/z | ¹H NMR (400 MHz, CDCl₃) δ 7.86 (d, J = 8.5 Hz, 1H), 7.76 (d, J = 8.4 Hz, |
| | | 326 | 1H), 7.62 (d, J = 8.0 Hz, 1H), 7.50 (d, J = 1.7 Hz, 1H), 7.36 (dd, J = 1.7, 8.1 |
| | | ([M + H]⁺) | Hz, 1H), 4.94 (q, J = 6.5 Hz, 1H), 4.01 (s, 3H), 1.51 (d, J = 6.4 Hz 3H) |
| F2 | 70- | ESIMS m/z | ¹H NMR (300 MHz, CDCl₃) δ 7.83 (d, J = 8.8 Hz, 1H), 7.78 (d, J = 8.1 Hz, |
| | 72 | 346 | 1H), 7.72 (d, J = 1.8 Hz, 1H), 7.63-7.57 (m, 1H), 7.45 (d, J = 8.8 Hz, 1H), |
| | | ([M + H]⁺) | 3.99 (s, 6H) |
| F3 | 101- | ESIMS m/z | ¹H NMR (400 MHz, CDCl₃) δ 8.23 (s, 1H), 7.82 (d, J = 1.2 Hz, 1H), 7.69 (d, |
| | 103 | 375 | J = 7.7 Hz, 1H), 7.61 (d, J = 8.0 Hz, 1H), 4.03 (s, 3H) |
| | | ([M + H]⁺) | |
| F4 | 74- | ESIMS m/z | ¹H NMR (300 MHz, CDCl₃) δ 8.03 (s, 1H), 7.75 (d, J = 1.5 Hz, 1H), 7.65- |
| | 76 | 374 | 7.60 (m, 1H), 7.55 (d, J = 8.1 Hz, 1H), 4.00 (s, 3H), 3.28 (s, 1H) |
| | | ([M + H]⁺) | |
| F5 | 91- | ESIMS m/z | ¹H NMR (300 MHz, CDCl₃) δ 7.78-7.72 (m, 2H), 7.64 (d, J = 7.9 Hz, 1H), |
| | 93 | 364 | 7.47 (d, J = 8.0 Hz, 1H), 3.97 (s, 3H), 2.21 (s, 3H) |
| | | ([M + H]⁺) | |
| F6 | 78- | ESIMS m/z | ¹H NMR (300 MHz, CDCl₃) δ 8.40 (d, J = 0.7 Hz, 1H), 7.76 (s, 1H), 7.68- |
| | 80 | 476 | 7.61 (m, 1H), 7.45 (d, J = 7.8 Hz, 1H), 3.98 (s, 3H) |
| | | ([M + H]⁺) | |
| F7 | 148- | ESIMS m/z | ¹H NMR (300 MHz, CDCl₃) δ 7.75 (d, J = 1.6 Hz, 1H), 7.65-7.59 (m, 2H), |
| | 150 | 396 | 7.47 (d, J = 7.9 Hz, 1H), 3.97 (s, 3H), 2.47 (s, 3H) |
| | | ([M + H]⁺) | |
| F8 | 97- | ESIMS m/z | ¹H NMR (400 MHz, CDCl₃) δ 8.23 (d, J = 7.8 Hz, 1H), 7.94 (q, J = 8.4 Hz, |
| | 99 | 351 | 2H), 7.76 (d, J = 7.8 Hz, 1H), 4.03 (s, 3H) |
| | | ([M + H]⁺) | |
| F9 | 81- | ESIMS m/z | ¹H NMR (300 MHz, CDCl₃) δ 8.18 (d, J = 8.3 Hz, 1H), 7.97 (d, J = 8.2 Hz, |
| | 83 | 384 | 1H), 7.85-7.74 (m, 2H), 7.66 (dd, J = 1.4, 8.1 Hz, 1H), 4.03 (s, 3H) |
| | | ([M + H]⁺) | |
| F10 | 158- | ESIMS m/z | ¹H NMR (300 MHz, CDCl₃) δ 8.36 (dd, J = 1.2, 8.2 Hz, 1H), 8.18 (dd, J = |
| | 160 | 340 | 1.3, 8.2 Hz, 1H), 8.15-8.10 (m, 2H), 8.02 (dd, J = 1.8, 8.4 Hz, 1H), 4.12 (s, |
| | | ([M + H]⁺) | 3H) |
| F11 | 153- | ESIMS m/z | ¹H NMR (400 MHz, CDCl₃) δ 8.33 (s, 1H), 7.87 (d, J = 8.1 Hz, 1H), 7.83 (s, |
| | 155 | 375 | 1H), 7.73 (d, J = 7.9 Hz, 1H), 4.12 (s, 3H) |
| | | ([M + H]⁺) | |
| F12 | 112- | ESIMS m/z | ¹H NMR (300 MHz, CDCl₃) δ 8.21 (t, J = 7.9 Hz, 1H), 7.97-7.82 (m, 2H), |
| | 114 | 334 | 7.50 (dd, J = 9.4, 28.3 Hz, 2H), 4.04 (s, 3H) |
| | | ([M + H]⁺) | |
| F13 | 150- | ESIMS m/z | ¹H NMR (300 MHz, CDCl₃) δ 7.86 (dd, J = 0.8, 8.4 Hz, 1H), 7.76 (dd, J = |
| | 152 | 358 | 0.1, 8.4 Hz, 1H), 7.61 (d, J = 7.9 Hz, 1H), 7.34 (d, J = 1.7 Hz, 1H), 7.25- |
| | | ([M + H]⁺) | 7.14 (m, 1H), 4.01 (s, 3H), 2.77 (td, J = 8.0, 12.2 Hz, 1H), 2.26-1.74 (m, 1H), |
| | | | 1.74-1.59 (m, 1H) |
| F14 | 80- | ESIMS m/z | ¹H NMR (400 MHz, CDCl₃) δ 7.87 (d, J = 8.4 Hz, 1H), 7.77 (d, J = 8.3 Hz, |
| | 82 | 328 | 1H), 7.65 (d, J = 7.9 Hz, 1H), 7.47 (s, 1H), 7.33 (d, J = 7.8 Hz, 1H), 5.64 (dq, |
| | | ([M + H]⁺) | J = 6.4, 47.4 Hz, 1H), 4.01 (s, 3H), 1.64 (dd, J = 6.4, 23.9 Hz, 3H) |
| F15 | 89- | ESIMS m/z | ¹H NMR (300 MHz, CDCl₃) δ 7.83 (d, J = 8.8 Hz, 1H), 7.78-7.71 (m, 1H), |
| | 91 | 322 | 7.52 (d, J = 8.2 Hz, 1H), 7.15 (t, J = 1.9 Hz, 1H), 7.05 (dd, J = 1.8, 8.0 Hz, |
| | | ([M + H]⁺) | 1H), 4.00 (s, 3H), 1.91 (m, 1H), 1.09-0.89 (m, 2H), 0.75-0.72 (m, 2H) |
| F16 | 83- | ESIMS m/z | ¹H NMR (400 MHz, CDCl₃) δ 8.07 (d, J = 8.5 Hz, 1H), 7.95-7.86 (m, 3H), |
| | 85 | 340 | 7.74-7.66 (m, 1H), 4.02 (s, 3H), 3.25 (s, 1H) |
| | | ([M + H]⁺) | |
| F17 | 95- | ESIMS m/z | ¹H NMR (300 MHz, CDCl₃) δ 8.03 (d, J = 1.7 Hz, 1H), 7.91 (dd, J = 3.0, 8.2 |
| | 97 | 384 | Hz, 2H), 7.69 (d, J = 8.0 Hz, 1H), 7.53 (d, J = 8.3 Hz, 1H), 4.01 (s, 3H) |
| | | ([M + H]⁺) | |
| F18 | 168- | ESIMS m/z | ¹H NMR (300 MHz, CDCl₃) δ 7.87 (d, J = 8.4 Hz, 1H), 7.78 (d, J = 8.4 Hz, |
| | 170 | 306 | 1H), 7.65-7.58 (m, 2H), 7.48 (dd, J = 1.6, 7.9 Hz, 1H), 4.01 (s, 3H), 3.19 (s, |
| | | ([M + H]⁺) | 1H) |
| F19 | 150- | ESIMS m/z | ¹H NMR (300 MHz, CDCl₃) δ 7.90 (d, J = 8.5 Hz, 1H), 7.82-7.68 (m, 2H), |
| | 152 | 332 | 7.64 (m, 1H), 7.51 (dq, J = 1.1, 7.8 Hz, 1H), 6.67 (t, J = 56.0 Hz, 1H), 4.02 |
| | | ([M + H]⁺) | (s, 3H) |
| F20 | 118- | ESIMS m/z | ¹H NMR (300 MHz, CDCl₃) δ 7.86-7.75 (m, 2H), 7.68 (d, J = 1.6 Hz, 1H), |
| | 120 | 352 | 7.60 (d, J = 8.05 Hz, 1H), 7.48 (dd, J = 1.6, 8.1 Hz, 1H), 7.10 (s, 1H), 4.01 |
| | | ([M + H]⁺) | (s, 3H), 3.03 (s, 6H) |
| F21 | 109- | ESIMS m/z | ¹H NMR (300 MHz, CDCl₃) δ 8.57 (d, J = 1.5 Hz, 1H), 8.00 (dd, J = 1.6, 8.2 |
| | 111 | 339 | Hz, 1H), 7.85 (m, 2H), 7.71 (d, J = 8.1 Hz, 1H), 7.40 (s, 1H), 4.01 (s, 3H), |
| | | ([M + H]⁺) | 3.92 (s, 3H) |
| F22 | 130- | ESIMS m/z | ¹H NMR (300 MHz, CDCl₃) δ 10.03 (s, 1H), 7.99 (d, J = 1.4 Hz, 1H), 7.94- |
| | 132 | 310 | 7.88 (m, 1H), 7.87 (d, J = 1.4 Hz, 1H), 7.82 (dd, J = 4.4, 8.1 Hz, 2H), 4.02 (s, |
| | | ([M + H]⁺) | 3H) |
| F23 | 105- | ESIMS m/z | ¹H NMR (400 MHz, CDCl₃) δ 7.86 (d, J = 8.4 Hz, 1H), 7.78 (d, J = 8.4 Hz, |
| | 107 | 308 | 1H), 7.62 (d, J = 7.9 Hz, 1H), 7.50 (d, J = 1.7 Hz, 1H), 7.40 (dd, J = 1.6, 7.9 |
| | | ([M + H]⁺) | Hz, 1H), 6.69 (dd, J = 10.9, 17.6 Hz, 1H), 5.83 (d, J = 17.5 Hz, 1H), 5.38 (d, |
| | | | J = 10.8 Hz, 1H), 4.01 (s, 3H) |
| F24 | 98- | ESIMS m/z | ¹H NMR (400 MHz, CDCl₃) δ 8.09-8.04 (m, 2H), 8.00 (d, J = 8.4 Hz, 1H), |
| | 100 | 341 | 7.95 (m, 2H), 4.04 (s, 3H) |
| | | ([M + H]⁺) | |
| F25 | 135- | ESIMS m/z | ¹H NMR (300 MHz, CDCl₃) δ 7.92-7.79 (m, 2H), 7.72 (d, J = 8.05 Hz, |
| | 137 | 376 | 1H), 7.65 (d, J = 1.7 Hz, 1H), 7.60-7.51 (m, 3H), 7.20-7.10 (m, 2H), 4.02 |
| | | ([M + H]⁺) | (s, 3H) |
| F26 | 147- | ESIMS m/z | ¹H NMR (300 MHz, CDCl₃) δ 8.05 (d, J = 1.8 Hz, 1H), 7.96-7.86 (m, 2H), |
| | 149 | 324 | 7.78 (dd, J = 8.2, 13.3 Hz, 2H), 4.02 (s, 3H), 2.64 (s, 3H) |
| | | ([M + H]⁺) | |
| F27 | 140- | ESIMS m/z | ¹H NMR (400 MHz, CDCl₃) δ 7.87 (d, J = 8.28 Hz, 1H), 7.76 (d, J = 8.58 |
| | 142 | 362 | Hz, 1H), 7.65 (d, J = 1.96 Hz, 1H), 7.55-7.49 (m, 2H), 4.01 (s, 3H) |
| | | ([M + H]⁺) | |
| F28 | 125- | ESIMS m/z | ¹H NMR (300 MHz, CDCl₃) δ 7.98 (s, 1H), 7.95 (s, 1H), 7.82 (d, J = 8.5 Hz, |
| | 127 | 346 | 1H), 7.37-7.31 (m, 1H), 7.20 (s, 1H), 4.01 (s, 3H), 3.92 (s, 3H) |
| | | ([M + H]⁺) | |
| F29 | 80- | ESIMS m/z | ¹H NMR (400 MHz, CDCl₃) δ 7.90 (d, J = 8.5 Hz, 1H), 7.54 (s, 1H), |
| | 82 | 330 | 7.53-7.47 (m, 3H), 4.01 (s, 3H), 2.42 (s, 3H) |
| | | ([M + H]⁺) | |
| F30 | 70- | ESIMS m/z | ¹H NMR (400 MHz, CDCl₃) δ 7.94 (s, 1H), 7.91 (d, J = 8.4 Hz, 1H), 7.74 (d, |
| | 72 | 396 | J = 8.4 Hz, 1H), 7.72-7.65 (m, 2H), 4.02 (s, 3H) |
| | | ([M + H]⁺) | |
| F31 | 101- | ESIMS m/z | ¹H NMR (400 MHz, CDCl₃) δ 8.04 (d, J = 8.3 Hz, 1H), 7.84 (d, J = 8.2 Hz, |
| | 103 | 340 | 1H), 7.80 (d, J = 8.2 Hz, 1H), 7.75 (d, J = 1.5 Hz, 1H), 7.64 (dd, J = 1.6, 8.3 |
| | | ([M + H]⁺) | Hz, 1H), 4.02 (s, 3H), 3.59 (s, 1H) |
| F32 | 107- | ESIMS m/z | ¹H NMR (300 MHz, CDCl₃) δ 8.09 (d, J = 8.3 Hz, 1H), 7.80-7.74 (m, 2H), |
| | 109 | 394 | 7.70 (d, J = 8.4 Hz, 1H), 7.65-7.61 (m, 1H), 4.02 (s, 3H) |
| | | ([M + H]⁺) | |
| F33 | 61- | ESIMS m/z | ¹H NMR (300 MHz, CDCl₃) δ 7.75-7.72 (m, 2H), 7.67 (s, 1H), 7.62 (d, = |
| | 63 | 364 | 8.1 Hz, 1H), 4.01 (s, 3H), 2.53 (s, 3H) |
| | | ([M + H]⁺) | |
| F34 | 185- | ESIMS m/z | ¹H NMR (300 MHz, CDCl₃) δ 7.76 (s, 1H), 7.64 (d, J = 8.1 Hz, 1H), 7.56 (d, |
| | 187 | 369 | J = 7.9 Hz, 1H), 7.45 (s, 1H), 3.96 (s, 3H) |
| | | ([M + H]⁺) | |
| F35 | 93- | ESIMS m/z | ¹H NMR (300 MHz, CDCl₃) δ 7.83 (d, J = 8.50 Hz, 1H), 7.76 (d, J = 8.49 |
| | 95 | 312 | Hz, 1H), 7.60 (d, J = 8.63 Hz, 1H), 7.00 (d, J = 2.52 Hz, 1H), 6.91 (dd, J = |
| | | ([M + H]⁺) | 2.55, 8.63 Hz, 1H), 4.01 (s, 3H), 3.84 (s, 3H) |
| F36 | | ESIMS m/z | ¹H NMR (300 MHz, CDCl₃) δ 7.88-7.82 (m, 1H), 7.77 (dd, J = 0.78, 8.41 |
| | | 354 | Hz, 1H), 7.55 (dd, J = 0.80, 8.54 Hz, 1H), 7.11 (dd, J = 0.78, 2.22 Hz, 1H), |
| | | ([M + H]⁺) | 7.00 (m, 1H), 4.02 (s, 3H). 1.39 (s, 9H) |
| F37 | 112- | ESIMS m/z | ¹H NMR (300 MHz, CDCl₃) δ 7.87 (d, J = 8.4 Hz, 1H), 7.74 (d, J = 8.4 Hz, |
| | 114 | 300 | 1H), 7.64 (dd, J = 6.1, 8.5 Hz, 1H), 7.25-7.18 (m, 1H), 7.11 (t, J = 8.4 Hz, |
| | | ([M + H]⁺) | 1H), 4.01 (s, 3H) |
| F38 | 110- | ESIMS m/z | ¹H NMR (300 MHz, CDCl₃) δ 7.97-7.84 (m, 2H), 7.78 (d, J = 8.4 Hz, 1H), |
| | 112 | 350 | 7.61 (d, J = 1.4 Hz, 2H), 4.03 (s, 3H) |
| | | ([M + H]⁺) | |
| F39 | 76- | ESIMS m/z | ¹H NMR (400 MHz, CDCl₃) δ 7.80-7.73 (m, 2H), 7.65-7.59 (m, 1H), 7.33 |
| | 78 | 380 | (s, 1H), 4.03 (s, 3H), 4.00 (s, 3H) |
| | | ([M + H]⁺) | |
| F40 | 110- | ESIMS m/z | ¹H NMR (400 MHz, CDCl₃) δ 7.91 (s, 1H), 7.78-7.74 (m, 2H), 7.63 (dd, J = |
| | 112 | 374 | 1.5, 8.1 Hz, 1H), 4.02 (s, 3H), 3.71 (s, 1H) |
| | | ([M + H]⁺) | |
| F41 | 113- | ESIMS m/z | ¹H NMR (300 MHz, CDCl₃) δ 8.09 (s, 1H), 7.77 (d, J = 7.0 Hz, 2H), 7.67- |
| | 115 | 430 | 7.59 (m, 1H), 4.02 (s, 3H) |
| | | ([M + H]⁺) | |
| F42 | 65- | ESIMS m/z | ¹H NMR (400 MHz, CDCl₃) δ 7.77 (d, J = 1.5 Hz, 1H), 7.64 (dd, J = 1.6, 7.9 |
| | 67 | 399 | Hz, 1H), 7.55 (d, J = 7.9 Hz, 1H), 3.99 (s, 3H), 3.69 (s, 3H) |
| | | ([M + H]⁺) | |
| F43 | 86- | ESIMS m/z | ¹H NMR (300 MHz, CDCl₃) δ 7.78 (s, 1H), 7.69-7.64 (m, 1H), 7.61 (d, J = |
| | 88 | 402 | 8.0 Hz, 1H), 4.01 (s, 3H) |
| | | ([M + H]⁺) | |
| F44 | 56- | ESIMS m/z | ¹H NMR (400 MHz, DMSO-d₆) δ 8.29 (d, J = 8.5 Hz, 1H), 8.07-8.03 (m, |
| | 57 | 426 | 1H), 7.97 (d, J = 8.5 Hz, 1H), 7.91-7.85 (m, 1H), 7.83 (d, J = 8.1 Hz, 1H), |
| | | ([M + H]⁺) | 7.51-7.46 (m, 2H), 7.44-7.32 (m, 3H), 5.45 (s, 2H); |
| | | | ¹⁹F NMR (376 MHz, DMSO-d₆) δ -61.32 |
| F45 | 76- | ESIMS m/z | ¹H NMR (400 MHz, DMSO-d₆) δ 8.31 (d, J = 8.5 Hz, 1H), 8.09-8.05 (m, |
| | 77 | 374 | 1H), 7.99 (d, J = 8.5 Hz, 1H), 7.91-7.86 (m, 1H), 7.83 (d, J = 8.1 Hz, 1H), |
| | | ([M + H]⁺) | 5.06 (d, J = 2.5 Hz, 2H), 3.69 (t, J = 2.4 Hz, 1H); |
| | | | ¹⁹F NMR (376 MHz, DMSO-d₆) δ -61.32 |
| F46 | 125- | ESIMS m/z | ¹H NMR (300 MHz, CDCl₃) δ 7.87 (d, J = 8.4 Hz, 1H), 7.79 (d, J = 8.4 Hz, |
| | 127 | 314 | 1H), 7.43-7.27 (m, 2H), 4.02 (s, 3H), 2.30 (s, 3H) |
| | | ([M + H]⁺) | |
| F47 | 172- | ESIMS m/z | ¹H NMR (300 MHz, CDCl₃) δ 7.88 (d, J = 8.4 Hz, 1H), 7.79 (d, J = 8.5 Hz, |
| | 174 | 348 | 1H), 7.49 (s, 1H), 7.20 (s, 1H), 4.02 (s, 3H), 3.94 (s, 3H) |
| | | ([M + H]⁺) | |
| F48 | 110- | ESIMS m/z | ¹H NMR (300 MHz, CDCl₃) δ 7.87 (d, J = 8.4 Hz, 1H), 7.67 (d, J = 8.4 Hz, |
| | 112 | 330 | 1H), 7.40 (d, J = 8.4 Hz, 1H), 7.34 (d, J = 8.4 Hz, 1H), 4.01 (s, 3H), 2.54 (s, |
| | | ([M + H]⁺) | 3H) |
| F49 | 88- | ESIMS m/z | ¹H NMR (300 MHz, CDCl₃) δ 7.83 (d, J = 8.4 Hz, 1H), 7.76 (d, J = 8.4 Hz, |
| | 90 | 310 | 1H), 7.41 (s, 1H), 7.23 (s, 1H), 4.00 (d, J = 3.4 Hz, 3H), 2.27 (s, 6H) |
| | | ([M + H]⁺) | |
| F50 | 160- | ESIMS m/z | ¹H NMR (300 MHz, CDCl₃) δ 7.94 (d, J = 8.5 Hz, 1H), 7.85 (s, 1H), 7.82 (d, |
| | 162 | 341 | J = 8.4 Hz, 1H), 7.79 (s, 1H), 4.04 (s, 3H) |
| | | ([M + H]⁺) | |
| F51 | 129- | ESIMS m/z | ¹H NMR (400 MHz, CDCl₃) δ 7.86 (d, J = 8.3 Hz, 1H), 7.75 (d, J = 8.3 Hz, |
| | 131 | 331 | 1H), 7.52 (s, 1H), 7.48 (s, 1H), 4.02 (s, 3H), 2.39 (s, 3H) |
| | | ([M + H]⁺) | |
| F52 | 129- | ESIMS m/z | ¹H NMR (400 MHz, CDCl₃) δ 7.69 (d, J = 1.9 Hz, 2H), 7.59 (d, J = 8.3 Hz, |
| | 131 | 296 | 1H), 7.48 (d, J = 1.9 Hz, 1H), 7.35 (dd, J = 2.1, 8.2 Hz, 1H), 3.98 (s, 3H), |
| | | ([M + H]⁺) | 2.62 (s, 3H) |
| F53 | 105- | ESIMS m/z | ¹H NMR (400 MHz, CDCl₃) δ 7.77 (d, J = 7.9 Hz, 1H), 7.73 (t, J = 1.2 Hz, |
| | 107 | 330 | 3H), 7.62 (dd, J = 1.6, 8.2 Hz, 1H), 3.98 (s, 3H), 2.64 (s, 3H) |
| | | ([M + H]⁺) | |
| F54 | | ESIMS m/z | ¹H NMR (500 MHz, CDCl₃) δ 8.64-8.60 (m, 1H), 7.92 (d, J = 8.4 Hz, 1H), |
| | | 427.1 | 7.83-7.79 (m, 2H), 7.76 (d, J = 1.7 Hz, 1H), 7.73 (td, J = 7.7, 1.8 Hz, 1H), |
| | | ([M + H]⁺) | 7.64 (ddd, J = 8.1, 1.8, 0.8 Hz, 1H), 7.53 (dt, J = 7.8, 1.0 Hz, 1H), 7.28- |
| | | | 7.23 (m, 1H), 5.58 (s, 2H); |
| | | | ¹⁹F NMR (471 MHz, CDCl₃) δ -62.91 |
| F55 | 85- | ESIMS m/z | ¹H NMR (400 MHz, CDCl₃) δ 8.19 (dd, J = 1.2, 7.8 Hz, 1H), 8.01-7.93 (m, |
| | 88 | 316 | 1H), 7.85 (dd, J = 1.2, 7.8 Hz, 1H), 7.82-7.73 (m, 2H), 7.64 (dd, J = 1.6, |
| | | ([M + H]⁺) | 8.0 Hz, 1H), 4.02 (s, 3H) |
| F56 | 125- | ESIMS m/z | ¹H NMR (300 MHz, CDCl₃) δ 8.14 (d, J = 1.8 Hz, 1H), 7.83 (d, J = 1.8 Hz, |
| | 127 | 316 | 1H), 7.59 (d, J = 8.3 Hz, 1H), 7.50 (d, J = 2.0 Hz, 1H), 7.37 (dd, J = 2.0, 8.3 |
| | | ([M + H]⁺) | Hz, 1H), 4.02 (s, 3H) |
| F57 | 133- | ESIMS m/z | ¹H NMR (300 MHz, CDCl₃) δ 8.19 (d, J = 1.9 Hz, 1H), 7.85 (d, J = 1.8 Hz, |
| | 135 | 350 | 1H), 7.81-7.73 (m, 2H), 7.64 (dt, J = 1.3, 6.9 Hz, 1H), 4.02 (s, 3H) |
| | | ([M + H]⁺) | |
| F58 | 90- | ESIMS m/z | ¹H NMR (300 MHz, CDCl₃) δ 7.89 (d, J = 8.4 Hz, 1H), 7.76 (d, J = 8.4 Hz, |
| | 92 | 366 | 1H), 7.69 (d, J = 8.5 Hz, 1H), 7.37 (dd, J = 1.0, 2.2 Hz, 1H), 7.29-7.23 (m, |
| | | ([M + H]⁺) | 1H), 4.02 (s, 3H) |
| F59 | 140- | ESIMS m/z | ¹H NMR (300 MHz, CDCl₃) δ 7.92 (d, J = 8.4 Hz, 1H), 7.83-7.73 (m, 3H), |
| | 142 | 307 | 7.69-7.63 (m, 1H), 4.02 (s, 3H) |
| | | ([M + H]⁺) | |
| F60 | 100- | ESIMS m/z | ¹H NMR (400 MHz, CDCl₃) δ 7.84 (d, J = 8.3 Hz, 1H), 7.75 (d, J = 8.4 Hz, |
| | 102 | 296 | 1H), 7.53 (d, J = 7.8 Hz, 1H), 7.29 (d, J = 1.5 Hz, 1H), 7.17 (dd, J = 1.5, 7.9 |
| | | ([M + H]⁺) | Hz, 1H), 4.00 (s, 3H), 2.38 (s, 3H) |
| F61 | 101- | ESIMS m/z | ¹H NMR (400 MHz, DMSO-d₆) δ 14.00 (s, 1H), 8.24 (d, J = 8.5 Hz, 1H), |
| | 103 | 336 | 8.08-8.03 (m, 1H), 7.91 (d, J = 8.4 Hz, 1H), 7.89-7.82 (m, 2H); |
| | | ([M + H]⁺) | ¹⁹F NMR (376 MHz, DMSO-d₆) δ -61.31 |
| F62 | 106- | ESIMS m/z | ¹H NMR (300 MHz, CDCl₃) δ 7.92 (d, J = 8.3 Hz, 1H), 7.47 (dd, J = 0.8, 8.2 |
| | 108 | 334 | Hz, 1H), 7.34 (t, J = 1.7 Hz, 1H), 7.15 (dd, J = 1.9, 8.8 Hz, 1H), 4.01 (s, 3H) |
| | | ([M + H]⁺) | |
| F63 | 165- | ESIMS m/z | ¹H NMR (400 MHz, CDCl₃) δ 7.90 (d, J = 8.0 Hz, 1H), 7.75 (d, J = 8.0, 1H), |
| | 167 | 334 | 7.42 (m, 2H), 4.05 (s, 3H) |
| | | ([M + H]⁺) | |
| F64 | | ESIMS m/z | ¹H NMR (400 MHz, CDCl₃) δ 7.83 (d, J = 8.4 Hz, 1H), 7.77 (d, J = 1.7 Hz, |
| | | 328 | 1H), 7.70 (dd, J = 2.0, 8.4 Hz, 2H), 7.13 (d, J = 8.4 Hz, 1H), 4.02 (s, 3H) |
| | | ([M + H]⁺) | |
| F65 | 185- | ESIMS m/z | ¹H NMR (400 MHz, CDCl₃) δ 7.89 (d, J = 8.4 Hz, 1H), 7.80 (d, J = 8.4 Hz, |
| | 187 | 334 | 1H), 7.58-7.47 (m, 2H), 4.03 (s, 3H) |
| | | ([M + H]⁺) | |
| F66 | 113- | ESIMS m/z | ¹H NMR (400 MHz, DMSO-d₆) δ 8.29 (d, J = 8.5 Hz, 1H), 8.06 (d, J = 1.7 |
| | 114 | 350 | Hz, 1H), 7.97 (d, J = 8.5 Hz, 1H), 7.88 (ddd, J = 8.1, 1.8, 0.7 Hz, 1H), 7.83 |
| | | ([M + H]⁺) | (dd, J = 8.1, 0.8 Hz, 1H), 3.93 (s, 3H); |
| | | | ¹⁹F NMR (376 MHz, DMSO-d₆) δ -61.32 |
| F67 | | ESIMS m/z | ¹H NMR (400 MHz, DMSO-d₆) δ 14.11 (s, 1H), 8.24 (d, J = 8.5 Hz, 1H), |
| | | 348 | 8.02 (d, J = 8.5 Hz, 1H), 7.95 (d, J = 8.9 Hz, 1H), 7.51 (d, J = 8.9 Hz, 1H); |
| | | ([M + H]⁺) | ¹⁹F NMR (376 MHz, DMSO-d₆) δ -47.95 |
| F68 | | ESIMS m/z | ¹H NMR (400 MHz, CDCl₃) δ 7.99 (m, 2H), 7.89 (d, J = 8.5 Hz, 1H), 7.21 |
| | | 363 | (d, J = 8.9 Hz, 1H), 4.04 (s, 3H); |
| | | ([M + H]⁺) | ¹⁹F NMR (376 MHz, CDCl₃) δ -49.01 |
| F69 | 95- | ESIMS m/z | ¹H NMR (500 MHz, CDCl₃) δ 8.47 (s, 1H), 7.92 (dd, J = 8.6, 1.7 Hz, 1H), |
| | 98 | 305 | 7.83 (d, J = 8.5 Hz, 1H), 7.75 (dd, J = 8.3, 7.0 Hz, 1H), 7.49 (d, J = 8.3 Hz, |
| | | ([M + H]⁺) | 1H), 7.31 (t, J = 2.8 Hz, 1H), 6.61 (td, J = 3.4, 2.1 Hz, 1H), 4.03 (s, 3H); |
| | | | ¹⁹F NMR (471 MHz, CDCl₃) δ -139.06 |
| F70 | 155- | ESIMS m/z | ¹H NMR (400 MHz, DMSO-d₆) δ 8.23 (d, J = 8.5 Hz, 1H), 7.96 (dd, J = 8.5, |
| | 157 | 303 | 2.0 Hz, 1H), 7.79 (td, J = 8.2, 7.6, 2.1 Hz, 1H), 7.60 (ddd, J = 8.7, 6.8, 1.9 |
| | | ([M − H]⁻) | Hz, 1H); |
| | | | ¹⁹F NMR (376 MHz, DMSO-d₆) |
| | | | δ -138.52, -138.57, -138.98, -139.04 |
| F71 | 150- | ESIMS m/z | ¹H NMR (400 MHz, CDCl₃) δ 7.88 (m, 2H), 7.83 (m, 1H), 7.30 (m, 1H), |
| | 152 | 319 | 4.03 (s, 3H); |
| | | ([M + H]⁺) | ¹⁹F NMR (376 MHz, CDCl₃) |
| | | | δ -137.83, -137.88, -138.60, -138.65 |
| F72 | 197- | ESIMS m/z | ¹H NMR (400 MHz, CDCl₃) δ 7.67-7.55 (m, 2H), 7.54-7.38 (m, 2H), 7.11 |
| | 200 | 296 | (s, 1H), 4.22 (s, 3H), 3.94 (s, 2H) |
| | | ([M − H]⁻) | |
| F73 | 162- | ESIMS m/z | ¹H NMR (300 MHz, DMSO-d₆) δ 7.52 (br d, J = 8 Hz, 1H), 7.38 (br t, J = 8 |
| | 163 | 368 | Hz, 1H), 3.93 (s, 3H) |
| | | ([M + H]⁺) | |
| F74 | 130- | ESIMS m/z | ¹H NMR (300 MHz, CDCl₃) δ 7.31 (dd, J = 8, 1 Hz, 1H), 7.25 (dd, J = 8, 6 |
| | 133 | 382 | Hz, 1H), 4.01 (s, 3H), 4.00 (d, J = 1.5 Hz, 3H) |
| | | ([M + H]⁺) | |
| F75 | 141- | ESIMS m/z | ¹H NMR (300 MHz, DMSO-d₆) δ 8.22 (d, J = 1 Hz, 1H), 7.64 (dd, J = 9, 8 |
| | 144 | 350 | Hz, 1H), 7.50 (dd, J = 9, 2 Hz, 1H), 3.95 (d, J = 1 Hz, 3H) |
| | | ([M + H]⁺) | |
| F76 | 97- | ESIMS m/z | ¹H NMR (300 MHz, CDCl₃) δ 8.01 (d, J = 1 Hz, 1H), 7.71 (dd, J = 9, 8 Hz, |
| | 99 | 364 | 1H), 7.27 (dd, J = 9, 2 Hz, 1H), 4.03 (s, 3H), 4.00 (d, J = 1 Hz, 3H) |
| | | ([M + H]⁺) | |
| F77 | 85- | ESIMS m/z | ¹H NMR (300 MHz, CDCl₃) δ 7.70 (dd, J = 8.6, 7.9 Hz, 1H), 7.28-7.21 (m, |
| | 86 | 369 | 2H), 6.09 (s, 2H), 4.46 (q, J = 7.1 Hz, 2H), 4.23 (q, J = 7.0 Hz, 2H), 3.98 (d, |
| | | ([M + H]⁺) | J = 1.0 Hz, 3H), 1.54 (t, J = 7.0 Hz, 3H), 1.48 (t, J = 7.1 Hz, 3H) |
| F78 | | ESIMS m/z | ¹H NMR (400 MHz, DMSO-d₆) δ 14.02 (s, 1H), 8.20 (d, J = 8.5 Hz, 1H), |
| | | 316 | 7.94 (dd, J = 8.5, 2.1 Hz, 1H), 7.65 (t, J = 8.2 Hz, 1H), 7.48 (dd, J = 8.6, 1.7 |
| | | ([M + H]⁺) | Hz, 1H), 4.15-3.81 (m, 3H); |
| | | | ¹⁹F NMR (376 MHz, DMSO-d₆) δ -131.57 |
| F79 | | EIMS m/z | ¹H NMR (400 MHz, CDCl₃) δ 8.75 (d, J = 2.1 Hz, 1H), 8.66-8.56 (m, 1H), |
| | | 426.1 | 7.90 (d, J = 8.4 Hz, 1H), 7.85-7.74 (m, 4H), 7.63 (dd, J = 8.2, 1.7 Hz, 1H), |
| | | | 7.33 (dd, J = 7.9, 4.8 Hz, 1H), 5.48 (s, 2H); |
| | | | ¹⁹F NMR (376 MHz, CDCl₃) δ -62.95 |
| F80 | 115- | ESIMS m/z | ¹H NMR (400 MHz, DMSO-d₆) δ 8.57 (s, 1H), 8.20-8.00 (m, 2H), 7.65- |
| | 116 | 316 | 7.49 (m, 2H), 3.97 (s, 3H) |
| | | ([M + H]⁺) | |
| F81 | | EIMS m/z | ¹H NMR (400 MHz, CDCl₃) δ 8.66-8.60 (m, 2H), 7.94 (d, J = 8.4 Hz, 1H), |
| | | 426.1 | 7.86-7.75 (m, 3H), 7.69-7.62 (m, 1H), 7.41-7.36 (m, 2H), 5.47 (s, 2H); |
| | | | ¹⁹F NMR (376 MHz, CDCl₃) δ -62.93 |
| F82 | 60- | ESIMS m/z | ¹H NMR (400 MHz, DMSO-d₆) δ 8.21 (s, 2H), 8.15-8.09 (m, 2H), 7.62- |
| | 63 | 282 | 7.56 (m, 2H), 3.95 (s, 3H) |
| | | ([M + H]⁺) | |
| F83 | 149- | ESIMS m/z | ¹H NMR (300 MHz, CDCl₃) δ 8.54 (d, J = 2.1 Hz, 1H), 8.18 (dd, J = 8.5, 2.2 |
| | 156 | 327 | Hz, 1H), 7.93 (d, J = 8.5 Hz, 1H), 7.82 (d, J = 8.5 Hz, 1H), 7.66 (d, J = 8.5 |
| | | ([M + H]⁺) | Hz, 1H), 4.05 (s, 3H); |
| | | | ¹³C NMR (126 MHz, CDCl₃) δ 164.79, 151.46, 148.43, 148.37, 139.64, 137.08, |
| | | | 132.42, 131.01, 130.30, 128.26, 123.88, 122.52, 53.16 |
| F84 | | ESIMS m/z | ¹H NMR (400 MHz, CDCl₃) δ 9.83 (s, 1H), 9.12 (s, 1H), 8.28 (d, J = 7.8 Hz, |
| | | 337 | 1H), 8.13 (s, 1H), 7.84 (d, J = 7.2 Hz, 1H); |
| | | ([M + H]⁺) | ¹³C NMR (101 MHz, CDCl₃) δ 161.2, 153.0, 142.4, 142.0, 139.3, 133.2, 132.5, |
| | | | 132.2, 126.3, 125.6, 125.1, 122.4 |
| F85 | | ESIMS m/z | ¹H NMR (400 MHz, CDCl₃) δ 7.82 (d, J = 8.5 Hz, 1H), 7.77 (dd, J = 8.5, 1.9 |
| | | 310 | Hz, 1H), 7.56 (dd, J = 8.3, 7.2 Hz, 1H), 6.76 (d, J = 8.3 Hz, 1H), 6.10 (s, |
| | | ([M + H]⁺) | 2H), 4.02 (s, 3H); |
| | | | ¹³C NMR (126 MHz, CDCl₃) δ 165.09, 151.30 (d, J = 5.1 Hz), 151.07 (d, J = |
| | | | 2.3 Hz), 147.66, 145.06 (d, J = 250.7 Hz), 138.84, 134.94 (d, J = 13.9 Hz), |
| | | | 128.78, 126.18 (d, J = 10.1 Hz), 124.29 (d, J = 1.6 Hz), 120.92 (d, J = 8.6 |
| | | | Hz), 105.24 (d, J = 3.4 Hz), 102.87, 53.00; |
| | | | ¹⁹F NMR (471 MHz, CDCl₃) δ -142.39 (dd, J = 7.2, 2.1 Hz) |
| F86 | | EIMS m/z | ¹H NMR (500 MHz, CDCl₃) δ 8.40 (d, J = 4.9 Hz, 1H), 7.95 (d, J = 8.4 Hz, |
| | | 316.1 | 1H), 7.80 (d, J = 8.4 Hz, 1H), 7.51 (d, J = 4.9 Hz, 1H), 4.03 (s, 3H) |
| F87 | | ESIMS m/z | ¹H NMR (400 MHz, DMSO-d₆) δ 13.58 (s, 1H), 9.23 (d, J = 2.0 Hz, 1H), |
| | | 333 | 8.54 (dd, J = 8.3, 2.1 Hz, 1H), 8.03 (d, J = 8.2 Hz, 1H), 7.94 (s, 1H), 4.00 (s, |
| | | ([M + H]⁺) | 3H); |
| | | | ¹³C NMR (101 MHz, DMSO-d₆) δ 165.6, 155.8, 150.6, 146.6, 146.3, 140.8, |
| | | | 140.3, 138.9, 135.2, 131.1, 122.7, 120.8, 57.6 |
| F88 | | ESIMS m/z | ¹H NMR (400 MHz, CDCl₃) δ 7.97 (d, J = 8.3 Hz, 1H), 7.68 (s, 2H), 7.42 (d, |
| | | 384 | J = 8.3 Hz, 1H), 4.01 (s, 3H); |
| | | ([M + H]⁺) | ¹³C NMR (126 MHz, CDCl₃) δ 164.46, 151.98, 147.92, 139.90, 139.36, |
| | | | 135.70, 132.98 (q, J = 34.2 Hz), 130.95, 127.61, 125.36 (q, J = 3.7 Hz), 122.38 |
| | | | (q, J = 273.3 Hz), 53.28; |
| | | | ¹⁹F NMR (471 MHz, CDCl₃) δ -63.17 |
| F89 | | ESIMS m/z | ¹H NMR (400 MHz, DMSO-d₆) δ 14.12 (s, 1H), 8.54 (s, 1H), 7.91-7.81 (m, |
| | | 368 | 4H); |
| | | ([M + H]⁺) | ¹³C NMR (101 MHz, DMSO-d₆) δ 165.5, 152.8, 148.0, 140.4, 139.5, 136.2, |
| | | | 131.6, 131.3, 128.5, 128.4, 124.9 |
| F90 | | ESIMS m/z | ¹H NMR (400 MHz, CDCl₃) δ 7.90 (d, J = 8.4 Hz, 1H), 7.83-7.76 (m, 2H), |
| | | 383 | 7.71 (d, J = 8.0 Hz, 1H), 7.66 (dd, J = 8.1, 1.7 Hz, 1H), 4.02 (s, 3H); |
| | | ([M + H]⁺) | ¹³C NMR (101 MHz, CDCl₃) δ 164.7, 153.3, 147.9, 139.5, 138.5, 137.2, |
| | | | 134.6, 132.9, 132.6, 130.7, 130.3, 127.6, 127.4, 126.7, 53.1 |
| F91 | | ESIMS m/z | ¹H NMR (400 MHz, CDCl₃) δ 8.04 (dd, J = 8.4, 1.6 Hz, 2H), 7.77 (d, J = 8.2 |
| | | 366 | Hz, 2H), 7.68 (d, J = 9.9 Hz, 1H), 4.02 (s, 3H); |
| | | ([M + H]⁺) | ¹³C NMR (101 MHz, CDCl₃) δ 164.0, 158.6, 155.9, 143.7 (d, J = 5.0 Hz), |
| | | | 142.8 (d, J = 11.0 Hz), 136.2, 135.8 (d, J = 5.9 Hz), 130.8 (d, J = 4.7 Hz), |
| | | | 129.74 (d, J = 6.2 Hz), 127.9, 126.9, 126.8, 126.5, 53.1 |
| F92 | | ESIMS m/z | ¹H NMR (400 MHz, CDCl₃) δ 9.33 (s, 1H), 8.51 (d, J = 8.3 Hz, 1H), 7.82 (d, |
| | | 335 | J = 8.2 Hz, 1H), 7.75 (d, J = 9.8 Hz, 1H), 4.03 (s, 3H); |
| | | ([M + H]⁺) | ¹³C NMR (101 MHz, CDCl₃) δ 163.8, 158.8, 156.1, 149.8 (d, J = 8.9 Hz), |
| | | | 144.3 (d, J = 5.1 Hz), 139.9 (d, J = 11.9 Hz), 137.5 (d, J = 5.4 Hz), 132.0 |
| | | | (dd, J = 32.1, 5.5 Hz), 127.3, 127.03, 120.3 (d, J = 3.0 Hz), 53.2 |
| F93 | | ESIMS m/z | ¹H NMR (400 MHz, CDCl₃) δ 8.15-8.07 (m, 2H), 7.75 (d, J = 8.2 Hz, 2H), |
| | | 334 | 7.69 (d, J = 9.9 Hz, 1H), 4.03 (s, 3H); |
| | | ([M + H]⁺) | ¹³C NMR (101 MHz, CDCl₃) δ 164.1, 158.6, 155.9, 143.7 (d, J = 4.9 Hz), |
| | | | 142.7 (d, J = 11.3 Hz), 136.8 (d, J = 5.9 Hz), 130.9 (d, J = 4.7 Hz), 129.2, |
| | | | 129.2, 127.0, 126.8, 125.6 (q, J = 3.8 Hz), 53.1 |
| F94 | | ESIMS m/z | ¹H NMR (400 MHz, CDCl₃) δ 8.04 (d, J = 8.5 Hz, 1H), 8.02-7.97 (m, 2H), |
| | | 334 | 7.96 (d, J = 8.5 Hz, 1H), 7.84-7.79 (m, 2H); |
| | | ([M + H]⁺) | ¹³C NMR (101 MHz, CDCl₃) δ 161.3, 153.1, 142.4, 142.0, 138.3, 136.7, |
| | | | 133.1, 130.9, 127.9, 127.1, 127.1, 125.5 |
| F95 | | ESIMS m/z | ¹H NMR (400 MHz, DMSO-d₆) δ 9.44 (d, J = 2.1 Hz, 1H), 8.74 (dd, J = 8.3, |
| | | 303 | 2.2 Hz, 1H), 8.34 (d, J = 8.6 Hz, 1H), 8.28 (d, J = 8.5 Hz, 1H), 8.07 (d, J = |
| | | ([M + H]⁺) | 8.3 Hz, 1H) |
| | | | ¹³C NMR (101 MHz, DMSO-d₆) δ 166.2, 150.9, 150.2, 148.9, 140.3, 136.9, |
| | | | 135.9, 128.7, 124.5, 123.4, 121.4, 120.7 |
| F96 | | ESIMS m/z | ¹H NMR (400 MHz, CDCl₃) δ 8.12 (s, 1H), 7.84 (d, J = 8.3 Hz, 2H), 7.79 (d, |
| | | 336 | J = 8.4 Hz, 2H); |
| | | ([M + H]⁺) | ¹³C NMR (101 MHz, CDCl₃) δ 152.4, 142.5, 138.6, 134.7, 133.1, 132.2, |
| | | | 131.9, 129.7, 125.5, 125.1, 122.4 |
| F97 | | ESIMS m/z | ¹H NMR (400 MHz, CDCl₃) δ 8.04 (d, J = 8.4 Hz, 2H), 7.88 (d, J = 8.5 Hz, |
| | | 348 | 1H), 7.80 (d, J = 8.5 Hz, 1H), 7.76 (d, J = 8.3 Hz, 2H), 4.04 (s, 3H); |
| | | ([M + H]⁺) | ¹³C NMR (101 MHz, CDCl₃) δ 165.0, 153.9, 148.1, 139.6, 139.3, 136.6, |
| | | | 131.0, 129.7, 128.0, 126.0, 123.0, 53.0 |
| F98 | | ESIMS m/z | ¹H NMR (400 MHz, CDCl₃) δ 7.98 (s, 1H), 7.80 (d, J = 8.4 Hz, 2H), 7.75 (d, |
| | | 382 | J = 8.2 Hz, 2H), 4.00 (s, 3H); |
| | | ([M + H]⁺) | ¹³C NMR (101 MHz, CDCl₃) δ 164.1, 153.0, 145.7, 140.1, 138.6, 135.9, |
| | | | 132.2, 131.0, 130.6, 130.1, 127.9, 126.0, 126.0, 53.2 |
| F99 | | ESIMS m/z | ¹H NMR (400 MHz, CDCl₃) δ 9.13-9.09 (m, 1H), 8.27 (dd, J = 8.1, 2.1 Hz, |
| | | 351 | 1H), 8.03 (s, 1H), 7.84-7.76 (m, 1H), 4.01 (s, 3H); |
| | | ([M + H]⁺) | ¹³C NMR (101 MHz, CDCl₃) δ 163.8, 150.4, 150.1, 146.2, 140.3, 138.4, |
| | | | 136.1, 134.9, 132.5, 131.0, 120.0 (q, J = 2.5 Hz), 53.3 |
| F100 | 80- | ESIMS m/z | ¹H NMR (400 MHz, CDCl₃) δ 7.86-7.79 (m, 2H), 7.72 (dd, J = 8.6, 1.6 Hz, |
| | 81 | 392 | 2H), 7.63 (d, J = 10.0 Hz, 1H), 4.01 (s, 3H); |
| | | ([M + H]⁺) | ¹⁹F NMR (376 MHz, CDCl₃) |
| | | | δ -116.13 |
| F101 | | ESIMS m/z | ¹H NMR (400 MHz, CDCl₃) δ 8.86 (d, J = 1.8 Hz, 1H), 8.09 (d, J = 1.9 Hz, |
| | | 351 | 1H), 7.98 (d, J = 8.4 Hz, 1H), 7.92 (d, J = 8.4 Hz, 1H), 4.02 (s, 3H); |
| | | ([M + H]⁺) | ¹⁹F NMR (376 MHz, CDCl₃) δ -62.39 |
| F102 | 58- | ESIMS m/z | ¹H NMR (400 MHz, CDCl₃) δ 8.00 (s, 1H), 7.76-7.59 (m, 3H), 4.01 (s, |
| | 61 | 368 | 3H); |
| | | ([M + H]⁺) | ¹⁹F NMR (376 MHz, CDCl₃) |
| | | | δ -61.49, -61.52, -113.63, -113.66 |
| F103 | 85- | ESIMS m/z | ¹H NMR (400 MHz, CDCl₃) δ 8.00 (s, 1H), 7.76 (d, J = 1.7 Hz, 1H), 7.65 |
| | 86 | 384 | (ddd, J = 8.0, 1.7, 0.8 Hz, 1H), 7.55-7.48 (m, 1H), 3.99 (s, 3H); |
| | | ([M + Na]⁺) | ¹⁹F NMR (376 MHz, CDCl₃) δ -62.98 |
| F104 | | ESIMS m/z | ¹H NMR (400 MHz, CDCl₃) δ 8.74 (s, 1H), 7.87 (d, J = 8.0 Hz, 1H), 7.76 (d, |
| | | 347 | J = 1.7 Hz, 1H), 7.69-7.54 (m, 1H), 4.10 (s, 3H), 4.01 (s, 3H); |
| | | ([M + H]⁺) | ¹⁹F NMR (376 MHz, CDCl₃) δ -62.94 |
| F105 | | ESIMS m/z | ¹H NMR (400 MHz, CDCl₃) δ 9.01 (s, 1H), 8.00-7.88 (m, 1H), 7.86-7.74 |
| | | 351 | (m, 1H), 7.74-7.58 (m, 1H), 4.06 (s, 3H); |
| | | ([M + H]⁺) | ¹⁹F NMR (376 MHz, CDCl₃) δ -63.06 |
| F106 | 133- | ESIMS m/z | ¹H NMR (400 MHz, CDCl₃) δ 7.99 (s, 1H), 7.91-7.83 (m, 2H), 7.80-7.69 |
| | 134 | 351 | (m, 2H), 4.00 (s, 3H); |
| | | ([M + H]⁺) | ¹⁹F NMR (376 MHz, DMSO-d₆) δ 61.26 |
| F107 | 141- | ESIMS m/z | ¹H NMR (400 MHz, DMSO-d₆) δ 8.55 (d, J = 9.0 Hz, 1H), 8.12 (d, J = 1.7 |
| | 142 | 368 | Hz, 1H), 7.93 (dd, J = 8.2, 1.8 Hz, 1H), 7.84 (d, J = 8.0 Hz, 1H), 3.92 (s, |
| | | ([M + H]⁺) | 3H); |
| | | | ¹⁹F NMR (376 MHz, DMSO-d₆) |
| | | | δ -61.40, -114.46 |
| F108 | 90- | ESIMS m/z | ¹H NMR (400 MHz, CDCl₃) δ 7.94 (s, 1H), 7.82-7.72 (m, 2H), 7.64 (ddd, J = |
| | 91 | 384 | 8.0, 1.8, 0.8 Hz, 1H), 4.03 (s, 3H); |
| | | ([M + H]⁺) | ¹⁹F NMR (376 MHz, CDCl₃) δ -63.01 |
| F109 | 127- | EIMS m/z | ¹H NMR (400 MHz, CDCl₃) δ 7.84-7.78 (m, 1H), 7.78-7.75 (m, 1H), 7.68 |
| | 128 | 368 | (d, J = 8.8 Hz, 1H), 7.67-7.62 (m, 1H), 4.03 (s, 3H); |
| | | | ¹⁹F NMR (376 MHz, CDCl₃) |
| | | | δ -63.02, -100.76 |
| F110 | 72- | ESIMS m/z | ¹H NMR (400 MHz, CDCl₃) δ 7.99-7.88 (m, 3H), 7.53-7.44 (m, 1H), 4.04 |
| | 73 | 352 | (s, 3H); |
| | | ([M + H]⁺) | ¹⁹F NMR (376 MHz, CDCl₃) |
| | | | δ -61.21, -61.25, -138.28, -138.31, -138.33, -138.35, -138.36, -138.38, -138.40, -138.43, |
| | | | -139.96, -140.01 |
| F111 | | ESIMS m/z | ¹H NMR (400 MHz, DMSO-d₆) δ 14.01 (s, 1H), 8.21 (d, J = 8.5 Hz, 1H), |
| | | 332 | 7.87 (d, J = 8.5 Hz, 1H), 7.80 (d, J = 1.6 Hz, 1H), 7.73 (d, J = 8.1 Hz, 1H), |
| | | ([M + H]⁺) | 7.68 (dd, J = 8.1, 1.7 Hz, 1H), 2.03 (t, J = 19.0 Hz, 3H); |
| | | | ¹⁹F NMR (376 MHz, DMSO-d₆) |
| | | | δ -85.10 |
| F112 | | ESIMS m/z | ¹H NMR (400 MHz, CDCl₃) δ 7.89 (d, J = 8.4 Hz, 1H), 7.77 (d, J = 8.4 Hz, |
| | | 346 | 1H), 7.70 (d, J = 8.0 Hz, 1H), 7.62 (d, J = 1.7 Hz, 1H), 7.53-7.47 (m, 1H), |
| | | ([M + H]⁺) | 4.02 (s, 3H), 1.93 (t, J = 18.1 Hz, 3H); |
| | | | ¹⁹F NMR (376 MHz, CDCl₃) δ -88.63 |
| F113 | | EIMS m/z | ¹H NMR (400 MHz, CDCl₃) δ 7.89 (dd, J = 8.7, 3.5 Hz, 1H), 7.80-7.74 (m, |
| | | 333.1 | 2H), 7.71-7.60 (m, 2H), 4.02 (s, 3H); |
| | | | ¹⁹F NMR (376 MHz, CDCl₃) |
| | | | δ -62.94, -118.77 |
| F114 | 114- | ESIMS m/z | ¹H NMR (400 MHz, CDCl₃) δ 9.27 (d, J = 2.08 Hz, 1H), 8.55 (dd, J = 2.13, |
| | 116 | 317 | 8.23 Hz, 1H), 7.95 (d, J = 8.44 Hz, 1H), 7.87 (d, J = 8.43 Hz, 1H), 7.80 (d, J = |
| | | ([M + H]⁺) | 8.33 Hz, 1H), 4.05 (s, 3H) |
| F115 | 93- | ESIMS m/z | ¹H NMR (300 MHz, CDCl₃) δ 8.97 (s, 1H), 7.80 (dd, J = 8.6, 7.5 Hz, 1H), |
| | 95 | 332 | 7.33-7.27 (m, 1H), 4.08 (s, 3H), 4.05 (d, J = 1.1 Hz, 3H) |
| | | ([M + H]⁺) | |
| F116 | | ESIMS m/z | ¹H NMR (400 MHz, CDCl₃) δ 7.91 (d, J = 8.4 Hz, 1H), 7.84-7.77 (m, 2H), |
| | | 494 | 7.76 (d, J = 1.7 Hz, 1H), 7.64 (dd, J = 8.2, 1.7 Hz, 1H), 7.52 (d, J = 8.3 Hz, |
| | | ([M + H]⁺) | 1H), 7.44 (d, J = 2.1 Hz, 1H), 7.30-7.25 (m, 1H), 5.53 (s, 2H); |
| | | | ¹⁹F NMR (376 MHz, CDCl₃) δ -62.93 |
| F117 | | ESIMS m/z | ¹H NMR (500 MHz, CDCl₃) δ 7.91 (d, J = 8.4 Hz, 1H), 7.82 (d, J = 8.4 Hz, |
| | | 475.2 | 1H), 7.81-7.74 (m, 4H), 7.64 (ddd, J = 13.8, 11.2, 7.5 Hz, 2H), 7.52 (t, J = |
| | | ([M − F]⁺) | 7.8 Hz, 1H), 5.51 (s, 2H); |
| | | | ¹⁹F NMR (471 MHz, CDCl₃) |
| | | | δ -62.68, -62.92 |
| F118 | | ESIMS m/z | ¹H NMR (300 MHz, CDCl₃) δ 8.02 (d, J = 8.2 Hz, 1H), 7.98 (d, J = 1.6 Hz, |
| | | 375 | 1H), 7.95 (d, J = 1.8 Hz, 1H), 7.62 (d, J = 8.2 Hz, 1H), 4.02 (s, 3H) |
| | | ([M + H]⁺) | |
| F119 | | ESIMS m/z | ¹H NMR (400 MHz, CDCl₃) δ 7.95 (d, J = 8.6 Hz, 1H), 7.80 (d, J = 2.1 Hz, |
| | | 412.1 | 2H), 7.72 (d, J = 8.6 Hz, 1H), 4.02 (s, 3H), 3.12 (s, 3H) |
| | | ([M + H]⁺) | |
| F120 | | ESIMS m/z | ¹H NMR (400 MHz, CDCl₃) δ 7.95 (d, J = 8.6 Hz, 1H), 7.80 (d, J = 2.1 Hz, |
| | | 412.1 | 2H), 7.72 (d, J = 8.6 Hz, 1H), 4.02 (s, 3H), 3.12 (s, 3H) |
| | | ([M + H]⁺) | |
| F121 | 166- | ESIMS m/z | ¹H NMR (300 MHz, CDCl₃) δ 7.92 (d, J = 8.3 Hz, 1H), 7.77-7.68 (m, 2H), |
| | 169 | 395.9 | 7.64 (d, J = 8.2 Hz, 1H), 4.01 (s, 3H), 2.56-2.25 (s, 3H) |
| | | ([M + H]⁺) | |
| F122 | 166- | ESIMS m/z | ¹H NMR (300 MHz, CDCl₃) δ 7.94 (d, J = 8.9 Hz, 1H), 7.51 (s, 1H), 7.41 (d, |
| | 169 | 396.0 | J = 8.4 Hz, 1H), 7.36 (d, J = 1.4 Hz, 1H), 4.00 (s, 3H), 2.42 (s, 3H) |
| | | ([M + H]⁺) | |
| F123 | | ESIMS m/z | ¹H NMR (500 MHz, CDCl₃) δ 7.85 (d, J = 8.5 Hz, 1H), 7.76 (d, J = 8.4 Hz, |
| | | 392 | 1H), 7.61 (d, J = 8.6 Hz, 1H), 7.11-7.06 (m, 2H), 7.06-7.01 (m, 3H), 6.96 |
| | | ([M + H]⁺) | (dd, J = 8.6, 2.5 Hz, 1H), 4.01 (s, 3H); |
| | | | ¹³C NMR (126 MHz, CDCl₃) δ 164.95, 159.43 (d, J = 243.3 Hz), 159.16, |
| | | | 154.13, 151.56 (d, J = 2.7 Hz), 147.55, 138.31, 133.10, 132.95, 131.80, |
| | | | 129.42, 127.51, 121.34 (d, J = 8.3 Hz), 119.09, 116.71, 116.70 (d, J = 23.5 |
| | | | Hz), 53.07; |
| | | | ¹⁹F NMR (471 MHz, CDCl₃) δ -118.29 |
| F124 | | EIMS m/z | ¹H NMR (500 MHz, CDCl₃) δ 7.88 (d, J = 8.4 Hz, 1H), 7.77 (d, J = 8.5 Hz, |
| | | 361 | 1H), 7.40 (s, 1H), 7.20 (s, 1H), 4.02 (s, 3H); |
| | | | ¹⁹F NMR (471 MHz, CDCl₃) δ -50.00 |
| F125 | | ESIMS m/z | ¹H NMR (500 MHz, CDCl₃) δ 8.21 (d, J = 1.7 Hz, 1H), 8.00 (dd, J = 8.2, 1.7 |
| | | 307 | Hz, 1H), 7.93 (d, J = 8.4 Hz, 1H), 7.82 (d, J = 8.5 Hz, 1H), 7.78 (d, J = 8.1 |
| | | ([M + H]⁺) | Hz, 1H), 4.05 (s, 3H) |
| F126 | | ESIMS m/z | ¹H NMR (500 MHz, CDCl₃) δ 8.05 (d, J = 8.5 Hz, 1H), 7.81 (d, J = 8.4 Hz, |
| | | 348 | 1H), 7.49 (s, 1H), 7.47 (s, 1H); |
| | | ([M + H]⁺) | ¹⁹F NMR (471 MHz, CDCl₃) δ -48.12 |
| F127 | | ESIMS m/z | ¹H NMR (300 MHz, CDCl₃) δ 8.12 (s, 1H), 7.85 (d, J = 1.7 Hz, 1H), 7.72 |
| | | 403 | (dd, J = 8.0, 1.7 Hz, 1H), 7.44 (d, J = 8.0 Hz, 1H); |
| | | ([M + H]⁺) | ¹⁹F NMR (376 MHz, CDCl₃) δ -41.73 |
| F128 | | | ¹H NMR (500 MHz, CDCl₃) δ 7.86 (dd, J = 1.7, 1.0 Hz, 2H), 7.75 (d, J = 7.2 |
| | | | Hz, 1H), 7.19 (d, J = 10.3 Hz, 1H), 4.03 (s, 3H); |
| | | | ¹³C NMR (126 MHz, CDCl₃) δ 164.92, 48.37, 148.35, 147.75, 139.03, |
| | | | 129.67, 126.75, 126.66, 117.42, 117.40, 116.99, 116.77, 53.07; |
| | | | ¹⁹F NMR (471 MHz, CDCl₃) δ -124.83 |
| F129 | | ESIMS m/z | ¹H NMR (500 MHz, CDCl₃) δ 8.34 (d, J = 2.6 Hz, 1H), 8.18-8.13 (m, 2H), |
| | | 313.01 | 7.83 (d, J = 8.5 Hz, 1H), 4.03 (d, J = 2.3 Hz, 6H); |
| | | ([M + H]⁺) | ¹³C NMR (126 MHz, CDCl₃) δ 165.00, 159.42, 150.53, 147.64, 145.89, |
| | | | 139.17, 138.62, 129.43, 127.00, 125.07, 121.67, 54.14, 53.04 |
| F130 | | ESIMS m/z | ¹H NMR (400 MHz, CDCl₃) δ 7.99 (s, 1H), 7.80 (d, J = 1.7 Hz, 1H), 7.67 |
| | | 417 | (dd, J = 8.0, 1.7 Hz, 1H), 7.44 (d, J = 7.9 Hz, 1H), 3.99 (s, 3H); |
| | | ([M + H]⁺) | ¹⁹F NMR (376 MHz, CDCl₃) δ -41.90 |
| F131 | | ESIMS m/z | ¹H NMR (400 MHz, CDCl₃) δ 7.86 (s, 1H), 7.84 (dd, J = 8.1, 1.1 Hz, 1H), |
| | | 387 | 7.72 (dd, J = 8.1, 1.7 Hz, 1H), 7.53 (d, J = 8.0 Hz, 1H); |
| | | ([M + H]⁺) | ¹³C NMR (101 MHz, CDCl₃) δ 160.38, 159.57, 156.87, 142.11, 141.94, |
| | | | 136.90, 135.16 (d, J = 5.7 Hz), 134.29, 134.01 (d, J = 4.4 Hz), 132.00 (d, J = |
| | | | 1.8 Hz), 128.83, 128.61, 128.43, 127.60 |
| F132 | | ESIMS m/z | ¹H NMR (500 MHz, CDCl₃) δ 7.95 (dd, J = 8.7, 3.5 Hz, 1H), 7.87-7.82 (m, |
| | | 318 | 2H), 7.80 (d, J = 8.1 Hz, 1H), 7.71 (dd, J = 8.1, 1.7 Hz, 1H); |
| | | ([M − H]⁻) | ¹⁹F NMR (471 MHz, CDCl₃) |
| | | | δ -60.43, -116.60, -116.60, -116.62, -116.62 |
| F133 | | ESIMS m/z | ¹H NMR (500 MHz, CDCl₃) δ 8.42 (d, J = 4.9 Hz, 1H), 8.13 (d, J = 8.4 Hz, |
| | | 303 | 1H), 7.90 (d, J = 8.4 Hz, 1H), 7.61 (d, J = 4.9 Hz, 1H) |
| | | ([M + H]⁺) | |
| F134 | 155.2- | EIMS m/z | ¹H NMR (400 MHz, Methanol-d₄) δ 8.28 (d, J = 2.6 Hz, 1H), 8.17-8.05 (m, |
| | 163.3 | 298 | 2H), 7.90 (d, J = 8.6 Hz, 1H), 3.94 (s, 3H) |
| F135 | | ESIMS m/z | ¹H NMR (400 MHz, CDCl₃) δ 7.80 (d, J = 1.7 Hz, 1H), 7.71 (d, J = 8.2 Hz, |
| | | 401 | 1H), 7.68 (dd, J = 8.0, 1.8 Hz, 1H), 7.54 (d, J = 8.0 Hz, 1H), 4.00 (s, 3H); |
| | | ([M + H]⁺) | ¹³C NMR (101 MHz, CDCl₃) δ 163.82, 158.25, 155.57, 143.63 (d, J = 5.0 |
| | | | Hz), 142.83 (d, J = 16.1 Hz), 136.68, 135.45 (d, J = 4.7 Hz), 134.38, 132.37, |
| | | | 132.25 (d, J = 4.8 Hz), 127.75-127.49 (m), 126.51, 126.29, 53.26 |
| F136 | 94- | ESIMS m/z | ¹H NMR (300 MHz, CDCl₃) δ 7.90 (d, J = 8.6 Hz, 1H), 7.66 (dd, J = 8.6, 7.0 |
| | 97 | 364 | Hz, 2H), 7.48 (d, J = 8.2 Hz, 1H), 4.00 (s, 3H), 2.57 (s, 3H) |
| | | ([M + H]⁺) | |
| F137 | 87- | ESIMS m/z | ¹H NMR (300 MHz, CDCl₃) δ 7.90 (d, J = 8.7 Hz, 1H), 7.70 (d, J = 8.4 Hz, |
| | 90 | 376 | 2H), 7.57 (d, J = 8.2 Hz, 1H), 6.84-6.69 (m, 1H), 5.70 (d, J = 11.5 Hz, 1H), |
| | | ([M + H]⁺) | 5.48 (d, J = 17.3 Hz, 1H), 4.01 (s, 3H) |
| F138 | 144- | ESIMS m/z | ¹H NMR (300 MHz, CDCl₃) δ 8.39 (d, J = 1.7 Hz, 1H), 8.04 (d, J = 1.8 Hz, |
| | 147 | 428 | 1H), 7.96 (d, J = 8.74 Hz, 1H), 7.52 (d, J = 9.5 Hz, 1H), 3.98 (s, 3H), 3.31 (s, |
| | | ([M + H]⁺) | 3H) |
| F139 | 170- | ESIMS m/z | ¹H NMR (600 MHz, CDCl₃) δ 7.97 (d, J = 8.4 Hz, 2H), 7.89 (d, J = 7.8 Hz, |
| | 173 | 428 | 1H), 7.70 (d, J = 8.4 Hz, 1H), 4.02 (s, 3H), 3.37 (s, 3H) |
| | | ([M + H]⁺) | |
| F140 | 103- | ESIMS m/z | ¹H NMR (300 MHz, CDCl₃) δ 7.94 (d, J = 8.1 Hz, 1H), 7.70 (dd, J = 8.1, 5.7 |
| | 107 | 430 | Hz, 2H), 7.63 (d, J = 8.2 Hz, 1H), 4.02 (s, 3H) |
| | | ([M + H]⁺) | |
| F141 | 104- | ESIMS m/z | ¹H NMR (300 MHz, CDCl₃) δ 8.30-8.26 (m, 1H), 7.91 (d, J = 8.5 Hz, 1H), |
| | 107 | 407 | 7.74 (d, J = 4.2 Hz, 2H), 7.70 (d, J = 1.7 Hz, 1H), 4.01 (s, 6H) |
| | | ([M + H]⁺) | |
| F142 | 78- | ESIMS m/z | ¹H NMR (300 MHz, CDCl₃) δ 7.88 (d, J = 8.2 Hz, 1H), 7.70 (dd, J = 8.6, 1.4 |
| | 81 | 420 | Hz, 2H), 7.56 (d, J = 8.2 Hz, 1H), 7.29 (s, 1H), 4.01 (s, 3H), 3.03 (s, 6H) |
| | | ([M + H]⁺) | |
| F143 | 169- | ESIMS m/z | ¹H NMR (300 MHz, CDCl₃) δ 8.30 (d, J = 8.5 Hz, 1H), 8.12 (d, J = 8.4 Hz, |
| | 172 | 375 | 1H), 8.08-7.96 (m, 2H), 4.04 (s, 3H) |
| | | ([M + H]⁺) | |
| F144 | | EIMS m/z | ¹H NMR (500 MHz, CDCl₃) δ 7.86 (dd, J = 1.7, 1.0 Hz, 2H), 7.75 (d, J = 7.2 |
| | | 325 | Hz, 1H), 7.19 (d, J = 10.3 Hz, 1H), 4.03 (s, 3H); |
| | | | ¹³C NMR (126 MHz, CDCl₃) δ 164.92, 154.93, 149.99, 148.35, 147.75, |
| | | | 139.03, 129.67, 126.75, 126.66, 117.42, 117.40, 116.99, 116.77, 53.07 |
| F145 | | ESIMS m/z | ¹H NMR (500 MHz, CDCl₃) δ 8.08 (s, 1H), 7.88 (d, J = 8.4 Hz, 1H), 7.79 (d, |
| | | 325 | J = 8.5 Hz, 1H), 7.69 (d, J = 1.6 Hz, 1H), 7.66 (d, J = 8.0 Hz, 1H), 7.53 (dd, |
| | | ([M + H]⁺) | J = 8.0, 1.6 Hz, 1H), 4.02 (s, 3H); |
| | | | ¹³C NMR (126 MHz, CDCl₃) δ 164.83, 154.04, 148.35, 147.56, 138.49, |
| | | | 138.03, 134.41, 132.64, 132.11, 129.98, 128.29, 127.61, 125.73, 53.14 |
| F146 | | ESIMS m/z | ¹H NMR (400 MHz, DMSO-d₆) δ 8.21 (d, J = 8.5 Hz, 1H), 8.12 (s, 1H), 7.94 |
| | | 359 | (dd, J = 8.2, 2.0 Hz, 1H), 7.88-7.82 (m, 2H), 7.79 (d, J = 8.5 Hz, 1H), 7.61 |
| | | ([M + H]⁺) | (s, 1H), 3.93 (s, 3H); |
| | | | ¹³C NMR (101 MHz, DMSO-d₆) δ 169.39, 165.06, 155.36, 147.70, 140.85, |
| | | | 139.40, 138.40, 131.55, 129.72, 129.40, 128.43, 127.08, 126.78 (d, J = 3.7 |
| | | | Hz), 125.05 (d, J = 3.9 Hz), 122.91, 53.35 |
| F147 | | ESIMS m/z | ¹H NMR (400 MHz, DMSO-d₆) δ 8.50 (s, 1H), 8.23 (s, 1H), 8.14-8.08 (m, |
| | | 394 | 1H), 8.02-7.97 (m, 1H), 7.68 (d, J = 8.0 Hz, 1H), 7.52 (s, 1H), 3.89 (s, 3H); |
| | | ([M + H]⁺) | ¹³C NMR (101 MHz, DMSO-d₆) δ 167.68, 164.36, 155.59, 144.90, 140.68, |
| | | | 139.00 (d, J = 4.3 Hz), 136.79, 132.89, 131.96, 128.83 |
| F148 | | ESIMS m/z | ¹H NMR (500 MHz, Methanol-d₄) δ 8.02 (d, J = 8.4 Hz, 1H), 7.79 (d, J = 8.4 |
| | | 309.9 | Hz, 1H), 7.12 (s, 1H), 7.03 (s, 1H), 6.09 (s, 2H); |
| | | ([M − H]⁻) | ¹³C NMR (126 MHz, Methanol-d₄) δ 166.40, 154.54, 149.21, 148.66, 147.31, |
| | | | 138.45, 130.47, 128.10, 127.51, 124.20, 110.32, 109.63, 102.46 |
| F149 | | ESIMS m/z | ¹H NMR (400 MHz, CDCl₃) δ 7.84 (d, J = 8.5 Hz, 1H), 7.74 (d, J = 8.6 Hz, |
| | | 325 | 1H), 7.72 (d, J = 2.1 Hz, 1H), 7.50 (dd, J = 8.3, 2.1 Hz, 1H), 7.43 (d, J = 8.2 |
| | | ([M + H]⁺) | Hz, 1H), 4.04 (s, 3H), 2.89 (s, 6H); |
| | | | ¹³C NMR (101 MHz, CDCl₃) δ 165.22, 154.69, 150.92, 147.84, 139.05, |
| | | | 136.67, 131.09, 129.88, 128.87, 122.80, 121.52, 118.68, 52.95, 43.69 |
| F150 | | ESIMS m/z | ¹H NMR (500 MHz, CDCl₃) δ 7.65 (d, J = 8.1 Hz, 1H), 6.92 (d, J = 13.5 Hz, |
| | | 343.9 | 2H), 6.05 (s, 2H), 4.00 (s, 3H); |
| | | ([M + H]⁺) | ¹⁹F NMR (471 MHz, CDCl₃) δ -112.19 |
| F151 | | ESIMS m/z | ¹H NMR (500 MHz, CDCl₃) δ 8.01 (s, 1H), 7.93 (d, J = 8.4 Hz, 1H), 7.80 (d, |
| | | 427.8 | J = 8.1 Hz, 2H), 4.04 (s, 3H); |
| | | ([M + H]⁺) | ¹⁹F NMR (471 MHz, CDCl₃) δ -63.09 |
| F152 | | ESIMS m/z | ¹H NMR (500 MHz, CDCl₃) δ 7.85-7.83 (m, 1H), 7.81 (d, J = 1.7 Hz, 1H), |
| | | 445.9 | 7.74 (dt, J = 8.2, 1.8 Hz, 1H), 4.12-3.86 (m, 3H); |
| | | ([M + H]⁺) | ¹⁹F NMR (471 MHz, CDCl₃) |
| | | | δ -63.21, -112.32 |
| F153 | | ESIMS m/z | ¹H NMR (500 MHz, CDCl₃) δ 7.70 (d, J = 8.1 Hz, 1H), 7.23 (s, 1H), 7.20 (s, |
| | | 380 | 1H), 4.00 (s, 3H); |
| | | ([M + H]⁺) | ¹⁹F NMR (471 MHz, CDCl₃) |
| | | | δ -49.69, -112.24, -112.26 |
| F154 | | ESIMS m/z | ¹H NMR (500 MHz, CDCl₃) δ 7.99 (s, 1H), 7.22 (s, 1H), 7.09 (s, 1H), 3.99 |
| | | 395.9 | (s, 3H); |
| | | ([M + H]⁺) | ¹⁹F NMR (471 MHz, CDCl₃) |
| | | | δ -49.48, -49.81 |
| F155 | | ESIMS m/z | ¹H NMR (500 MHz, CDCl₃) δ 7.95 (s, 1H), 6.93 (s, 1H), 6.80 (s, 1H), 6.05 |
| | | 359.9 | (s, 2H), 3.99 (s, 3H); |
| | | ([M + H]⁺) | ¹³C NMR (126 MHz, CDCl₃) δ 164.07, 153.41, 149.18, 146.82, 145.08, |
| | | | 139.22, 134.40, 130.52, 128.63, 125.75, 110.03, 110.01, 102.21, 53.28 |
| F156 | | ESIMS m/z | ¹H NMR (400 MHz, DMSO-d₆) δ 8.29 (d, J = 8.4 Hz, 1H), 8.12 (s, 2H), 7.76 |
| | | 368 | (d, J = 8.4 Hz, 1H); |
| | | ([M − H]⁻) | ¹⁹F NMR (376 MHz, DMSO-d₆) δ -61.38 |
| F157 | 86- | ESIMS m/z | ¹H NMR (500 MHz, CDCl₃) δ 7.87 (d, J = 8.5 Hz, 1H), 7.76 (d, J = 8.4 Hz, |
| | 89 | 349 | 1H), 7.67 (d, J = 8.5 Hz, 1H), 7.28 (d, J = 2.5 Hz, 1H), 7.16 (dd, J = 8.5, 2.4 |
| | | ([M + H]⁺) | Hz, 1H), 6.55 (t, J = 72.9 Hz, 1H), 4.01 (s, 3H); |
| | | | ¹⁹F NMR (471 MHz, CDCl₃) |
| | | | δ -81.49, -81.65 |
| F158 | | ESIMS m/z | ¹H NMR (500 MHz, CDCl₃) δ 7.77 (t, J = 7.4 Hz, 1H), 7.71 (d, J = 8.4 Hz, |
| | | 352 | 1H), 7.59-7.55 (m, 1H), 7.49-7.44 (m, 1H), 4.01 (s, 3H); |
| | | ([M + H]⁺) | ¹⁹F NMR (471 MHz, CDCl₃) |
| | | | δ -63.01, -111.12, -111.14, -111.16, -111.20, -111.22, -111.23, -113.59, -113.61, -113.66, -113.68 |
| F159 | 128- | ESIMS m/z | ¹H NMR (500 MHz, CDCl₃) δ 8.07 (dd, J = 8.8, 1.5 Hz, 2H), 7.67 (d, J = 9.8 |
| | 130 | 360 | Hz, 1H), 7.45-7.39 (m, 2H), 4.02 (s, 3H), 3.18 (s, 3H); |
| | | ([M + H]⁺) | ¹⁹F NMR (471 MHz, CDCl₃) |
| | | | δ -116.47, -116.49 |
| F160 | | ESIMS m/z | ¹H NMR (400 MHz, DMSO-d₆) δ 14.12 (s, 1H), 8.46 (d, J = 9.3 Hz, 1H), |
| | | 336 | 7.93 (d, J = 11.3 Hz, 1H), 7.89 (d, J = 7.4 Hz, 1H), 7.80 (d, J = 7.7 Hz, 1H); |
| | | ([M − H]⁻) | ¹⁹F NMR (376 MHz, DMSO) |
| | | | δ -61.36, -112.22, -112.30, -116.13, -116.21 |
| F161 | 105- | ESIMS m/z | ¹H NMR (500 MHz, CDCl₃) δ 7.60 (d, J = 8.3 Hz, 1H), 7.44 (d, J = 8.3 Hz, |
| | 107 | 362 | 1H), 6.86-6.80 (m, 1H), 6.74 (d, J = 2.2 Hz, 1H), 6.56 (t, J = 73.6 Hz, 1H), |
| | | ([M + H]⁺) | 3.99 (s, 3H), 3.81 (s, 3H); |
| | | | ¹⁹F NMR (471 MHz, CDCl₃) |
| | | | δ -80.84, -81.00, -112.41, -112.43 |
| F162 | | ESIMS m/z | ¹H NMR (400 MHz, CDCl₃) δ 9.79 (s, 1H), 8.16 (d, J = 8.3 Hz, 1H), 8.05 |
| | | 382.9 | (dd, J = 1.7, 0.8 Hz, 1H), 8.04-8.01 (m, 1H), 7.83 (d, J = 8.4 Hz, 1H); |
| | | ([M + H]⁺) | ¹⁹F NMR (376 MHz, CDCl₃) |
| | | | δ -63.27 |
| F163 | 79- | ESIMS m/z | ¹H NMR (500 MHz, CDCl₃) δ 8.06-7.98 (m, 2H), 7.62 (d, J = 10.1 Hz, |
| | 81 | 364 | 1H), 7.08-7.02 (m, 2H), 4.42 (q, J = 8.1 Hz, 2H), 4.02 (s, 3H); |
| | | ([M + H]⁺) | ¹⁹F NMR (471 MHz, CDCl₃) |
| | | | δ -73.83, -73.85, -73.87, -116.74, -116.76 |
| F164 | | ESIMS m/z | ¹H NMR (400 MHz, CDCl₃) δ 8.98 (br s, 1H), 8.87 (s, 1H), 8.19 (d, J = 8.3 |
| | | 335 | Hz, 1H), 8.14 (s, 1H), 8.07 (d, J = 8.4 Hz, 1H); |
| | | ([M − H]⁻) | ¹⁹F NMR (376 MHz, CDCl₃) |
| | | | δ -62.42 |
| F165 | 88- | ESIMS m/z | ¹H NMR (500 MHz, CDCl₃) δ 8.02 (dd, J = 8.9, 1.5 Hz, 2H), 7.65 (d, J = |
| | 90 | 332 | 10.0 Hz, 1H), 7.23 (d, J = 8.8 Hz, 2H), 6.58 (t, J = 73.5 Hz, 1H), 4.02 (s, |
| | | ([M + H]⁺) | 3H); |
| | | | ¹⁹F NMR (471 MHz, CDCl₃) |
| | | | δ -81.13, -81.28, -116.65, -116.68 |
| F166 | 108- | ESIMS m/z | ¹H NMR (500 MHz, CDCl₃) δ 7.63 (d, J = 8.2 Hz, 1H), 7.56 (dd, J = 7.7, 1.0 |
| | 110 | 364 | Hz, 1H), 7.38-7.33 (m, 1H), 7.22-7.18 (m, 1H), 3.99 (s, 3H), 3.87 (s, 3H); |
| | | ([M + H]⁺) | ¹⁹F NMR (471 MHz, CDCl₃) |
| | | | δ -62.84, -62.84, -112.46, -112.48 |
| F167 | 80- | ESIMS m/z | ¹H NMR (500 MHz, CDCl₃) δ 8.82 (t, J = 8.5 Hz, 1H), 8.04 (dd, J = 8.6, 1.5 |
| | 82 | 335 | Hz, 1H), 7.95 (d, J = 8.5 Hz, 1H), 7.74 (dd, J = 7.8, 1.6 Hz, 1H), 4.05 (s, |
| | | ([M + H]⁺) | 3H); |
| | | | ¹⁹F NMR (471 MHz, CDCl₃) |
| | | | δ -66.59, -66.61, -68.09 |
| F168 | 107- | ESIMS m/z | ¹H NMR (500 MHz, CDCl₃) δ 8.14 (dd, J = 8.5, 1.4 Hz, 2H), 7.79 (d, J = 8.5 |
| | 109 | 291 | Hz, 2H), 7.71 (d, J = 10.0 Hz, 1H), 4.03 (s, 3H); |
| | | ([M + H]⁺) | ¹⁹F NMR (471 MHz, CDCl₃) |
| | | | δ -115.94, -115.96 |
| F169 | 132- | ESIMS m/z | ¹H NMR (500 MHz, CDCl₃) δ 8.22-8.18 (m, 2H), 8.07 (d, J = 8.6 Hz, 2H), |
| | 134 | 344 | 7.72 (d, J = 9.9 Hz, 1H), 4.03 (s, 3H), 3.09 (s, 3H); |
| | | ([M + H]⁺) | ¹⁹F NMR (471 MHz, CDCl₃) |
| | | | δ -116.04, -116.07 |
| F170 | 106- | ESIMS m/z | ¹H NMR (500 MHz, CDCl₃) δ 7.70 (d, J = 8.3 Hz, 1H), 7.56 (d, J = 9.4 Hz, |
| | 108 | 348 | 2H), 7.49 (d, J = 7.9 Hz, 1H), 4.00 (s, 3H), 2.33 (s, 3H); |
| | | ([M + H]⁺) | ¹⁹F NMR (471 MHz, CDCl₃) |
| | | | δ -62.87, -114.90, -114.91 |
| F171 | | ESIMS m/z | ¹H NMR (400 MHz, CDCl₃) δ 7.85 (d, J = 8.5 Hz, 1H), 7.81 (dd, J = 8.6, 2.1 |
| | | 371 | Hz, 1H), 7.72 (dd, J = 8.5, 7.9 Hz, 1H), 7.30 (dd, J = 8.6, 1.5 Hz, 1H), 4.03 |
| | | ([M + H]⁺) | (s, 3H), 3.19 (qd, J = 7.2, 1.5 Hz, 4H), 1.03 (td, J = 7.2, 0.8 Hz, 6H); |
| | | | ¹³C NMR (101 MHz, CDCl₃) δ 164.97, 160.24 (d, J = 254.4 Hz), 150.84, |
| | | | 147.87, 138.75, 137.80 (d, J = 6.0 Hz), 135.39 (d, J = 15.0 Hz), 129.30, |
| | | | 127.08 (d, J = 3.9 Hz), 126.82 (d, J = 10.8 Hz), 125.88 (d, J = 3.6 Hz), |
| | | | 125.10 (d, J = 12.2 Hz), 52.98, 47.43 (d, J = 3.9 Hz), 13.45; |
| | | | ¹⁹F NMR (376 MHz, CDCl₃) |
| | | | δ -121.62 |
| F172 | | | ¹H NMR (500 MHz, CDCl₃) δ 7.86 (dd, J = 1.7, 1.0 Hz, 2H), 7.75 (d, J = 7.2 |
| | | | Hz, 1H), 7.19 (d, J = 10.3 Hz, 1H), 4.03 (s, 3H); |
| | | | ¹⁹F NMR (471 MHz, CDCl₃) |
| | | | δ -124.83 |
| F173 | | | ¹H NMR (400 MHz, CDCl₃) δ 7.69 (d, J = 8.4 Hz, 1H), 7.33 (d, J = 5.5 Hz, |
| | | | 1H), 6.96 (d, J = 8.5 Hz, 1H), 4.01 (s, 3H); |
| | | | ¹³C NMR (101 MHz, CDCl₃) δ 163.86, 158.42, 157.60, 155.72, 155.11, |
| | | | 145.34, 145.20, 143.83, 143.79, 140.49, 140.03, 139.86, 134.59, 132.03, |
| | | | 131.71, 131.66, 126.56, 126.34, 116.96, 116.74, 111.30, 111.27, 99.40, |
| | | | 99.09, 53.20; |
| | | | ¹⁹F NMR (376 MHz, CDCl₃) |
| | | | δ -49.80, -113.48, -115.06, -115.17 |
| F174 | | ESIMS m/z | ¹H NMR (400 MHz, CDCl₃) δ 7.98 (s, 1H), 7.17 (d, J = 5.5 Hz, 1H), 6.95 (d, |
| | | 379.9 | J = 8.2 Hz, 1H), 4.00 (s, 3H); |
| | | ([M + H]⁺) | ¹⁹F NMR (376 MHz, CDCl₃) |
| | | | δ -49.72, -114.22 |
| F175 | | ESIMS m/z | ¹H NMR (400 MHz, CDCl₃) δ 7.81 (d, J = 8.1 Hz, 1H), 7.29 (s, 1H), 7.19 (s, |
| | | 366 | 1H); |
| | | ([M + H]⁺) | ¹⁹F NMR (376 MHz, CDCl₃) |
| | | | δ -49.59, -110.33 |
| F176 | 150- | ESIMS m/z | ¹H NMR (400 MHz, DMSO-d₆) δ 8.44 (br s, 1H), 8.27 (d, J = 8.8 Hz, 1H), |
| | 154 | 375 | 8.12 (br s, 1H), 7.94 (d, J = 8.4 Hz, 1H), 7.79-7.75 (m, 2H), 7.69-7.67 (m, |
| | | ([M + H]⁺) | 1H), 3.93 (s, 3H) |
| F177 | | ESIMS m/z | ¹H NMR (400 MHz, DMSO-d₆) δ 8.26 (d, J = 8.4 Hz, 1H), 7.94 (d, J = 8.4 |
| | | 376 | Hz, 1H), 7.71 (s, 1H), 7.74-7.71 (m, 1H), 7.67-7.64 (m, 1H), 3.99 (t, J = |
| | | ([M + H]⁺) | 14.0 Hz, 2H), 3.93 (s, 3H), 3.36 (s, 3H); |
| | | | ¹⁹F NMR (376 MHz, DMSO-d₆) |
| | | | δ -101.50 |
| F178 | 64- | ESIMS m/z | ¹H NMR (400 MHz, CDCl₃) δ 7.92 (d, J = 8.4 Hz, 1H), 7.85 (d, J = 8.4 Hz, |
| | 67 | 357 | 1H), 7.81-7.78 (m, 2H), 7.69-7.67 (m, 1H), 4.01 (s, 3H) |
| | | ([M + H]⁺) | |
| F179 | | ESIMS m/z | ¹H NMR (400 MHz, DMSO-d₆) δ 14.01 (br s, 1H), 8.21 (d, J = 8.4 Hz, 1H), |
| | | 362 | 7.88 (d, J = 8.4 Hz, 1H), 7.78 (s, 1H), 7.74 (d, J = 7.6 Hz, 1H), 7.66 (d, J = |
| | | ([M + H]⁺) | 8.0 Hz, 1H), 3.99 (t, J = 13.6 Hz, 2H), 3.36 (s, 3H); |
| | | | ¹⁹F NMR (376 MHz, DMSO-d₆) |
| | | | δ -101.50 |
| F180 | 128- | ESIMS m/z | ¹H NMR (400 MHz, DMSO-d₆) δ 13.98 (br s, 1H), 8.22 (d, J = 8.4 Hz, 1H), |
| | 132 | 362 | 7.90 (d, J = 8.4 Hz, 1H), 7.89-7.75 (m, 2H), 7.69 (dd, J = 1.6, 8.4 Hz, 1H) |
| | | ([M + H]⁺) | |
| F181 | | ESIMS m/z | 1H NMR (400 MHz, CDCl₃) δ 7.88-7.84 (m, 2H), 7.83 (s, 1H); |
| | | 431 | ¹⁹F NMR (376 MHz, CDCl₃) |
| | | ([M + H]⁺) | δ -63.29, -110.30 |
| F182 | | ESIMS m/z | ¹H NMR (400 MHz, CDCl₃) δ 8.10 (s, 1H), 7.28 (s, 1H), 7.10 (s, 1H); |
| | | 381.9 | ¹⁹F NMR (376 MHz, CDCl₃) |
| | | ([M + H]⁺) | δ -49.55 |
| F183 | 138- | ESIMS m/z | ¹H NMR (400 MHz, CDCl₃) δ 8.03 (s, 1H), 6.93 (s, 1H), 6.77 (s, 1H), 6.07 |
| | 153 | 343.9 | (s, 2H) |
| | | ([M + H]⁺) | |
| F184 | | ESIMS m/z | ¹H NMR (400 MHz, CDCl₃) δ 8.68 (s, 1H), 7.98 (d, J = 8.4 Hz, 1H), 7.81 (d, |
| | | 367 | J = 8.4 Hz, 1H), 7.27 (t, J = 52.6 Hz, 1H), 4.02 (s, 3H); |
| | | ([M + H]⁺) | ¹³C NMR (101 MHz, CDCl₃) δ 164.42, 153.14, 152.80, 148.51, 147.00, |
| | | | 139.32, 137.21 (t, J = 23.2 Hz), 131.37, 131.36 (t, J = 2.7 Hz), 130.16 (t, J = |
| | | | 3.6 Hz), 127.28, 111.10 (t, J = 242.9 Hz), 53.15; |
| | | | ¹⁹F NMR (376 MHz, CDCl₃) |
| | | | δ -117.87 |
| F185 | | ESIMS m/z | ¹H NMR (500 MHz, CDCl₃) δ 8.04 (dd, J = 8.8, 1.5 Hz, 2H), 7.67 (d, J = 9.9 |
| | | 350 | Hz, 1H), 7.34 (dd, J = 9.1, 1.1 Hz, 2H), 4.02 (s, 3H); |
| | | ([M + H]⁺) | ¹⁹F NMR (471 MHz, CDCl₃) |
| | | | δ -57.70, -116.59, -116.61 |
| F186 | | ESIMS m/z | ¹H NMR (500 MHz, CDCl₃) δ 7.80 (d, J = 1.6 Hz, 1H), 7.72 (d, J = 8.2 Hz, |
| | | 326 | 1H), 7.69 (dd, J = 8.0, 1.5 Hz, 1H), 7.61 (d, J = 7.9 Hz, 1H), 4.01 (s, 3H); |
| | | ([M + H]⁺) | ¹⁹F NMR (471 MHz, CDCl₃) |
| | | | δ -112.54, -112.56 |
| F187 | 90- | ESIMS m/z | ¹H NMR (500 MHz, CDCl₃) δ 7.72-7.63 (m, 2H), 7.18 (ddt, J = 8.7, 2.3, |
| | 92 | 368 | 1.0 Hz, 1H), 7.09 (dd, J = 10.2, 1.3 Hz, 1H), 4.01 (s, 3H); |
| | | ([M + H]⁺) | ¹⁹F NMR (471 MHz, CDCl₃) |
| | | | δ -57.92, -109.30, -109.32, -109.32, -109.34, -109.38, -109.39, -109.40, -109.42, -113.76, -113.78, |
| | | | -113.84, -113.85 |
| F188 | 69- | ESIMS m/z | ¹H NMR (500 MHz, CDCl₃) δ 8.24 (d, J = 1.8 Hz, 1H), 7.96 (dt, J = 8.6, 1.7 |
| | 70 | 306 | Hz, 1H), 7.68 (d, J = 2.2 Hz, 1H), 7.65 (d, J = 10.0 Hz, 1H), 7.60 (d, J = 8.6 |
| | | ([M + H]⁺) | Hz, 1H), 6.86 (dd, J = 2.1, 0.8 Hz, 1H), 4.03 (s, 3H); |
| | | | ¹⁹F NMR (471 MHz, CDCl₃) |
| | | | δ -116.57, -116.59 |
| F189 | 163- | ESIMS m/z | ¹H NMR (500 MHz, CDCl₃) δ 8.30 (s, 1H), 8.27 (s, 1H) 7.87 (dt, J = 8.6, 1.7 |
| | 165 | 305 | Hz, 1H), 7.62 (d, J = 10.1 Hz, 1H), 7.48 (dt, J = 8.6, 0.8 Hz, 1H), 7.29-7.24 |
| | | ([M + H]⁺) | (m, 1H), 6.65 (ddd, J = 3.1, 2.0, 0.9 Hz, 1H), 4.02 (s, 3H); |
| | | | ¹⁹F NMR (471 MHz, CDCl₃) |
| | | | δ -116.50, -116.52 |
| F190 | 122- | ESIMS m/z | ¹H NMR (400 MHz, DMSO-d₆) δ 13.96 (s, 1H), 8.32 (d, J = 9.1 Hz, 1H), |
| | 123 | 350 | 7.62 (d, J = 7.7 Hz, 1H), 7.49-7.44 (m, 2H), 3.88 (s, 3H); |
| | | ([M + H]⁺) | ¹⁹F NMR (376 MHz, DMSO) |
| | | | δ -61.16, -114.86 |
| F191 | | ESIMS m/z | ¹H NMR (400 MHz, CDCl₃) δ 8.57 (d, J = 2.1 Hz, 1H), 7.94 (d, J = 8.4 Hz, |
| | | 317 | 1H), 7.89-7.86 (m, 2H), 4.01 (s, 3H); |
| | | ([M + H]⁺) | ¹³C NMR (101 MHz, CDCl₃) δ 164.54, 153.00, 150.94, 146.93, 146.57, |
| | | | 139.09, 138.10, 132.30, 131.06, 127.13, 53.07 |
| F192 | 132- | ESIMS m/z | ¹H NMR (500 MHz, CDCl₃) δ 8.15 (d, J = 7.8 Hz, 1H), 8.11 (d, J = 8.4 Hz, |
| | 135 | 337 | 1H), 7.99 (d, J = 8.4 Hz, 1H), 7.82 (d, J = 7.8 Hz, 1H); |
| | | ([M + H]⁺) | ¹³C NMR (126 MHz, CDCl₃) δ 160.93, 150.65, 149.55, 148.85 (q, J = 36.6 |
| | | | Hz), 142.53, 141.91, 141.38, 135.64, 134.26, 129.22, 120.38 (q, J = 274.6 |
| | | | Hz), 119.72 (q, J = 2.7 Hz); |
| | | | ¹⁹F NMR (471 MHz, CDCl₃) |
| | | | δ -68.07 |
| F193 | | ESIMS m/z | ¹H NMR (400 MHz, Acelone-d₆) δ 11.46 (br s, 1H), 8.83 (s, 1H), 8.26 (d, J = |
| | | 353 | 8.5 Hz, 1H), 8.03 (d, J = 8.4 Hz, 1H), 7.53 (t, J = 52.1 Hz, 1H); |
| | | ([M + H]⁺) | ¹³C NMR (101 MHz, Acelone-d₆) δ 163.78, 153.35, 152.93, 148.59, 146.51, |
| | | | 140.06, 136.97 (t, J = 23.1 Hz), 130.87 (t, J = 2.6 Hz), 130.43, 129.73 (t, J = |
| | | | 3.5 Hz), 127.81, 111.68 (t, J = 241.2 Hz); |
| | | | ¹⁹F NMR (376 MHz, Acetone) |
| | | | δ -118.78 |
| F194 | 112- | ESIMS m/z | ¹H NMR (400 MHz, CDCl₃) δ 8.94 (s, 1H), 7.97 (d, J = 8.4 Hz, 1H), 7.82 (s, |
| | 116 | 351 | 1H), 7.81 (d, J = 8.4 Hz, 2H), 4.04 (s, 3H); |
| | | ([M + H]⁺) | ¹³C NMR (101 MHz, CDCl₃) δ 164.44, 152.38, 150.22, 149.20 (q, J = 35.7 |
| | | | Hz), 148.45, 143.49, 139.07, 135.56, 131.16, 127.42, 120.78 (q, J = 274.7 |
| | | | Hz), 122.09 (q, J = 3.0 Hz 53.23; |
| | | | ¹⁹F NMR (376 MHz, CDCl₃) |
| | | | δ -68.11 |
| F195 | | ESIMS m/z | ¹H NMR (400 MHz, CDCl₃) δ 8.61 (d, J = 2.1 Hz, 1H), 8.22 (d, J = 8.5 Hz, |
| | | 303 | 1H), 8.07 (d, J = 8.4 Hz, 1H), 7.94 (d, J = 2.1 Hz, 1H); |
| | | ([M + H]⁺) | ¹³C NMR (101 MHz, CDCl₃) δ 161.29, 151.60, 148.49, 146.74, 141.97, |
| | | | 140.59, 138.86, 134.04, 132.95, 130.84, 128.98 |
| F196 | | ESIMS m/z | ¹H NMR (500 MHz, CDCl₃) δ 7.77 (d, J = 8.1 Hz, 1H), 6.99 (s, 1H), 6.88 (s, |
| | | 329.9 | 1H), 6.09 (s, 2H); |
| | | ([M + H]⁺) | ¹⁹F NMR (471 MHz, CDCl₃) |
| | | | δ -109.95 |
| F197 | | ESIMS m/z | ¹H NMR (500 MHz, CDCl₃) δ 8.35 (d, J = 8.9 Hz, 2H), 8.21 (dd, J = 8.9, 1.3 |
| | | 311 | Hz, 2H), 7.73 (d, J = 10.0 Hz, 1H), 4.04 (s, 3H); |
| | | ([M + H]⁺) | ¹⁹F NMR (471 MHz, CDCl₃) |
| | | | δ -115.69, -115.69 |
| F198 | 157- | ESIMS m/z | ¹H NMR (400 MHz, DMSO-d₆) δ 13.97 (s, 1H), 8.34 (d, J = 10.7 Hz, 1H), |
| | 159 | 292 | 8.23 (t, J = 1.7 Hz, 1H), 8.10 (d, J = 2.2 Hz, 1H), 7.89 (dt, J = 8.7, 1.7 Hz, |
| | | ([M + H]⁺) | 1H), 7.77 (dt, J = 8.7, 0.8 Hz, 1H), 7.11 (dd, J = 2.2, 1.0 Hz, 1H); |
| | | | ¹⁹F NMR (376 MHz, DMSO) |
| | | | δ -118.00 |
| F199 | 176- | ESIMS m/z | ¹H NMR (400 MHz, DMSO-d₆) δ 13.88 (s, 1H), 11.34 (s, 1H), 8.25 (d, J = |
| | 178 | 291 | 10.9 Hz, 1H), 8.17 (d, J = 1.8 Hz, 1H), 7.71 (dt, J = 8.6, 1.7 Hz, 1H), 7.57- |
| | | ([M + H]⁺) | 7.48 (m, 1H), 7.44 (t, J = 2.7 Hz, 1H), 6.58 (ddd, J = 3.0, 1.9, 0.9 Hz, 1H); |
| | | | ¹⁹F NMR (376 MHz, DMSO) |
| | | | δ -118.09 |
| F200 | 106- | ESIMS m/z | ¹H NMR (500 MHz, CDCl₃) δ 7.68 (d, J = 8.1 Hz, 1H), 7.49 (d, J = 8.4 Hz, |
| | 109 | 367 | 1H), 7.29 (d, J = 2.4 Hz, 1H), 7.17 (dd, J = 8.5, 2.4 Hz, 1H), 6.56 (t, J = 72.8 |
| | | ([M + H]⁺) | Hz, 1H), 4.00 (s, 3H); |
| | | | ¹⁹F NMR (471 MHz, CDCl₃) |
| | | | δ -81.55, -81.70, -112.64, -112.66 |
| F201 | | ESIMS m/z | ¹H NMR (500 MHz, CDCl₃) δ 8.51 (d, J = 8.5 Hz, 1H), 8.05 (d, J = 8.6 Hz, |
| | | 361 | 1H), 7.85 (d, J = 8.0 Hz, 1H), 7.81-7.77 (m, 1H), 7.68 (ddd, J = 8.2, 1.6, |
| | | ([M + H]⁺) | 0.8 Hz, 1H), 4.05 (s, 3H); |
| | | | ¹⁹F NMR (471 MHz, CD₃CN) |
| | | | δ -68.3 |
| F202 | | EIMS m/z | ¹H NMR (500 MHz, CDCl₃) δ 8.07 (d, J = 2.1 Hz, 1H), 8.02-7.97 (m, 2H), |
| | | 411 | 7.25 (d, J = 6.9 Hz, 1H), 4.01 (s, 3H); |
| | | | ¹³C NMR (126 MHz, CDCl₃) δ 163.95, 151.60, 145.85, 140.27, 135.34, |
| | | | 133.52, 132.00, 130.30, 127.16, 116.60, 53.26 |
| F203 | | ESIMS m/z | ¹H NMR (500 MHz, CDCl₃) δ 8.31-8.20 (m, 2H), 7.89 (d, J = 8.5 Hz, 1H), |
| | | 378 | 7.79 (d, J = 8.5 Hz, 1H), 7.32-7.18 (m, 1H), 4.05 (s, 3H); |
| | | ([M + H]⁺) | ¹³C NMR (126 MHz, CDCl₃) δ 164.96, 152.50, 148.21, 139.47, 134.70, |
| | | | 132.60, 129.68, 124.30, 122.40, 117.23, 53.09 |
| F204 | | ESIMS m/z | ¹H NMR (500 MHz, CDCl₃) δ 8.31 (d, J = 2.0 Hz, 1H), 8.28 (ddd, J = 8.7, |
| | | 395.9 | 2.2, 1.0 Hz, 1H), 7.70 (d, J = 10.1 Hz, 1H), 7.34-7.22 (m, 1H), 4.03 (s, 3H); |
| | | ([M + H]⁺) | ¹⁹F NMR (471 MHz, CDCl₃) |
| | | | δ -58.29, -116.64, -116.66 |
| F205 | 149- | ESIMS m/z | ¹H NMR (400 MHz, CDCl₃) δ 8.91 (s, 1H), 8.13 (d, J = 8.4 Hz, 1H), 7.92 (d, |
| | 153 | 337 | J = 8.4 Hz, 1H), 7.89 (s, 1H); |
| | | ([M + H]⁺) | ¹³C NMR (101 MHz, CDCl₃) δ 160.94, 151.63, 149.77 (q, J = 35.9 Hz), |
| | | | 149.27, 143.79, 142.79, 142.03, 134.53, 134.31, 129.19, 122.47 (q, J = 3.3 |
| | | | Hz), 120.62 (q, J = 274.8 Hz); |
| | | | ¹⁹F NMR (376 MHz, CDCl₃) |
| | | | δ -68.12 |
| F206 | | ESIMS m/z | ¹H NMR (400 MHz, CDCl₃) δ 8.23 (dd, J = 1.8, 0.8 Hz, 1H), 8.02 (dd, J = |
| | | 394.9 | 1.7, 0.8 Hz, 1H), 7.99 (d, J = 8.4 Hz, 1H), 7.56 (d, J = 8.4 Hz, 1H), 3.98 (s, |
| | | ([M + H]⁺) | 3H); |
| | | | ¹⁹F NMR (376 MHz, CDCl₃) |
| | | | δ -63.21 |
| F207 | | ESIMS m/z | ¹H NMR (400 MHz, CDCl₃) δ 8.40 (s, 1H), 8.05 (dd, J = 8.5, 1.3 Hz, 1H), |
| | | 281 | 7.92 (d, J = 8.5 Hz, 1H), 7.36 (d, J = 11.4 Hz, 1H), 4.02 (s, 3H), 2.43 (s, 3H); |
| | | ([M + H]⁺) | ¹³C NMR (101 MHz, CDCl₃) δ 164.80, 157.97 (d, J = 265.7 Hz), 152.17 (d, J = |
| | | | 6.6 Hz), 147.38, 146.15 (d, J = 4.6 Hz), 140.37 (d, J = 8.8 Hz), 139.02, |
| | | | 136.67 (d, J = 4.4 Hz), 130.50, 126.36 (d, J = 4.3 Hz), 125.23 (d, J = 19.4 |
| | | | Hz), 53.01, 18.01; |
| | | | ¹⁹F NMR (376 MHz, CDCl₃) |
| | | | δ -122.78 |
| F208 | 137- | ESIMS m/z | ¹H NMR (400 MHz, CDCl₃) δ 8.42 (s, 1H), 8.35 (d, J = 8.5 Hz, 1H), 8.03 (d, |
| | 141 | 265 | J = 8.5 Hz, 1H), 7.44 (d, J = 11.9 Hz, 1H), 2.47 (s, 3H); |
| | | ([M − H]⁻) | ¹³C NMR (101 MHz, CDCl₃) δ 161.82, 158.30 (d, J = 267.2 Hz), 150.82 (d, J = |
| | | | 7.1 Hz), 146.04 (d, J = 4.7 Hz), 141.65, 141.61, 138.48 (d, J = 7.1 Hz), |
| | | | 137.71 (d, J = 4.8 Hz), 133.26, 127.58 (d, J = 3.6 Hz), 125.89 (d, J = 19.7 |
| | | | Hz), 18.10; |
| | | | ¹⁹F NMR (376 MHz, CDCl₃) |
| | | | δ -121.75 |
| F209 | 41- | ESIMS m/z | ¹H NMR (400 MHz, CDCl₃) δ 8.08 (d, J = 8.0 Hz, 2H), 7.80-7.54 (m, 3H), |
| | 43 | 316 | 6.71 (t, J = 56.3 Hz, 1H), 4.02 (d, J = 1.0 Hz, 3H); |
| | | ([M + H]⁺) | ¹⁹F NMR (376 MHz, CDCl₃) |
| | | | δ -111.56, -116.42 |
| F210 | 119- | ESIMS m/z | ¹H NMR (500 MHz, CDCl₃) δ 7.70 (d, J = 8.2 Hz, 1H), 7.52 (d, J = 8.4 Hz, |
| | 121 | 385 | 1H), 7.39 (d, J = 2.2 Hz, 1H), 7.30-7.25 (m, 1H), 4.00 (s, 3H); |
| | | ([M + H]⁺) | ¹⁹F NMR (471 MHz, CDCl₃) |
| | | | δ -57.85, -112.68, -112.70 |
| F211 | | ESIMS m/z | ¹H NMR (400 MHz, CDCl₃) δ 7.88 (d, J = 8.4 Hz, 1H), 7.78 (d, J = 8.4 Hz, |
| | | 366 | 1H), 7.52 (s, 1H), 7.04 (s, 1H), 4.28 (s, 2H), 4.02 (s, 3H); |
| | | ([M + H]⁺) | ¹³C NMR (101 MHz, CDCl₃) δ 164.81, 153.43, 147.79, 143.46, 140.97, |
| | | | 138.40, 130.14, 128.25 (d, J = 5.4 Hz), 127.42, 120.14, 119.99 |
| F212 | | ESIMS m/z | ¹H NMR (400 MHz, CDCl₃) δ 7.89 (d, J = 8.4 Hz, 1H), 7.64 (d, J = 8.4 Hz, |
| | | 366 | 1H), 7.46 (d, J = 8.1 Hz, 1H), 6.95 (dd, J = 8.2, 0.9 Hz, 1H), 4.82 (s, 2H), |
| | | ([M + H]⁺) | 4.01 (s, 3H); |
| | | | ¹³C NMR (101 MHz, CDCl₃) δ 164.76, 154.51, 147.70, 141.51 (d, J = 34.0 |
| | | | Hz), 138.55, 130.06, 127.19, 125.61, 124.97 (q, J = 5.3 Hz), 122.90, 119.10, |
| | | | 118.80, 114.99, 114.69, 53.08 |
| F213 | | ESIMS m/z | ¹H NMR (400 MHz, CDCl₃) δ 7.95 (d, J = 8.4 Hz, 1H), 7.65 (d, J = 8.4 Hz, |
| | | 364.9 | 1H), 7.10 (s, 1H), 6.90 (s, 1H), 4.61 (s, 2H), 4.01 (s, 3H); |
| | | ([M + H]⁺) | ¹⁹F NMR (376 MHz, CDCl₃) |
| | | | δ -63.54 |
| F214 | | EIMS m/z | ¹H NMR (400 MHz, CDCl₃) δ 8.09 (s, 1H), 7.97 (d, J = 8.3 Hz, 1H), 7.74 (d, |
| | | 475 | J = 1.6 Hz, 1H), 7.38 (d, J = 8.3 Hz, 1H), 4.01 (s, 3H); |
| | | | ¹⁹F NMR (376 MHz, CDCl₃) |
| | | | δ -63.02 |
| F215 | 97- | ESIMS m/z | ¹H NMR (500 MHz, CDCl₃) δ 7.61 (d, J = 8.3 Hz, 1H), 7.47 (d, J = 8.4 Hz, |
| | 99 | 380 | 1H), 6.95 (dq, J = 8.4, 1.2 Hz, 1H), 6.82 (d, J = 2.1 Hz, 1H), 3.99 (s, 3H), |
| | | ([M + H]⁺) | 3.82 (s, 3H); |
| | | | ¹⁹F NMR (471 MHz, CDCl₃) |
| | | | δ -57.64, -112.43, -112.45 |
| F216 | | EIMS m/z | ¹H NMR (400 MHz, CDCl₃) δ 7.94 (d, J = 8.3 Hz, 1H), 7.80-7.75 (m, 1H), |
| | | 377.1 | 7.65 (d, J = 1.6 Hz, 1H), 7.39 (d, J = 8.3 Hz, 1H), 6.34 (dd, J = 17.4, 11.0 |
| | | | Hz, 1H), 5.74 (d, J = 17.4 Hz, 1H), 5.32 (d, J = 11.0 Hz, 1H), 4.00 (s, 3H); |
| | | | ¹⁹F NMR (376 MHz, CDCl₃) |
| | | | δ -63.11 |
| F217 | | EIMS m/z | ¹H NMR (400 MHz, CDCl₃) δ 7.89 (dd, J = 8.3, 1.0 Hz, 1H), 7.44-7.33 (m, |
| | | 379.1 | 2H), 7.08 (s, 1H), 4.00 (d, J = 1.1 Hz, 3H), 3.79 (s, 3H); |
| | | | ¹⁹F NMR (376 MHz, CDCl₃) |
| | | | δ -63.09 |
| F218 | | ESIMS m/z | ¹H NMR (400 MHz, CDCl₃) δ 7.85 (d, J = 8.5 Hz, 1H), 7.75 (d, J = 8.5 Hz, |
| | | 331 | 1H), 7.51 (d, J = 8.2 Hz, 1H), 7.48-7.44 (m, 1H), 7.28 (ddd, J = 8.2, 1.8, |
| | | ([M + H]⁺) | 0.9 Hz, 1H), 4.31 (s, 2H), 4.03 (s, 3H); |
| | | | ¹³C NMR (101 MHz, CDCl₃) δ 165.11, 154.02, 147.87, 145.03 (d, J = 1.9 |
| | | | Hz), 141.45, 139.16, 129.59, 127.25 (q, J = 5.2 Hz), 123.08, 115.82, 115.67, |
| | | | 52.98 |
| F219 | | ESIMS m/z | ¹H NMR (500 MHz, CDCl₃) δ 8.01-7.93 (m, 2H), 7.64 (d, J = 9.8 Hz, 1H), |
| | | 266 | 7.53-7.41 (m, 3H), 4.02 (s, 3H); |
| | | ([M + H]⁺) | ¹⁹F NMR (471 MHz, CDCl₃) |
| | | | δ -116.59, -116.62 |
| F220 | | ESIMS m/z | ¹H NMR (400 MHz, CDCl₃) δ 8.15 (s, 1H), 8.03-7.93 (m, 2H), 4.03 (s, |
| | | 385 | 3H); |
| | | ([M + H]⁺) | ¹³C NMR (101 MHz, CDCl₃) δ 164.36, 154.86, 151.44, 147.01, 146.03 (q, J = |
| | | | 1.01 Hz), 139.54 (q, J = 4.9 Hz), 139.45, 132.00, 129.57, 127.23, 126.15 |
| | | | (q, J = 34.1 Hz), 121.27 (q, J = 273.7 Hz), 53.16; |
| | | | ¹⁹F NMR (376 MHz, CDCl₃) |
| | | | δ -63.77 |
| F221 | 106- | ESIMS m/z | ¹H NMR (400 MHz, CDCl₃) δ 7.68 (d, J = 8.5 Hz, 1H), 7.64 (t, J = 8.3 Hz, |
| | 108 | 350 | 1H), 7.11-7.05 (m, 1H), 6.99 (dd, J = 10.6, 2.4 Hz, 1H), 6.57 (t, J = 72.8 |
| | | ([M + H]⁺) | Hz, 1H), 4.01 (s, 3H) |
| F222 | | ESIMS m/z | ¹H NMR (400 MHz, CDCl₃) δ 8.07 (d, J = 8.3 Hz, 1H), 7.84-7.80 (m, 1H), |
| | | 362 | 7.71 (d, J = 1.7 Hz, 1H), 7.58 (d, J = 8.3 Hz, 1H), 6.26 (dd, J = 17.4, 11.0 |
| | | (M + H]⁺) | Hz, 1H), 5.78 (d, J = 17.3 Hz, 1H), 5.38 (d, J = 11.0 Hz, 1H); |
| | | | ¹⁹F NMR (376 MHz, CDCl₃) |
| | | | δ -63.13 |
| F223 | | ESIMS m/z | ¹H NMR (400 MHz, CDCl₃) δ 8.02 (d, J = 8.3 Hz, 1H), 7.57 (d, J = 8.3 Hz, |
| | | 361.9 | 1H), 7.42 (dd, J = 1.5, 0.7 Hz, 1H), 7.19-7.14 (m, 1H), 3.83 (s, 3H); |
| | | ([M + H]⁺) | ¹⁹F NMR (376 MHz, CDCl₃) |
| | | | δ -63.14 |
| F224 | | ESIMS m/z | ¹H NMR (400 MHz, CDCl₃) δ 8.30-8.27 (m, 1H), 8.15-8.03 (m, 2H), 7.65 |
| | | 378.9 | (d, J = 8.3 Hz, 1H); |
| | | ([M − H]⁻) | ¹⁹F NMR (376 MHz, CDCl₃) |
| | | | δ -63.18 |
| F225 | | ESIMS m/z | ¹H NMR (400 MHz, CDCl₃) δ 8.32 (d, J = 8.5 Hz, 1H), 8.22 (s, 1H), 8.13 (d, |
| | | 369 | J = 8.4 Hz, 1H); |
| | | ([M − H]⁻) | ¹³C NMR (101 MHz, CDCl₃) δ 161.09, 152.44, 149.95, 146.36, 142.33, |
| | | | 140.97, 140.27 (q, J = 4.9 Hz), 135.17, 129.28, 129.15, 126.66 (q, J = 34.4 |
| | | | Hz), 121.12 (q, J = 273.7 Hz); |
| | | | ¹⁹F NMR (376 MHz, CDCl₃) |
| | | | δ -63.81 |
| F226 | 86- | ESIMS m/z | ¹H NMR (400 MHz, DMSO-d₆) δ 14.01 (s, 1H), 8.21 (d, J = 8.5 Hz, 1H), |
| | 88 | 353 | 7.87 (d, J = 8.4 Hz, 1H), 7.79-7.72 (m, 2H), 7.54 (ddt, J = 7.4, 2.4, 1.2 Hz, |
| | | ([M + H]⁺) | 1H); |
| | | | ¹⁹F NMR (376 MHz, DMSO) |
| | | | δ -56.90 |
| F227 | | ESIMS m/z | ¹H NMR (500 MHz, CDCl₃) δ 8.01 (ddd, J = 9.0, 5.3, 1.6 Hz, 2H), 7.64 (d, J = |
| | | 284 | 9.9 Hz, 1H), 7.18 (t, J = 8.7 Hz, 2H), 4.02 (s, 3H); |
| | | ([M + H]⁺) | ¹⁹F NMR (471 MHz, CDCl₃) |
| | | | δ -110.33, -110.34, -110.34, -110.35, -110.36, -110.37, -110.37, -110.38, -110.39, -116.69, |
| | | | -116.72 |
| F228 | | ESIMS m/z | ¹H NMR (500 MHz, DMSO-d₆) δ 10.64 (s, 1H), 7.36 (s, 1H), 6.69 (d, J = 6.9 |
| | | 354 | Hz, 1H), 6.46 (d, J = 7.1 Hz, 1H), 5.51 (s, 1H), 3.97 (s, 3H), 3.71 (s, 3H); |
| | | ([M − H]⁻) | ¹³C NMR (126 MHz, CDCl₃) δ 172.75, 170.15, 155.59, 137.11, 129.48, |
| | | | 128.92, 127.71, 125.00, 119.75, 106.54, 58.22 |
| F229 | | ESIMS m/z | ¹H NMR (500 MHz, CDCl₃) δ 7.70 (d, J = 8.4 Hz, 1H), 7.38 (dd, J = 8.4, 6.2 |
| | | 363.9 | Hz, 1H), 7.04 (dd, J = 8.4, 0.8 Hz, 1H), 4.01 (s, 3H); |
| | | ([M + H]⁺) | ¹³C NMR (126 MHz, CDCl₃) δ 163.90, 158.05, 155.90, 146.80, 146.76, |
| | | | 144.60, 143.92, 143.89, 142.56, 139.81, 139.68, 134.66, 133.99, 131.92, |
| | | | 131.76, 131.72, 131.37, 131.26, 129.86, 128.05, 126.51, 126.35, 126.33, |
| | | | 119.04, 119.01, 118.95, 118.91, 105.92, 105.89, 53.22; |
| | | | ¹⁹F NMR (471 MHz, CDCl₃) |
| | | | δ -49.30, -49.31, -49.32, -114.24, -114.25, -114.31, -114.32, -135.01, -135.02, -135.08, -135.09 |
| F230 | 139- | ESIMS m/z | ¹H NMR (500 MHz, DMSO-d₆) δ 14.02 (s, 1H), 8.39 (d, J = 9.1 Hz, 1H), |
| | 142 | 350 | 7.48 (dd, J = 8.0, 6.1 Hz, 1H), 7.42 (d, J = 8.4 Hz, 1H), 7.30 (t, J = 54.2 Hz, |
| | | ([M + H]⁺) | 1H), 3.86 (d, J = 1.9 Hz, 3H); |
| | | | ¹⁹F NMR (471 MHz, DMSO) |
| | | | δ -113.66, -113.67, -113.77, -113.79, -115.72, -135.26 |
| F231 | | ESIMS m/z | ¹H NMR (500 MHz, DMSO-d₆) δ 7.20 (d, J = 1.4 Hz, 1H), 7.11 (t, J = 1.6 |
| | | 340 | Hz, 1H), 6.87 (dd, J = 21.9, 1.6 Hz, 1H), 6.11 (s, 2H), 3.94 (s, 3H), 3.89 (s, |
| | | ([M + H]⁺) | 3H); |
| | | | ¹⁹F NMR (471 MHz, DMSO) |
| | | | δ -116.01 |
| F232 | | | ¹H NMR (500 MHz, CDCl₃) δ 8.97 (s, 1H), 7.56 (s, 1H), 7.24 (s, 1H), 4.05 |
| | | | (s, 3H); |
| | | | ¹³C NMR (126 MHz, CDCl₃) δ 163.14, 161.97, 158.76, 154.11, 145.00, |
| | | | 142.74, 131.82, 131.40, 128.61, 127.63, 112.54, 112.10, 53.56; |
| | | | ¹⁹F NMR (471 MHz, CDCl₃) |
| | | | δ -49.91 |
| F233 | | ESIMS m/z | ¹H NMR (500 MHz, CDCl₃) δ 7.68 (d, J = 8.4 Hz, 1H), 7.15 (dd, J = 8.3, 1.0 |
| | | 360 | Hz, 1H), 7.00 (d, J = 8.3 Hz, 1H), 3.99 (s, 3H), 2.23 (d, J = 1.2 Hz, 3H); |
| | | ([M + H]⁺) | ¹⁹F NMR (471 MHz, CDCl₃) |
| | | | δ -49.61, -114.90, -114.91 |
| F234 | | ESIMS m/z | ¹H NMR (500 MHz, CDCl₃) δ 7.71 (d, J = 8.7 Hz, 1H), 7.46 (dd, J = 9.1, 4.6 |
| | | 363.9 | Hz, 1H), 7.06 (t, J = 9.1 Hz, 1H), 4.02 (s, 3H); |
| | | ([M + H]⁺) | ¹⁹F NMR (471 MHz, CDCl₃) |
| | | | δ -49.29, -114.07, -114.09, -132.42, -132.43, -132.44 |
| F235 | | ESIMS m/z | ¹H NMR (500 MHz, CDCl₃) δ 7.61 (d, J = 8.3 Hz, 1H), 7.18 (d, J = 8.3 Hz, |
| | | 376 | 1H), 6.84 (d, J = 8.3 Hz, 1H), 4.06 (s, 3H), 3.99 (s, 3H); |
| | | ([M + H]⁺) | ¹⁹F NMR (471 MHz, CDCl₃) |
| | | | δ -49.80, -49.81, -49.82, -113.26, -113.27, -113.28 |
| F236 | | ESIMS m/z | ¹H NMR (500 MHz, CDCl₃) δ 7.87 (d, J = 8.5 Hz, 1H), 7.76 (d, J = 8.4 Hz, |
| | | 347 | 1H), 7.65 (d, J = 8.1 Hz, 1H), 7.41 (d, J = 1.9 Hz, 1H), 7.28 (dd, J = 8.1, 2.0 |
| | | ([M + H]⁺) | Hz, 1H), 4.01 (s, 3H), 1.86-1.77 (m, 2H), 1.50-1.41 (m, 2H; |
| | | | ¹³C NMR (126 MHz, CDCl₃) δ 206.93, 164.85, 153.78, 147.73, 138.87, |
| | | | 138.40, 136.46, 132.79, 132.30, 129.83, 127.42, 124.40, 121.62, 53.09, |
| | | | 30.94, 18.75, 13.51 |
| F237 | | ESIMS m/z | ¹H NMR (500 MHz, CDCl₃) δ 7.68 (d, J = 8.2 Hz, 1H), 7.47 (d, J = 8.0 Hz, |
| | | 364.9 | 1H), 7.40 (d, J = 1.9 Hz, 1H), 7.32 (dd, J = 8.1, 2.0 Hz, 1H), 3.99 (s, 3H), |
| | | ([M + H]⁺) | 1.90-1.71 (m, 2H), 1.56-1.39 (m, 2H); |
| | | | ¹⁹F NMR (471 MHz, CDCl₃) |
| | | | δ -112.73, -112.75 |
| F238 | | ESIMS m/z | ¹H NMR (500 MHz, CDCl₃) δ 7.97 (s, 1H), 7.41-7.34 (m, 3H), 7.31 (dd, J = |
| | | 380.9 | 8.1, 1.9 Hz, 1H), 3.98 (s, 3H), 1.86-1.77 (m, 3H), 1.53-1.42 (m, 2H); |
| | | ([M + H]⁺) | ¹³C NMR (126 MHz, CDCl₃) δ 164.00, 152.88, 145.33, 139.24, 139.22, |
| | | | 135.15, 134.02, 133.96, 131.20, 126.78, 124.28, 121.59, 53.26, 30.94, 18.78, |
| | | | 13.61 |
| F239 | | ESIMS m/z | ¹H NMR (400 MHz, CDCl₃) δ 7.94 (d, J = 8.4 Hz, 1H), 7.85-7.69 (m, 2H), |
| | | 373 | 7.49 (d, J = 8.3 Hz, 1H), 4.01 (s, 3H), 3.09 (s, 1H); |
| | | ([M + H]⁺) | ¹⁹F NMR (376 MHz, CDCl₃) |
| | | | δ -63.25 |
| F240 | | ESIMS m/z | ¹H NMR (500 MHz, CDCl₃) δ 7.83-7.58 (m, 3H), 4.03 (s, 3H); |
| | | 363.9 | ¹⁹F NMR (471 MHz, CDCl₃) |
| | | ([M + H]⁺) | δ -49.49, -116.24, -116.26, -134.07 |
| F241 | | ESIMS m/z | ¹H NMR (500 MHz, CDCl₃) δ 7.93-7.62 (m, 2H), 7.38 (d, J = 9.7 Hz, 1H), |
| | | 385.9 | 4.01 (d, J = 0.7 Hz, 3H; |
| | | ([M + H]⁺) | ¹⁹F NMR (471 MHz, CDCl₃) |
| | | | δ -61.78, -112.50, -115.55 |
| F242 | | ESIMS m/z | ¹H NMR (500 MHz, CDCl₃) δ 8.03-7.98 (m, 2H), 7.90 (d, J = 8.5 Hz, 1H), |
| | | 328 | 7.21 (t, J = 8.1 Hz, 1H), 7.13 (dd, J = 7.9, 1.2 Hz, 1H), 4.04 (s, 3H); |
| | | ([M + H]⁺) | ¹⁹F NMR (471 MHz, CDCl₃) |
| | | | δ -49.62 |
| F243 | | ESIMS m/z | ¹H NMR (500 MHz, CDCl₃) δ 7.93 (d, J = 8.4 Hz, 1H), 7.83 (d, J = 8.4 Hz, |
| | | 367.9 | 1H), 7.73 (d, J = 6.2 Hz, 1H), 7.58 (d, J = 10.2 Hz, 1H), 4.03 (s, 3H); |
| | | ([M + H]⁺) | ¹⁹F NMR (471 MHz, CDCl₃) |
| | | | δ -61.71, -115.61 |
| F244 | | ESIMS m/z | ¹H NMR (500 MHz, CDCl₃) δ 8.24 (d, J = 1.6 Hz, 1H), 7.84 (d, J = 1.7 Hz, |
| | | 404 | 1H), 7.74 (d, J = 8.6 Hz, 1H), 4.03 (s, 3H), 3.95 (s, 3H); |
| | | ([M + H]⁺) | ¹⁹F NMR (471 MHz, CDCl₃) |
| | | | δ -49.42, -113.83 |
| F245 | | ESIMS m/z | ¹H NMR (500 MHz, CDCl₃) δ 8.72 (d, J = 1.6 Hz, 1H), 8.01 (d, J = 8.5 Hz, |
| | | 386 | 1H), 7.92 (d, J = 8.5 Hz, 1H), 7.79 (d, J = 1.6 Hz, 1H), 4.06 (s, 3H), 3.97 (s, |
| | | ([M + H]⁺) | 3H); |
| | | | ¹⁹F NMR (471 MHz, CDCl₃) |
| | | | δ -49.22 |
| F246 | | ESIMS m/z | ¹H NMR (500 MHz, CDCl₃) δ 8.01 (s, 1H), 7.73 (d, J = 6.2 Hz, 1H), 7.27 (d, |
| | | 401.9 | J = 10.5 Hz, 1H), 4.00 (s, 3H); |
| | | ([M + H]⁺) | ¹³C NMR (126 MHz, CDCl₃) δ 163.71, 158.98, 156.92, 151.05, 145.56, |
| | | | 141.05, 139.59, 133.64, 131.72, 128.86, 128.43, 119.76, 119.58, 53.38 |
| F247 | 127- | ESIMS m/z | ¹H NMR (500 MHz, CDCl₃) δ 7.86 (dd, J = 8.7, 1.7 Hz, 2H), 7.55 (d, J = |
| | 130 | 281 | 10.3 Hz, 1H), 6.77-6.73 (m, 2H), 4.00 (s, 3H), 3.92 (s, 2H); |
| | | ([M + H]⁺) | ¹⁹F NMR (471 MHz, CDCl₃) |
| | | | δ -116.87, -116.89 |
| F248 | 91- | ESIMS m/z | ¹H NMR (500 MHz, CDCl₃) δ 7.94 (dd, J = 8.5, 1.7 Hz, 2H), 7.62 (d, J = |
| | 93 | 312 | 10.1 Hz, 1H), 7.33 (d, J = 8.6 Hz, 2H), 4.01 (s, 3H), 2.53 (s, 3H); |
| | | ([M + H]⁺) | ¹⁹F NMR (471 MHz, CDCl₃) |
| | | | δ -116.42, -116.44 |
| F249 | 58- | ESIMS m/z | ¹H NMR (500 MHz, CDC) δ 7.88 (dd, J = 8.2, 1.8 Hz, 2H), 7.62 (d, J = |
| | 61 | 280 | 10.0 Hz, 1H), 7.29 (d, J = 8.0 Hz, 2H), 4.01 (s, 3H), 2.41 (s, 3H); |
| | | ([M + H]⁺) | ¹⁹F NMR (471 MHz, CDCl₃) |
| | | | δ -116.58, -116.60 |
| F250 | | ESIMS m/z | ¹H NMR(400 MHz, CDCl₃) δ 7.95 (d, J = 8.3 Hz, 1H), 7.59 (dd, J = 2.0, 1.0 |
| | | 364 | Hz, 1H), 7.46 (dt, J = 1.7, 0.8 Hz, 1H), 7.41 (d, J = 8.3 Hz, 1H), 4.00 (s, 3H), |
| | | ([M + H]⁺) | 2.19 (s, 3H); |
| | | | ¹⁹F NMR (376 MHz, CDCl₃) |
| | | | δ -63.05 |
| F251 | | ESIMS m/z | ¹H NMR (400 MHz, CDCl₃) δ 7.80 (s, 1H), 7.67 (s, 1H), 7.49 (s, 1H), 3.96 |
| | | 391.9 | (s, 3H), 3.47-3.44 (m, 1H); |
| | | ([M + H]⁺) | ¹⁹F NMR (376 MHz, CDCl₃) |
| | | | δ -62.70, -110.58 |
| F252 | | ESIMS m/z | ¹H NMR (400 MHz, CDCl₃) δ 8.36 (d, J = 8.5 Hz, 1H), 8.10 (d, J = 8.4 Hz, |
| | | 347 | 1H), 7.87-7.79 (m, 2H), 7.74-7.67 (m, 1H); |
| | | ([M + H]⁺) | ¹⁹F NMR (376 MHz, CDCl₃) |
| | | | δ -63.08 |
| F253 | 58- | ESIMS m/z | ¹H NMR (500 MHz, CDCl₃) δ 7.67 (d, J = 8.3 Hz, 1H), 7.43-7.32 (m, 2H), |
| | 68 | 364 | 6.92 (t, J = 54.8 Hz, 1H), 4.00 (s, 3H), 3.96 (d, J = 2.5 Hz, 3H); |
| | | ([M + H]⁺) | ¹⁹F NMR (471 MHz, CDCl₃) |
| | | | δ -113.42, -113.44, -114.30, -114.31, -114.42, -114.42, -136.20 |
| F254 | | ESIMS m/z | ¹H NMR (400 MHz, CDCl₃) δ 7.86 (d, J = 8.1 Hz, 1H), 7.81 (d, J = 6.2 Hz, |
| | | 393.9 | 1H), 7.38 (d, J = 9.6 Hz, 1H); |
| | | ([M + Na]⁺) | ¹⁹F NMR (376 MHz, CDCl₃) |
| | | | δ -61.83, -110.53, -114.91 |
| F255 | | ESIMS m/z | ¹H NMR (400 MHz, CDCl₃) δ 11.27 (s, 1H), 8.12 (d, J = 8.5 Hz, 1H), 8.05 |
| | | 312 | (d, J = 8.5 Hz, 1H), 7.77 (dd, J = 8.1, 1.3 Hz, 1H), 7.26-7.17 (m, 2H); |
| | | ([M − H]⁻) | ¹⁹F NMR (376 MHz, CDCl₃) |
| | | | δ -49.49 |
| F256 | | ESIMS m/z | ¹H NMR (400 MHz, CDCl₃) δ 7.71 (d, J = 8.7 Hz, 1H), 7.46 (dd, J = 7.8, 1.6 |
| | | 346 | Hz, 1H), 7.25-7.14 (m, 2H), 4.02 (s, 3H); |
| | | ([M + H]⁺) | ¹⁹F NMR (376 MHz, CDCl₃) δ 49.8, 113.7 |
| F257 | | ESIMS m/z | ¹H NMR (400 MHz, CDCl₃) δ 8.01 (s, 1H), 7.80 (s, 1H), 7.73 (s, 1H), 4.00 |
| | | 462 | (s, 3H); |
| | | ([M + H]⁺) | ¹⁹F NMR (376 MHz, CDCl₃) |
| | | | δ -63.17 |
| F258 | | ESIMS m/z | ¹H NMR (400 MHz, CDCl₃) δ 7.89-7.71 (m, 2H), 7.66 (d, J = 10.2 Hz, |
| | | 346 | 1H), 7.16 (d, J = 8.4 Hz, 1H), 4.02 (s, 3H); |
| | | ([M + H]⁺) | ¹⁹F NMR (376 MHz, CDCl₃) δ -50.00, -116.53 |
| F259 | | | ¹H NMR (400 MHz, CDCl₃) δ 10.27 (s, 1H), 8.09 (d, J = 8.3 Hz, 1H), 7.63 |
| | | | (d, J = 1.7 Hz, 1H), 7.57 (d, J = 8.3 Hz, 1H), 7.51 (d, J = 1.9 Hz, 1H), 2.20 |
| | | | (s, 3H); |
| | | | ¹³C NMR (101 MHz, CDCl₃) δ 161.92, 153.07, 142.73, 141.83, 139.78, |
| | | | 139.59, 133.86, 133.08, 132.20, 129.68, 125.80, 124.34, 121.73, 20.70; |
| | | | ¹⁹F NMR (376 MHz, CDCl₃) |
| | | | δ -63.06 |
| F260 | | ESIMS m/z | ¹H NMR (400 MHz, CDCl₃) δ 8.06 (dd, J = 8.3, 7.2 Hz, 1H), 7.96 (dd, J = |
| | | 322 | 8.5, 1.8 Hz, 1H), 7.85 (d, J = 8.5 Hz, 1H), 7.69 (d, J = 8.4 Hz, 1H), 7.57- |
| | | ([M + H]⁺) | 7.53 (m, 1H), 7.37 (dd, J = 5.3, 4.0 Hz, 1H), 4.04 (s, 3H); |
| | | | ¹⁹F NMR (376 MHz, CDCl₃) |
| | | | δ -118.25 |
| F261 | | ESIMS m/z | ¹H NMR (400 MHz, CDCl₃) δ 8.11 (d, J = 0.8 Hz, 1H), 8.06 (d, J = 0.8 Hz, |
| | | 321.9 | 1H), 7.96 (d, J = 8.4 Hz, 1H), 7.70 (d, J = 8.4 Hz, 1H), 4.04 (s, 3H); |
| | | ([M + H]⁺) | ¹³C NMR (101 MHz, CDCl₃) δ 164.46, 152.66, 148.65, 147.96, 147.82, |
| | | | 141.54, 138.85, 136.63, 131.14, 127.25, 119.55, 116.77, 53.25 |
| F262 | | ESIMS m/z | ¹H NMR (500 MHz, CDCl₃) δ 8.31 (dd, J = 1.7, 0.6 Hz, 1H), 8.17 (s, 1H), |
| | | 289 | 8.01 (dd, J = 8.4, 1.6 Hz, 1H), 7.91-7.83 (m, 3H), 4.05 (s, 3H); |
| | | ([M + H]⁺) | ¹³C NMR (126 MHz, CDCl₃) δ 165.16, 154.61, 153.86, 150.68, 147.85, |
| | | | 141.37, 139.29, 135.42, 129.13, 123.76, 123.02, 120.76, 110.00, 53.04 |
| F263 | | ESIMS m/z | ¹H NMR (400 MHz, CDCl₃) δ 8.16 (dd, J = 8.5, 1.6 Hz, 2H), 7.80-7.75 (m, |
| | | 328 | 2H), 7.70 (d, J = 9.9 Hz, 1H), 4.03 (s, 3H), 2.77 (s, 3H); |
| | | ([M + H]⁺) | ¹⁹F NMR (376 MHz, CDCl₃) |
| | | | δ -116.29 |
| F264 | | ESIMS m/z | ¹H NMR (500 MHz, CDCl₃) δ 7.64 (d, J = 8.3 Hz, 1H), 7.25-7.18 (m, 2H), |
| | | 314 | 7.15-7.09 (m, 1H), 3.99 (s, 3H), 3.93 (d, J = 2.4 Hz, 3H); |
| | | ([M + H]⁺) | ¹⁹F NMR (471 MHz, CDCl₃) |
| | | | δ -113.34, -113.36, -129.54, -129.57 |
| F265 | | EIMS m/z | ¹H NMR (500 MHz, CDCl₃) δ 9.02 (s, 1H), 8.38 (d, J = 6.2 Hz, 1H), 7.77 (d, |
| | | 341 | J = 9.1 Hz, 1H), 7.72 (d, J = 8.4 Hz, 1H), 4.02 (s, 3H); |
| | | | ¹⁹F NMR(471 MHz, CDCl₃) |
| | | | δ -113.90, -116.98 |
| F266 | | EIMS m/z | ¹H NMR (500 MHz, CDCl₃) δ 7.71 (dd, J = 15.6, 8.2 Hz, 2H), 7.63-7.57 |
| | | 339.9 | (m, 2H), 7.41 (dd, J = 5.3, 3.8 Hz, 1H), 4.01 (s, 3H); |
| | | | ¹⁹F NMR (471 MHz, CDCl₃) |
| | | | δ -113.47, -115.06 |
| F267 | 102- | ESIMS m/z | ¹H NMR (400 MHz, CDCl₃) δ 7.83 (d, J = 8.1 Hz, 1H), 7.65-7.58 (m, 2H), |
| | 104 | 334 | 7.48 (d, J = 7.9 Hz, 1H), 2.41-2.27 (m, 3H); |
| | | ([M + H]⁺) | ¹⁹F NMR (471 MHz, CDCl₃) |
| | | | δ -62.94, -111.86, -111.88 |
| F268 | | ESIMS m/z | ¹H NMR (600 MHz, CDCl₃) δ 8.08 (td, J = 8.9, 6.6 Hz, 1H), 7.88-7.78 (m, |
| | | 284 | 2H), 7.01 (dddd, J = 8.8, 7.7, 2.5, 1.0 Hz, 1H), 6.91 (ddd, J = 11.3, 8.7, 2.5 |
| | | ([M + H]⁺) | Hz, 1H), 4.03 (s, 3H); |
| | | | ¹⁹F NMR (564 MHz, CDCl₃) |
| | | | δ -107.52, -112.27 |
| F269 | | ESIMS m/z | ¹H NMR (600 MHz, CDCl₃) δ 8.74 (dd, J = 2.5, 0.7 Hz, 1H), 8.26 (dd, J = |
| | | 278.9 | 8.7, 2.5 Hz, 1H), 7.83 (d, J = 8.5 Hz, 1H), 7.72 (d, J = 8.5 Hz, 1H), 6.84 (dd, |
| | | ([M + H]⁺) | J = 8.7, 0.7 Hz, 1H), 4.03 (s, 3H), 4.00 (s, 3H); |
| | | | ¹³C NMR (151 MHz, CDCl₃) δ 165.18, 165.09, 153.07, 147.87, 145.78, |
| | | | 139.19, 137.47, 128.62, 126.66, 121.98, 111.15, 53.79, 52.98 |
| F270 | | ESIMS m/z | ¹H NMR (600 MHz, CDCl₃) δ 8.04 (t, J = 8.5 Hz, 1H), 7.85 (d, J = 1.0 Hz, |
| | | 300 | 2H), 7.30-7.23 (m, 1H), 7.20 (dd, J = 11.1, 2.0 Hz, 1H), 4.03 (s, 3H); |
| | | ([M + H]⁺) | ¹⁹F NMR (564 MHz, CDCl₃) |
| | | | δ -114.09 |
| F271 | | ESIMS m/z | ¹H NMR (600 MHz, CDCl₃) δ 8.06 (d, J = 7.3 Hz, 1H), 7.88-7.81 (m, 2H), |
| | | 314 | 7.04 (dd, J = 11.6, 0.9 Hz, 1H), 4.04 (s, 3H), 2.40 (s, 3H); |
| | | ([M + H]⁺) | ¹³C NMR (151 MHz, CDCl₃) δ 164.97, 159.53, 157.87, 149.98, 147.85, |
| | | | 139.80, 138.92, 130.81, 130.26, 129.32, 126.60, 123.99, 118.39, 53.04, 20.13 |
| F272 | | ESIMS m/z | ¹H NMR (600 MHz, CDCl₃) δ 7.87-7.84 (m, 2H), 7.83-7.78 (m, 1H), 7.28 |
| | | 313.9 | (dd, J = 8.5, 1.2 Hz, 1H), 4.02 (s, 3H), 2.38 (d, J = 2.6 Hz, 3H); |
| | | ([M + H]⁺) | ¹⁹F NMR (564 MHz, CDCl₃) |
| | | | δ -115.98 |
| F273 | | ESIMS m/z | ¹H NMR (600 MHz, CDCl₃) δ 8.15 (t, J = 8.4 Hz, 1H), 7.89 (d, J = 1.1 Hz, |
| | | 350 | 2H), 7.36 (dd, J = 8.6, 1.2 Hz, 1H), 7.24-6.98 (m, 1H), 4.03 (s, 3H); |
| | | ([M + H]⁺) | ¹⁹F NMR (564 MHz, CDCl₃) |
| | | | δ -114.42, -117.38 (d, J = 8.1 Hz) |
| F274 | | ESIMS m/z | ¹H NMR (600 MHz, CDCl₃) δ 8.08 (td, J = 8.5, 2.4 Hz, 1H), 7.87 (d, J = 1.1 |
| | | 332 | Hz, 2H), 7.37 (dt, J = 8.5, 1.1 Hz, 1H), 5.68 (d, J = 2.4 Hz, 1H), 5.60 (d, J = |
| | | ([M + H]⁺) | 2.4 Hz, 1H), 4.03 (s, 3H); |
| | | | ¹⁹F NMR (564 MHz, CDCl₃) |
| | | | δ -117.00 |
| F275 | 104- | ESIMS m/z | ¹H NMR (500 MHz, CDCl₃) δ 7.61 (d, J = 8.3 Hz, 1H), 7.53 (dt, J = 7.8, 1.0 |
| | 106 | 346 | Hz, 1H), 7.20 (dt, J = 7.8, 1.3 Hz, 1H), 7.13 (s, 1H), 6.68 (t, J = 56.4 Hz, |
| | | ([M + H]⁺) | 1H), 3.99 (s, 3H), 3.86 (s, 3H); |
| | | | ¹⁹F NMR (471 MHz, CDCl₃) |
| | | | δ -111.19, -111.31, -112.40, -112.42 |
| F276 | | ESIMS m/z | ¹H NMR (600 MHz, CDCl₃) δ 7.87 (d, J = 8.4 Hz, 1H), 7.72 (d, J = 8.4 Hz, |
| | | 360 | 1H), 7.40 (d, J = 8.4 Hz, 1H), 7.32 (d, J = 8.4 Hz, 1H), 4.12 (q, J = 7.0 Hz, |
| | | ([M + H]⁺) | 2H), 4.01 (s, 3H), 1.49 (t, J = 7.0 Hz, 3H); |
| | | | ¹³C NMR (151 MHz, CDCl₃) δ 164.80, 153.86, 152.13, 147.60, 138.46, |
| | | | 137.44, 130.46, 129.92, 128.69, 128.25, 127.59, 126.87, 69.66, 53.11, 15.49 |
| F277 | | ESIMS m/z | ¹H NMR (600 MHz, CDCl₃) δ 7.92 (ddd, J = 7.9, 6.9, 1.7 Hz, 1H), |
| | | 300 | 7.89-7.84 (m, 2H), 7.47 (ddd, J = 7.9, 6.9, 1.7 Hz, 1H), 7.21 (td, J = 7.9, 1.1 Hz, |
| | | ([M + H]⁺) | 1H), 4.03 (s, 3H); |
| | | | ¹⁹F NMR (564 MHz, CDCl₃) |
| | | | δ -119.05 |
| F278 | | ESIMS m/z | ¹H NMR (600 MHz, CDCl₃) δ 7.86 (d, J = 8.5 Hz, 1H), 7.73 (d, J = 8.5 Hz, |
| | | 330 | 1H), 7.43 (t, J = 1.6 Hz, 1H), 7.38 (dd, J = 9.5, 1.9 Hz, 1H), 4.04 (s, 3H), |
| | | ([M + H]⁺) | 4.01 (s, 3H); |
| | | | ¹⁹F NMR (564 MHz, CDCl₃) |
| | | | δ -112.94 |
| F279 | | ESIMS m/z | ¹H NMR (600 MHz, CDCl₃) δ 7.86 (d, J = 8.5 Hz, 1H), 7.83 (dd, J = 8.5, 1.8 |
| | | 372 | Hz, 1H), 7.68 (dd, J = 8.6, 7.7 Hz, 1H), 7.31-7.24 (m, 1H), 4.12 (t, J = 6.5 |
| | | ([M + H]⁺) | Hz, 2H), 4.02 (s, 3H), 1.81 (dq, J = 7.7, 6.5 Hz, 2H), 1.60-1.51 (m, 2H), |
| | | | 0.99 (t, J = 7.4 Hz, 3H); |
| | | | ¹⁹F NMR (564 MHz, CDCl₃) |
| | | | δ -131.50 |
| F280 | | | ¹H NMR (600 MHz, CDCl₃) δ 7.88-7.80 (m, 2H), 7.73 (dd, J = 8.7, 7.7 Hz, |
| | | | 1H), 7.32-7.23 (m, 1H), 4.44 (t, J = 6.7 Hz, 2H), 3.99 (d, J = 0.9 Hz, 3H), |
| | | | 2.03-1.71 (m, 2H), 1.56-1.41 (m, 2H), 0.99 (t, J = 7.4 Hz, 3H); |
| | | | ¹³C NMR (151 MHz, CDCl₃) δ 164.81, 155.46, 153.77, 150.15, 148.69, |
| | | | 144.83, 138.76, 129.97, 129.09, 126.34, 125.54, 125.51, 66.16, 61.73, 30.57, |
| | | | 19.16, 13.71; |
| | | | ¹⁹F NMR (564 MHz, CDCl₃) |
| | | | δ -131.96 |
| F281 | | | ¹H NMR (600 MHz, CDCl₃) δ 7.90 (d, J = 8.4 Hz, 1H), 7.69 (d, J = 8.4 Hz, |
| | | | 1H), 7.61 (dt, J = 8.3, 1.1 Hz, 1H), 7.49 (d, J = 8.4 Hz, 1H), 7.33 (t, J = 52.9 |
| | | | Hz, 1H), 4.01 (s, 3H); |
| | | | ¹³C NMR (151 MHz, CDCl₃) δ 164.67, 153.34, 147.89, 138.64, 137.91, |
| | | | 135.83, 134.12, 133.13, 130.34, 129.68, 129.41, 127.67, 113.97, 112.38, |
| | | | 110.78, 53.18; |
| | | | ¹⁹F NMR (564 MHz, CDCl₃) |
| | | | δ -115.26 |
| F282 | | ESIMS m/z | ¹H NMR (600 MHz, CDCl₃) δ 7.88 (d, J = 8.4 Hz, 1H), 7.66 (d, J = 8.4 Hz, |
| | | 361.89 | 1H), 7.49-7.39 (m, 2H), 6.37 (dq, J = 46.2, 6.7 Hz, 1H), 4.01 (s, 3H), 1.79 |
| | | ([M + H]⁺) | (dd, J = 22.2, 6.7 Hz, 3H); |
| | | | ¹⁹F NMR (564 MHz, CDCl₃) |
| | | | δ -176.82 |
| F283 | | ESIMS m/z | ¹H NMR (600 MHz, CDCl₃) δ 7.91-7.80 (m, 2H), 7.67 (dd, J = 8.6, 7.5 Hz, |
| | | 415.9 | 1H), 7.43 (dd, J = 8.7, 1.7 Hz, 1H), 4.44 (t, J = 6.7 Hz, 2H), 3.98 (d, J = 1.0 |
| | | ([M + H]⁺) | Hz, 3H), 1.87-1.75 (m, 2H), 1.57-1.41 (m, 2H), 0.99 (t, J = 7.4 Hz, 3H); |
| | | | ¹⁹F NMR (564 MHz, CDCl₃) |
| | | | δ -131.36 |
| F284 | | ESIMS m/z | ¹H NMR (600 MHz, CDCl₃) δ 8.04 (d, J = 8.0 Hz, 1H), 7.98-7.85 (m, 2H), |
| | | 316.9 | 7.42 (d, J = 8.0 Hz, 1H), 4.02 (s, 3H); |
| | | ([M + H]⁺) | ¹³C NMR (151 MHz, CDCl₃) δ 164.62, 151.66, 150.69, 148.10, 147.87, |
| | | | 142.75, 138.83, 132.47, 130.51, 127.27, 123.58, 53.18 |
| F285 | | ESIMS m/z | ¹H NMR (600 MHz, CDCl₃) δ 7.96 (td, J = 8.3, 1.1 Hz, 1H), 7.87 (d, J = 1.1 |
| | | 359.9 | Hz, 2H), 7.31 (dt, J = 8.6, 1.2 Hz, 1H), 6.00-5.86 (m, 1H), 4.03 (s, 3H), |
| | | ([M + H]⁺) | 2.35-2.19 (m, 1H), 2.10-1.93 (m, 1H), 1.04 (t, J = 7.5 Hz, 3H); |
| | | | ¹⁹F NMR (564 MHz, CDCl₃) |
| | | | δ -116.23, -181.88 |
| F286 | | ESIMS m/z | ¹H NMR (600 MHz, CDCl₃) δ 7.90-7.80 (m, 2H), 7.73 (dd, J = 8.7, 7.7 Hz, |
| | | 357.9 | 1H), 7.27 (d, J = 8.4 Hz, 1H), 4.40 (t, J = 6.7 Hz, 2H), 3.99 (d, J = 0.9 Hz, |
| | | ([M + H]⁺) | 3H), 1.84 (q, J = 7.0 Hz, 2H), 1.05 (t, J = 7.4 Hz, 3H); |
| | | | ¹⁹F NMR (564 MHz, CDCl₃) |
| | | | δ -131.99 |
| F287 | | ESIMS m/z | ¹H NMR (600 MHz, CDCl₃) δ 7.80 (d, J = 8.5 Hz, 1H), 7.72-7.66 (m, 2H), |
| | | 306 | 7.64 (dd, J = 8.1, 1.8 Hz, 1H), 6.94 (t, J = 7.9 Hz, 1H), 4.03 (s, 3H), 2.13 |
| | | ([M + H]⁺) | (ddd, J = 8.5, 5.2, 3.3 Hz, 1H), 1.07-0.98 (m, 2H), 0.83-0.72 (m, 2H); |
| | | | ¹⁹F NMR (564 MHz, CDCl₃) |
| | | | δ -119.71 |
| F288 | 122- | ESIMS m/z | ¹H NMR (500 MHz, CDCl₃) δ 7.89 (d, J = 8.4 Hz, 1H), 7.76 (d, J = 8.4 Hz, |
| | 124 | 377 | 1H), 7.72 (d, J = 8.5 Hz, 1H), 7.46 (d, J = 2.4 Hz, 1H), 7.33 (dd, J = 8.5, 2.4 |
| | | ([M + H]⁺) | Hz, 1H), 4.02 (s, 3H), 3.21 (s, 3H) |
| F289 | | ESIMS m/z | ¹H NMR (600 MHz, CDCl₃) δ 8.26 (td, J = 7.6, 1.8 Hz, 1H), 7.93-7.88 (m, |
| | | 334 | 2H), 7.72-7.66 (m, 1H), 7.41-7.34 (m, 1H), 4.03 (s, 3H); |
| | | ([M + H]⁺) | ¹⁹F NMR (564 MHz, CDCl₃) |
| | | | δ -61.30, 119.39 |
| F290 | | ESIMS m/z | ¹H NMR (600 MHz, CDCl₃) δ 7.99 (ddd, J = 8.3, 6.8, 1.7 Hz, 1H), 7.92-7.85 (m, 2H), |
| | | 349.9 | 7.39 (ddt, J = 8.3, 6.9, 1.4 Hz, 1H), 7.28 (td, J = 8.1, 1.4 Hz, 1H), 4.03 (s, 3H); |
| | | ([M + H]⁺) | ¹⁹F NMR (564 MHz, CDCl₃) δ -58.79, -133.60 |
| F291 | | ESIMS m/z | ¹H NMR (600 MHz, CDCl₃) δ 7.89 (dd, J = 12.1, 7.4 Hz, 1H), 7.86 (dd, J = |
| | | 313.9 | 8.6, 1.2 Hz, 1H), 7.81 (d, J = 8.5 Hz, 1H), 6.75 (dd, J = 12.4, 6.9 Hz, 1H), |
| | | ([M + H]⁺) | 4.03 (s, 3H), 3.92 (s, 3H); |
| | | | ¹⁹F NMR (564 MHz, CDCl₃) |
| | | | δ -118.08, -139.68 |
| F292 | | ESIMS m/z | ¹H NMR (600 MHz, CDCl₃) δ 7.82 (d, J = 8.5 Hz, 1H), 7.72 (d, J = 8.5 Hz, |
| | | 296 | 1H), 7.68 (dd, J = 8.2, 2.2 Hz, 1H), 7.45 (ddd, J = 8.4, 4.2, 2.2 Hz, 1H), 7.15 |
| | | ([M + H]⁺) | (dd, J = 10.8, 8.4 Hz, 1H), 4.03 (s, 3H), 3.98 (s, 3H); |
| | | | ¹⁹F NMR (564 MHz, CDCl₃) |
| | | | δ -133.28 |
| F293 | | ESIMS m/z | ¹H NMR (600 MHz, CDCl₃) δ 8.69 (t, J = 1.6 Hz, 1H), 7.86 (t, J = 7.9 Hz, |
| | | 331.9 | 1H), 7.77 (t, J = 1.3 Hz, 1H), 7.53 (d, J = 8.3 Hz, 1H), 2.46 (dd, J = 3.0, 1.6 |
| | | ([M + H]⁺) | Hz, 3H); |
| | | | ¹⁹F NMR (564 MHz, CDCl₃) |
| | | | δ -61.19, -118.37 |
| F294 | | ESIMS m/z | ¹H NMR (600 MHz, CDCl₃) δ 7.87 (dd, J = 9.5, 7.4 Hz, 2H), 7.75 (d, J = 8.2 |
| | | 387.9 | Hz, 1H), 7.07 (t, J = 52.1 Hz, 1H); |
| | | ([M + H]⁺) | ¹³CNMR (151 MHz, CDCl₃) δ 160.75, 160.07, 159.28, 158.34, 157.46, |
| | | | 139.43, 139.40, 138.34, 138.23, 135.64, 135.60, 133.90, 128.96, 128.81, |
| | | | 126.23, 123.32, 122.32, 122.28, 122.24, 122.21, 121.50, 116.37, 111.53, |
| | | | 109.93, 108.34 |
| F295 | | ESIMS m/z | ¹H NMR (600 MHz, CDCl₃) δ 7.69 (d, J = 9.9 Hz, 1H), 7.66 (d, J = 8.1 Hz, |
| | | 363.9 | 1H), 7.63 (s, 1H), 7.62-7.59 (m, 1H), 4.03 (s, 3H), 3.99 (s, 3H); |
| | | ([M + H]⁺) | ¹⁹F NMR (564 MHz, CDCl₃) |
| | | | δ -62.64, -115.92 |
| F296 | | ESIMS m/z | ¹H NMR (600 MHz, CDCl₃) δ 7.87 (d, J = 8.5 Hz, 1H), 7.80 (d, J = 8.5 Hz, |
| | | 345.9 | 1H), 7.70 (d, J = 1.5 Hz, 1H), 7.64 (d, J = 8.1 Hz, 1H), 7.54-7.50 (m, 1H), |
| | | ([M + H]⁺) | 4.04 (s, 3H), 4.00 (s, 3H); |
| | | | ¹⁹F NMR (564 MHz, CDCl₃) |
| | | | δ -62.48 |
| F297 | | ESIMS m/z | ¹H NMR (600 MHz, CDCl₃) δ 7.78 (d, J = 2.2 Hz, 1H), 7.76 (d, J = 8.5 Hz, |
| | | 373.9 | 1H), 7.71 (d, J = 7.9 Hz, 1H), 7.67 (d, J = 7.7 Hz, 1H), 7.09-7.01 (m, 1H), |
| | | ([M + H]⁺) | 4.02 (s, 3H); |
| | | | ¹⁹F NMR (564 MHz, CDCl₃) |
| | | | δ -61.54, -113.00 |
| F298 | | ESIMS m/z | ¹H NMR (600 MHz, CDCl₃) δ 7.88 (d, J = 8.4 Hz, 1H), 7.45 (d, J = 8.4 Hz, |
| | | 313.9 | 1H), 7.35-7.29 (m, 1H), 7.16 (dd, J = 8.3, 1.4Hz, 1H), 4.01 (s, 3H), 2.31 |
| | | ([M + H]⁺) | (d, J = 2.7 Hz, 3H); |
| | | | ¹⁹F NMR (564 MHz, CDCl₃) |
| | | | δ -116.39 |
| F299 | | ESIMS m/z | ¹H NMR (600 MHz, CDCl₃) δ 7.84 (t, J = 1.1 Hz, 2H), 7.73 (dd, J = 8.7, 7.7 |
| | | 405.9 | Hz, 1H), 7.53-7.47 (m, 2H), 7.43-7.37 (m, 2H), 7.37-7.32 (m, 1H), 7.27 |
| | | ([M + H]⁺) | (d, J = 1.7 Hz, 1H), 5.47 (s, 2H), 3.99 (d, J = 0.9 Hz, 3H); |
| | | | ¹⁹F NMR (564 MHz, CDCl₃) |
| | | | δ -131.97 |
| F300 | | ESIMS m/z | ¹H NMR (600 MHz, CDCl₃) δ 7.70 (d, J = 7.9 Hz, 1H), 7.49-7.45 (m, 2H), |
| | | 457.9 | 7.41-7.32 (m, 4H), 5.45 (d, J = 1.9 Hz, 2H), 3.97 (d, J = 1.3 Hz, 3H); |
| | | ([M + H]⁺) | ¹⁹F NMR (564 MHz, CDCl₃) |
| | | | δ -113.89, -125.20 |
| F301 | | ESIMS m/z | ¹H NMR (600 MHz, CDCl₃) δ 7.64 (d, J = 7.8 Hz, 1H), 7.34 (t, J = 8.3 Hz, |
| | | 329.9 | 1H), 7.10 (dd, J = 8.2, 0.9 Hz, 1H), 6.89 (dd, J = 8.5, 0.9 Hz, 1H), 3.98 (s, |
| | | ([M + H]⁺) | 3H), 3.74 (s, 3H); |
| | | | ¹⁹F NMR (564 MHz, CDCl₃) |
| | | | δ -114.28 |
| F302 | | ESIMS m/z | ¹H NMR (600 MHz, CDCl₃) δ 7.94 (dd, J = 8.7, 7.7 Hz, 1H), 7.89 (d, J = 8.5 |
| | | 365.9 | Hz, 1H), 7.86 (dd, J = 8.5, 1.8 Hz, 1H), 7.37 (dd, J = 8.7, 1.7 Hz, 1H), 6.80-6.42 |
| | | ([M + H]⁺) | (m, 1H), 4.03 (s, 3H); |
| | | | ¹⁹F NMR (564 MHz, CDCl₃) |
| | | | δ -81.69, -128.66 |
| F303 | | EIMS m/z | ¹H NMR (400 MHz, CDCl₃) δ 7.80-7.69 (m, 3H), 7.05 (ddq, J = 17.7, 11.0, |
| | | 393.9 | 2.4 Hz, 1H), 5.78 (d, J = 17.2 Hz, 1H), 5.50 (dd, J = 11.0, 0.7 Hz, 1H), 4.01 |
| | | | (d, J = 2.9 Hz, 3H); |
| | | | ¹⁹F NMR (376 MHz, CDCl₃) |
| | | | δ -60.17, -112.77 |
| F304 | | ESIMS m/z | ¹H NMR (400 MHz, CDCl₃) δ 8.20-8.16 (m, 1H), 7.97 (ddd, J = 8.3, 1.8, |
| | | 350 | 0.9 Hz, 1H), 7.91 (d, J = 8.5 Hz, 1H), 7.80 (dd, J = 9.2, 8.2 Hz, 2H), 4.05 (s, |
| | | ([M + H]⁺) | 3H); |
| | | | ¹³C NMR (101 MHz, CDCl₃) δ 164.87, 152.24, 148.37, 141.78, 139.49, |
| | | | 133.18 (d, J = 2.1 Hz), 130.33, 129.87, 128.08 (q, J = 5.2 Hz), 124.96, |
| | | | 123.03, 53.08 |
| F305 | | ESIMS m/z | ¹H NMR (400 MHz, CDCl₃) δ 8.18 (s, 1H), 8.00 (ddd, J = 8.2, 1.7, 0.8 Hz, |
| | | 367 | 1H), 7.80 (d, J = 8.3 Hz, 1H), 7.71 (d, J = 10.0 Hz, 1H), 4.03 (s, 3H); |
| | | ([M + H]⁺) | ¹³C NMR (101 MHz, CDCl₃) δ 163.93, 158.74, 156.03, 144.00 (d, J = 4.9 |
| | | | Hz), 141.09 (d, J = 10.9 Hz), 138.10 (d, J = 5.8 Hz), 132.92, 131.57, 131.50, |
| | | | 127.83 (q, J = 5.2 Hz), 127.25, 127.04-126.47 (m), 124.04, 121.32, 53.20 |
| F306 | | ESIMS m/z | ¹H NMR (400 MHz, CDCl₃) δ 7.94-7.83 (m, 3H), 7.81 (d, J = 8.5 Hz, 1H), |
| | | 334 | 7.71 (t, J = 7.7 Hz, 1H), 4.05 (s, 3H); |
| | | ([M + H]⁺) | ¹³C NMR (101 MHz, CDCl₃) δ 164.86, 161.51, 158.96, 152.30 (d, J = 2.4 |
| | | | Hz), 148.30, 143.11 (d, J = 7.7 Hz), 139.50, 130.37, 128.38-127.24 (m), |
| | | | 122.27 (d, J = 3.6 Hz), 115.49 (d, J = 22.5 Hz), 53.07 |
| F307 | | ESIMS m/z | ¹H NMR (400 MHz, CDCl₃) δ 7.94-7.87 (m, 2H), 7.75-7.69 (m, 2H), 4.03 |
| | | 352 | (s, 3H); |
| | | ([M + H]⁺) | ¹³C NMR (101 MHz, CDCl₃) δ 163.90, 161.12, 158.71, 156.01, 143.90 (d, J = |
| | | | 4.6 Hz), 141.03, 131.57 (d, J = 4.6 Hz), 127.48 (d, J = 5.3 Hz), 127.16 (d, J = |
| | | | 23.8 Hz), 124.32 (dd, J = 7.4, 3.7 Hz), 117.24 (dd, J = 23.0, 6.2 Hz), 53.17 |
| F308 | | ESIMS m/z | ¹H NMR (400 MHz, CDCl₃) δ 7.96-7.92 (m, 1H), 7.90-7.83 (m, 2H), 7.80 |
| | | 330 | (d, J = 8.5 Hz, 1H), 7.70 (d, J = 8.2 Hz, 1H), 4.05 (s, 3H), 2.57 (d, J = 1.8 |
| | | ([M + H]⁺) | Hz, 3H); |
| | | | ¹³C NMR (101 MHz, CDCl₃) δ 165.10, 153.97, 148.10, 140.25, 139.24, |
| | | | 137.50 (d, J = 1.9 Hz), 130.44, 129.62, 126.44 (q, J = 5.6 Hz), 124.28, |
| | | | 123.12, 53.01, 19.47 (d, J = 2.3 Hz) |
| F309 | | ESIMS m/z | ¹H NMR (400 MHz, CDCl₃) δ 7.90 (s, 1H), 7.87 (d, J = 8.3 Hz, 1H), 7.72 (d, |
| | | 348 | J = 8.2 Hz, 1H), 7.68 (d, J = 9.9 Hz, 1H), 4.03 (s, 3H), 2.57 (d, J = 1.9 Hz, |
| | | ([M + H]⁺) | 3H); |
| | | | ¹³C NMR (101 MHz, CDCl₃) δ 164.13, 158.64, 155.95, 143.71 (d, J = 4.9 |
| | | | Hz), 142.95 (d, J = 11.2 Hz), 137.26, 136.57 (d, J = 5.3 Hz), 132.08 (d, J = |
| | | | 5.2 Hz), 130.72 (d, J = 4.5 Hz), 130.18 (d, J = 30.2 Hz), 126.92, 126.68, |
| | | | 126.16 (t, J = 6.2 Hz), 125.68, 53.09, 19.46 (d, J = 2.2 Hz) |
| F310 | 100- | ESIMS m/z | ¹H NMR (500 MHz, CDCl₃) δ 7.74 (d, J = 8.4 Hz, 1H), 7.52 (d, J = 3.7 Hz, |
| | 102 | 370 | 2H), 4.02 (s, 3H) |
| | | ([M + H]⁺) | |
| F311 | 92- | ESIMS m/z | ¹H NMR (500 MHz, CDCl₃) δ 7.63 (d, J = 8.4 Hz, 1H), 7.57 (d, J = 7.8 Hz, |
| | 95 | 378 | 1H), 7.36-7.29 (m, 1H), 7.17 (d, J = 1.4 Hz, 1H), 4.12 (q, J = 7.0 Hz, 2H), |
| | | ([M + H]⁺) | 3.99 (s, 3H), 1.34 (t, J = 7.0 Hz, 3H); |
| | | | ¹⁹F NMR (471 MHz, CDCl₃) |
| | | | δ -62.84, -111.95, -111.97 |
| F312 | | ESIMS m/z | ¹H NMR (500 MHz, CDCl₃) δ 8.99 (s, 1H), 8.24 (t, J = 7.8 Hz, 2H), 7.51 |
| | | 335 | (dd, J = 27.7, 9.4 Hz, 3H), 4.07 (s, 3H); |
| | | ([M + H]⁺) | ¹⁹F NMR (471 MHz, CDCl₃) |
| | | | δ -63.11, -110.89, -110.91, -110.92, -110.93 |
| F313 | 104- | ESIMS m/z | ¹H NMR (500 MHz, CDCl₃) δ 8.96 (s, 1H), 7.83 (dd, J = 8.0, 1.0 Hz, 1H), |
| | 106 | 347 | 7.33 (ddd, J = 7.9, 1.6, 0.8 Hz, 1H), 7.26-7.22 (m, 1H), 4.04 (s, 3H), 3.92 |
| | | ([M + H]⁺) | (s, 3H); |
| | | | ¹⁹F NMR (471 MHz, CDCl₃) |
| | | | δ -62.92 |
| F314 | | ESIMS m/z | ¹H NMR (400 MHz, CDCl₃) δ 8.08 (d, J = 8.7 Hz, 2H), 8.00-7.90 (m, 2H), |
| | | 337 | 7.86 (d, J = 8.1 Hz, 1H); |
| | | ([M + H]⁺) | ¹³C NMR (101 MHz, CDCl₃) δ 151.65, 142.52, 140.65, 133.77, 130.04, |
| | | | 129.78, 128.44 (t, J = 5.2 Hz), 125.57, 125.01, 123.92, 121.21 |
| F315 | | ESIMS m/z | ¹H NMR (400 MHz, CDCl₃) δ 8.08 (d, J = 8.5 Hz, 1H), 7.96 (d, J = 8.4 Hz, |
| | | 320 | 1H), 7.87-7.74 (m, 3H); |
| | | ([M + H]⁺) | ¹³C NMR (101 MHz, CDCl₃) δ 161.56, 161.30, 151.70 (d, J = 2.5 Hz), |
| | | | 142.52, 142.44, 141.89 (d, J = 8.2 Hz), 133.75, 128.19 (dd, J = 4.6, 1.9 Hz), |
| | | | 125.50, 122.41 (d, J = 3.7 Hz), 115.44 (d, J = 22.6 Hz) |
| F316 | | ESIMS m/z | ¹H NMR (400 MHz, CDCl₃) δ 8.04 (d, J = 8.4 Hz, 1H), 7.96 (d, J = 8.4 Hz, |
| | | 316 | 1H), 7.88-7.80 (m, 2H), 7.77 (d, J = 8.2 Hz, 1H), 2.61 (q, J = 1.8 Hz, 3H); |
| | | ([M + H]⁺) | ¹³C NMR (101 MHz, CDCl₃) δ 161.17, 153.16, 142.37, 141.86, 139.02, |
| | | | 137.95 (d, J = 1.8 Hz), 133.12, 130.83, 130.53, 130.30, 126.81 (q, J = 5.6 |
| | | | Hz), 125.70, 125.56, 124.31, 122.84, 19.55 (d, J = 2.2 Hz) |
| F317 | | ESIMS m/z | ¹H NMR (400 MHz, CDCl₃) δ 8.05 (d, J = 8.5 Hz, 1H), 7.95 (d, J = 8.4 Hz, |
| | | 332 | 1H), 7.76-7.69 (m, 1H), 7.56-7.49 (m, 2H), 4.03 (s, 3H); |
| | | ([M + H]⁺) | ¹³C NMR (101 MHz, CDCl₃) δ 161.11, 158.24, 153.24, 142.38, 141.97, |
| | | | 141.01, 133.27, 129.25-127.40 (m), 125.82, 118.63, 110.36, 56.21 |
| F318 | | ESIMS m/z | ¹H NMR (400 MHz, CDCl₃) δ 8.21 (s, 1H), 7.78 (d, J = 8.1 Hz, 1H), 7.58 (d, |
| | | 324 | J = 8.4 Hz, 1H), 4.04 (s, 3H); |
| | | ([M − H]⁻) | ¹⁹F NMR (376 MHz, CDCl₃) |
| | | | δ -106.09, -113.71 |
| F319 | 96- | ESIMS m/z | ¹H NMR (500 MHz, CDCl₃) δ 8.91 (s, 1H), 8.53 (dt, J = 8.9, 0.9 Hz, 2H), |
| | 98 | 299 | 7.75-7.59 (m, 2H), 6.72 (t, J = 56.3 Hz, 1H), 4.07 (s, 3H); |
| | | ([M + H]⁺) | ¹⁹F NMR (471 MHz, CDCl₃) |
| | | | δ -111.66, -111.78 |
| F320 | 127- | ESIMS m/z | ¹H NMR (500 MHz, CDCl₃) δ 8.34 (s, 1H), 8.10 (p, J = 1.0 Hz, 1H), 7.79 |
| | 131 | 305 | (dt, J = 8.4, 1.7 Hz, 1H), 7.75-7.70 (m, 1H), 7.62 (d, J = 10.2 Hz, 1H), 7.32 |
| | | ([M + H]⁺) | (dd, J = 3.2, 2.4 Hz, 1H), 6.62-6.57 (m, 1H), 4.02 (s, 3H) |
| F321 | | EIMS m/z | ¹H NMR (400 MHz, CDCl₃) δ 7.99 (s, 1H), 7.94 (s, 1H), 7.77 (d, J = 8.3 Hz, |
| | | 392 | 1H), 4.02 (s, 3H); |
| | | | ¹⁹F NMR (376 MHz, CDCl₃) |
| | | | δ -62.28, -112.23 |
| F322 | | EIMS m/z | ¹H NMR (400 MHz, CDCl₃) δ 8.12 (s, 1H), 7.97 (s, 1H), 7.78 (d, J = 8.3 Hz, |
| | | 413 | 1H), 4.02 (s, 3H); |
| | | | ¹⁹F NMR (376 MHz, CDCl₃) |
| | | | δ -60.16, -112.18 |
| F323 | | ESIMS m/z | ¹H NMR (500 MHz, CDCl₃) δ 8.05-7.98 (m, 1H), 7.96 (dd, J = 8.5, 1.5 Hz, |
| | | 352 | 1H), 7.92 (d, J = 8.5 Hz, 1H), 7.44 (dd, J = 10.4, 5.6 Hz, 1H), 4.05 (s, 3H); |
| | | ([M + H]⁺) | ¹⁹F NMR (471 MHz, CDCl₃) |
| | | | δ -61.79, -109.32--119.29 (m), -119.92 (dd, J = 11.1, 6.7 Hz) |
| F324 | | EIMS m/z | ¹H NMR (500 MHz, CDCl₃) δ 7.73 (d, J = 8.4 Hz, 1H), 7.55-7.50 (m, 1H), |
| | | 369.9 | 7.45 (dd, J = 8.9, 5.5 Hz, 1H), 4.02 (s, 3H); |
| | | | ¹⁹F NMR (471 MHz, CDCl₃) |
| | | | δ -61.91, -113.26 (d, J = 37.3 Hz), -116.64 |
| | | | (dd, J = 9.0, 5.5 Hz), -117.53--119.51 (m) |
| F325 | | ESIMS m/z | ¹H NMR (500 MHz, CDCl₃) δ 8.01 (s, 1H), 7.44 (dd, J = 8.7, 5.5 Hz, 1H), |
| | | 385.9 | 7.36 (dd, J = 9.5, 5.3 Hz, 1H), 4.01 (s, 3H); |
| | | ([M + H]⁺) | ¹⁹F NMR (471 MHz, CDCl₃) |
| | | | δ -61.88, -115.74 (d, J = 5.4 Hz), -117.53--119.29 (m) |
| F326 | | EIMS m/z | ¹H NMR (400 MHz, CDCl₃) δ 8.20 (s, 1H), 7.97 (d, J = 8.4 Hz, 1H), 7.92 (s, |
| | | 374 | 1H), 7.86 (d, J = 8.4 Hz, 1H), 4.05 (s, 3H); |
| | | | ¹⁹F NMR (376 MHz, CDCl₃) |
| | | | δ -62.22 |
| F327 | | EIMS m/z | ¹H NMR (400 MHz, CDCl₃) δ 8.07-8.02 (m, 2H), 7.56 (dd, J = 8.4, 0.7 Hz, |
| | | 341 | 1H), 4.02 (s, 3H); |
| | | | ¹⁹F NMR (376 MHz, CDCl₃) |
| | | | δ -104.96 |
| F328 | 180- | ESIMS m/z | ¹H NMR (400 MHz, CDCl₃) δ 7.70-7.61 (m, 2H), 7.57 (dd, J = 9.3, 1.6 Hz, |
| | 182 | 410 | 1H), 7.34 (t, J = 7.8 Hz, 1H), 4.00 (s, 3H) |
| | | ([M + H]⁺) | |
| F329 | | ESIMS m/z | ¹H NMR (400 MHz, CDCl₃) δ 8.02-7.97 (m, 2H), 7.89 (d, J = 8.1 Hz, 1H); |
| | | 378.9 | ¹⁹F NMR (376 MHz, CDCl₃) |
| | | ([M + H]⁺) | δ -62.32, -110.52 |
| F330 | | ESIMS m/z | ¹H NMR (500 MHz, CDCl₃) δ 7.99 (s, 1H), 7.71 (d, J = 8.1 Hz, 1H), 7.58 (d, |
| | | 356 | J = 5.3 Hz, 1H), 7.47-7.40 (m, 2H), 3.99 (s, 3H); |
| | | ([M + H]⁺) | ¹³C NMR (126 MHz, CDCl₃) δ 164.15, 155.81, 153.81, 151.02, 145.45, |
| | | | 144.09, 139.33, 134.40, 130.34, 129.03, 127.29, 124.10, 119.46, 118.71, |
| | | | 53.21; |
| | | | ¹⁹F NMR (471 MHz, CDCl₃) |
| | | | δ -114.30 |
| F331 | | EIMS m/z | ¹H NMR (400 MHz, CDCl₃) δ 7.72 (d, J = 8.2 Hz, 1H), 7.68 (s, 1H), 7.08 (s, |
| | | 397.1 | 1H), 4.01 (s, 3H), 3.93 (s, 3H); |
| | | | ¹⁹F NMR (376 MHz, CDCl₃) |
| | | | δ -62.99, -112.56 |
| F332 | | EIMS m/z | H NMR (400 MHz, CDCl₃) δ 7.74 (s, 1H), 7.72 (d, J = 8.2 Hz, 1H), 7.39 (s, |
| | | 413 | 1H), 4.01 (s, 3H), 2.52 (s, 3H); |
| | | | ¹⁹F NMR (376 MHz, CDCl₃) |
| | | | δ -62.43, -112.62 |
| F333 | | EIMS m/z | ¹H NMR (400 MHz, CDCl₃) δ 7.61 (d, J = 8.4 Hz, 1H), 7.19 (s, 1H), 6.79 (s, |
| | | 375.1 | 1H), 3.99 (s, 3H), 3.80 (s, 3H); |
| | | | ¹⁹F NMR (376 MHz, CDCl₃) |
| | | | δ -50.02, -112.07 |
| F334 | | EIMS m/z | ¹H NMR (400 MHz, CDCl₃) δ 7.82 (d, J = 8.3 Hz, 1H), 7.30 (d, J = 5.5 Hz, |
| | | 347.9 | 1H), 7.03 (d, J = 8.5 Hz, 1H); |
| | | | ¹⁹F NMR (376 MHz, CDCl₃) |
| | | | δ -49.61, -110.96, -111.05, -115.04, -115.13 |
| F335 | | EIMS m/z | ¹H NMR (400 MHz, CDCl₃) δ 7.36 (s, 1H), 7.20 (d, J = 5.5 Hz, 1H), 6.90 (d, |
| | | 375.1 | J = 8.3 Hz, 1H), 3.97 (s, 3H), 3.91 (s, 3H); |
| | | | ¹⁹F NMR (376 MHz, CDCl₃) |
| | | | δ -49.80, -114.66 |
| F336 | | EIMS m/z | ¹H NMR (400 MHz, CDCl₃) δ 7.31 (d, J = 5.6 Hz, 1H), 7.20 (d, J = 10.6 Hz, |
| | | 359.1 | 1H), 6.93 (d, J = 8.4 Hz, 1H), 3.98 (s, 3H), 3.97 (s, 3H); |
| | | | ¹⁹F NMR (376 MHz, CDCl₃) |
| | | | δ -49.85, -112.13, -116.17 |
| F337 | | EIMS m/z | ¹H NMR (400 MHz, Methanol-d₄) δ 8.00 (d, J = 8.7 Hz, 1H), 7.65 (s, 1H), |
| | | 382.9 | 7.29 (s, 1H), 3.86 (s, 3H); |
| | | | ¹⁹F NMR (376 MHz, Methanol-d₄) |
| | | | δ -64.29, -115.94 |
| F338 | | ESIMS m/z | ¹H NMR (400 MHz, CDCl₃) δ 7.71 (dd, J = 8.2, 5.4 Hz, 2H), 7.47 (dt, J = |
| | | 395 | 8.1, 0.8 Hz, 1H), 6.76 (ddd, J = 17.9, 11.8, 1.8 Hz, 1H), 5.72 (dd, J = 11.7, |
| | | ([M + H]⁺) | 1.2 Hz, 1H), 5.52 (dq, J = 17.7, 0.9 Hz, 1H), 4.00 (s, 3H); |
| | | | ¹⁹F NMR (376 MHz, CDCl₃) |
| | | | δ -58.46, -112.89 |
| F339 | | ESIMS m/z | ¹H NMR (400 MHz, CDCl₃) δ 7.70 (d, J = 8.1 Hz, 1H), 7.67 (d, J = 8.2 Hz, |
| | | 383 | 1H), 7.39 (d, J = 8.1 Hz, 1H), 3.99 (s, 3H), 2.58 (d, J = 1.6 Hz, 3H); |
| | | ([M + H]⁺) | ¹⁹F NMR (376 MHz, CDCl₃) |
| | | | δ -61.22, -113.05 |
| F340 | 72- | ESIMS m/z | ¹H NMR (500 MHz, CDCl₃) δ 8.51-8.48 (m, 1H), 8.12 (dt, J = 8.6, 1.7 Hz, |
| | 74 | 316 | 1H), 7.97-7.93 (m, 2H), 7.89-7.86 (m, 1H), 7.68 (d, J = 10.0 Hz, 1H), |
| | | ([M + H]⁺) | 7.60-7.49 (m, 2H), 4.04 (s, 3H); |
| | | | ¹⁹F NMR (471 MHz, CDCl₃) |
| | | | δ -116.05, -116.07 |
| F341 | | ESIMS m/z | ¹H NMR (400 MHz, CDCl₃) δ 7.73 (dd, J = 8.1, 3.3 Hz, 1H), 7.53 (d, J = |
| | | 448 | 8.1, 1 Hz, 1H), 4.00 (s, 3H); |
| | | ([M + H]⁺) | ¹³C NMR (101 MHz, CDCl₃) δ 163.69, 158.07, 155.39, 142.97, 142.81, |
| | | | 136.90, 132.64, 132.59, 129.95, 126.64, 126.42, 125.88, 125.83, 123.65, |
| | | | 122.30, 120.93, 53.26 |
| F342 | | ESIMS m/z | ¹H NMR (400 MHz, CDCl₃) δ 8.10 (d, J = 8.7 Hz, 1H), 7.82 (s, 1H), 7.62 (s, |
| | | 397.9 | 1H), 2.57 (s, 3H); |
| | | ([M − H]⁻) | ¹⁹F NMR (376 MHz, CDCl₃) |
| | | | δ -59.84, -112.04 |
| F343 | 124- | ESIMS m/z | ¹H NMR (500 MHz, CDCl₃) δ 7.70 (dd, J = 8.3, 2.1 Hz, 2H), 7.28-7.24 (m, |
| | 126 | 378 | 3H), 7.19 (dd, J = 9.9, 2.4 Hz, 1H), 4.01 (s, 3H), 3.21 (s, 3H); |
| | | ([M + H]⁺) | ¹⁹F NMR (471 MHz, CDCl₃) |
| | | | δ -108.83, -108.85, -108.87, -108.91, -108.92, -108.94, -113.57, -113.59, -113.65, -113.66 |
| F344 | | EIMS m/z | ¹H NMR (400 MHz, CDCl₃) δ 7.93 (d, J = 8.8 Hz, 1H), 7.30 (s, 1H), 7.16 (s, |
| | | 361 | 1H), 3.83 (s, 3H); |
| | | | ¹⁹F NMR (376 MHz, CDCl₃) |
| | | | δ -48.27, -110.91 |
| F345 | 175- | ESIMS m/z | ¹H NMR (500 MHz, CDCl₃) δ 7.64 (d, J = 8.4 Hz, 1H), 7.48 (t, J = 8.2 Hz, |
| | 177 | 298 | 1H), 6.74 (dd, J = 8.4, 2.5 Hz, 1H), 6.66 (dd, J = 11.1, 2.4 Hz, 1H), 5.36 (d, J = |
| | | ([M − H]⁻) | 1.1 Hz, 1H), 4.00 (s, 3H); |
| | | | ¹⁹F NMR(471 MHz, CDCl₃) |
| | | | δ -111.32, -111.33, -111.34, -111.36, -111.39, -111.41, -111.42, -111.44, -113.71, -113.73, -113.79, |
| | | | -113.81 |
| F346 | 114- | ESIMS m/z | ¹H NMR (500 MHz, CDCl₃) δ 8.41-8.35 (m, 1H), 7.75 (d, J = 8.2 Hz, 1H), |
| | 116 | 351 | 7.73-7.66 (m, 2H), 7.65 (ddd, J = 8.4, 6.9, 1.2 Hz, 1H), 7.59-7.49 (m, |
| | | ([M + H]⁺) | 2H), 3.99 (s, 3H); |
| | | | ¹⁹F NMR (471 MHz, CDCl₃) |
| | | | δ -113.46, -113.48 |
| F347 | 145- | ESIMS m/z | ¹H NMR (500 MHz, DMSO-d₆) δ 14.05 (s, 1H), 8.40 (d, J = 9.1 Hz, 1H), |
| | 147 | 351 | 8.15 (d, J = 7.5 Hz, 1H), 7.69 (d, J = 7.5 Hz, 1H), 3.94 (s, 3H); |
| | | ([M + H]⁺) | ¹⁹F NMR (471 MHz, DMSO) |
| | | | δ -66.96, -114.64 |
| F348 | 143- | ESIMS m/z | ¹H NMR (500 MHz, DMSO-d₆) δ 8.28 (d, J = 9.3 Hz, 1H), 7.25 (t, J = 8.4 |
| | 146 | 299 | Hz, 1H), 6.52 (dd, J = 8.4, 2.1 Hz, 1H), 6.41 (dd, J = 13.2, 2.1 Hz, 1H), 5.92 |
| | | ([M + H]⁺) | (s, 2H), 3.91 (s, 3H); |
| | | | ¹⁹F NMR (471 MHz, DMSO) |
| | | | δ -114.37, -114.38, -114.39, -114.41, -114.45, -114.46, -114.47, -114.49, -114.77, -114.79, -114.85, |
| | | | -114.87 |
| F349 | | ESIMS m/z | ¹H NMR (400 MHz, Methanol-d₄) δ 8.03-7.99 (m, 2H), 7.85 (s, 1H), 7.81 |
| | | 411.8 | (d, J = 8.4 Hz, 1H); |
| | | ([M − H]⁻) | ¹⁹F NMR (376 MHz, Methanol-d₄) |
| | | | δ -64.32 |
| F350 | | ESIMS m/z | ¹H NMR (400 MHz, Methanol-d₄) δ 8.00 (d, J = 8.7 Hz, 1H), 7.84 (s, 1H), |
| | | 377.9 | 7.76 (s, 1H), 7.03-6.89 (m, 1H), 5.83 (d, J = 17.1 Hz, 1H), 5.43 (dd, J = |
| | | ([M − H]⁻) | 11.1, 0.7 Hz, 1H); |
| | | | ¹⁹F NMR (376 MHz, Methanol-d₄) |
| | | | δ -61.41, -116.08 |
| F351 | | ESIMS m/z | ¹H NMR (500 MHz, CDCl₃) δ 7.64 (d, J = 8.3 Hz, 1H), 7.56 (d, J = 7.7 Hz, |
| | | 321 | 1H), 7.39 (dd, J = 7.7, 1.4 Hz, 1H), 7.22 (d, J = 1.4 Hz, 1H), 3.99 (s, 3H), |
| | | ([M + H]⁺) | 3.86 (s, 3H); |
| | | | ¹⁹F NMR (471 MHz, CDCl₃) |
| | | | δ -112.17, -112.18 |
| F352 | | ESIMS m/z | ¹H NMR (300 MHz, CDCl₃) δ 7.87-7.79 (m, 2H), 7.72-7.66 (m, 1H), 7.62 |
| | | 354 | (d, J = 8.1 Hz, 1H); |
| | | ([M + H]⁺) | ¹³C NMR (126 MHz, CDCl₃) δ 161.10, 159.14, 156.98, 141.98 (d, J = 17.3 |
| | | | Hz), 139.18, 135.13 (d, J = 15.7 Hz), 135.02, 134.01 (d, J = 58.3 Hz), |
| | | | 133.51, 131.95, 128.61 (d, J = 22.0 Hz), 127.25 (d, J = 3.7 Hz), 124.07 (q, J = |
| | | | 3.8 Hz), 123.98, 121.81; |
| | | | ¹⁹F NMR (471 MHz, CDCl₃) |
| | | | δ -63.08, -110.82 |
| F353 | | EIMS m/z | ¹H NMR (500 MHz, Methanol-d₄) δ 8.06 (d, J = 8.6 Hz, 1H), 7.95 (d, J = 8.6 |
| | | 358 | Hz, 1H), 7.66 (s, 1H), 7.16 (s, 1H), 3.99 (s, 3H), 3.90 (s, 3H) |
| | | | ¹⁹F NMR (471 MHz, Methanol-d₄) |
| | | | δ -52.44 |
| F354 | 84- | ESIMS m/z | ¹H NMR (500 MHz, CDCl₃) δ 8.31 (ddd, J = 8.4, 7.6, |
| | 86 | 353 | 0.8 Hz, 1H), 7.80-7.73 (m, 2H), 4.02 (s, 3H); |
| | | ([M + H]⁺) | ¹⁹F NMR (471 MHz, CDCl₃) |
| | | | δ -64.76, -64.78, -64.83, -64.85, -68.17, -113.08, -113.10, -113.16, -113.18 |
| 355 | 182- | ESIMS m/z | ¹H NMR (500 MHz, CDCl₃) δ 7.83 (d, J = 8.5 Hz, 1H), 7.74 (d, J = 8.5 Hz, |
| | 184 | 299 | 1H), 7.51 (d, J = 8.5 Hz, 1H), 6.94 (d, J = 2.5 Hz, 1H), 6.82 (dd, J = 8.5, 2.5 |
| | | ([M + H]⁺) | Hz, 1H), 5.48 (s, 1H), 4.02 (s, 3H) |
| F356 | | EIMS m/z | ¹H NMR (500 MHz, CDCl₃) δ 8.05 (d, J = 8.5 Hz, 1H), 7.93 (d, J = 8.5 Hz, |
| | | 342 | 1H), 7.46 (s, 1H), 6.86 (s, 1H), 3.89 (s, 3H); |
| | | | ¹⁹F NMR (471 MHz, CDCl₃) |
| | | | δ -50.09 |
| F357 | 136- | ESIMS m/z | ¹H NMR (500 MHz, CDCl₃) δ 10.40 (d, J = 0.8 Hz, 1H), 7.68 (d, J = 8.4 Hz, |
| | 138 | 342 | 1H), 7.65 (dd, J = 8.1, 6.0 Hz, 1H), 7.37 (dt, J = 8.2, 1.2 Hz, 1H), 4.00 (s, |
| | | ([M + H]⁺) | 3H), 4.00 (d, J = 0.9 Hz, 3H); |
| | | | ¹⁹F NMR (471 MHz, CDCl₃) |
| | | | δ -113.12, -113.14, -138.97 |
| F358) | | ESIMS m/z | ¹H NMR (500 MHz, CDCl₃) δ 7.61 (d, J = 8.2 Hz, 1H), 7.26 (d, 1H), 6.77 (d, |
| | | 316 | J = 2.3 Hz, 1H), 6.65 (dd, J = 8.3, 2.3 Hz, 1H), 3.99 (s, 3H), 3.91 (s, 2H); |
| | | (M + H]⁺) | ¹⁹F NMR (471 MHz, CDCl₃) |
| | | | δ -112.16, -112.18 |
| F359 | | ESIMS m/z | ¹H NMR (600 MHz, CDCl₃) δ 7.65 (d, J = 8.3 Hz, 1H), 7.35 (dd, J = 9.9, 0.8 |
| | | 382 | Hz, 1H), 7.15 (d, J = 5.4 Hz, 1H), 4.00 (s, 3H), 3.85 (s, 3H); |
| | | ([M + H]⁺) | ¹⁹F NMR (564 MHz, CDCl₃) |
| | | | δ -61.50, -112.15, -124.52 |
| F360 | | ESIMS m/z | ¹H NMR (600 MHz, CDCl₃) δ 7.70 (d, J = 8.3 Hz, 1H), 7.54 (d, J = 6.7 Hz, |
| | | 366 | 1H), 7.44 (d, J = 10.0 Hz, 1H), 4.02 (s, 3H), 2.52 (dq, J = 2.3, 1.1 Hz, 3H); |
| | | ([M + H]⁺) | ¹⁹F NMR (564 MHz, CDCl₃) |
| | | | δ -62.46, -113.52, -116.41 |
| F361 | | ESIMS m/z | ¹H NMR (600 MHz, CDCl₃) δ 7.97 (d, J = 7.5 Hz, 1H), 7.92-7.85 (m, 2H), |
| | | 348 | 7.43 (d, J = 11.3 Hz, 1H), 4.04 (s, 3H), 2.70-2.36 (m, 3H); |
| | | ([M + H]⁺) | ¹⁹F NMR (564 MHz, CDCl₃) |
| | | | δ -62.36, -119.79 |
| F362 | | ESIMS m/z | ¹H NMR (600 MHz, CDCl₃) δ 7.65 (d, J = 8.3 Hz, 1H), 7.35 (dd, J = 9.9, 0.8 |
| | | 364 | Hz, 1H), 7.15 (d, J = 5.4 Hz, 1H), 4.00 (s, 3H), 3.85 (s, 3H); |
| | | ([M + H]⁺) | ¹⁹F NMR (564 MHz, CDCl₃) |
| | | | δ -61.38, -124.40 |
| F363 | 76- | ESIMS m/z | ¹H NMR (500 MHz, CDCl₃) δ 7.60 (d, J = 8.3 Hz, 1H), 7.57 (dd, J = 8.0, 0.9 |
| | 78 | 440 | Hz, 1H), 7.49-7.43 (m, 2H), 7.42-7.31 (m, 4H), 7.19 (t, J = 1.0 Hz, 1H), |
| | | ([M + H]⁺) | 5.44 (s, 2H), 3.86 (s, 3H); |
| | | | ¹⁹F NMR (471 MHz, CDCl₃) |
| | | | δ -62.82, -112.57, -112.59 |
| F364 | 43- | ESIMS m/z | ¹H NMR (500 MHz, CDCl₃) δ 7.91 (d, J = 8.4 Hz, 1H), 7.76 (dd, J = 8.5, 5.6 |
| | 45 | 431 | Hz, 2H), 7.44 (d, J = 2.5 Hz, 1H), 7.32 (dd, J = 8.6, 2.5 Hz, 1H), 4.02 (s, |
| | | ([M + H]⁺) | 3H); |
| | | | ¹⁹F NMR (471 MHz, CDCl₃) |
| | | | δ -72.55 |
| F365 | | ESIMS m/z | ¹H NMR (500 MHz, CDCl₃) δ 8.01-7.94 (m, 2H), 7.60 (d, J = 10.1 Hz, |
| | | 296 | 1H), 7.03-6.97 (m, 2H), 4.01 (s, 3H), 3.87 (s, 3H); |
| | | ([M − H]⁻) | ¹⁹F NMR (471 MHz, CDCl₃) |
| | | | δ -116.77, -116.79 |
| F366 | 126- | ESIMS m/z | ¹H NMR (500 MHz, DMSO-d₆) δ 14.59 (s, 1H), 9.21 (s, 1H), 7.79 (d, J = 8.4 |
| | 128 | 333 | Hz, 1H), 7.48 (d, J = 1.7 Hz, 1H), 7.45 (dd, J = 8.2, 1.7 Hz, 1H), 3.87 (s, |
| | | ([M + H]⁺) | 3H); |
| | | | ¹⁹F NMR (471 MHz, DMSO) |
| | | | δ -61.19 |
| F367 | | ESIMS m/z | ¹H NMR (500 MHz, CDCl₃) δ 7.87 (d, J = 8.5 Hz, 1H), 7.70 (d, J = 8.5 Hz, |
| | | 346 | 1H), 7.65-7.55 (m, 2H), 4.04 (s, 3H); |
| | | ([M + H]⁺) | ¹⁹F NMR (471 MHz, CDCl₃) |
| | | | δ -49.52, -134.11 |
| F368 | | ESIMS m/z | ¹H NMR (500 MHz, CDCl₃) δ 8.03 (d, J = 8.4 Hz, 1H), 7.85 (d, J = 8.4 Hz, |
| | | 331.9 | 1H), 7.57-7.46 (m, 2H); |
| | | ([M + H]⁺) | ¹⁹F NMR (471 MHz, CDCl₃) |
| | | | δ -49.32, -133.12 (d, J = 10.4 Hz) |
| F369 | | ESIMS m/z | ¹H NMR (500 MHz, CDCl₃) δ 7.87-7.75 (m, 2H), 7.59 (d, J = 8.4 Hz, 1H), |
| | | 358 | 6.84 (d, J = 8.4 Hz, 1H), 4.06 (s, 3H), 4.01 (s, 3H); |
| | | ([M + H]⁺) | ¹⁹F NMR (471 MHz, CDCl₃) |
| | | | δ -49.82 |
| F370 | | ESIMS m/z | ¹H NMR (500 MHz, CDCl₃) δ 7.72 (d, J = 8.4 Hz, 1H), 7.63 (s, 1H), 7.53 (s, |
| | | 381.9 | 1H), 4.01 (s, 3H), 2.50-2.22 (m, 3H); |
| | | ([M + H]⁺) | ¹⁹F NMR (471 MHz, CDCl₃) |
| | | | δ -62.72, -114.72 |
| F371 | | ESIMS m/z | ¹H NMR (500 MHz, CDCl₃) δ 7.92 (d, J = 8.4 Hz, 1H), 7.61 (s, 1H), 7.56 (s, |
| | | 364 | 1H), 7.49 (d, J = 8.3 Hz, 1H), 4.02 (s, 3H), 2.39 (s, 3H); |
| | | ([M + H]⁺) | ¹⁹F NMR (471 MHz, CDCl₃) |
| | | | δ -62.56 |
| F372 | | EIMS m/z | ¹H NMR (500 MHz, CDCl₃) δ 7.62 (d, J = 8.3 Hz, 1H), 7.19 (s, 1H), 6.62 |
| | | 401.1 | (dd, J = 17.8, 11.3 Hz, 1H), 5.88 (dd, J = 17.7, 0.7 Hz, 1H), 5.45 (dd, J = |
| | | | 11.2, 0.8 Hz, 1H), 4.05 (s, 3H), 4.00 (s, 3H); |
| | | | ¹⁹F NMR (471 MHz, CDCl₃) |
| | | | δ -49.38, -113.26 |
| F373 | | ESIMS m/z | ¹H NMR (300 MHz, CDCl₃) δ 7.77 (dt, J = 1.5, 0.8 Hz, 1H), 7.71-7.57 (m, |
| | | 352 | 2H), 7.48 (dd, J = 9.1, 8.1 Hz, 1H), 4.02 (s, 3H); |
| | | ([M + H]⁺) | ¹³C NMR (126 MHz, CDCl₃) δ 162.29 (d, J = 6.5 Hz), 160.77 (d, J = 6.2 |
| | | | Hz), 159.41 (d, J = 6.3 Hz), 158.56 (d, J = 6.3 Hz), 157.25 (d, J = 6.2 Hz), |
| | | | 141.93-139.65 (m), 136.62, 134.40, 133.15-133.05 (m), 133.01, 132.33, |
| | | | 126.84 (q, J = 3.9 Hz). 124.14, 123.97 (q, J = 3.7 Hz), 121.97, 113.93 (t, J = |
| | | | 22.7 Hz), 53.16 |
| F374 | | ESIMS m/z | ¹H NMR (300 MHz, CDCl₃) δ 7.95 (dt, J = 1.9, 0.6 Hz, 1H), 7.71 (ddd, J = |
| | | 397 | 8.0, 1.8, 0.7 Hz, 1H), 7.57 (d, J = 8.0 Hz, 1H), 7.48 (dd, J = 9.1, 8.1 Hz, 1H), |
| | | ([M + H]⁺) | 4.02 (s, 3H); |
| | | | ¹³C NMR (126 MHz, CDCl₃) δ 162.28 (d, J = 6.5 Hz), 160.76 (d, J = 5.9 |
| | | | Hz), 159.14 (d, J = 6.4 Hz), 158.54 (d, J = 6.1 Hz), 156.95, 142.51, 138.65, |
| | | | 133.12 (d, J = 33.3 Hz), 132.15, 129.96 (d, J = 4.0 Hz), 124.54 (d, J = 3.6 |
| | | | Hz), 123.98, 123.50, 121.81, 113.97 (t, J = 22.7 Hz), 53.17 |
| F375 | | ESIMS m/z | ¹H NMR (300 MHz, CDCl₃) δ 7.78 (ddd, J = 7.9, 6.9, 1.0 Hz, 1H), 7.58 (dt, J = |
| | | 336 | 8.1, 1.1 Hz, 1H), 7.53-7.43 (m, 2H), 4.02 (s, 3H); |
| | | ([M + H]⁺) | ¹³C NMR (126 MHz, CDCl₃) δ 162.32 (d, J = 6.7 Hz), 160.85, 160.68 (d, J = |
| | | | 6.1 Hz), 159.67 (d, J = 6.3 Hz), 158.83, 158.46 (d, J = 6.3 Hz), 157.49 (d, J = |
| | | | 6.0 Hz), 138.10 (dd, J = 16.3, 4.5 Hz), 133.99 (d, J = 8.1 Hz), 133.72 (d, J = |
| | | | 8.2 Hz), 133.41 (dd, J = 9.1, 4.6 Hz), 132.61 (d, J = 3.1 Hz), 125.51 (d, J = |
| | | | 15.4 Hz), 124.12 (d, J = 2.6 Hz), 121.96, 121.48 (q, J = 3.8 Hz), 114.00 (t, J = |
| | | | 22.8 Hz), 113.50 (dd, J = 25.0, 4.0 Hz), 53.13 |
| F376 | | ESIMS m/z | ¹H NMR (300 MHz, CDCl₃) δ 7.56 (ddt, J = 7.3, 1.9, 0.7 Hz, 1H), |
| | | 332 | 7.53-7.43 (m, 1H), 4.01 (s, 2H), 2.32 (s, 3H); |
| | | ([M + H]⁺) | ¹³C NMR (126 MHz, CDCl₃) δ 162.44 (d, J = 6.6 Hz), 160.16 (d, J = 6.0 |
| | | | Hz), 159.15 (d, J = 5.7 Hz), 157.95 (d, J = 5.8 Hz), 157.00 (d, J = 5.6 Hz), |
| | | | 144.24-142.69 (m), 137.99, 136.69, 133.62-132.71 (m), 131.56 (q, J = |
| | | | 32.3 Hz), 130.58 (d, J = 2.2 Hz), 127.38 (q, J = 3.9 Hz), 125.02, 122.81 (q, J = |
| | | | 3.5 Hz), 113.83 (t, J = 22.9 Hz), 53.09, 19.72 (d, J = 2.8 Hz) |
| F377 | | ESIMS m/z | ¹H NMR (300 MHz, CDCl₃) δ 7.55 (dd, J = 7.8, 1.0 Hz, 1H), 7.39 (dd, J = |
| | | 348 | 9.3, 8.2 Hz, 1H), 7.36-7.32 (m, 1H), 7.21-7.16 (m, 1H), 4.00 (s, 3H), 3.86 |
| | | ([M + H]⁺) | (s, 3H); |
| | | | ¹³C NMR (126 MHz, CDCl₃) δ 162.62 (d, J = 7.0 Hz), 160.48 (d, J = 6.3 |
| | | | Hz), 159.89 (d, J = 5.6 Hz), 158.27 (d, J = 6.4 Hz), 157.72 (d, J = 5.9 Hz), |
| | | | 157.54, 141.88-140.03 (m), 132.74-132.52 (m), 131.86, 127.00, 124.89, |
| | | | 122.71, 117.78 (q, J = 4.0 Hz), 113.38 (t, J = 23.1 Hz), 107.93 (q, J = 3.9 |
| | | | Hz), 55.97, 53.02 |
| F378 | 156- | ESIMS m/z | ¹H NMR (500 MHz, CDCl₃) δ 7.78 (dd, J = 7.9, 6.8 Hz, 1H), 7.73 (d, J = 8.4 |
| | 159 | 309 | Hz, 1H), 7.61 (dd, J = 8.0, 1.5 Hz, 1H), 7.50 (dd, J = 9.3, 1.5 Hz, 1H), 4.02 |
| | | ([M + H]⁺) | (s, 3H); |
| | | | ¹⁹F NMR (471 MHz, CDCl₃) |
| | | | δ -110.08, -110.10, -110.10, -110.12, -110.16, -110.17, -110.18, -110.19, -113.25, -113.27, -113.33, |
| | | | -113.35 |
| F379 | 179- | ESIMS m/z | ¹H NMR (500 MHz, CDCl₃) δ 7.88 (d, J = 4.9 Hz, 2H), 7.81-7.77 (m, 1H), |
| | 181 | 362 | 7.74 (d, J = 8.4 Hz, 1H), 4.02 (s, 3H), 3.10 (s, 3H); |
| | | ([M + H]⁺) | ¹⁹F NMR (471 MHz, CDCl₃) |
| | | | δ -109.22, -109.23, -109.24, -109.25, -109.29, -109.30, -109.31, -109.32, -113.31, -113.33, -113.39, |
| | | | -113.41 |
| F380 | | ESIMS m/z | ¹H NMR (500 MHz, CDCl₃) δ 7.91 (d, J = 8.4 Hz, 1H), 7.84-7.79 (m, 2H), |
| | | 475.3 | 7.77-7.69 (m, 3H), 7.64 (ddd, J = 8.1, 1.7, 0.8 Hz, 1H), 7.58 (t, J = 7.6 Hz, |
| | | ([M − F]⁺) | 1H), 7.46 (t, J = 7.6 Hz, 1H), 5.67 (s, 2H); |
| | | | ¹⁹F NMR (471 MHz, CDCl₃) |
| | | | δ -59.88, -62.91 |
| F381 | | HRMS-ESI (m/z) | ¹H NMR (500 MHz, CDCl₃) δ 7.90 (d, J = 8.4 Hz, 1H), 7.80 (d, J = 8.3 Hz, |
| | | [M + H]⁺ | 2H), 7.76 (d, J = 1.7 Hz, 1H), 7.63 (dd, J = 8.1, 1.7 Hz, 1H), 5.17-5.13 (m, |
| | | calcd for | 1H), 5.02 (t, J = 1.5 Hz, 1H), 4.85 (s, 2H), 1.86 (t, J = 1.1 Hz, 3H); |
| | | C₁₇H₁₂Cl₂F₃NO₂, | ¹⁹F NMR (471 MHz, CDCl₃) |
| | | 390.027; found | δ -62.93 |
| | | 390.0267 | |

mp = melting point

### Example A. Evaluation of Postemergent Herbicidal Activity

Post-emergent Test: Seeds or nutlets of the desired test plant species were planted in Sun Gro Metro-Mix® 360 planting mixture, which typically has a pH of 6.0 to 6.8 and an organic matter content of about 30 percent, in plastic pots with a surface area of 64 square centimeters. In some aspects, to ensure good germination and healthy plants, a fungicide treatment and/or other chemical or physical treatment was applied. The plants were grown for 7-21 d in a greenhouse with an approximate 15 h photoperiod which was maintained at 23-29° C. during the day and 22-28° C. during the night. Nutrients and water were added on a regular basis and supplemental lighting was provided with overhead metal halide 1000-Watt lamps as necessary. Plants were used for testing when they reached the first or second true leaf stage.

A weighed amount, determined by the highest rate to be tested, of each test compound was placed in a 25 mL glass vial and was dissolved in 4 mL of a 97:3 v/v mixture of acetone and DMSO to obtain concentrated stock solutions. If the test compound did not dissolve readily, the mixture was warmed and/or sonicated. The concentrated stock solutions obtained were diluted with 20 mL of an aqueous mixture containing acetone, water, isopropyl alcohol, DMSO, AgriDex crop oil concentrate, and X-77 surfactant in a 48.5:39:10:1.5:1.0:0.02 v/v ratio to obtain spray solutions containing the application rate. Compound requirements are based upon a 12 mL application volume at a rate of 187 liters per hectare (L/ha). Formulated compounds were applied to the plant material with an overhead Mandel track sprayer equipped with 8002E nozzles calibrated to deliver 187 L/ha over an application area of 0.64 square meters at a spray height of 18 inches (43 cm) above the average plant canopy height. Control plants were sprayed in the same manner with the solvent blank.

The treated plants and control plants were placed in a greenhouse as described above and watered by subirrigation to prevent wash-off of the test compounds. After 14 d, the condition of the test plants as compared with that of the nontreated and control plants was determined visually and scored on a scale of 0 to 100 percent where 0 corresponds to no injury and 100 corresponds to complete control. In the reporting of the results, Table A: Percent Growth Reduction Conversion Table was used. Some of the compounds tested, application rates employed, plant species tested, and results are given in Table B in FIG. 3.

TABLE A
Percent Growth Reduction Conversion Table

| | % Visual Growth |
|---|---|
| Rating | Reduction |
| A | 95-100 |
| B | 85-94 |
| C | 75-84 |
| D | 60-74 |
| E | 45-59 |
| F | 30-44 |
| G | 0-29 |

TABLE B
Table B: Visual Growth Reduction (%) 14 Days After Application
at 140 grams active ingredient per hectare (g ai/ha)
Part A

| Cmpd ID | ALOMY | AMARE | AVEFA | CHEAL | CIRAR | CYPES | DIGSA |
|---|---|---|---|---|---|---|---|
| F1 | G | G | G | G | G | E | G |
| F2 | G | D | G | G | G | G | B |
| F3 | G | G | G | C | G | G | D |
| F4 | G | G | G | B | C | G | G |
| F5 | G | G | G | E | G | F | G |
| F6 | G | G | G | G | D | G | G |
| F7 | G | G | G | G | G | G | G |
| F8 | G | A | G | A | B | E | D |
| F9 | G | F | G | G | G | G | G |
| F10 | G | F | G | E | F | E | E |
| F11 | G | G | G | G | F | G | G |
| F12 | G | B | G | A | D | D | C |
| F13 | G | C | G | B | B | G | G |
| F14 | G | G | G | B | G | G | G |
| F15 | G | F | G | E | G | F | G |
| F16 | G | F | G | G | F | G | G |
| F17 | G | D | G | D | G | G | E |
| F18 | G | G | G | G | G | G | G |
| F19 | G | A | G | A | A | F | G |
| F20 | G | G | G | G | G | G | G |
| F21 | G | G | G | G | G | G | G |
| F22 | G | G | G | G | G | G | G |
| F23 | G | G | G | G | G | G | G |
| F24 | G | B | G | A | A | G | B |
| F25 | G | G | G | G | G | G | G |
| F26 | G | G | G | G | G | G | G |
| F27 | G | D | G | E | C | G | D |
| F28 | G | D | G | D | C | G | E |
| F29 | G | D | G | D | G | G | E |
| F30 | D | A | E | A | B | G | B |
| F31 | G | E | G | G | G | G | G |
| F32 | G | D | G | D | C | G | D |
| F33 | G | G | G | E | E | G | F |
| F34 | G | G | G | E | G | G | G |
| F35 | G | F | G | G | G | G | G |
| F36 | G | G | G | G | G | G | G |
| F37 | G | B | G | D | G | G | G |
| F38 | G | G | G | G | G | G | G |
| F39 | G | G | G | G | G | G | G |
| F40 | G | G | G | G | G | G | G |
| F41 | G | G | G | G | G | G | G |
| F42 | G | F | G | B | D | G | G |
| F43 | G | G | G | G | G | G | G |
| F44 | F | A | E | A | A | G | C |
| F45 | F | A | E | A | A | G | A |
| F46 | G | G | G | D | G | G | G |
| F47 | G | G | G | G | G | G | G |
| F48 | G | D | G | A | G | G | G |
| F49 | G | F | G | E | G | G | G |
| F50 | G | G | G | D | G | G | G |
| F51 | G | E | G | E | G | G | G |
| F52 | G | G | G | G | G | G | G |
| F53 | D | A | C | A | A | G | A |
| F54 | G | G | G | G | G | G | G |
| F55 | G | A | G | D | G | G | G |
| F56 | G | G | G | G | G | G | G |
| F57 | G | G | G | G | G | G | G |
| F58 | F | A | E | D | A | G | B |
| F59 | G | B | G | D | F | G | D |
| F60 | G | D | G | G | G | G | G |
| F61 | D | A | D | A | B | G | A |
| F62 | G | A | G | C | C | G | C |
| F63 | G | A | G | A | B | G | B |
| F64 | G | E | G | D | G | D | B |
| F65 | G | A | G | D | B | G | A |
| F66 | E | A | D | B | A | G | A |
| F67 | G | E | G | G | G | G | G |
| F68 | G | G | G | G | G | n/t | G |
| F69 | G | G | G | G | G | G | G |
| F70 | G | A | G | A | C | C | D |
| F71 | G | A | G | A | C | D | C |
| F72 | G | G | G | G | G | G | G |
| F73 | G | G | G | A | G | G | G |
| F74 | G | G | G | F | G | G | G |
| F75 | G | B | G | A | F | G | G |
| F76 | G | F | G | E | G | F | G |
| F77 | G | n/t | G | E | G | G | G |
| F78 | G | E | G | C | D | G | G |
| F79 | D | A | C | A | B | G | A |
| F80 | G | D | G | F | G | G | G |
| F81 | D | A | D | C | A | G | A |
| F82 | G | A | G | D | C | C | G |
| F83 | G | G | G | G | G | G | G |
| F84 | G | G | G | E | G | G | F |
| F85 | G | G | G | C | G | G | G |
| F86 | n/t | n/t | n/t | n/t | n/t | n/t | n/t |
| F87 | G | G | G | G | G | G | G |
| F88 | D | A | G | A | B | G | B |
| F89 | G | G | G | E | G | G | F |
| F90 | G | C | G | C | B | G | D |
| F91 | G | G | G | G | F | G | G |
| F92 | G | G | G | G | G | G | F |
| F93 | G | G | G | E | F | G | D |
| F94 | G | G | G | G | F | G | G |
| F95 | G | G | G | G | G | G | G |
| F96 | G | F | G | D | F | G | G |
| F97 | G | G | G | G | G | G | G |
| F98 | G | G | G | G | G | G | G |
| F99 | G | G | G | G | G | G | G |
| F100 | G | G | G | G | E | F | G |
| F101 | G | A | G | A | B | C | A |
| F102 | G | E | G | D | G | G | G |
| F103 | G | D | G | C | B | G | F |
| F104 | G | G | G | G | G | G | D |
| F105 | G | A | G | B | B | G | B |
| F106 | G | E | G | F | G | G | E |
| F107 | F | A | E | A | A | G | A |
| F108 | G | G | G | G | G | G | G |
| F109 | G | G | G | G | G | G | G |
| F110 | G | B | G | C | B | G | G |
| F111 | D | A | D | A | A | G | B |
| F112 | D | A | A | A | A | E | B |
| F113 | G | A | E | A | B | G | B |
| F114 | G | G | G | G | G | G | G |
| F115 | G | G | G | G | D | G | G |
| F116 | F | A | E | B | B | G | B |
| F117 | E | A | D | A | B | G | A |
| F118 | F | A | D | A | A | E | B |
| F119 | G | G | G | G | G | G | G |
| F120 | G | E | G | F | D | G | G |
| F121 | G | F | G | G | G | G | G |
| F122 | G | A | G | D | D | G | F |
| F123 | G | G | G | G | G | G | G |
| F124 | E | C | F | B | C | G | B |
| F125 | G | G | G | E | G | G | G |
| F126 | G | A | G | B | C | G | B |
| F127 | G | A | G | C | B | G | D |
| F128 | G | G | G | G | G | G | G |
| F129 | G | G | G | G | G | G | G |
| F130 | G | B | G | C | C | G | D |
| F131 | E | A | C | A | A | G | A |
| F132 | G | A | G | A | B | G | C |
| F133 | G | G | G | C | G | G | E |
| F134 | G | G | G | G | G | G | G |
| F135 | F | A | D | B | A | G | A |
| F136 | G | A | G | C | G | G | D |
| F137 | G | D | G | C | G | G | F |
| F138 | G | B | G | D | D | G | G |
| F139 | G | G | G | G | G | F | G |
| F140 | G | B | G | B | E | G | B |
| F141 | G | C | G | G | G | G | D |
| F142 | G | D | G | F | G | G | G |
| F143 | G | E | G | F | G | G | C |
| F144 | G | E | G | C | G | G | G |
| F145 | n/t | n/t | n/t | n/t | n/t | n/t | n/t |
| F146 | G | G | G | G | G | G | G |
| F147 | G | E | G | F | G | G | G |
| F148 | G | C | G | C | G | n/t | G |
| F149 | G | C | G | A | D | G | G |
| F150 | G | C | G | C | B | G | G |
| F151 | G | A | G | A | E | G | G |
| F152 | G | A | G | A | E | G | D |
| F153 | D | A | D | B | B | G | B |
| F154 | G | B | G | B | G | G | D |
| F155 | G | D | G | C | G | G | G |
| F156 | E | A | G | B | C | G | B |
| F157 | G | A | E | B | B | G | B |
| F158 | G | A | F | A | B | G | B |
| F159 | G | D | G | E | F | G | D |
| F160 | C | A | B | A | B | D | B |
| F161 (F393) | G | A | C | B | B | G | E |
| F162 | G | A | F | A | B | G | C |
| F163 | G | F | G | E | G | G | G |
| F164 | D | A | D | A | B | G | A |
| F165 | G | B | G | E | D | G | C |
| F166 | G | A | C | B | B | G | C |
| F167 | G | D | G | C | G | G | c |
| F168 | G | F | G | E | G | G | D |
| F169 | G | F | G | F | G | G | F |
| F170 | E | A | F | A | B | D | B |
| F171 | G | G | G | D | G | G | G |
| F172 | F | A | F | B | D | C | B |
| F173 | D | A | C | A | B | C | A |
| F174 | G | B | G | B | G | G | B |
| F175 | D | A | D | A | B | G | A |
| F176 | G | G | G | G | G | G | G |
| F177 | G | G | G | D | E | G | G |
| F178 | G | G | G | G | G | G | G |
| F179 | G | D | G | G | G | G | G |
| F180 | G | G | G | G | G | G | G |
| F181 | F | A | F | A | C | E | A |
| F182 | G | C | G | B | G | F | D |
| F183 | G | D | G | G | F | G | G |
| F184 | G | D | G | D | E | G | D |
| F185 | G | G | G | E | G | G | C |
| F186 | G | C | G | B | C | E | C |
| F187 | G | B | F | C | C | F | B |
| F188 | G | G | G | D | E | G | F |
| F189 | G | G | G | F | F | G | G |
| F190 | G | A | D | A | B | G | B |
| F191 | G | B | G | A | B | E | B |
| F192 | G | B | G | A | C | G | B |
| F193 | G | A | F | B | G | G | B |
| F194 | G | G | G | F | C | G | G |
| F195 | G | A | F | A | G | G | D |
| F196 | G | C | G | B | G | G | G |
| F197 | G | G | G | E | G | G | F |
| F198 | G | G | G | G | G | G | G |
| F199 | G | G | G | F | G | G | G |
| F200 | E | A | D | A | A | G | A |
| F201 | G | E | G | G | G | G | G |
| F202 | G | G | G | G | G | G | G |
| F203 | G | G | G | G | G | G | G |
| F204 | G | G | G | G | G | G | G |
| F205 | G | F | G | C | C | G | E |
| F206 | G | E | G | F | F | G | E |
| F207 | n/t | n/t | n/t | n/t | n/t | n/t | n/t |
| F208 | G | G | G | B | G | G | G |
| F209 | G | E | G | B | D | G | G |
| F210 | E | A | D | B | B | G | B |
| F211 | G | B | G | A | B | G | F |
| F212 | G | D | G | A | B | G | F |
| F213 | G | E | G | B | G | G | F |
| F214 | E | C | D | A | B | G | A |
| F215 | G | B | D | B | B | G | E |
| F216 | G | F | G | A | D | G | G |
| F217 | n/t | n/t | n/t | n/t | n/t | n/t | n/t |
| F218 | G | G | G | D | F | G | G |
| F219 | G | G | G | E | G | G | F |
| F220 | n/t | n/t | n/t | n/t | n/t | n/t | n/t |
| F221 | C | A | C | A | A | C | A |
| F222 | G | C | G | C | B | G | C |
| F223 | G | A | D | A | A | E | D |
| F224 | G | B | G | A | E | G | C |
| F225 | E | A | A | A | B | E | A |
| F226 | E | D | E | A | B | G | A |
| F227 | G | G | G | C | F | G | F |
| F228 | G | G | G | G | G | G | G |
| F229 | B | A | A | A | A | E | A |
| F230 | G | A | G | A | A | G | G |
| F231 | G | G | G | G | G | G | G |
| F232 | G | D | G | A | B | G | A |
| F233 | C | A | E | A | A | E | A |
| F234 | G | G | G | F | G | G | F |
| F235 | D | A | A | A | A | G | A |
| F236 | G | F | G | B | B | G | C |
| F237 | G | A | E | B | A | E | B |
| F238 | G | E | G | A | D | G | D |
| F239 | G | G | G | F | G | G | G |
| F240 | G | F | G | D | G | G | A |
| F241 | D | A | B | A | A | D | A |
| F242 | G | G | G | D | G | G | G |
| F243 | B | A | C | A | A | B | A |
| F244 | G | G | G | D | G | G | G |
| F245 | G | G | G | B | G | E | G |
| F246 | E | A | G | A | C | D | B |
| F247 | G | G | G | D | G | G | G |
| F248 | G | G | G | G | G | G | D |
| F249 | G | G | G | D | G | G | G |
| F250 | G | G | G | C | D | G | B |
| F251 | G | B | G | A | B | G | C |
| F252 | G | G | G | D | G | G | F |
| F253 | G | A | G | A | A | G | G |
| F254 | B | A | A | A | A | D | A |
| F255 | G | G | G | G | G | G | G |
| F256 | G | G | G | G | G | G | G |
| F257 | G | G | G | C | G | G | G |
| F258 | G | D | G | C | G | F | B |
| F259 | G | A | G | C | C | G | D |
| F260 | G | G | G | G | G | G | G |
| F261 | G | G | G | D | F | G | D |
| F262 | G | G | G | F | G | G | G |
| F263 | G | G | G | G | G | G | G |
| F264 | G | G | G | C | F | G | G |
| F265 | G | G | G | C | G | G | G |
| F266 | G | G | G | G | G | G | G |
| F267 | F | A | E | A | B | G | B |
| F268 | G | G | G | D | F | G | G |
| F269 | G | G | G | G | F | G | G |
| F270 | G | E | G | B | C | E | B |
| F271 | G | G | G | D | G | G | G |
| F272 | G | G | G | G | G | G | G |
| F273 | G | G | G | C | F | G | D |
| F274 | G | G | G | G | D | G | G |
| F275 | G | D | G | B | A | G | G |
| F276 | G | G | G | G | G | G | G |
| F277 | G | G | G | G | G | G | G |
| F278 | G | G | G | G | G | G | G |
| F279 | G | G | G | G | G | G | G |
| F280 | G | F | G | B | F | D | G |
| F281 | G | C | G | B | G | G | G |
| F282 | G | E | G | B | D | G | G |
| F283 | G | F | G | B | B | F | G |
| F284 | G | G | G | G | F | G | G |
| F285 | G | G | G | G | F | G | G |
| F286 | G | G | G | C | D | G | G |
| F287 | G | G | G | G | G | G | G |
| F288 | G | B | G | B | A | G | C |
| F289 | G | G | G | F | F | G | G |
| F290 | G | G | G | G | G | D | G |
| F291 | G | G | G | E | F | G | G |
| F292 | G | G | G | B | D | D | G |
| F293 | G | G | G | E | F | G | G |
| F294 | C | A | D | B | C | G | B |
| F295 | G | G | G | F | D | C | E |
| F296 | G | G | G | G | D | G | G |
| F297 | G | B | G | B | B | G | C |
| F298 | G | F | G | C | D | G | G |
| F299 | G | D | G | C | C | C | G |
| F300 | G | G | G | C | E | G | G |
| F301 | G | G | G | G | G | G | G |
| F302 | n/t | n/t | n/t | n/t | n/t | n/t | n/t |
| F303 | G | G | G | C | G | G | G |
| F304 | G | G | G | G | G | G | F |
| F305 | G | F | G | E | G | E | B |
| F306 | G | F | G | G | G | E | C |
| F307 | G | C | G | C | G | G | B |
| F308 | G | E | G | G | G | G | G |
| F309 | G | E | G | E | G | G | G |
| F310 | E | A | D | A | A | C | A |
| F311 | G | G | G | F | G | G | G |
| F312 | G | B | G | B | B | E | B |
| F313 | G | E | G | F | E | G | C |
| F314 | G | G | G | F | E | G | C |
| F315 | G | D | G | D | D | D | C |
| F316 | G | G | G | F | F | G | G |
| F317 | G | G | G | G | G | G | G |
| F318 | G | D | G | D | G | G | B |
| F319 | G | G | G | F | G | G | G |
| F320 | G | G | G | D | F | G | G |
| F321 | G | F | C | A | C | G | B |
| F322 | G | D | G | C | F | G | G |
| F323 | G | C | G | B | E | E | C |
| F324 | F | A | F | B | n/t | D | A |
| F325 | G | D | G | A | B | G | C |
| F326 | G | G | G | D | E | G | C |
| F327 | G | F | G | F | G | G | G |
| F328 | G | F | G | G | G | G | C |
| F329 | G | D | D | C | B | G | C |
| F330 | G | G | G | G | n/t | F | G |
| F331 | G | E | G | E | n/t | G | G |
| F332 | G | G | G | G | n/t | G | G |
| F333 | G | F | G | G | n/t | G | D |
| F334 | C | C | D | C | n/t | D | A |
| F335 | G | G | G | F | n/t | G | G |
| F336 | G | F | G | F | n/t | G | D |
| F337 | G | F | G | C | n/t | G | E |
| F338 | G | A | F | B | n/t | G | D |
| F339 | G | A | G | A | n/t | D | G |
| F340 | G | G | G | G | n/t | G | G |
| F341 | F | A | E | B | n/t | G | B |
| F342 | G | G | G | D | n/t | G | G |
| F343 | G | A | F | B | A | F | B |
| F344 | G | G | G | F | n/t | G | D |
| F345 | G | G | G | G | G | G | G |
| F346 | G | G | G | G | n/t | G | G |
| F347 | D | A | D | A | B | G | A |
| F348 | G | G | G | G | n/t | G | G |
| F349 | D | D | D | A | n/t | G | C |
| F350 | G | F | G | D | n/t | G | F |
| F351 | G | D | G | C | G | G | F |
| F352 | C | A | B | A | A | G | A |
| F353 | G | G | G | E | G | G | G |
| F354 | G | D | F | C | C | G | B |
| F355 | G | G | G | G | C | G | G |
| F356 | G | G | G | D | G | G | G |
| F357 | G | G | G | G | G | G | G |
| F358 | G | G | G | G | G | G | G |
| F359 | F | A | C | A | C | G | F |
| F360 | G | A | G | A | G | G | G |
| F361 | G | E | G | B | G | G | G |
| F362 | G | A | G | B | G | G | E |
| F363 | G | A | B | A | B | G | G |
| F364 | G | D | G | C | D | G | C |
| F365 | G | G | G | G | G | G | G |
| F366 | G | C | G | B | F | G | C |
| F367 | G | G | G | G | G | G | C |
| F368 | G | G | G | G | G | G | C |
| F369 | F | D | F | F | B | G | B |
| F370 | G | B | G | E | C | G | E |
| F371 | G | F | G | F | G | G | E |
| F372 | G | E | G | G | G | G | G |
| F373 | G | A | E | A | A | G | A |
| F374 | E | A | E | A | A | G | B |
| F375 | E | A | D | B | B | G | B |
| F376 | G | A | G | B | D | G | D |
| F377 | G | A | F | B | C | D | D |
| F378 | G | E | G | C | E | G | D |
| F379 | G | D | G | D | G | G | D |
| F380 | E | A | D | A | B | G | A |
| F381 | E | B | E | A | B | G | B |

Part B

| Cmpd ID | ECHCG | IPOHE | KCHSC | ORYSA | SETFA | SORVU | STEME | TRZAS |
|---|---|---|---|---|---|---|---|---|
| F1 | G | G | G | G | G | G | G | G |
| F2 | D | B | G | G | F | A | G | E |
| F3 | G | D | G | G | G | G | G | G |
| F4 | G | D | G | G | G | F | G | G |
| F5 | G | F | F | G | G | F | n/t | G |
| F6 | G | E | E | G | G | E | G | G |
| F7 | G | G | G | G | G | G | G | G |
| F8 | F | A | B | G | G | B | G | F |
| F9 | G | E | G | G | G | G | G | G |
| F10 | G | D | G | G | G | F | G | G |
| F11 | G | F | G | G | G | G | n/t | G |
| F12 | G | A | G | G | G | C | G | G |
| F13 | G | A | D | G | G | D | G | G |
| F14 | G | E | B | G | G | G | G | G |
| F15 | G | A | G | G | G | G | G | G |
| F16 | G | A | G | G | G | C | G | G |
| F17 | G | A | G | G | G | B | G | G |
| F18 | G | G | G | G | G | G | G | G |
| F19 | G | A | B | G | G | F | G | G |
| F20 | G | D | G | G | G | G | G | G |
| F21 | G | G | G | G | G | G | G | G |
| F22 | G | G | G | G | G | G | G | G |
| F23 | G | F | G | G | G | G | G | G |
| F24 | B | A | D | G | E | B | G | G |
| F25 | G | G | G | G | G | F | G | G |
| F26 | G | G | G | G | G | G | G | G |
| F27 | G | B | F | G | G | D | G | G |
| F28 | G | A | F | G | F | C | G | G |
| F29 | G | A | G | G | G | D | G | G |
| F30 | E | A | C | G | E | B | G | D |
| F31 | G | D | G | G | G | E | G | G |
| F32 | G | A | E | G | F | B | G | G |
| F33 | G | B | G | G | G | D | G | G |
| F34 | G | G | G | G | G | G | G | G |
| F35 | G | G | G | G | G | G | G | G |
| F36 | G | G | G | G | G | G | G | G |
| F37 | G | C | G | G | G | C | G | G |
| F38 | G | G | G | G | G | G | G | G |
| F39 | G | G | G | G | G | F | G | G |
| F40 | G | F | G | G | G | D | G | G |
| F41 | G | G | G | G | G | G | G | G |
| F42 | G | B | E | G | G | C | G | G |
| F43 | G | G | G | G | G | E | G | G |
| F44 | E | A | B | G | B | B | G | C |
| F45 | F | n/t | B | D | B | B | C | B |
| F46 | G | n/t | G | G | G | G | G | G |
| F47 | G | n/t | G | G | G | G | G | G |
| F48 | G | n/t | E | G | G | G | G | G |
| F49 | G | G | G | G | G | G | G | G |
| F50 | G | n/t | G | G | G | G | G | G |
| F51 | G | n/t | G | G | G | G | G | G |
| F52 | G | n/t | G | G | G | G | G | G |
| F53 | G | n/t | G | G | G | G | G | G |
| F54 | C | A | A | G | B | A | G | B |
| F55 | G | F | G | G | G | G | G | G |
| F56 | G | G | G | G | G | G | G | G |
| F57 | G | G | G | G | G | G | G | G |
| F58 | G | A | F | G | C | B | n/t | n/t |
| F59 | E | A | E | G | E | C | G | G |
| F60 | G | E | G | n/t | G | G | G | n/t |
| F61 | B | A | B | E | B | B | G | B |
| F62 | n/t | A | D | G | C | B | G | n/t |
| F63 | n/t | A | D | G | C | B | G | n/t |
| F64 | G | A | G | G | G | D | G | G |
| F65 | D | A | G | F | B | D | G | n/t |
| F66 | A | A | D | E | A | B | G | n/t |
| F67 | G | G | G | G | G | G | n/t | n/t |
| F68 | G | G | G | G | G | G | G | n/t |
| F69 | G | G | n/t | G | n/t | G | G | n/t |
| F70 | E | B | n/t | G | F | B | D | n/t |
| F71 | G | A | n/t | F | n/t | C | E | n/t |
| F72 | G | n/t | n/t | G | G | G | G | G |
| F73 | G | n/t | n/t | G | G | G | G | G |
| F74 | G | G | n/t | G | G | G | G | G |
| F75 | A | F | n/t | G | E | G | F | G |
| F76 | G | F | n/t | G | G | G | G | G |
| F77 | G | G | n/t | G | G | G | G | G |
| F78 | G | E | n/t | G | G | G | F | G |
| F79 | A | A | A | F | A | A | G | C |
| F80 | G | E | n/t | G | G | G | G | G |
| F81 | A | A | A | D | A | B | G | C |
| F82 | G | C | D | G | n/t | G | F | n/t |
| F83 | G | G | G | G | G | G | G | G |
| F84 | G | G | G | G | G | G | G | G |
| F85 | F | G | E | G | G | G | G | G |
| F86 | n/t | n/t | n/t | n/t | n/t | n/t | n/t | n/t |
| F87 | G | G | G | G | G | G | G | G |
| F88 | C | C | E | G | B | B | G | F |
| F89 | G | G | G | G | G | G | G | G |
| F90 | G | C | F | G | E | D | G | G |
| F91 | G | F | G | G | G | G | G | G |
| F92 | G | F | G | G | G | G | G | G |
| F93 | G | D | G | G | G | G | G | G |
| F94 | G | D | G | G | G | G | G | G |
| F95 | G | E | G | G | G | G | G | G |
| F96 | G | C | G | G | E | D | G | G |
| F97 | G | G | G | G | G | G | G | G |
| F98 | G | G | G | G | G | G | G | G |
| F99 | G | G | G | G | G | G | G | G |
| F100 | G | F | G | G | G | F | G | G |
| F101 | B | A | A | G | C | A | G | D |
| F102 | G | D | G | G | G | D | G | G |
| F103 | G | A | C | G | D | C | G | G |
| F104 | G | D | G | G | G | D | G | G |
| F105 | D | A | D | G | D | C | G | G |
| F106 | G | E | G | G | G | F | G | G |
| F107 | G | A | D | G | E | A | G | E |
| F108 | G | F | G | G | G | G | n/t | G |
| F109 | G | G | G | G | G | G | n/t | G |
| F110 | G | A | G | G | G | D | n/t | G |
| F111 | A | A | A | G | C | B | n/t | C |
| F112 | C | A | A | G | D | A | n/t | D |
| F113 | C | A | D | F | C | B | G | G |
| F114 | G | F | G | G | G | G | G | G |
| F115 | G | F | n/t | G | G | G | G | G |
| F116 | C | B | C | G | D | B | G | D |
| F117 | B | A | A | G | A | A | G | B |
| F118 | B | A | A | D | D | B | G | G |
| F119 | G | G | G | G | G | G | G | G |
| F120 | G | F | D | G | G | G | G | G |
| F121 | G | G | G | G | G | G | G | G |
| F122 | E | A | C | G | B | C | G | G |
| F123 | G | F | G | G | G | G | G | G |
| F124 | C | A | E | G | F | C | G | D |
| F125 | G | F | G | G | G | G | G | G |
| F126 | D | A | D | G | D | C | G | C |
| F127 | G | E | C | G | C | D | G | G |
| F128 | G | G | G | G | G | G | G | G |
| F129 | G | G | G | G | G | G | G | G |
| F130 | G | F | D | G | D | F | G | G |
| F131 | D | A | A | F | B | B | G | D |
| F132 | D | A | C | G | C | B | G | G |
| F133 | G | E | G | G | G | D | G | G |
| F134 | G | G | G | G | G | G | G | G |
| F135 | E | A | B | G | B | A | G | E |
| F136 | G | A | E | G | G | C | G | G |
| F137 | G | A | G | G | G | D | G | G |
| F138 | G | C | D | G | G | G | G | G |
| F139 | G | G | G | G | G | G | G | G |
| F140 | G | A | G | G | C | B | G | G |
| F141 | G | G | D | G | G | G | G | G |
| F142 | G | D | G | G | G | D | G | G |
| F143 | G | C | G | G | G | D | G | G |
| F144 | G | C | G | G | G | G | G | G |
| F145 | n/t | n/t | n/t | n/t | n/t | n/t | n/t | n/t |
| F146 | G | F | G | G | G | G | G | G |
| F147 | G | F | G | G | G | G | G | G |
| F148 | G | F | G | G | G | G | G | G |
| F149 | G | F | C | G | G | G | G | G |
| F150 | G | B | E | G | F | G | G | G |
| F151 | G | A | E | G | G | A | G | G |
| F152 | G | A | B | G | G | A | G | G |
| F153 | C | A | B | G | B | A | G | F |
| F154 | G | A | G | G | E | D | G | G |
| F155 | G | G | G | G | G | G | G | G |
| F156 | C | A | B | E | A | C | G | D |
| F157 | B | A | B | G | B | A | G | D |
| F158 | E | A | D | G | C | C | G | D |
| F159 | F | E | G | G | D | F | G | G |
| F160 | A | A | A | D | B | A | F | B |
| F161 | B | A | B | G | B | A | F | D |
| F162 | A | A | A | G | B | D | G | E |
| F163 | G | E | G | G | G | G | G | G |
| F164 | B | A | A | D | A | B | F | B |
| F165 | D | C | F | G | C | B | G | G |
| F166 | A | A | A | G | B | B | F | C |
| F167 | G | D | F | G | D | C | G | G |
| F168 | F | D | G | G | F | F | G | G |
| F169 | E | D | F | G | D | D | G | G |
| F170 | D | A | D | G | B | B | G | D |
| F171 | G | G | F | G | G | G | G | G |
| F172 | C | A | F | G | E | A | G | E |
| F173 | B | A | D | G | C | A | G | D |
| F174 | G | A | G | G | F | B | G | G |
| F175 | B | A | B | E | B | A | G | B |
| F176 | G | G | G | G | G | G | G | G |
| F177 | G | D | E | G | G | F | G | G |
| F178 | G | G | G | G | G | G | G | G |
| F179 | G | A | D | G | G | G | G | G |
| F180 | G | G | G | G | G | G | G | G |
| F181 | D | A | D | G | F | A | G | E |
| F182 | G | A | E | G | D | B | G | G |
| F183 | G | C | G | G | G | G | G | G |
| F184 | B | A | G | F | B | A | G | F |
| F185 | G | C | G | G | F | D | G | G |
| F186 | D | A | C | G | B | B | G | F |
| F187 | E | A | E | G | C | A | G | F |
| F188 | G | F | G | G | G | G | G | G |
| F189 | G | D | G | G | G | G | G | G |
| F190 | A | A | C | F | A | A | G | D |
| F191 | C | A | D | G | B | B | G | G |
| F192 | C | A | D | G | C | B | G | G |
| F193 | B | A | G | G | C | A | G | G |
| F194 | E | A | G | G | G | E | G | G |
| F195 | C | A | D | F | D | B | G | E |
| F196 | G | A | D | G | G | E | G | G |
| F197 | G | D | G | G | G | F | G | G |
| F198 | G | D | G | G | G | G | G | G |
| F199 | G | G | G | G | G | G | G | G |
| F200 | C | A | A | G | B | A | F | C |
| F201 | G | G | G | G | G | G | G | G |
| F202 | G | G | G | G | G | G | G | G |
| F203 | G | F | G | G | G | G | G | G |
| F204 | G | F | G | G | G | G | G | G |
| F205 | E | A | G | G | C | C | G | G |
| F206 | G | A | F | C | C | C | G | G |
| F207 | n/t | n/t | n/t | n/t | n/t | n/t | n/t | n/t |
| F208 | G | G | G | G | G | G | G | G |
| F209 | G | B | G | G | G | G | G | G |
| F210 | D | A | C | G | B | A | G | E |
| F211 | D | A | C | G | E | D | G | G |
| F212 | C | A | D | G | D | D | G | G |
| F213 | G | A | D | G | F | D | G | G |
| F214 | G | A | C | G | C | B | F | C |
| F215 | B | A | C | G | A | A | F | E |
| F216 | G | A | F | G | D | D | G | E |
| F217 | n/t | n/t | n/t | n/t | n/t | n/t | n/t | n/t |
| F218 | G | G | G | G | G | G | G | G |
| F219 | G | E | G | G | G | D | G | G |
| F220 | n/t | n/t | n/t | n/t | n/t | n/t | n/t | n/t |
| F221 | B | A | B | G | B | A | G | C |
| F222 | D | A | E | G | F | C | G | E |
| F223 | C | A | A | F | B | B | D | C |
| F224 | G | A | E | E | E | D | G | G |
| F225 | A | A | A | B | A | A | E | C |
| F226 | D | A | D | G | B | B | G | E |
| F227 | G | D | G | G | G | D | G | G |
| F228 | G | G | G | G | G | G | G | G |
| F229 | B | A | A | G | B | A | E | B |
| F230 | D | A | A | G | F | D | G | G |
| F231 | G | G | G | G | G | G | G | G |
| F232 | C | A | E | G | C | B | E | E |
| F233 | E | A | B | G | B | B | E | D |
| F234 | G | D | G | G | G | G | G | G |
| F235 | A | A | A | E | A | A | B | A |
| F236 | G | A | C | G | C | C | E | G |
| F237 | F | A | B | G | C | B | F | E |
| F238 | G | C | G | G | E | D | G | G |
| F239 | G | C | G | G | G | D | G | G |
| F240 | G | A | G | G | G | C | G | G |
| F241 | D | A | A | G | A | A | G | C |
| F242 | G | G | G | G | G | C | G | G |
| F243 | A | A | C | G | A | A | G | B |
| F244 | G | G | G | G | G | G | G | G |
| F245 | G | B | G | G | G | C | G | G |
| F246 | G | A | C | G | C | A | G | G |
| F247 | G | G | G | G | G | G | G | G |
| F248 | G | F | G | G | G | G | G | G |
| F249 | G | G | G | G | G | G | G | G |
| F250 | G | A | G | G | E | B | G | E |
| F251 | G | A | D | G | B | B | G | G |
| F252 | G | F | G | G | G | E | G | G |
| F253 | D | A | A | G | G | E | G | G |
| F254 | A | A | B | C | A | A | A | A |
| F255 | G | G | G | G | G | G | G | G |
| F256 | G | G | G | G | G | G | G | G |
| F257 | G | B | G | G | G | G | G | G |
| F258 | G | A | G | G | G | C | G | G |
| F259 | E | A | D | G | C | B | G | E |
| F260 | G | G | G | G | G | G | G | G |
| F261 | G | C | G | G | G | G | G | G |
| F262 | G | F | G | G | G | G | G | G |
| F263 | G | G | G | G | G | G | G | G |
| F264 | G | A | G | G | G | G | G | G |
| F265 | G | G | G | G | G | G | G | G |
| F266 | G | G | G | G | G | G | G | G |
| F267 | C | A | D | G | B | A | G | D |
| F268 | G | G | G | G | G | D | G | G |
| F269 | G | G | F | G | G | G | G | G |
| F270 | G | A | C | G | G | B | F | E |
| F271 | G | G | G | G | G | G | G | G |
| F272 | G | G | G | G | G | G | G | G |
| F273 | G | C | G | G | G | C | G | G |
| F274 | G | G | G | G | G | G | G | G |
| F275 | G | A | B | G | E | E | G | G |
| F276 | G | G | G | G | G | G | G | G |
| F277 | G | G | G | G | G | G | G | G |
| F278 | G | G | G | G | G | G | G | G |
| F279 | G | G | G | G | G | G | G | G |
| F280 | G | F | D | G | G | G | G | G |
| F281 | G | B | G | G | F | B | G | G |
| F282 | G | E | G | G | G | G | G | G |
| F283 | G | G | C | G | G | G | G | G |
| F284 | G | D | G | G | G | B | G | G |
| F285 | G | G | G | G | G | G | G | G |
| F286 | G | D | D | G | G | G | G | G |
| F287 | G | D | G | G | G | G | G | G |
| F288 | D | A | C | G | D | B | G | F |
| F289 | G | E | G | G | G | G | G | G |
| F290 | G | C | G | G | G | G | G | G |
| F291 | G | G | G | G | G | G | G | G |
| F292 | G | F | E | G | G | G | G | G |
| F293 | G | G | G | G | G | G | G | G |
| F294 | D | A | C | G | B | A | G | D |
| F295 | G | D | G | G | G | G | G | G |
| F296 | G | E | G | G | G | G | G | G |
| F297 | G | A | E | G | D | D | G | G |
| F298 | G | A | G | G | C | C | G | G |
| F299 | G | D | C | G | G | G | D | G |
| F300 | G | D | F | G | G | G | G | G |
| F301 | G | G | G | G | G | G | G | G |
| F302 | n/t | n/t | n/t | n/t | n/t | n/t | n/t | n/t |
| F303 | G | A | G | G | G | G | G | G |
| F304 | G | A | G | G | G | G | G | G |
| F305 | G | A | G | G | G | C | G | G |
| F306 | G | A | G | G | G | D | G | G |
| F307 | G | A | G | G | G | B | G | G |
| F308 | G | G | G | G | G | G | G | G |
| F309 | G | G | G | G | G | G | G | G |
| F310 | D | A | E | G | B | A | G | D |
| F311 | G | A | G | G | G | C | G | G |
| F312 | C | A | G | G | D | B | G | F |
| F313 | D | A | G | G | D | B | G | G |
| F314 | G | A | G | G | G | C | G | G |
| F315 | G | A | G | G | D | B | G | D |
| F316 | G | D | G | G | G | G | G | G |
| F317 | G | D | G | G | G | G | G | G |
| F318 | B | A | G | G | C | B | G | G |
| F319 | G | F | G | G | G | G | G | G |
| F320 | G | D | G | G | G | G | G | G |
| F321 | F | B | G | G | C | A | G | F |
| F322 | G | A | G | G | G | G | G | G |
| F323 | G | A | F | G | F | B | G | G |
| F324 | G | A | B | G | D | C | G | E |
| F325 | G | A | G | G | F | B | G | G |
| F326 | C | G | G | G | F | A | G | G |
| F327 | G | C | G | G | F | G | G | G |
| F328 | G | B | G | G | F | C | G | G |
| F329 | D | A | D | G | B | C | G | E |
| F330 | G | G | G | G | G | G | G | G |
| F331 | G | A | D | G | G | E | G | G |
| F332 | G | G | F | G | G | G | G | G |
| F333 | F | A | F | G | F | B | G | E |
| F334 | C | A | D | E | C | A | G | C |
| F335 | G | G | G | G | G | G | G | G |
| F336 | G | G | G | G | G | C | G | F |
| F337 | D | A | B | G | B | C | G | F |
| F338 | G | A | E | G | C | B | G | F |
| F339 | G | A | B | G | G | D | G | G |
| F340 | G | G | G | G | G | G | G | G |
| F341 | G | A | D | G | B | A | F | E |
| F342 | G | B | B | G | F | F | G | F |
| F343 | B | A | B | F | B | A | G | D |
| F344 | G | A | E | G | D | C | G | E |
| F345 | G | G | G | G | G | G | G | G |
| F346 | G | F | G | G | G | G | G | G |
| F347 | B | A | B | F | B | B | G | C |
| F348 | G | G | G | G | G | G | G | G |
| F349 | G | A | B | G | C | B | G | B |
| F350 | G | B | B | G | G | G | G | G |
| F351 | F | A | F | G | C | C | G | G |
| F352 | A | A | B | B | A | A | D | A |
| F353 | G | A | G | G | G | E | G | G |
| F354 | C | A | D | G | C | B | G | D |
| F355 | G | G | G | G | G | G | G | G |
| F356 | G | A | G | G | G | F | G | G |
| F357 | G | G | G | G | G | G | G | G |
| F358 | G | G | G | G | G | G | G | G |
| F359 | A | A | B | G | A | B | G | D |
| F360 | G | A | E | G | G | G | G | G |
| F361 | G | C | G | G | G | G | G | G |
| F362 | G | A | F | G | D | B | G | G |
| F363 | A | A | C | F | B | A | F | C |
| F364 | G | B | G | G | D | B | G | G |
| F365 | G | G | G | G | G | G | G | G |
| F366 | C | A | G | G | C | C | G | G |
| F367 | G | A | G | G | G | G | G | G |
| F368 | G | A | G | G | E | C | G | G |
| F369 | E | A | F | G | D | B | G | D |
| F370 | G | A | G | G | D | B | G | G |
| F371 | G | A | G | G | G | C | G | G |
| F372 | G | A | G | G | G | F | G | G |
| F373 | C | A | C | G | C | A | F | F |
| F374 | C | A | C | E | B | A | G | D |
| F375 | B | A | D | G | B | B | G | C |
| F376 | G | A | E | G | D | B | G | G |
| F377 | D | A | D | G | C | B | G | G |
| F378 | G | A | E | G | D | C | G | G |
| F379 | C | A | B | G | C | C | G | D |
| F380 | C | A | C | G | A | A | G | D |
| F381 | B | A | A | G | B | B | G | D |

ALOMY: blackgrass (Alopecurus myosuroides)
AMARE: redroot pigweed (Amaranthus retroflexus)
AVEFA: wild oat (Avena fatua)
CHEAL: lambsquarters (Chenopodium album)
CIRAR: canada thistle (Cirsium arvense)
CYPES: yellow nutsedge (Cyperus esculentus)
DIGSA: crabgrass (Digitaria sanguinalis)
ECHCG: barnyardgrass (Echinochloa crus-galli)
IPOHE: ivy-leaf morning glory (Ipomoea hederacea)
KCHSC: kochia (Bassia scoparia)
ORYSA: rice (Oryza sativa)
SETFA: giant foxtail (Setaria faberi)
SORVU: johnsongrass (Sorghum vulgare)
STEME: common chickweed (Stellaria media)
TRZAS: wheat, spring (Triticum aestivum)
g ai/ha: grams active ingredient per hectare
n/t: not tested

1. A compound of Formula (I): 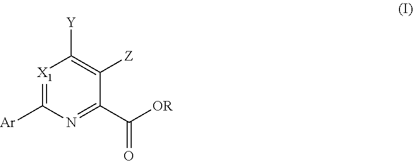
wherein
Ar is 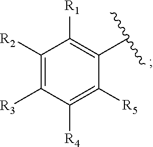
X₁ is N or CR₆;
R is hydrogen, substituted or unsubstituted alkyl, substituted or unsubstituted phenyl, substituted or unsubstituted benzyl, substituted or unsubstituted pyridinylmethyl, substituted or unsubstituted alkenyl, or substituted or unsubstituted alkynyl;
R₁, R₂, R₃, R₄, and R₅ are independently selected from the group consisting of hydrogen, halogen, hydroxy, substituted or unsubstituted alkyl, substituted or unsubstituted haloalkyl, substituted or unsubstituted alkenyl, substituted or unsubstituted haloalkenyl, substituted or unsubstituted alkynyl, substituted or unsubstituted cycloalkyl, substituted or unsubstituted halocycloalkyl, substituted or unsubstituted aryl, substituted or unsubstituted aryloxy, substituted or unsubstituted alkoxy, substituted or unsubstituted haloalkoxy, substituted or unsubstituted alkylsulfanyl, substituted or unsubstituted haloalkylsulfanyl, substituted or unsubstituted alkylsulfinyl, substituted or unsubstituted haloalkylsulfinyl, substituted or unsubstituted alkylsulfonyl, substituted or unsubstituted haloalkylsulfonyl, substituted or unsubstituted alkylsulfonyl(oxy), substituted or unsubstituted haloalkylsulfonyl(oxy), amino, alkylamino, dialkylamino, amido, formyl, 2,2-dimethylhydrazono, methoxyimino, hydroxyimino, alkylcarbonyl, alkoxycarbonyl, nitro, and cyano, or R₁ and R₂, R₂ and R₃, R₃ and R₄ or R₄ and R₅ together can form a substituted or unsubstituted 5- or 6-membered aliphatic or aromatic ring, containing 0 to 3 heteroatoms selected from the group consisting of O, N, and S;
R₆ is hydrogen, halogen, substituted or unsubstituted alkyl, substituted or unsubstituted haloalkyl, substituted or unsubstituted alkynyl, substituted or unsubstituted alkoxy, substituted or unsubstituted alkylsulfanyl, hydroxy, amino, cyano, or acylamino;
Y is selected from the group consisting of hydrogen, halogen, substituted or unsubstituted alkyl, substituted or unsubstituted haloalkyl, substituted or unsubstituted alkenyl, substituted or unsubstituted haloalkenyl, substituted or unsubstituted alkynyl, substituted or unsubstituted alkoxy, substituted or unsubstituted haloalkoxy, substituted or unsubstituted alkylsulfanyl, substituted or unsubstituted alkylsulfinyl, substituted or unsubstituted alkylsulfonyl, nitro, and cyano;
Z is selected from the group consisting of hydrogen, halogen, alkyl, haloalkyl, alkynyl, alkoxy, haloalkoxy, alkylsulfanyl, amino, nitro, and cyano;
with the proviso that when X₁=N, Y is not alkoxy;
with the proviso that the compound of Formula (I) is not

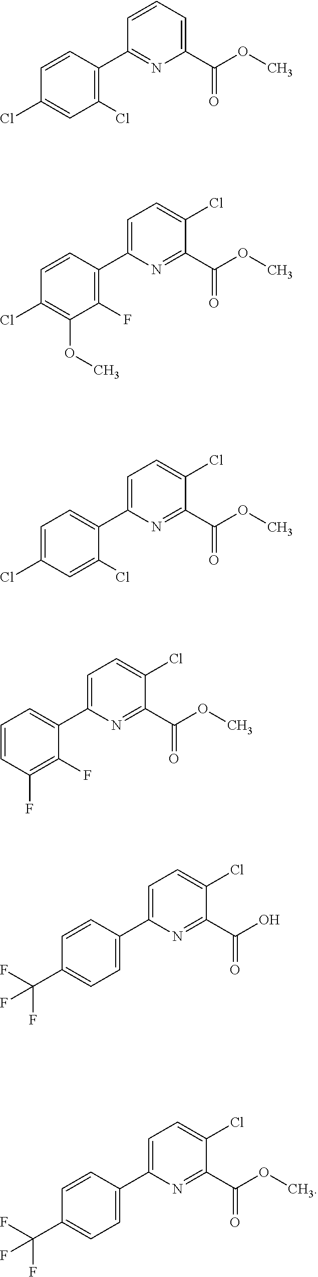 [SMILES: COC(=O)c1cccc(-c2ccc(Cl)cc2Cl)n1 ; COC(=O)c1nc(-c2ccc(Cl)c(OC)c2F)ccc1Cl ; COC(=O)c1nc(-c2ccc(Cl)cc2Cl)ccc1Cl ; COC(=O)c1nc(-c2cccc(F)c2F)ccc1Cl ; O=C(O)c1nc(-c2ccc(C(F)(F)F)cc2)ccc1Cl ; COC(=O)c1nc(-c2ccc(C(F)(F)F)cc2)ccc1Cl]

2. A compound of Formula (I): 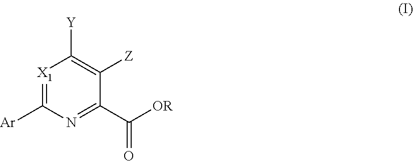
wherein
Ar is 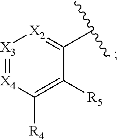
X₁ is N or CR₆;
X₂ is N or CR₁;
X₃ is N or CR₂;
X₄ is N or CR₃;
R is hydrogen, substituted or unsubstituted alkyl, phenyl, substituted or unsubstituted benzyl, substituted or unsubstituted pyridinylmethyl, substituted or unsubstituted alkenyl, or substituted or unsubstituted alkynyl;
R₁, R₂, R₃, R₄, and R₅ are independently selected from the group consisting of hydrogen, halogen, hydroxy, substituted or unsubstituted alkyl, substituted or unsubstituted haloalkyl, substituted or unsubstituted alkenyl, substituted or unsubstituted haloalkenyl, substituted or unsubstituted alkynyl, substituted or unsubstituted cycloalkyl, substituted or unsubstituted halocycloalkyl, substituted or unsubstituted aryl, substituted or unsubstituted aryloxy, substituted or unsubstituted alkoxy, substituted or unsubstituted haloalkoxy, substituted or unsubstituted alkylsulfanyl, substituted or unsubstituted haloalkylsulfanyl, substituted or unsubstituted alkylsulfinyl, substituted or unsubstituted haloalkylsulfinyl, substituted or unsubstituted alkylsulfonyl, substituted or unsubstituted haloalkylsulfonyl, substituted or unsubstituted alkylsulfonyl(oxy), substituted or unsubstituted haloalkylsulfonyl(oxy), amino, alkylamino, dialkylamino, amido, formyl, 2,2-dimethylhydrazono, methoxyimino, hydroxyimino, alkylcarbonyl, alkoxycarbonyl, nitro, and cyano, or R₁ and R₂, R₂ and R₃, R₃ and R₄ or R₄ and R₅ together can form a substituted or unsubstituted 5- or 6-membered aliphatic or aromatic ring, containing 0 to 3 heteroatoms selected from the group consisting of O, N, and S;
R₆ is hydrogen, halogen, substituted or unsubstituted alkyl, substituted or unsubstituted haloalkyl, substituted or unsubstituted alkynyl, substituted or unsubstituted alkoxy, substituted or unsubstituted alkylsulfanyl, hydroxy, amino, cyano, or acylamino;
Y is selected from the group consisting of hydrogen, halogen, substituted or unsubstituted alkyl, substituted or unsubstituted haloalkyl, substituted or unsubstituted alkenyl, substituted or unsubstituted haloalkenyl, substituted or unsubstituted alkynyl, substituted or unsubstituted alkoxy, substituted or unsubstituted haloalkoxy, substituted or unsubstituted alkylsulfanyl, substituted or unsubstituted alkylsulfinyl, substituted or unsubstituted alkylsulfonyl, nitro, and cyano; and
Z is selected from the group consisting of hydrogen, halogen, alkyl, haloalkyl, alkynyl, alkoxy, haloalkoxy, alkylsulfanyl, amino, nitro, and cyano;
with the proviso that when X₁ is N, then Y is not alkoxy.

3. The compound of claim 3, wherein R is selected from the group consisting of hydrogen, C₁-C₈ alkyl, substituted C₁-C₈ alkyl, C₂-C₈ alkenyl, C₂-C₈ alkynyl, benzyl, substituted benzyl, and pyridinylmethyl.

4. The compound of claim 3, wherein R₁ or R₅ is selected from the group consisting of hydrogen, halogen, cyano, amino, amido, nitro, C₁-C₈ alkyl, C₁-C₈ haloalkyl, C₂-C₈ alkenyl, C₂-C₈ alkynyl, C₁-C₈ alkoxy, C₁-C₈ alkylsulfanyl, C₁-C₈ alkylsulfinyl, C₁-C₈ alkylsulfonyl, formyl,

5. The compound of claim 3, wherein R₂ or R₄ is selected from the group consisting of hydrogen, halogen, nitro C₁-C₈ alkyl, C₁-C₈ haloalkyl, C₁-C₈ alkoxy, C₁-C₈ alkylsulfanyl, and C₁-C₈ alkylsulfinyl.

6. The compound of claim 3, wherein R₃ is selected from the group consisting of halogen, substituted or unsubstituted haloalkyl, substituted or unsubstituted cycloalkyl, substituted or unsubstitutedhalocycloalkyl, substituted or unsubstitutedhaloalkysulfanyl, substituted or unsubstitutedalkylsulfonyl(oxy), and cyano.

7. The compound of claim 3, wherein R₁ and R₂, R₂ and R₃, R₃ and R₄ or R₄ and R₅ together can form a substituted or unsubstituted 5- or 6-membered aliphatic or aromatic ring, containing 0 to 3 heteroatoms selected from the group consisting of O, N, or S which is selected from the group consisting of

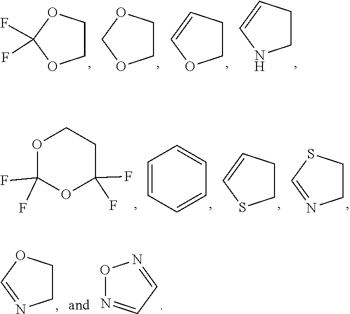

8. The compound of claim 2, wherein R is selected from the group consisting of hydrogen, C₁-C₈ alkyl, substituted C₁-C₈ alkyl, C₂-C₈ alkenyl, C₂-C₈ alkynyl, benzyl, substituted benzyl, and pyridinylmethyl.

9. The compound of claim 2, wherein R₁, R₂, R₃, R₄, and R₅ are independently selected from the group consisting of hydrogen, halogen, cyano, amino, amido, nitro, C₁-C₈ alkyl, C₁-C₈ haloalkyl, C₂-C₈ alkenyl, C₂-C₈ alkynyl, C₁-C₈ alkoxy, C₁-C₈ alkylsulfanyl, C₁-C₈ alkylsulfinyl, C₁-C₈ alkylsulfonyl, and formyl.

10. The compound of claim 3, wherein R₂ or R₄ is selected from the group consisting of hydrogen, halogen, nitro C₁-C₈ alkyl, C₁-C₈ haloalkyl, C₁-C₈ alkoxy, C₁-C₈ alkylsulfanyl, and C₁-C₈ alkylsulfinyl.

11. The compound of claim 3, wherein R is selected from the group consisting of hydrogen, methyl, cyanomethyl, 2-methylallyl, propargyl, benzyl, substituted benzyl, and pyridinylmethyl;
R₁ or R₅ is selected from the group consisting of hydrogen, halogen, cyano, (C₁-C₄)alkyl, and (C₁-C₄)alkoxy;
R₂ or R₄ is selected from the group consisting of hydrogen, halogen, (C₂-C₄)alkynyl, and (C₁-C₄)haloalkyl;
R₃ is selected from the group consisting of halogen, substituted (C₃-C₆)cycloalkyl, (C₁-C₄)haloalkylsulfanyl, (C₁-C₄)haloalkyl, and (C₁-C₄)haloalkoxy or R₁ and R₂, R₂ and R₃, R₃ and R₄ or R₄ and R₅ together can form a substituted or unsubstituted 5- or 6-membered aliphatic or aromatic ring, containing 0 to 3 heteroatoms selected from the group consisting of O, N, and S;
R₆ is selected from the group consisting of hydrogen, halogen, and hydroxy;
Y is H; and
Z is a halogen.
The compound of claim 2, wherein R is selected from the group consisting of hydrogen, methyl, cyanomethyl, 2-methylallyl, propargyl, benzyl, substituted benzyl, and pyridinylmethyl;
R₁, R₂, R₃, R₄, and R₅ are independently selected from the group consisting of hydrogen, halogen, (C₁-C₄)haloalkyl, and (C₁-C₄)alkoxy;
R₆ is selected from the group consisting of hydrogen, halogen, and hydroxy;
Y is H; and
Z is a halogen.

12. A compound selected from the group consisting of

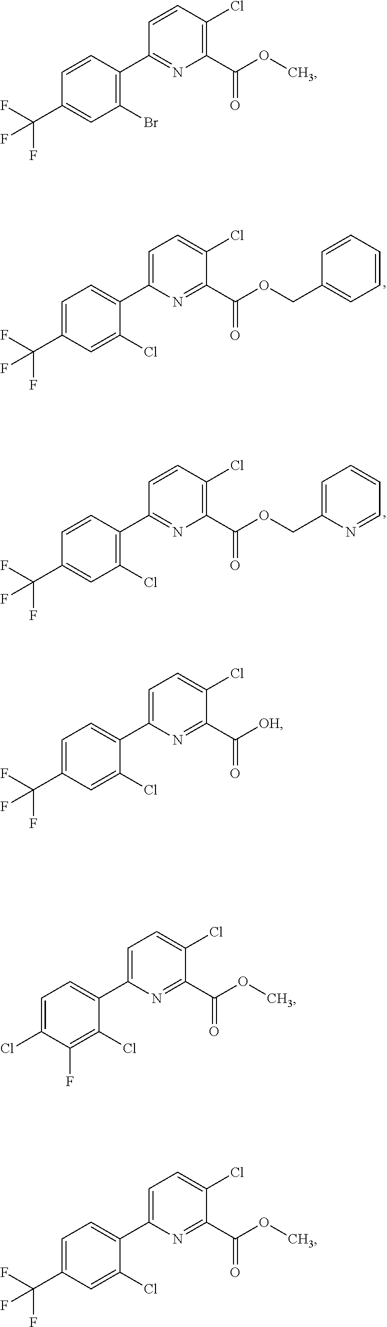 [SMILES: COC(=O)c1nc(-c2ccc(C(F)(F)F)cc2Br)ccc1Cl ; O=C(OCc1ccccc1)c1nc(-c2ccc(C(F)(F)F)cc2Cl)ccc1Cl ; O=C(OCc1ccccn1)c1nc(-c2ccc(C(F)(F)F)cc2Cl)ccc1Cl ; O=C(O)c1nc(-c2ccc(C(F)(F)F)cc2Cl)ccc1Cl ; COC(=O)c1nc(-c2ccc(Cl)c(F)c2Cl)ccc1Cl ; COC(=O)c1nc(-c2ccc(C(F)(F)F)cc2Cl)ccc1Cl]

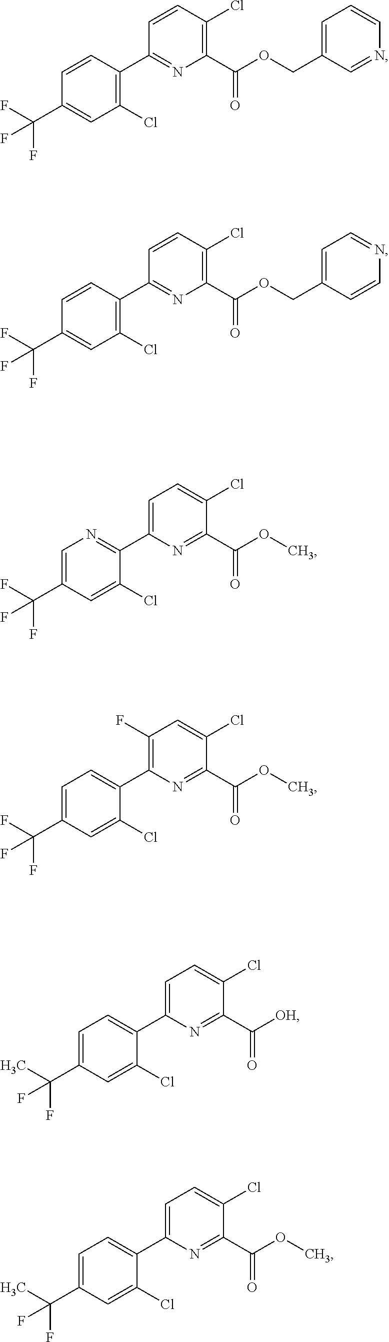 [SMILES: O=C(OCc1cccnc1)c1nc(-c2ccc(C(F)(F)F)cc2Cl)ccc1Cl ; O=C(OCc1ccncc1)c1nc(-c2ccc(C(F)(F)F)cc2Cl)ccc1Cl ; COC(=O)c1nc(-c2ncc(C(F)(F)F)cc2Cl)ccc1Cl ; COC(=O)c1nc(-c2ccc(C(F)(F)F)cc2Cl)c(F)cc1Cl ; CC(F)(F)c1ccc(-c2ccc(Cl)c(C(=O)O)n2)c(Cl)c1 ; COC(=O)c1nc(-c2ccc(C(C)(F)F)cc2Cl)ccc1Cl]

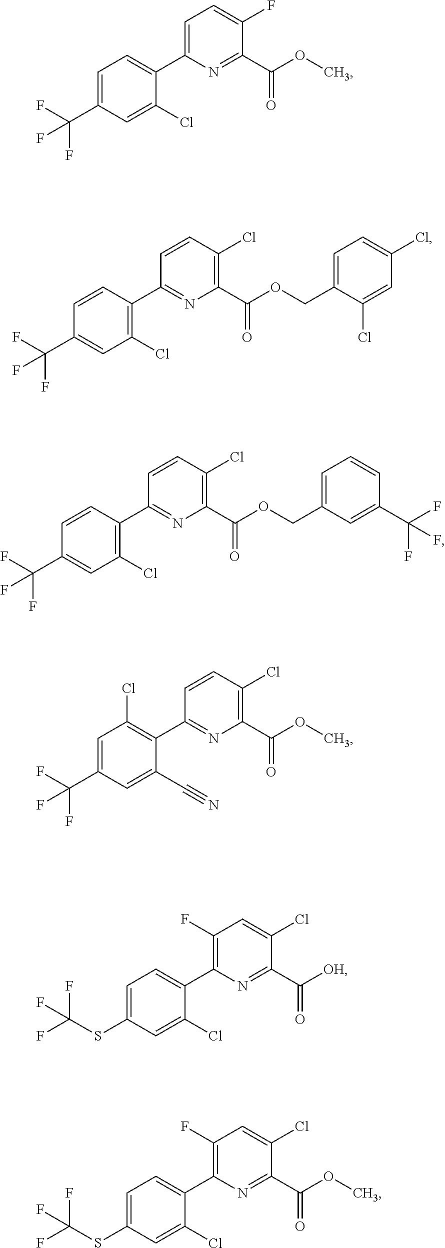 [SMILES: COC(=O)c1nc(-c2ccc(C(F)(F)F)cc2Cl)ccc1F ; O=C(OCc1ccc(Cl)cc1Cl)c1nc(-c2ccc(C(F)(F)F)cc2Cl)ccc1Cl ; O=C(OCc1cccc(C(F)(F)F)c1)c1nc(-c2ccc(C(F)(F)F)cc2Cl)ccc1Cl ; COC(=O)c1nc(-c2c(Cl)cc(C(F)(F)F)cc2C#N)ccc1Cl ; O=C(O)c1nc(-c2ccc(SC(F)(F)F)cc2Cl)c(F)cc1Cl ; COC(=O)c1nc(-c2ccc(SC(F)(F)F)cc2Cl)c(F)cc1Cl]

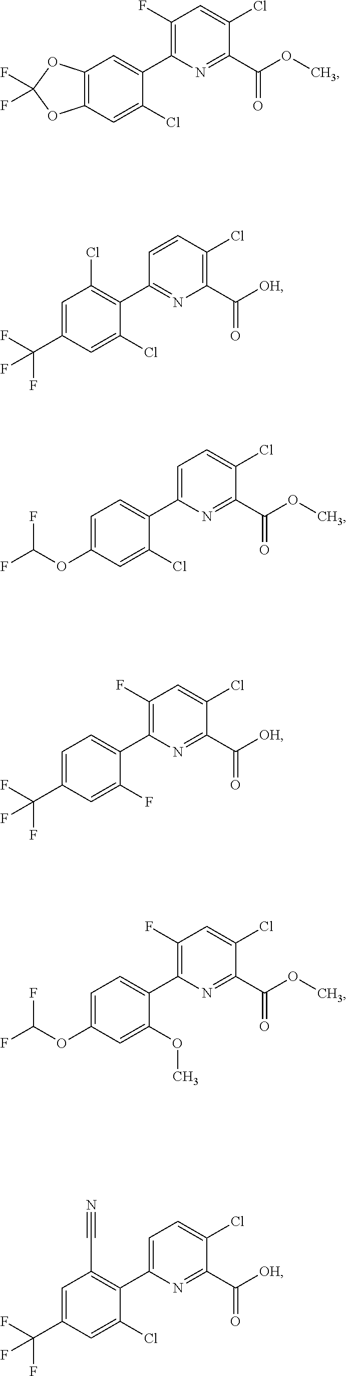 [SMILES: COC(=O)c1nc(-c2cc3c(cc2Cl)OC(F)(F)O3)c(F)cc1Cl ; O=C(O)c1nc(-c2c(Cl)cc(C(F)(F)F)cc2Cl)ccc1Cl ; COC(=O)c1nc(-c2ccc(OC(F)F)cc2Cl)ccc1Cl ; O=C(O)c1nc(-c2ccc(C(F)(F)F)cc2F)c(F)cc1Cl ; COC(=O)c1nc(-c2ccc(OC(F)F)cc2OC)c(F)cc1Cl ; N#Cc1cc(C(F)(F)F)cc(Cl)c1-c1ccc(Cl)c(C(=O)O)n1]

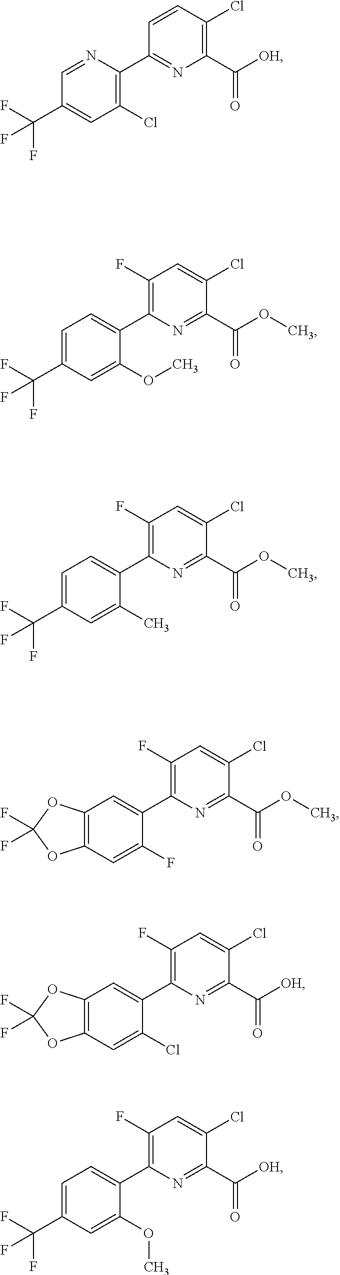 [SMILES: O=C(O)c1nc(-c2ncc(C(F)(F)F)cc2Cl)ccc1Cl ; COC(=O)c1nc(-c2ccc(C(F)(F)F)cc2OC)c(F)cc1Cl ; COC(=O)c1nc(-c2ccc(C(F)(F)F)cc2C)c(F)cc1Cl ; COC(=O)c1nc(-c2cc3c(cc2F)OC(F)(F)O3)c(F)cc1Cl ; O=C(O)c1nc(-c2cc3c(cc2Cl)OC(F)(F)O3)c(F)cc1Cl ; COc1cc(C(F)(F)F)ccc1-c1nc(C(=O)O)c(Cl)cc1F]

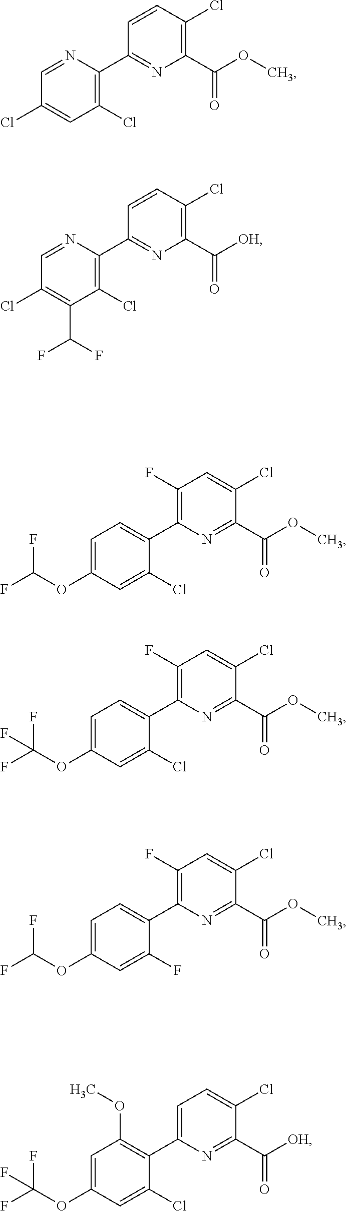 [SMILES: COC(=O)c1nc(-c2ncc(Cl)cc2Cl)ccc1Cl ; O=C(O)c1nc(-c2ncc(Cl)c(C(F)F)c2Cl)ccc1Cl ; COC(=O)c1nc(-c2ccc(OC(F)F)cc2Cl)c(F)cc1Cl ; COC(=O)c1nc(-c2ccc(OC(F)(F)F)cc2Cl)c(F)cc1Cl ; COC(=O)c1nc(-c2ccc(OC(F)F)cc2F)c(F)cc1Cl ; COc1cc(OC(F)(F)F)cc(Cl)c1-c1ccc(Cl)c(C(=O)O)n1]

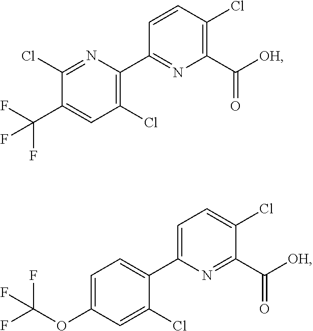 [SMILES: O=C(O)c1nc(-c2nc(Cl)c(C(F)(F)F)cc2Cl)ccc1Cl ; O=C(O)c1nc(-c2ccc(OC(F)(F)F)cc2Cl)ccc1Cl]

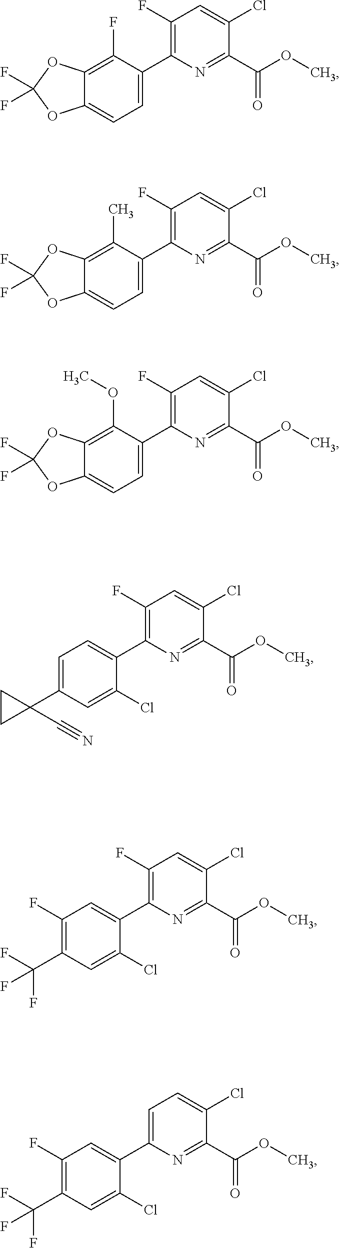 [SMILES: COC(=O)c1nc(-c2ccc3c(c2F)OC(F)(F)O3)c(F)cc1Cl ; COC(=O)c1nc(-c2ccc3c(c2C)OC(F)(F)O3)c(F)cc1Cl ; COC(=O)c1nc(-c2ccc3c(c2OC)OC(F)(F)O3)c(F)cc1Cl ; COC(=O)c1nc(-c2ccc(C3(C#N)CC3)cc2Cl)c(F)cc1Cl ; COC(=O)c1nc(-c2cc(F)c(C(F)(F)F)cc2Cl)c(F)cc1Cl ; COC(=O)c1nc(-c2cc(F)c(C(F)(F)F)cc2Cl)ccc1Cl]

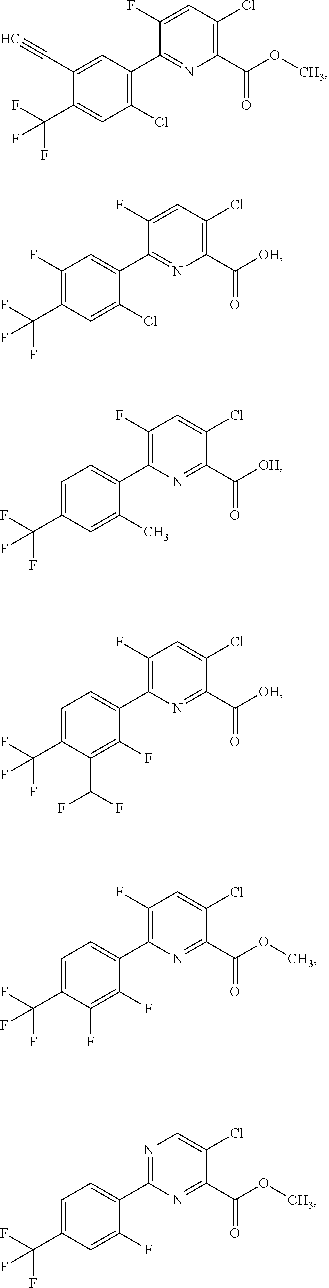 [SMILES: C#Cc1cc(-c2nc(C(=O)OC)c(Cl)cc2F)c(Cl)cc1C(F)(F)F ; O=C(O)c1nc(-c2cc(F)c(C(F)(F)F)cc2Cl)c(F)cc1Cl ; Cc1cc(C(F)(F)F)ccc1-c1nc(C(=O)O)c(Cl)cc1F ; O=C(O)c1nc(-c2ccc(C(F)(F)F)c(C(F)F)c2F)c(F)cc1Cl ; COC(=O)c1nc(-c2ccc(C(F)(F)F)c(F)c2F)c(F)cc1Cl ; COC(=O)c1nc(-c2ccc(C(F)(F)F)cc2F)ncc1Cl]

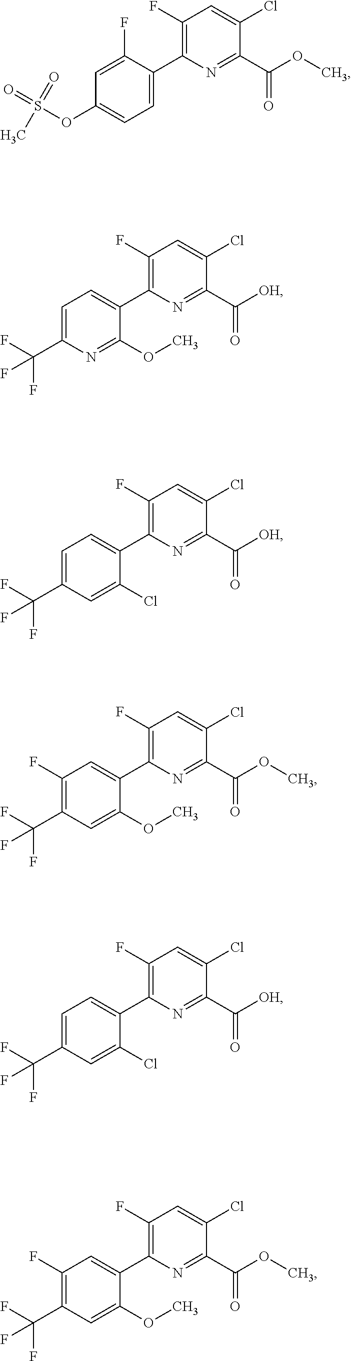 [SMILES: COC(=O)c1nc(-c2ccc(OS(C)(=O)=O)cc2F)c(F)cc1Cl ; COc1nc(C(F)(F)F)ccc1-c1nc(C(=O)O)c(Cl)cc1F ; O=C(O)c1nc(-c2ccc(C(F)(F)F)cc2Cl)c(F)cc1Cl ; COC(=O)c1nc(-c2cc(F)c(C(F)(F)F)cc2OC)c(F)cc1Cl ; O=C(O)c1nc(-c2ccc(C(F)(F)F)cc2Cl)c(F)cc1Cl ; COC(=O)c1nc(-c2cc(F)c(C(F)(F)F)cc2OC)c(F)cc1Cl]

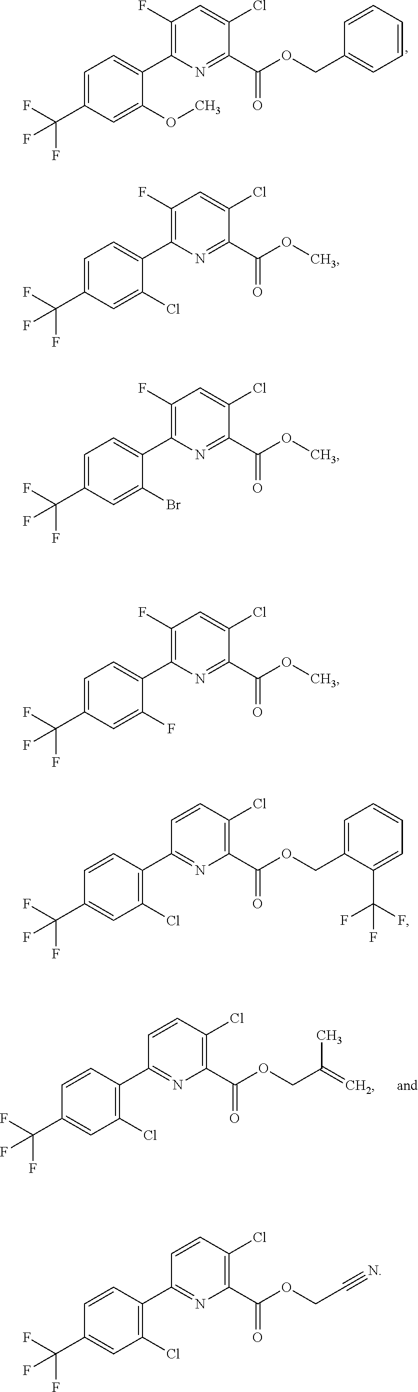

13. A composition comprising the compound of claim 1, further comprising an agriculturally acceptable adjuvant or carrier.

14. A composition comprising the compound of claim 1, further comprising an additional herbicidal compound.

15. A composition comprising the compound of claim 1, further comprising a safener.

16. The composition or herbicidal composition of claim 1, wherein R is H, alkyl, substituted alkyl, benzyl, and alkynyl.

17. The composition or herbicidal composition of claim 1, wherein R₁ is cyano, C₁-C₄ alkyl, C₂-C₄ alkynyl, or substituted phenyl.

18. The composition or herbicidal composition of claim 1, wherein R₁ is methyl, ethynyl, 4-chlorophenyl, or 2,4-dichlorophenyl.

19. The composition or herbicidal composition of claim 1, wherein R₄ is cyano, trifluoromethyl, or fluoro.

20. A method for controlling undesirable vegetation, which comprises (a) contacting the undesirable vegetation or area adjacent to the undesirable vegetation, or (b) pre-emergently contacting soil or water, with a compound of claim 1.

21. The method of claim 20, wherein the herbicidal composition further comprises an additional herbicidal compound.

22. The method of claim 20, wherein the herbicidal composition further comprises a safener.

23. The method of claim 21, wherein R is H, alkyl, substituted alkyl, benzyl, and alkynyl.

24. The method of claim 21, wherein R₁ is cyano, C₁-C₄ alkyl, C₂-C₄ alkynyl, or substituted phenyl.

25. The method of claim 21, wherein R₁ is Me, ethynyl, 4-chlorophenyl, or 2,4-dichlorophenyl.

26. The method of claim 21, wherein R₄ is cyano, trifluoromethyl, or fluoro.

27. The method of claim 11, wherein the compound or composition is applied pre-emergent.

28. The method of claim 11, wherein the compound or composition is applied post-emergent.
